(19) United States
(12) Patent Application Publication
(10) Pub. No.: US 2026/0234543 A1
(43) Pub. Date: Aug. 13, 2026

(54) MICROORGANISMS AND METHODS FOR REDUCING BY-PRODUCTS

(71) Applicant: Genomatica, Inc., San Diego, CA (US)

(72) Inventors: Harish NAGARAJAN, San Diego, CA (US); Gionata SCALCINATI, Carlsbad, CA (US); Michael Angus NOBLE, La Mesa, CA (US)

(21) Appl. No.: 19/154,193

(22) PCT Filed: Feb. 7, 2024
(86) PCT No.: PCT/US2024/014854

§ 371 Date: Aug. 6, 2025

Related U.S. Application Data
(60) Provisional application No. 63/483,736, filed on Feb. 7, 2023.

(51) Int. Cl. C12N 1/20; C12N 9/00; C12N 9/02; C12P 7/04; C12P 7/18; C12P 7/62; C12R 1/19

(52) U.S. Cl. CPC C12N 1/20; C12N 9/0008; C12N 9/93; C12P 7/04; C12P 7/18; C12P 7/62; C12R 2001/19; C12Y 102/0103; C12Y 602/01001; C12Y 602/01002

(57) ABSTRACT

The present disclosure provides nonrnaturally occurring microbial organisms having eliminate, substantially eliminate, or reduced production of unwanted by-products (e.g, 3HB, acetate, and others) and/or to enhance or increase carbon flux through 3 HB—CoA, and/or 3HBAId, and/or the CoA thioesters or acyl-CoAs of unwanted by-products, which can increase production of acetyl-CoA derived compounds (e.g, 1,3-BDO, MMA, and (3R)-hydroxybutyl (3R)-hydroxybutyrate, or any other acetyl-CoA derived compounds), and products made from any of these compounds. Also provided are one or more exogenous nucleic acids encoding enzymes that can eliminate, substantially eliminate, or reduce production of unwanted by-products (e.g, 3HB, acetate, and others) and/or that can enhance or increase carbon flux through 3HB—CoA, 3HBAId, and/or the CoA thioesters/acyl-CoAs of unwanted by-products.

### CROSS-REFERENCE

This application claims benefit of U.S. Provisional Application No. 63/483,736, filed on Feb. 7, 2023, which is incorporated herein by reference in its entirety.

### INCORPORATION BY REFERENCE OF SEQUENCE LISTING

The instant application contains a Sequence Listing, which has been submitted via Patent Center. The Sequence Listing titled 199683-805002_PCT_SL.xml, which was created on Feb. 6, 2024, and is 6,313 bytes in size, is hereby incorporated by reference in its entirety.

### FIELD

The present invention relates generally to organisms engineered to produce desired products, engineered enzymes, or polypeptides or proteins with a desired enzymatic activity, that facilitate or allow for the production of desired products, and more specifically, to non-naturally occurring organisms that can reduce and/or recycle by-products, such as 3-hydroxybutyrate (3-HB or 3HB), thereby increasing the yield, titer, and/or productivity of one or more products or product intermediates, including, but not limited to, 1,3-butanediol (1,3-BDO, 1,3BDO, or 13BDO; also referred to as butylene glycol, 1,3-butylene glycol, BG, 1,3-BG, 1,3BG, or 13BG), methacrylic acid (MAA), methacrylic acid esters, such as methyl methacrylate (MMA) and ethyl methacrylate (EMA), (3R)-hydroxybutyl (3R)-hydroxybutyrate, butyrate, butanol, propane, formate, 3-hydroxybutyryl-coenzyme A (3HB—CoA), and related products derived therefrom.

The present invention also relates to non-naturally occurring (i.e., engineered or recombinant) microbial organisms (or microorganisms), modified biosynthetic or enzymatic pathways, including by-product reducing and/or by-product recycling pathways and loops, and methods, for the reduction and/or recycling of carboxylic acid or carboxylate by-products, particularly short-chain (e.g., C2-C4, C2-C6, or C2-C8) carboxylic acids or carboxylate by-products of acetyl-CoA derived product pathways. Such by-products, include, but are not limited to, for example, acetate, acetoacetate, succinate, crotonate, butyrate, isobutyrate, 3-hydroxybutryate (3-HB), (S)-3HB, (R)-3HB, malonate, hexanoate, and 6-aminocaproic acid/6-aminocaproate (6ACA). The reduction of (i.e., decreasing) these by-products, e.g., by recycling or reintroducing them back into the acetyl-CoA derived product pathway, can increase the carbon flux through the acetyl-CoA derived product pathway precursors and/or intermediates, and/or increases the yield, titer, and/or productivity of the acetyl-CoA derived products. The acetyl-CoA derived products include, but are not limited to, for example, 1,3-butanediol (1,3-BDO), (R)-1,3-BDO, (S)-1,3-BDO, methacrylic acid (MAA), methacrylic acid esters (e.g., methyl methacrylate (MMA) and ethyl methacrylate (EMA)), (3R)-hydroxybutyl (3R)-hydroxybutyrate, butyrate, butanol, propane, formate, hexamethylenediamine (HMDA or HMD), caprolactam (CPL), adipate (or adipic acid), 6-aminocaproic acid (6-ACA), and/or 1,6-hexanediol (HDO), and related products derived therefrom.

### BACKGROUND

Microbial organisms can be used for the production of chemical compounds and intermediates thereof that are derived from acetyl-CoA and other precursors and/or starting materials, such as, for example, alpha-ketoglutarate or 4-hydroxybutyryl-CoA. These chemical compounds and intermediates thereof include, for example, 1,3-butanediol (1,3-BDO, 1,3BDO, or 13BDO; also referred to as butylene glycol, 1,3-butylene glycol, BG, 1,3-BG, 1,3BG, or 13BG), methacrylic acid (MAA), esters of MAA, such as methyl methacrylate (MMA) and ethyl methacrylate (EMA), ketone esters (e.g., (3R)-hydroxybutyl (3R)-hydroxybutyrate), butyrate, butanol, propane, formate, and 3-hydroxybutyryl-coenzyme A (3HB—CoA), among others. The titer, rate, and/or yield of such production can be limited by the generation of unwanted by-products. In particular, the generation of unwanted by-products, such as 3-hydroxybutyrate (3-HB or 3HB) can limit the amount of the intermediates, such as 3HB—CoA, that are available for the generation of the desired product(s). Accordingly, decreased production of unwanted by-products, such as 3HB, for example, by recycling and/or bringing the by-product back into the product biosynthetic pathway, increases the titer, rate, productivity, and/or yield of products, such as, for example, 1,3-BDO, MAA, methacrylic acid esters (e.g., MMA and EMA), butyrate, butanol, propane, formate, and (3R)-hydroxybutyl (3R)-hydroxybutyrate, whose biosynthetic pathways include 3HB-derived intermediates.

For example, the generation of 3HB by-product reduces or limits the amounts of the intermediates 3HB—CoA and/or 3-hydroxybutyraldehyde (3HBAld; which can also be referred to herein as 3HB-Ald, 3-HBAld, or 3-HB-Ald) that are available for the generation of the desired acetyl-CoA derived product(s), such as, for example, 1,3-BDO, R-1,3-BDO, S-1,3-BDO, MAA, methacrylic acid esters (e.g., MMA and EMA), butyrate, butanol, propane, formate, and (3R)-hydroxybutyl (3R)-hydroxybutyrate, whose biosynthetic pathways include 3HB-derived intermediates such as 3HB—CoA and/or 3HBAld. Further, the generation of unwanted by-products of acetyl-CoA derived product pathways, such as, for example, one or more of acetate, acetoacetate, succinate, crotonate, butyrate, isobutyrate, 3-hydroxybutryate (3-HB), (S)-3HB, (R)-3HB, malonate, hexanoate, and/or 6-aminocaproic acid/6-aminocaproate (6ACA), limits the amounts of, and the carbon flux through, precursors and/or intermediates of acetyl-CoA derived product pathways, which thereby reduces the production (e.g., titer, yield, and/or productivity (rate)) of acetyl-CoA derived products. For example, the generation of acetate by-product reduces the amount of acetyl-CoA and/or the carbon flux through acetyl-CoA, which reduces the production (e.g., titer, yield, and/or productivity (rate)) of acetyl-CoA derived products, including, for example, 1,3-BDO, R-1,3-BDO, S-1,3-BDO, MAA, methacrylic acid esters (e.g., MMA and EMA), butyrate, butanol, propane, formate, (3R)-hydroxybutyl (3R)-hydroxybutyrate, hexamethylenediamine (HMDA or HMD), caprolactam (CPL), adipate (or adipic acid), 6-aminocaproic acid (6-ACA), and/or 1,6-hexanediol (HDO), and related products derived therefrom. Similarly, the generation of the by-products acetoacetate, succinate, crotonate, butyrate, isobutyrate, (S)-3HB, (R)-3HB, malonate, and hexanoate, reduces the amounts of and/or the carbon flux through the precursors and/or intermediates acetoacetyl-CoA, succinyl-CoA, crotonyl-CoA, butyryl-CoA, isobutyryl-CoA, (S)-3HB—CoA, (R)-3HB—CoA, malonyl-CoA, and hexanoyl-CoA, respectively, which reduces the production of acetyl-CoA derived products.

1,3-BDO is a four-carbon diol that is traditionally produced from acetylene via its hydration. The resulting acetaldehyde is then converted to 3-hydroxybutyraldehyde, which is subsequently reduced to form 1,3-BDO. More recently, acetylene has been replaced by the less expensive ethylene as a source of acetaldehyde. 1,3-BDO is commonly used as an organic solvent for food flavoring agents. It is also used as a co-monomer for polyurethane and polyester resins, and is widely employed as a hypoglycemic agent. Optically active 1,3-BDO is a useful starting material for the synthesis of biologically active compounds and liquid crystals. Additionally, the dehydration of 1,3-butanediol affords 1,3-butadiene (see, e.g., Ichikawa et al. (2006) Journal of Molecular Catalysis A-Chemical 256:106-112; and Ichikawa et al. (2005) Journal of Molecular Catalysis A-Chemical 231:181-189), which is useful in the manufacture of synthetic rubbers (e.g., tires), latex, and resins. The reliance on petroleum-based feedstocks for either acetylene or ethylene warrants the development of a renewable feedstock-based route to 1,3-butanediol and to butadiene. 1,3-BDO, particularly (R)-1,3-BDO (also known as (R)-1,3-BG), which is a precursor of the ketone body (R)-β-hydroxybutyrate ((R)—BHB; also known as (R)-3-hydroxybutyrate or (3R)-hydroxybutyrate), is also useful as an exogenous ketogenic agent or ketogenic compound, that, upon ingestion or consumption, can result in an increase in blood BHB levels. In particular, (R)-1,3-BDO ingestion or consumption can result in blood BHB levels sufficient to result in various clinical benefits, including, for example, an enhancement of physical and cognitive performance, as well as the treatment of cardiovascular conditions, diabetes, certain types of epilepsy, mitochondrial dysfunction disorders, metabolic disorders, and muscle fatigue and impairment, among other diseases, conditions and disorders. It can also be used to achieve, maintain, increase, or support ketosis, for example, when taken as part of a ketogenic diet, or other type of specialized diet, or even when added to a non-ketogenic diet. 1,3-BDO or (R)-1,3-BDO can be formulated into or added to, for example, ketogenic foods, supplements, compositions, beverages, and/or drinks (e.g., carbonated, energy, and/or caffeinated drinks).

Methacrylic acid (MAA), or 2-methyl-2-propenoic acid, is a low molecular weight carboxylic acid that occurs naturally in small amounts in the oil of Roman chamomile. It is a corrosive liquid with an acrid unpleasant odor that is soluble in warm water and miscible with most organic solvents. Methacrylic acid polymerizes readily upon heating or treatment with a catalytic amount of strong acid, such as hydrochloric acid. The resulting polymer is a ceramic-looking plastic. Methacrylic acid is used industrially in the preparation of its esters, known collectively as methacrylates, such as methyl methacrylate (MMA) and ethyl methacrylate (EMA). The methacrylates have numerous uses, most notably in the manufacture of polymers, such as those with the trade names Lucite™ and Plexiglas™. Other than MMA polymers, the other major product of this industry is crude methacrylic acid, which accounts for about 20 percent of the total production of MMA. Crude MAA is processed into butyl methacrylates and/or “glacial” MAA, which is highly purified crude MAA. Glacial MAA can be used directly as a co-monomer in various polymers and is also used to make a variety of small volume methacrylates. MAA can also be converted into MMA via esterification with methanol.

Methyl methacrylate (MMA) is an organic compound with the formula CH₂═C(CH₃)CO₂CH₃. This colorless liquid is the methyl ester of methacrylic acid (MAA), and is the monomer for the production of the transparent plastic poly(methyl methacrylate) (PMMA). Methyl methacrylate (MMA) is a key intermediate chemical with a global demand in excess of 4.5 billion pounds per year, much of which is converted to polyacrylates. The principal application of methyl methacrylate is the production of poly(methyl methacrylate) acrylic plastics. Methyl methacrylate also is used for the production of the co-polymer methyl methacrylate-butadiene-styrene (MBS or MMBS), which is used as a modifier for polyvinyl chloride (PVC). Methyl methacrylate polymers and co-polymers are used for waterborne coatings, such as latex paint. Uses are also found in adhesive formulations. Contemporary applications include the use in plates that keep light spread evenly across liquid crystal display (LCD) computer and TV screens. Methyl methacrylate is also used to prepare corrosion casts of anatomical organs, such as coronary arteries of the heart.

Most commercial producers apply an acetone cyanohydrin (ACH) route to produce methacrylic acid (MAA), with acetone and hydrogen cyanide as raw materials. The intermediate cyanohydrin is converted with sulfuric acid to a sulfate ester of the methacrylamide, the hydrolysis of which yields ammonium bisulfate and MAA. Some producers start with isobutylene or, equivalently, tert-butanol, which is oxidized to methacrolein, and then further oxidized to methacrylic acid. MAA can then be esterified with methanol to produce methyl methacrylate (MMA), or with other alcohols to produce other esters of MAA (e.g., esterification with ethanol or butanol produces ethyl methacrylate and butyl methacrylate, respectively).

The conventional production process, using the acetone cyanohydrin route, involves the conversion of hydrogen cyanide (HCN) and acetone to acetone cyanohydrin, which then undergoes acid-assisted hydrolysis and esterification with methanol to give MMA. Difficulties in handling potentially deadly HCN, along with the high costs of by-product disposal (1.2 tons of ammonium bisulfate are formed per ton of MMA), have sparked a great deal of research aimed at cleaner and more economical production processes, with several new processes commercialized over the last two decades. For example, the Asahi “Direct Metha” route, which involves the oxidation of isobutylene to methacrolein, which is then mixed with methanol, oxidized with air, and esterified to MMA, has been described as an economical process. There exists a need for alternative methods for effectively producing compounds such as methacrylic acid.

The intake of compounds and compositions containing (R)-3-hydroxybutyrate derivatives, e.g., (3R)-hydroxybutyl (3R)-hydroxybutyrate, has been shown to boost the levels of ketone bodies in the blood. Ketone bodies are chemical compounds which are produced when fatty acids are metabolized by the body for energy, which can in turn lead to the ketone bodies themselves being used for energy. Ketone bodies have been shown to reduce the levels of free fatty acids circulating in the plasma of an individual. Ingestion of ketone bodies can lead to various clinical benefits, including an enhancement of physical and cognitive performance, as well as the treatment of cardiovascular conditions, diabetes, mitochondrial dysfunction disorders, and muscle fatigue and impairment. To gain desirable therapeutic and other benefits, the ketone bodies generally needs to be present in the blood plasma of an individual at a threshold level, for example, at least 1 mM. However, direct administration of ketone bodies is impractical and dangerous. For example, direct administration of (R)-3-hydroxybutyrate can result in significant acidosis following rapid absorption from the gastrointestinal tract. Administration of the sodium salt of these compounds is also unsuitable due to a potentially dangerous sodium overload that can result from the administration of therapeutically relevant amounts of these compounds. Administration of (R)-3-hydroxybutyrate derivatives in oligomeric form has been used to circumvent this problem.

Synthesis of stereogenic mixtures of 3-hydroxybutyl 3-hydroxybutyrate has been previously shown. Because the (3R,3′R) isomer is the most effective precursor of (3R)-hydroxybutyrate, and is a ketone body that is biosynthesized and utilized in vivo, various synthetic approaches have been developed for the production of the desired (3R,3′R) isomeric rich product. However, low yields, the production of impure product, or impracticability on a large scale, have hindered production. For example, a classical synthesis, from poly [(3R)-hydroxybutyric acid], gives pure product, but involves six chemical steps. Accordingly, there exists a need to produce a stereogenic purified product of (3R)-hydroxybutyl (3R)-hydroxybutyrate on a large scale.

Isopropanol is a colorless, flammable, three-carbon alcohol that mixes completely with most solvents, including water. The largest use for isopropanol is as a solvent, including its well known yet small use as “rubbing alcohol,” which is a mixture of isopropanol and water. As a solvent, isopropanol is found in many everyday products such as paints, lacquers, thinners, inks, adhesives, general-purpose cleaners, disinfectants, cosmetics, toiletries, deicers, and pharmaceuticals. Low-grade isopropanol is also used in motor oils. The second largest use is as a chemical intermediate for the production of isopropylamines (e.g. in agricultural products), isopropylethers, and isopropyl esters. Isopropanol is manufactured by two petrochemical routes. The predominant process entails the hydration of propylene either with or without sulfuric acid catalysis. Secondarily, isopropanol is produced via hydrogenation of acetone, which is a byproduct formed in the production of phenol and propylene oxide. High-priced propylene is currently driving costs up and margins down throughout the chemical industry motivating the need for an expanded range of low cost feedstocks.

Butanol, or equivalently, n-butanol, is a four carbon alcohol that is currently manufactured almost exclusively through the use of petrochemical raw materials. The main petrochemical process entails carbonylation of propylene to butyraldehyde, followed by catalytic hydrogenation to butanol. The demand for butanol is driven by its use for production of butyl acrylate and butyl methacrylate, both of which are employed in emulsified and solution polymers used in water-based latex coatings, enamels, and lacquers. Other applications include its use as an intermediate for large volume chemicals such as butyl acetate and glycol butyl ethers, as well as its direct use as a solvent. Butanol also is being considered for potential application as a biofuel derived from renewable resources. Butanol has a wide range of properties that make it better suited as a fuel than ethanol. For example, butanol has higher energy content, lower volatility and hygroscopicity, can be shipped through pipeline infrastructure, can be used directly without blending, and can be blended with diesel or biodiesel.

Adipic acid (or adipate) is a dicarboxylic acid that can be used to produce polyamides, including nylon 6,6, which is a a linear polyamide made by condensing adipic acid with hexamethylenediamine (HMDA or HMD). This is employed for manufacturing different kinds of fibers. Other uses of adipic acid include its use in plasticizers, unsaturated polyesters, and polyester polyols. Additional uses include for production of polyurethane, lubricant components, and as a food ingredient as a flavorant and gelling aid. Historically, adipic acid was prepared from various fats using oxidation. Some current processes for adipic acid synthesis rely on the oxidation of KA oil, a mixture of cyclohexanone, the ketone or K component, and cyclohexanol, the alcohol or A component, or of pure cyclohexanol using an excess of strong nitric acid. There are several variations of this theme which differ in the routes for production of KA or cyclohexanol. For example, phenol is an alternative raw material in KA oil production, and the process for the synthesis of adipic acid from phenol has been described. The other versions of this process tend to use oxidizing agents other than nitric acid, such as hydrogen peroxide, air or oxygen.

In addition to hexamethylenediamine (HMDA or HMD) being used in the production of nylon-6,6 as described above, it is also utilized to make hexamethylene diisocyanate, a monomer feedstock used in the production of polyurethane. The diamine also serves as a cross-linking agent in epoxy resins. HMDA is traditionally produced by the hydrogenation of adiponitrile.

Caprolactam is an organic compound which is a lactam of 6-aminohexanoic acid (ε-aminohexanoic acid, 6-aminocaproic acid). It can alternatively be considered as a cyclic amide of caproic acid. One use of caprolactam is as a monomer in the production of nylon-6. Caprolactam can be synthesized from cyclohexanone via an oximation process using hydroxylammonium sulfate followed by catalytic rearrangement using the Beckmann rearrangement process step. In addition to being used for the production of caprolactam, 6-ACA also can be used directly as raw material for the production of nylon-6. 6-ACA is also an antifibrinolytic agent that is used to control bleeding (e.g., postoperative bleeding) and for the treatment of bleeding disorders. Thus, there exists a need for the development of methods, engineered (i.e., non-naturally occurring) organisms, and/or engineered enzymes, polypeptides, or proteins to decrease the production of unwanted by-products, such as 3HB, for increasing the efficiency and effectively producing commercial quantities of acetyl-CoA derived compounds such as 1,3-BDO, (3R)-hydroxybutyl (3R)-hydroxybutyrate, MAA, methacrylic acid esters (e.g., MMA or EMA), butyrate, butanol, propane, formate, and/or isopropanol. The present invention satisfies these needs and provides related advantages as well. There also exists a need for the development of methods, engineered microorganisms, and biosynthetic pathways, to decreased the production of other unwanted by-products, such as acetate, acetoacetate, succinate, crotonate, butyrate, isobutyrate, (S)-3HB, (R)-3HB, malonate, and/or hexanoate, for increasing the efficiency and effectively producing commercial quantities of acetyl-CoA derived compounds, such as, for example, 1,3-BDO, (R)-1,3-BDO, (S)-1,3-BDO, (3R)-hydroxybutyl (3R)-hydroxybutyrate, MAA, methacrylic acid esters (e.g., MMA or EMA), butyrate, butanol, propane, formate, isopropanol, hexamethylenediamine (HMDA or HMD), caprolactam (CPL), adipate (or adipic acid), 6-aminocaproic acid (6-ACA), and/or 1,6-hexanediol (HDO), and related products derived therefrom.

### SUMMARY

Provided herein are non-naturally occurring (or engineered) microbial organisms having eliminated, substantially eliminated or reduced 3-hydroxybutyrate (3HB) by-product, comprising a microbial organism having an acetyl Coenzyme A (acetyl-CoA) derived product pathway and an enhanced or increased carbon flux through 3-hydroxybutyryl Coenzyme-A (3HB—CoA), where the microbial organism comprises a 3HB recycling loop. In some embodiments, the 3HB recycling loop comprises at least one exogenous nucleic acid encoding an acyl-CoA synthetase (ACS) or homolog thereof. In some embodiments, said ACS or homolog thereof is expressed in a sufficient amount to reduce production of 3HB. In some embodiments, said ACS or homolog thereof is expressed in a sufficient amount to increase the yield of the acetyl-CoA derived product. In some embodiments, the at least one exogenous nucleic acid is a heterologous nucleic acid. In some embodiments, the non-naturally occurring microbial organism has reduced concentration of intracellular 3HB. In some embodiments, the non-naturally occurring microbial organism has reduced concentration of intracellular 3HB compared to a microbial organism without a 3HB recycling loop. In some embodiments, the non-naturally occurring microbial organism has reduced concentration of intracellular 3HB compared to a microbial organism with wild-type expression of ACS. In some embodiments, the non-naturally occurring microbial organism has increased yield of the acetyl-CoA derived product. In some embodiments, the non-naturally occurring microbial organism has increased yield of the acetyl-CoA derived product compared to a microbial organism without a 3HB recycling loop. In some embodiments, the non-naturally occurring microbial organism has increased yield of the acetyl-CoA derived product compared to a microbial organism with wild-type expression of ACS. In some embodiments, the acetyl-CoA derived product is 1,3-BDO, optionally an R-1,3 BDO, (3R)-hydroxybutyl (3R)-hydroxybutyrate, butyrate, butanol, propane, formate, isopropanol, MAA, or an MAA ester.

In some embodiments, the ACS or homolog thereof is an AMP-forming ACS or homolog thereof. In particular embodiments, the ACS or homolog thereof catalyzes the activation of short-chain (e.g., C2-C4, C2-C6, or C2-C8) carboxylic acids or carboxylates to their corresponding CoA thioesters or acyl-CoAs. In other words, the ACS catalyzes the ligation of short-chain carboxylic acids or carboxylates with CoA to produce the corresponding CoA thioesters, which are also referred to herein as acyl-CoAs or short-chain acyl-CoAs. In some embodiments, the ACS is heterologous to the non-naturally occurring microorganism and is expressed or overexpressed. In other embodiments, the ACS is native to the non-naturally occurring microorganism and is overexpressed.

In general, the carboxylates are by-products that are generated from (the corresponding) acyl-CoAs, and the acyl-CoAs are precursors or intermediates of acetyl-CoA derived product pathways. Since an acetyl-CoA derived product pathway can include one or more acyl-CoA precursors or intermediates, a cell or microorganism, such as the non-naturally occurring microbial organisms described herein, comprising at least one acetyl-CoA derived product pathway, can produce one or more carboxylate by-products. For example, the microorganism can produce 1, 2, 3, 4, 5, 6, 7, 8, 9, or 10, or more, carboxylate by-products. Thus, the microorganism expressing an ACS as described herein, can comprise more than one carboxylate by-product recycling loop. For example, the non-naturally occurring microbial organism can comprise 1, 2, 3, 4, 5, 6, 7, 8, 9, or 10, or more, by-product recycling loops. In some embodiments, the non-naturally occurring microbial organism comprises one by-product recycling loop. In some embodiments, the one by-product recycling loop is a 3HB recycling loop. In other embodiments, the non-naturally occurring microbial organism comprises two, or at least two, by-product recycling loops. In some embodiments, the two, or at least two, by-product recycling loops comprise a 3HB recycling loop and an acetate recycling loop.

For example, in some embodiments, the ACS catalyzes the conversion of at least one of a 3HB, (R)-3HB, (S)-3HB, acetate, acetoacetate, succinate, crotonate, butyrate, isobutyrate, malonate, and/or hexanoate by-product to the corresponding CoA thioesters/acyl-CoAs, i.e., 3HB—CoA, (R)-3HB—CoA, (S)-3HB—CoA, acetyl-CoA, acetoacetyl-CoA, succinyl-CoA, crotonyl-CoA, butyryl-CoA, isobutyryl-CoA, malonyl-CoA, and/or hexanoyl-CoA. For example, in some embodiments, the engineered or recombinant microorganism (or non-naturally occurring microbial organism) comprises one or more acetyl-CoA derived product pathways and at least one by-product recycling loop. In some embodiments, the by-product recycling loop comprises one or more of a 3HB recycling loop, an (R)-3HB recycling loop, an (S)-3HB recycling loop, an acetate recycling loop, an acetoacetate recycling loop, a succinate recycling loop, a crotonate recycling loop, a butyrate recycling loop, an isobutyrate recycling loop, a malonate recycling loop, and/or a hexanoate recycling loop (collectively referred to herein as by-product recycling loops or pathways, or carboxylate by-product recycling loops or pathways), wherein, in some embodiments, each of the aforementioned by-product recycling loops comprises an acyl-CoA synthetase (ACS) or homolog thereof, and/or comprises at least one nucleic acid encoding an acyl-CoA synthetase (ACS) or homolog thereof. In some embodiments, the nucleic acid encoding the ACS or homolog thereof is an exogenous nucleic acid. In some embodiments, such engineered microorganisms have reduced intracellular concentrations and/or produce reduced/decreased amounts (e.g., titers) of 3HB, (R)-3HB, (S)-3HB, acetate, acetoacetate, succinate, crotonate, butyrate, isobutyrate, malonate, and/or hexanoate by-product(s), and/or have increased production (e.g., increased titer, yield, rate, and/or productivity) of acetyl-CoA derived products. In some embodiments, the acetyl-CoA derived product is 1,3-BDO, (R)-1,3-BDO, (S)-1,3-BDO, (3R)-hydroxybutyl (3R)-hydroxybutyrate, MAA, methacrylic acid esters (e.g., MMA or EMA), butyrate, butanol, propane, formate, isopropanol, hexamethylenediamine (HMDA or HMD), caprolactam (CPL), adipate (or adipic acid), 6-aminocaproic acid (6-ACA), and/or 1,6-hexanediol (HDO), and/or related products derived therefrom. In some embodiments, the non-naturally occurring microbial organism has reduced intracellular concentrations and/or has reduced/decreased, eliminated, or substantially eliminated production of 3HB and acetate by-products, and has increased production of 1,3-BDO. In some embodiments, the non-naturally occurring microbial organism has reduced intracellular concentrations and/or has reduced/decreased, eliminated, or substantially eliminated production of (R)-3HB and acetate by-products, and has increased production of (R)-1,3-BDO.

Thus, also provided herein are non-naturally occurring microbial organisms (or engineered or recombinant microorganisms) having eliminated, substantially eliminated, or reduced by-product(s) and, optionally, increased production of an acetyl-CoA derived product or products, wherein: (i) the microbial organism comprises at least one by-product recycling loop and at least one acetyl-CoA derived product pathway; (ii) the by-product recycling loop comprises an ACS or a homolog or a variant thereof; (iii) the by-product(s) comprise(s) one or more of 3HB, R-3HB, (S)-3HB, acetate, acetoacetate, succinate, crotonate, butyrate, isobutyrate, malonate, and/or hexanoate by-product(s); and (iv) the acetyl-CoA derived product (or product pathway) is one or more of a 1,3-BDO, R-1,3-BDO, (S)-1,3-BDO, (3R)-hydroxybutyl (3R)-hydroxybutyrate, MAA, methacrylic acid esters (e.g., MMA or EMA), butyrate, butanol, propane, formate, isopropanol, hexamethylenediamine (HMDA or HMD), caprolactam (CPL), adipate (or adipic acid), 6-aminocaproic acid (6-ACA), and/or 1,6-hexanediol (HDO) product (or product pathway). In some embodiments, the ACS is heterologous to the non-naturally occurring microorganism and is expressed or overexpressed. In other embodiments, the ACS is native to the non-naturally occurring microorganism and is overexpressed.

In some embodiments, the ACS or homolog thereof is derived from a species of Ruegeria, Rhodobacteraceae, Cribrihabitans, Falsiruegeria, Tropicibacter, Thioclava, Paracoccus, Celeribacter, Gemmobacter, Marivivens, Actibacterium, Salipiger, Youngimonas, Poseidonocella, Nitratireductor, Rhizobium, Aestuarium, Pseudodonghicola, Rhodobacterales, Ciceribacter, Polymorphum, Rhizobiales, Salipiger, Rhizobiaceae, or Roseobacter.

In some embodiments, the ACS comprises the amino acid sequence set forth in SEQ ID NO: 1. In some embodiments, the ACS is a homolog of the ACS set forth in SEQ ID NO: 1. In some embodiments, the ACS is a variant of the ACS set forth in SEQ ID NO: 1. In some embodiments, the ACS or homolog thereof has activity corresponding to EC 6.2.1.-, EC 6.2.1.1, and/or EC 6.2.2.2. In some embodiments, the ACS is heterologous to the engineered microorganism and is expressed or overexpressed. In other embodiments, the ACS is native to the engineered microorganism and is overexpressed. In some embodiments, the non-naturally occurring microbial organisms provided herein comprise an exogenous nucleic acid encoding the heterologous ACS. In other embodiments, the non-naturally occurring microbial organisms provided herein comprise an exogenous nucleic acid encoding the native ACS.

The ACS homolog or variant can have 1 or more modifications (or alterations or mutations) to its amino acid sequence, compared to the template or wild-type sequence. The template sequence can be a variant of a wild-type sequence. For example, the ACS homolog or variant can have 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, or more, amino acid modifications (or alterations or mutations) compared to the template of wild-type sequence, wherein an amino acid modification (or alteration or mutation) can include an amino acid addition, deletion, and/or substitution or replacement. The ACS homolog or variant can have at least about 20-99% sequence identity with the wild-type or template sequence, for example, with the amino acid sequence set forth in SEQ ID NO: 1. For example, the ACS homolog or variant can have at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99%, or higher, sequence identity with the template or wild-type sequence, such as, for example, with the sequence set forth in SEQ ID NO: 1. In some embodiments, the ACS homolog or variant can have at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or higher, sequence identity with an ACS derived from a species of Ruegeria, Rhodobacteraceae, Cribrihabitans, Falsiruegeria, Tropicibacter, Thioclava, Paracoccus, Celeribacter, Gemmobacter, Marivivens, Actibacterium, Salipiger, Youngimonas, Poseidonocella, Nitratireductor, Rhizobium, Aestuarium, Pseudodonghicola, Rhodobacterales, Ciceribacter, Polymorphum, Rhizobiales, Salipiger, Rhizobiaceae, or Roseobacter. In some embodiments, the ACS homolog or variant can have at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or higher, sequence identity with an ACS derived from a species of Ruegeria, such as Ruegeria pomeroyi, and/or can have at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or higher, sequence identity with any one of the ACS homologs listed in TABLE 2.

Also provided herein are non-naturally occurring (or engineered or recombinant) microbial organisms (or microorganisms) having eliminated, substantially eliminated, or reduced 3-hydroxybutyrate (3HB) by-product, wherein the microbial organism has an acetyl Coenzyme A (acetyl-CoA) derived product pathway and an enhanced or increased carbon flux through 3-hydroxybutyraldehyde (3HB-Ald), and wherein the microbial organism comprises a 3HB to 3HB-Ald conversion step. In some embodiments, the 3HB to 3HB-Ald conversion step comprises at least one nucleic acid encoding a carboxylic acid reductase (CAR) or a variant or a homolog thereof. In some embodiments, the nucleic acid is an exogenous nucleic acid. In some embodiments, the CAR or variant or homolog thereof is expressed in a sufficient amount to reduce production of 3HB. In some embodiments, the CAR or variant or homolog thereof is expressed in a sufficient amount to increase the yield of the acetyl-CoA derived product. In some embodiments, the at least one exogenous nucleic acid is a heterologous nucleic acid. In some embodiments, the non-naturally occurring microbial organism has reduced concentration of intracellular 3HB. In some embodiments, the non-naturally occurring microbial organism has reduced concentration of intracellular 3HB compared to a microbial organism without a 3HB to 3HB-Ald conversion step. In some embodiments, the non-naturally occurring microbial organism has reduced concentration of intracellular 3HB compared to a microbial organism with wild-type expression of CAR. In some embodiments, the non-naturally occurring microbial organism has increased production (e.g., titer, yield, rate, and/or productivity) of the acetyl-CoA derived product. In some embodiments, the non-naturally occurring microbial organism has increased yield of the acetyl-CoA derived product compared to a microbial organism without a 3HB to 3HB-Ald conversion step. In some embodiments, the non-naturally occurring microbial organism has increased yield of the acetyl-CoA derived product compared to a microbial organism with wild-type expression of CAR. In some embodiments, the acetyl-CoA derived product is 1,3-BDO, optionally an R-1,3 BDO, (3R)-hydroxybutyl (3R)-hydroxybutyrate, butyrate, butanol, propane, formate, isopropanol, MAA, or an MAA ester. In some embodiments, the CAR or variant or homolog thereof is heterologous to the non-naturally occurring microbial organism and is expressed or overexpressed. In other embodiments, the CAR or variant or homolog thereof is native to the non-naturally occurring microbial organism and is overexpressed.

In some embodiments, the CAR or variant or homolog thereof is derived from a species or strain of Mycobacterium, Mycolicibacterium, Mycolicibacillus, Mycolicibacter, Pseudofrankia, Norcadia, Pseudonocardiaceae, or Longimycelium. In some embodiments, the CAR or variant or homolog thereof is derived from a species or strain of Mycobacterium. In some embodiments, the CAR or variant or homolog thereof is derived from a species or strain of Mycolicibacterium. In some embodiments, the CAR or variant or homolog thereof is derived from a strain or species of Mycobacterium avium, Mycobacterium tuberculosis, Mycobacterium paratuberculosis, Mycobacterium helveticum, Mycobacterium kiyosense, Mycobacterium fragrae, Mycobacterium asiaticum, Mycobacterium heidelbergence, Mycobacterium sp. 1164966.3, Mycobacterium haemophilum, Mycobacterium talmoniae, Mycobacterium gordonae, Mycobacterium pseudokansasii, Mycobacterium kansasii, Mycobacterium botniense, Mycobacterium cookii, Mycobacterium sherrisii, Mycobacterium xenopi RIVM700367, Mycobacterium leprae, Mycolicibacterium wolinskyi, Mycolicibacillus trivialis, Mycolicibacterium fortuitum (Mycobacterium fortuitum), Mycobacterium dioxanotrophicus, Mycolicibacter sinensis, Mycobacterium sp. 1164966.3, Mycobacterium intermedium, Mycolicibacterium wolinskyi, Mycolicibacterium cosmeticum, Mycobacterium marinum, Mycolicibacterium smegmatis (Mycobacterium smegmatis), Mycobacterium lacus, Mycolicibacterium llatzerense, Mycobacterium simulans, Mycobacterium sp. 1274761.0, Mycolicibacterium fortuitum (Mycobacterium fortuitum), Mycolicibacterium chubuense (Mycobacterium chubuense), Mycolicibacterium chubuense (Mycobacterium chubuense) (strain NBB4), Mycobacterium sp. GA-2829, Mycobacterium decipiens, Mycobacterium dioxanotrophicus, Mycolicibacterium cosmeticum, Mycolicibacterium agri (Mycobacterium agri), Mycobacterium shinjukuense, Mycobacterium spongiae, Mycolicibacterium doricum, Mycobacterium sp. NAZ190054, Mycolicibacterium aurum (Mycobacterium aurum), Mycolicibacterium sarraceniae, Mycolicibacterium sp. P9-64, Mycolicibacterium holsaticum, Mycobacterium tuberculosis, Mycobacterium tuberculosis, Mycobacterium tuberculosis, Mycobacterium tuberculosis, Mycobacterium bovis, Pseudofrankia asymbiotica, Mycolicibacterium tokaiense, Mycolicibacterium madagascariense, Mycolicibacterium confluentis, Mycolicibacterium phlei, Mycobacterium sp. shizuoka-1, Mycobacterium adipatum, Mycolicibacterium madagascariense, Mycolicibacterium sediminis, Mycobacterium sp. ACS1612, Mycobacterium grossiae, Mycolicibacterium neoaurum, Mycolicibacterium rhodesiae (Mycobacterium rhodesiae) (strain NBB3), Mycobacterium sp. 852013-51886 SCH5428379, Mycolicibacterium anyangense, Mycolicibacterium sp. P9-64, Mycolicibacterium insubricum, Mycolicibacterium sediminis, Mycobacterium sp. Root135, Mycolicibacterium llatzerense, Mycobacterium sp. Root135, Mycobacterium grossiae, Mycolicibacterium fallax (Mycobacterium fallax), Mycolicibacterium poriferae, Nocardia jinanensis, Mycobacterium sp. ACS1612, Mycobacterium sp. GA-2829, Nocardia speluncae, Nocardia terpenica, Mycolicibacterium brumae, Pseudonocardiaceae bacterium YIM PH 21723, Nocardia brasiliensis, Nocardia iowensis, Mycobacterium marinum, Nocardia arthritidis, Longimycelium tulufanense, Nocardia terpenica, Nocardia brasiliensis, or Nocardia panacis. In some embodiments, the CAR or variant or homolog thereof is derived from a species or strain of Mycobacterium avium. In some embodiments, the CAR or variant or homolog thereof is derived from a species or strain of Mycobacterium smegmatis or Mycolicibacterium smegmatis. In some embodiments, the CAR comprises the amino acid sequence set forth in SEQ ID NO: 2 (WT). In some embodiments, the CAR is encoded by a car or a fadD9 gene. In some embodiments, the CAR or variant or homolog thereof is a CAR listed in TABLE 3.1, or the CAR is a variant or homolog of any one of the CARs listed in TABLE 3.1. In some embodiments, the CAR is a variant or homolog of the wild-type (WT) CAR of SEQ ID NO: 2. In some embodiments, the CAR variant comprises the amino acid sequence set forth in SEQ ID NO: 3. In some embodiments, the CAR or variant or homolog thereof comprises activity corresponding to EC 1.2.1.- and/or EC 1.2.1.30.

The CAR variant or homolog can have 1 or more modifications (or alterations or mutations) to its amino acid sequence, compared to the template or wild-type sequence. A template sequence is any sequence (e.g., a nucleotide and/or amino acid sequence) that serves as a starting point for modification or mutagenesis, whereby modifying or mutating the template sequence results in a variant, modified, or genetically engineered sequence (e.g., a nucleic acid and/or amino acid sequence), such as, for example, a variant CAR polypeptide, or a modified/genetically engineered nucleic acid sequence that encodes a CAR variant. The template sequence can be a variant of a wild-type sequence, and can be used to generate further variants. For example, the CAR variant or homolog can have at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, or more, amino acid modifications (or alterations or mutations) compared to the template of wild-type sequence, wherein an amino acid modification (or alteration or mutation) can include an amino acid addition, deletion, and/or substitution or replacement. The CAR variant or homolog can have 20-99% sequence identity with the wild-type or template sequence, for example the amino acid sequence set forth in SEQ ID NO: 2 or SEQ ID NO: 3. For example, the CAR variant or homolog can have at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99%, or higher, sequence identity with the template or wild-type sequence, such as, for example, with the sequence set forth in SEQ ID NO: 2 or SEQ ID NO: 3. In some embodiments, the CAR or variant or homolog thereof can have at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99%, or higher, sequence identity with a CAR derived from a species of Mycobacterium. In some embodiments, the CAR or variant or homolog thereof can have at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99%, or higher, sequence identity to a CAR derived from Mycobacterium avium, or to a CAR derived from Mycobacterium smegmatis. In some embodiments, the CAR or variant or homolog thereof can have at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99%, or higher, sequence identity to a CAR derived from a strain or species of Mycobacterium, Mycolicibacterium, Mycolicibacillus, Mycolicibacter, Pseudofrankia, Norcadia, Pseudonocardiaceae, or Longimycelium. In some embodiments, the CAR or variant or homolog thereof is derived from a strain or species listed in TABLE 3.1, or the CAR or variant or homolog thereof is one or more of the CARs listed in TABLE 3.1. In some embodiments, the CAR is a variant or homolog of any one of more of the CARs listed in TABLE 3.1. In some embodiments, the CAR is a variant or homolog, having at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99%, or higher, sequence identity to any one or more of the CARs listed in TABLE 3.1.

Also provided herein are non-naturally occurring microbial organisms further comprising a 1,3-BDO, optionally an R-1,3 BDO, (3R)-hydroxybutyl (3R)-hydroxybutyrate, butyrate, butanol, propane, formate, isopropanol, MAA, or an MAA ester pathway. In some embodiments, said microbial organism comprises a 1,3-BDO, optionally a R-1,3 BDO pathway. In some embodiments, said 1,3-BDO pathway comprises:

AcAc—COA reductase (a CoA-dependent, aldehyde forming oxidoreductase); 3-oxobutyraldehyde reductase (a ketone reducing/alcohol forming oxidoreductase); 3HB-Ald reductase (an aldehyde reducing/alcohol forming oxidoreductase), acetaldehyde-alcohol dehydrogenase, 1,3-BDO or 1,3-butylene glycol dehydrogenase, or ADH; AcAc—COA reductase (a CoA-dependent, alcohol forming oxidoreductase); 3-oxobutyraldehyde reductase (an aldehyde reducing/alcohol forming oxidoreductase); 4-hydroxy-2-butanone reductase (a ketone reducing/alcohol forming oxidoreductase); AcAc—COA reductase (ketone reducing/alcohol forming oxidoreductase); 3HB—CoA reductase (a CoA-dependent, aldehyde forming oxidoreductase), ALD, or acetaldehyde-alcohol dehydrogenase; 3HB—CoA reductase (an alcohol forming, CoA dependent oxidoreductase); a 3HB—CoA transferase, or a 3HB—CoA hydrolase; 3HB dehydrogenase (a ketone reducing, alcohol forming oxidoreductase); AcAc—CoA transferase, AcAc—COA hydrolase, AcAc—COA synthetase; acetoacetate reductase (an aldehyde forming oxidoreductase); and a Ac—CoA thiolase;
AcAc—COA thiolase; HBD, optionally a R—HBD, or AcAc—COA reductase (ketone reducing/alcohol forming oxidoreductase); butyraldehyde dehydrogenase or acetaldehyde-alcohol dehydrogenase; acetaldehyde-alcohol dehydrogenase; PTB; and BUK;
AcAc—COA thiolase; HBD, optionally R—HBD; (R)-3HB—CoA reductas; (R)-3HB-Ald reductase; Ac—COA carboxylase; AcAc—COA synthase; HBD, optionally S—HBD; and 3HB—CoA epimerase;
4HB—CoA dehydratase (a hydro-lyase); CRT (a hydro-lyase); 3HB—CoA reductase (a Co-A dependent, aldehyde forming oxidoreductase); 3HB-Ald reductase (an alcohol forming oxidoreductase); and 3HB—CoA reductase (an alcohol forming oxidoreductase); and/or a SucCoA transferase, SucCoA hydrolase, and/or a SucCoA synthetase (or SucCoA ligase); a SucCoA reductase (aldehyde forming); a 4HB dehydrogenase; a 4HB kinase; a phosphotrans-4-hydroxybutyrylase; a Succ reductase; a SucCoA reductase (alcohol forming); a 4HB—CoA transferase, or a 4HB—CoA synthetase, or a 4HB—CoA ligase; an AKG decarboxylase; a 4HB—CoA dehydratase; a CRT; a 3HB—CoA reductase (aldehyde forming); a 3-hydroxybutanal reductase; a 3HB—CoA reductase (alcohol forming); a 3HB—CoA hydrolase, and/or a 3HB—CoA transferas; an alcohol forming reductase; a glutamate dehydrogenase and/or a glutamate transaminase; a glutamate decarboxylase; a 4-aminobutyrate dehydrogenase and/or a 4-aminobutyrate transaminase; and an AKGD.

In some embodiments, said microbial organism comprises an (3R)-hydroxybutyl (3R)-hydroxybutyrate pathway. In some embodiments, said (3R)-hydroxybutyl (3R)-hydroxybutyrate pathway comprises
AcAc—COA thiolase; HBD, optionally R—HBD; (R)-3HB—CoA reductase; (R)-3HB-Ald reductase; Ac-COA carboxylase; AcAc—COA synthase; HBD, optionally S—HBD; 3HB—CoA epimerase; and (3R)-hydroxybutyryl-CoA:(R)-1,3-butanediol alcohol transferase; and/or
AcAc—COA thiolase; HBD, optionally a R—HBD, or AcAc—COA reductase (ketone reducing/alcohol forming oxidoreductase); butyraldehyde dehydrogenase or acetaldehyde-alcohol dehydrogenase; acetaldehyde-alcohol dehydrogenase; PTB; BUK; and (3R)-hydroxybutyryl-CoA:(R)-1,3-butanediol alcohol transferase.

In some embodiments, said microbial organism comprises a butyrate, butanol, propane, and/or formate pathway. In some embodiments, said butyrate, butanol, propane, and/or formate pathway comprises: AcAc—COA thiolase; HBD, optionally, S—HBD; CRT; BCD; ALDH; AHR; PTB; BUK; and ADO.

In some embodiments, said microbial organism comprises an isopropanol pathway. In some embodiments, said isopropanol pathway comprises: CRT; HBD; acetoacetyl-CoA synthetase, acetyl-CoA:acetoacetate-CoA transferase, and/or acetoacetyl-CoA hydrolase; acetoacetate decarboxylase; and acetone reductase.

In some embodiments, said microbial organism comprises a MAA pathway. In some embodiments, said MAA pathway comprises:
AcAc—CoA thiolase; AcAc—COA reductase (ketone reducing/alcohol forming oxidoreductase); 3HB—CoA mutase; 2HIB—CoA dehydratase; methacrylyl-CoA transferase, methacrylyl-CoA hydrolase and/or methacrylyl-CoA synthetase; 4HB—CoA dehydratase; vinylacetyl-CoA A-isomerase; and/CRT; and/or
AcAc—COA thiolase; AcAc—COA reductase (ketone reducing/alcohol forming oxidoreductase); methacrylyl-CoA transferase, methacrylyl-CoA hydrolase and/or methacrylyl-CoA synthetase; CRT; butyryl-CoA dehydrogenase; isobutyryl-CoA mutase; and isobutyryl-CoA dehydrogenase.

In some embodiments, said microbial organism comprises a MAA ester pathway. In some embodiments, said MAA ester pathway comprises:
a methacrylyl-CoA transferase or a methacrylyl-CoA synthetase; and an alcohol transferase; and/or
a methacrylate ester-forming enzyme.

Also provided herein are non-naturally occurring microbial organisms comprising at least one by-product recycling loop (e.g., a carboxylate by-product recycling loop), such as a 3HB recycling loop, an (R)-3HB recycling loop, an (S)-3HB recycling loop, an acetate recycling loop, an acetoacetate recycling loop, a succinate recycling loop, a crotonate recycling loop, a butyrate recycling loop, an isobutyrate recycling loop, a malonate recycling loop, and/or a hexanoate recycling loop, and further comprising at least one acetyl-CoA derived product pathway. In some embodiments, the non-naturally occurring microbial organism comprises one or more of a by-product recycling loop, a 3HB to 3HB-Ald conversion step, and an acetyl-CoA derived product pathway. In some embodiments, the acetyl-CoA derived product pathways a 1,3-BDO, (R)-1,3-BDO, (S)-1,3-BDO, (3R)-hydroxybutyl (3R)-hydroxybutyrate, MAA, methacrylic acid esters (e.g., MMA or EMA), butyrate, butanol, propane, formate, isopropanol, hexamethylenediamine (HMDA or HMD), caprolactam (CPL), adipate (or adipic acid), 6-aminocaproic acid (6-ACA), and/or 1,6-hexanediol (HDO) pathway. Methods and pathways for the biosynthesis of adipate, hexamethylenediamine, 6-aminocaproic acid, caprolactam, and 1,6-hexanediol (HDO) are known in the art, and are described, for example, in WO 2010/129936, WO 2016/209883, WO 2009/151728, WO 2014/099725, WO 2012/177721, WO 2009/113853, WO 2005/068643, WO 2009/113855, and WO 2011/078668, which are incorporated herein by reference in their entirety.

In some embodiments, the non-naturally occurring microbial organism (or the engineered or recombinant microorganism) comprises a 3HB recycling loop, a 3HB to 3HB-Ald conversion step, and a 1,3-BDO product pathway. In some embodiments, the non-naturally occurring microbial organism comprises an (R)-3HB recycling loop, an (R)-3HB to (R)-3HB-Ald conversion step, and an (R)-1,3-BDO product pathway. In some embodiments, the non-naturally occurring microbial organism comprises a 3HB to 3HB-Ald conversion step and a 1,3-BDO product pathway. In some embodiments, the non-naturally occurring microbial organism comprises an (R)-3HB to (R)-3HB-Ald conversion step and an (R)-1,3-BDO product pathway. In some embodiments, the non-naturally occurring microbial organism has reduced, eliminated, or substantially eliminated 3HB by-product and increased production of 1,3-BDO. In some embodiments, the non-naturally occurring microbial organism further comprises an acetate by-product recycling loop (or an acetate recycling loop). In some embodiments, the microbial organism has reduced, eliminated, or substantially eliminated acetate and 3HB by-products, and has increased production of 1,3-BDO product. In some embodiments, the 3HB by-product is R-3HB and the 1,3-BDO product is R-1,3-BDO.

In some embodiments, the microbial organism is a species of bacteria, yeast, or fungus. In some embodiments, said non-naturally occurring microbial organism is in a substantially anaerobic culture medium. In some embodiments, the non-naturally occurring microbial organism is in a microaerobic culture medium. In some embodiments, the non-naturally occurring microbial organism is cultured under substantially anaerobic conditions, or is cultured under microaerobic conditions.

Also provided herein are methods for increasing the production of 1,3-BDO, comprising culturing the non-naturally occurring microbial organism described herein under conditions and for a sufficient period of time to increase the availability of 1,3-BDO.

Also provided herein are methods for increasing the production of 3R-hydroxybutyric acid-3R-hydroxybutryrate, comprising culturing the non-naturally occurring microbial organism described herein, under conditions and for a sufficient period of time to increase the availability of 3R-hydroxybutyric acid-3R-hydroxybutryrate.

Also provided herein are methods for increasing the production of butyrate, butanol, propane, and/or formate comprising culturing the non-naturally occurring microbial organism described herein, under conditions and for a sufficient period of time to increase the availability of butyrate, butanol, propane, and/or formate.

Also provided herein are methods for increasing the production of isopropanol comprising culturing the non-naturally occurring microbial organism described herein, under conditions and for a sufficient period of time to increase the availability of isopropanol.

Also provided herein are methods for increasing the production of MAA, comprising culturing the non-naturally occurring microbial organism described herein, under conditions and for a sufficient period of time to increase the availability of MAA.

Also provided herein are methods for increasing the production of an MAA ester, comprising culturing the non-naturally occurring microbial organism described herein, under conditions and for a sufficient period of time to increase the availability of an MAA ester.

Also provided herein are methods for increasing the production of an acetyl-CoA derived product, the method comprising culturing the non-naturally occurring microbial organisms described herein, under conditions and for a sufficient period of time to increase the titer, yield, production, productivity, and/or availability of the acetyl-CoA derived product. In some embodiments, the acetyl-CoA derived product is 1,3-BDO, R-1,3-BDO, (S)-1,3-BDO, (3R)-hydroxybutyl (3R)-hydroxybutyrate, MAA, methacrylic acid esters (e.g., MMA or EMA), butyrate, butanol, propane, formate, isopropanol, hexamethylenediamine (HMDA or HMD), caprolactam (CPL), adipate (or adipic acid), 6-aminocaproic acid (6-ACA), and/or 1,6-hexanediol (HDO).

Also provided herein are methods for enhancing or increasing the carbon flux through 3HB—CoA in a non-naturally occurring microbial organism to increase the yield of an acetyl-CoA derived product and to eliminate, substantially eliminate, or reduce the production of 3HB by-product, the method comprising culturing any non-naturally occurring microbial organism described herein under conditions and for a sufficient period of time to produce the acetyl-CoA derived product. Also provided herein are methods for enhancing or increasing the carbon flux through 3HB—CoA and/or 3HB-Ald in a non-naturally occurring microbial organism to increase the yield of an acetyl-CoA derived product and to eliminate, substantially eliminate, or reduce the production of 3HB by-product, the method comprising culturing any non-naturally occurring microbial organism described herein under conditions and for a sufficient period of time to produce the acetyl-CoA derived product. In some embodiments, the acetyl-CoA derived product is selected from a group consisting of 1,3-BDO optionally an R-1,3 BDO, (3R)-hydroxybutyl (3R)-hydroxybutyrate, butyrate, butanol, propane, formate, MAA, or an MAA ester pathway. In some embodiments, the acetyl-CoA derived product comprises 1,3-BDO, optionally R-1,3 BDO. In some embodiments, the acetyl-CoA derived product comprises 3R-hydroxybutyric acid-3R-hydroxybutryrate. In some embodiments, the acetyl-CoA derived product comprises butyrate, butanol, propane, and/or formate. In some embodiments, the acetyl-CoA derived product comprises isopropanol. In some embodiments, the acetyl-CoA derived product comprises MAA. In some embodiments, the acetyl-CoA derived product comprises an MAA ester.

Also provided herein are methods for enhancing or increasing the carbon flux through one or more of 3HB-Ald, (R)-3HAld, 3HB—CoA, (R)-3HB—CoA, (S)-3HB—CoA, (S)-3HB-Ald, acetyl-CoA, acetoacetyl-CoA, succinyl-CoA, crotonyl-CoA, butyryl-CoA, isobutyryl-CoA, malonyl-CoA, and/or hexanoyl-CoA, in a non-naturally occurring microbial organism, to increase the yield of an acetyl-CoA derived product, and to eliminate, substantially eliminate, or reduce the production of one or more by-products, where the by-products comprise one or more of 3HB, (R)-3HB, (S)-3HB, acetate, acetoacetate, succinate, crotonate, butyrate, isobutyrate, malonate, and/or hexanoate, the method comprising culturing any non-naturally occurring microbial organism described herein under conditions and for a sufficient period of time to produce the acetyl-CoA derived product. In some embodiments, carbon flux is increased through (R)-3HB—CoA, (R)-3HB-Ald and acetyl-CoA. In some embodiments, the carbon flux is increased through (R)-3HB—CoA and acetyl-CoA. In some embodiments, the carbon flux is increased through (R)-3HB-Ald, or through (R)-3HB-Ald and acetyl-CoA. In some embodiments, the acetyl-CoA derived product is 1,3-BDO. In some embodiments, the acetyl-CoA derived product is (R)-1,3-BDO.

### CERTAIN EMBODIMENTS

Provided herein is a non-naturally occurring microbial organism having eliminated, substantially eliminated, or decreased production of at least one carboxylate by-product, wherein: the non-naturally occurring microbial organism comprises at least one carboxylate by-product recycling loop and at least one acetyl-CoA derived product pathway; the at least one carboxylate by-product is generated from at least one acyl-CoA precursor or intermediate of the acetyl-CoA derived product pathway; and the carboxylate by-product recycling loop converts the carboxylate by-product back to the acyl-CoA precursor or intermediate of the acetyl-CoA derived product pathway. In some embodiments, the carboxylate by-product recycling loop comprises an acyl-CoA synthetase (ACS) or a homolog thereof, or comprises at least one exogenous nucleic acid encoding an acyl-CoA synthetase (ACS) or homolog thereof, wherein the ACS or homolog thereof has activity to convert the carboxylate by-product back to the acyl-CoA precursor or intermediate of the acetyl-CoA derived product pathway. In some embodiments, the ACS or homolog thereof has activity corresponding to EC 6.2.1.-, EC 6.2.1.1, and/or EC 6.2.2.2. In some embodiments, the ACS or homolog thereof is heterologous to the microbial organism and is expressed or overexpressed in the microbial organism, or wherein the ACS or homolog thereof is native to the microbial organism and is overexpressed in the microbial organism. In some embodiments, the ACS comprises the amino acid sequence set forth in SEQ ID NO: 1, or is a variant or homolog of an ACS comprising the amino acid sequence set forth in SEQ ID NO: 1, or is a variant or homolog comprising at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%, sequence identity to SEQ ID NO: 1. In some embodiments, the enzymatic reaction catalyzed by the ACS or the homolog thereof results in the formation of AMP. In some embodiments, the ACS or homolog thereof has activity to convert a short-chain carboxylate to an acyl-CoA precursor or intermediate of the acetyl-CoA derived product pathway. In some embodiments, the at least one carboxylate by-product is a short-chain carboxylate. In some embodiments, the short-chain carboxylate is a C2-C4, a C2-C6, or a C2-C8 carboxylate. In some embodiments, the non-naturally occurring microbial organism has eliminated, substantially eliminated, or decreased production of at least one, at least, two, at least three, at least four, at least five, at least six, at least seven, at least eight, at least nine, or at least 10 carboxylate by-products. In some embodiments, the carboxylate by-product is one or more of acetate, acetoacetate, succinate, crotonate, butyrate, isobutyrate, 3-hydroxybutryate (3HB), (S)-3HB, (R)-3HB, malonate, and/or hexanoate. In some embodiments, the carboxylate by-product is 3-hydroxybutyrate (3HB) or (R)-3HB. In some embodiments, the carboxylate by-products are (i) 3HB or (R)-3HB; and (ii) acetate. In some embodiments, the non-naturally occurring microbial organism has increased or enhanced carbon flux through at least one acyl-CoA precursor or intermediate. In some embodiments, the acyl-CoA precursor or intermediate is one or more of acetyl-CoA, acetoacetyl-CoA, succinyl-CoA, crotonyl-CoA, butyryl-CoA, isobutyryl-CoA, 3HB—CoA, (S)-3HB—CoA, (R)-3HB—CoA, malonyl-CoA, and/or hexanoyl-CoA. In some embodiments, the non-naturally occurring microbial organism has increased or enhanced carbon flux through (i) 3HB—CoA or (R)-3HB—CoA; or (ii) acetyl-CoA; or (iii) both (i) and (ii). In some embodiments, the at least one carboxylate by-product recycling loop is any one or more of a 3HB recycling loop, an (R)-3HB recycling loop, an (S)-3HB recycling loop, an acetate recycling loop, an acetoacetate recycling loop, a succinate recycling loop, a crotonate recycling loop, a butyrate recycling loop, an isobutyrate recycling loop, a malonate recycling loop, and/or a hexanoate recycling loop. In some embodiments, the non-naturally occurring microbial organism has increased production of, or produces an increased amount, titer, and/or yield of, at least one acetyl-CoA derived product. In some embodiments, the acetyl-CoA derived product is one or more of 1,3-butanediol (1,3-BDO), (3R)-hydroxybutyl (3R)-hydroxybutyrate, methacrylic acid (MAA), MAA ester, butyrate, butanol, propane, formate, hexamethylenediamine (HMD), caprolactam (CPL), adipate (adipic acid), 6-aminocaproic acid (6-ACA), and/or 1,6-hexanediol (HDO). In some embodiments, the non-naturally occurring microbial organism further comprises a 3HB to 3-hydroxybutyraldehyde (3HBAld) conversion step, and/or an (R)-3HB to (R)-3HBAld conversion step. In some embodiments, the 3HB to 3HBAld conversion step or the (R)-3HB to (R)-3HBAld conversion step comprises a carboxylic acid reductase (CAR), or a variant or a homolog thereof, or comprises at least one exogenous nucleic acid encoding a CAR or a variant or a homolog thereof. In some embodiments, the CAR or variant or homolog thereof has activity characterized by EC 1.2.1.- and/or EC 1.2.1.30. In some embodiments, the CAR or variant or homolog thereof has activity to convert 3HB to 3HBAld and/or has activity to convert (R)-3HB to (R)-3HBAld. In some embodiments, the CAR or variant or homolog thereof is heterologous to the microbial organism and is expressed or overexpressed in the microbial organism, or wherein the CAR or variant or homolog thereof is native to the microbial organism and is overexpressed in the microbial organism. In some embodiments, the CAR comprises the amino acid sequence set forth in SEQ ID NO: 2, or SEQ ID NO: 3, or is a variant or homolog thereof, or wherein the CAR is a variant or homolog comprising at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%, sequence identity to SEQ ID NO: 2 or SEQ ID NO: 3. In some embodiments, the 3HB to 3HBAld conversion step or the (R)-3HB to (R)-3HBAld conversion step reduces the amount, production, titer, yield, and/or productivity of 3HB or (R)-3HB by-product; and/or increases or enhances the carbon flux through 3HBAld or (R)-3HBAld, and/or increases the amount, production, titer, yield, and/or productivity of an acetyl-CoA derived product that is 1,3-BDO or (R)-1,3-BDO. In some embodiments, the non-naturally occurring microbial organism has eliminated, substantially eliminated, or decreased production of at least one carboxylate by-product, and/or has increased production, titer, yield and/or productivity of at least one acetyl-CoA derived product, and/or has increased or enhanced carbon flux through an acyl-CoA precursor or intermediate, compared to a microbial organism that lacks a carboxylate by-product recycling loop. In some embodiments, the non-naturally occurring microbial organism has eliminated, substantially eliminated, or decreased production of at least one carboxylate by-product, and/or has increased production, titer, yield and/or productivity of at least one acetyl-CoA derived product, and/or has increased or enhanced carbon flux through an acyl-CoA precursor or intermediate, compared to a microbial organism that does not express an ACS or a homolog or variant thereof and/or compared to a microbial organism with wild-type expression of an ACS or a homolog or a variant thereof. In some embodiments, the non-naturally occurring microbial organism has eliminated, substantially eliminated, or decreased production of 3HB and/or (R)-3HB, and/or has increased or enhanced carbon flux through 3HBAld and/or (R)-3HBAld, and/or has increased production, titer, yield and/or productivity of at least one acetyl-CoA derived product having a 3HB—CoA, (R)-3HB—CoA, 3HBAld, and/or (R)-3HBAld intermediate or precursor, compared to a microbial organism that does not comprise a 3HB to 3HBAld conversion step or a (R)-3HB to (R)-3HBAld conversion step, and/or compared to a microbial organism that does not express a CAR or a variant or homolog thereof, and/or compared to a microbial organism with wild-type expression of a CAR or a variant or a homolog thereof. In some embodiments: the non-naturally occurring microbial organism has eliminated, substantially eliminated, or decreased production of 3HB, (R)-3HB, and/or acetate by-products; the non-naturally occurring microbial organism has increased or enhanced carbon flux through 3HB—CoA, (R)-3HB—CoA, acetyl-CoA, 3HBAld, and/or (R)-3HBAld; and the non-naturally occurring microbial organism has increased production, titer, yield and/or productivity of 1,3-BDO and/or (R)-1,3-BDO. In some embodiments, the at least one carboxylate by-product recycling loop is any one or more of a 3HB recycling loop, an (R)-3HB recycling loop, an (S)-3HB recycling loop, an acetate recycling loop, an acetoacetate recycling loop, a succinate recycling loop, a crotonate recycling loop, a butyrate recycling loop, an isobutyrate recycling loop, a malonate recycling loop, and/or a hexanoate recycling loop; the at least one acetyl-CoA derived product pathway is one or more of a 1,3-butanediol (1,3-BDO), (R)-1,3-BDO, (3R)-hydroxybutyl (3R)-hydroxybutyrate, methacrylic acid (MAA), MAA ester, butyrate, butanol, propane, formate, hexamethylenediamine (HMD), caprolactam (CPL), adipate (adipic acid), 6-aminocaproic acid (6-ACA), and/or 1,6-hexanediol (HDO) product pathway; the at least one carboxylate by-product is one or more of acetate, acetoacetate, succinate, crotonate, butyrate, isobutyrate, 3-hydroxybutryate (3HB), (S)-3HB, (R)-3HB, malonate, and/or hexanoate; and the at least one acyl-CoA precursor or intermediate is one or more of acetyl-CoA, acetoacetyl-CoA, succinyl-CoA, crotonyl-CoA, butyryl-CoA, isobutyryl-CoA, (S)-3HB—CoA, (R)-3HB—CoA, malonyl-CoA, and/or hexanoyl-CoA. In some embodiments, the non-naturally occurring microbial organism comprises an (R)-3HB recycling loop and an (R)-1,3-BDO product pathway, wherein the microbial organism has eliminated, substantially eliminated, or decreased production of (R)-3HB by-product. In some embodiments, the (R)-3HB recycling loop comprises an acyl-CoA synthetase (ACS) or a homolog thereof, or comprises at least one exogenous nucleic acid encoding an acyl-CoA synthetase (ACS) or homolog thereof, wherein: the ACS or homolog thereof comprises activity to convert (R)-3HB to (R)-3HB—CoA; and the ACS or homolog thereof is heterologous to the non-naturally occurring microbial organism and is expressed or overexpressed, or is native to the non-naturally occurring microbial organism and is overexpressed. In some embodiments, the microbial organism has enhanced or increased carbon flux through (R)-3HB—CoA, and/or has increased production, titer, yield, and/or productivity of (R)-1,3-BDO. In some embodiments, the microbial organism has enhanced or increased carbon flux through (R)-3HB—CoA, and/or increased production, titer, yield, and/or productivity of (R)-1,3-BDO, compared to a microbial organism without an (R)-3HB recycling loop and/or compared to a microbial organism without expression of an ACS or homolog thereof, and/or compared to a microbial organism with wild-type expression of an ACS or homolog thereof. In some embodiments: the microbial organism additionally comprises an acetate recycling loop, wherein the microbial organism has eliminated, substantially eliminated, or decreased production of acetate by-product and/or has increased or enhanced carbon flux through acetyl-CoA; and/or the microbial organism additionally comprises an (R)-3HB to (R)-3HBAld conversion step, wherein the microbial organism optionally has eliminated, substantially eliminated, or decreased production of (R)-3HB by-product and/or increased or enhanced carbon flux through (R)-3HBAld. In some embodiments, the (R)-3HB to (R)-3HBAld conversion step comprises a carboxylic acid reductase (CAR), or a variant or a homolog thereof, or comprises at least one exogenous nucleic acid encoding a CAR or a variant or a homolog thereof, and wherein the CAR or variant or homolog thereof is heterologous to the non-naturally occurring microbial organism and is expressed or overexpressed, or the CAR or variant or homolog thereof is native to the non-naturally occurring microbial organism and is overexpressed. In some embodiments, the non-naturally occurring microbial organism comprises more than one carboxylate by-product recycling loop, and the more than one carboxylate recycling loops are additive and/or synergistic, and result in a greater reduction of carboxylate by-products, and/or a greater increase in carbon flux through the acyl-CoA intermediate or precursor, and/or an increased production of an acetyl-CoA derived product, compared to a microbial organism comprising one carboxylate by-product recycling loop. In some embodiments, the at least one carboxylate by-product recycling loop and the 3HB to 3HBAld conversion step are additive and/or synergistic, and result in a greater reduction of carboxylate by-products, and/or a greater increase in carbon flux through one or more intermediates or precursors of the acetyl-CoA derived product pathway, and/or an increased production of an acetyl-CoA derived product, compared to a microbial organism comprising only the at least one carboxylate by-product recycling loop or only the 3HB to 3HBAld conversion step. In some embodiments, (i) the 3HB recycling loop, and (ii) the one or more of an acetate recycling loop, an acetoacetate recycling loop, a succinate recycling loop, a crotonate recycling loop, a butyrate recycling loop, an isobutyrate recycling loop, a malonate recycling loop, and/or a hexanoate recycling loop are additive and/or synergistic, and result in a greater reduction of carboxylate by-products, and/or a greater increase in carbon flux through one or more intermediates or precursors of the acetyl-CoA derived product pathway, and/or an increased production of an acetyl-CoA derived product, compared to a microbial organism comprising only (i) or (ii). In some embodiments, (i) the 3HB recycling loop; (ii) the one or more of an acetate recycling loop, an acetoacetate recycling loop, a succinate recycling loop, a crotonate recycling loop, a butyrate recycling loop, an isobutyrate recycling loop, a malonate recycling loop, and/or a hexanoate recycling loop; and/or (iii) the 3HB to 3HBAld conversion step, are additive and/or synergistic, and result in a greater reduction of carboxylate by-products, and/or a greater increase in carbon flux through one or more intermediates or precursors of the acetyl-CoA derived product pathway, and/or an increased production of an acetyl-CoA derived product, compared to a microbial organism comprising only (i), only (ii), only (iii), or only two of (i), (ii), and (iii).

Also provided herein is a non-naturally occurring microbial organism having eliminated, substantially eliminated, or decreased 3-hydroxybutyrate (3HB) by-product, wherein the non-naturally occurring microbial organism comprises (i) an acetyl Coenzyme A (acetyl-CoA) derived product pathway; (ii) an enhanced or increased carbon flux through 3-hydroxybutyryl Coenzyme-A (3HB—CoA); and (iii) a 3HB recycling loop. In some embodiments, the 3HB recycling loop comprises at least one exogenous nucleic acid encoding an acyl-CoA synthetase (ACS) or homolog thereof, or comprises an acyl-CoA synthetase or homolog thereof. In some embodiments, said ACS or homolog thereof is expressed in a sufficient amount to reduce production of 3HB. In some embodiments, said ACS or homolog thereof is expressed in a sufficient amount to increase the yield of the acetyl-CoA derived product. In some embodiments, the at least one exogenous nucleic acid is a heterologous nucleic acid; or the ACS is heterologous to the non-naturally occurring microbial organism and is expressed or overexpressed; or the ACS is native to the non-naturally occurring microbial organism and is overexpressed. In some embodiments, the non-naturally occurring microbial organism has reduced concentration of intracellular 3HB. In some embodiments, the non-naturally occurring microbial organism has reduced concentration of intracellular 3HB compared to a microbial organism without a 3HB recycling loop. In some embodiments, the non-naturally occurring microbial organism has reduced concentration of intracellular 3HB compared to a microbial organism with wild-type expression of ACS. In some embodiments, the non-naturally occurring microbial organism has increased yield of the acetyl-CoA derived product. In some embodiments, the non-naturally occurring microbial organism has increased yield of the acetyl-CoA derived product compared to a microbial organism without a 3HB recycling loop. In some embodiments, the non-naturally occurring microbial organism has increased yield of the acetyl-CoA derived product compared to a microbial organism with wild-type expression of ACS. In some embodiments, the non-naturally occurring microbial organism further comprises one or more of an acetate recycling loop, an acetoacetate recycling loop, a succinate recycling loop, a crotonate recycling loop, a butyrate recycling loop, an isobutyrate recycling loop, a malonate recycling loop, and/or a hexanoate recycling loop. In some embodiments: the acetate recycling loop comprises at least one exogenous nucleic acid encoding an acyl-CoA synthetase (ACS) or homolog thereof, or comprises an acyl-CoA synthetase or homolog thereof; the ACS or homolog thereof catalyzes the conversion of acetate by-product to acetyl-CoA; the non-naturally occurring microbial organism has reduced concentration of intracellular acetate compared to a microbial organism without an acetate recycling loop; the non-naturally occurring microbial organism has enhanced or increased carbon flux through acetyl-CoA; and/or the non-naturally occurring microbial organism has increased production of an acetyl-CoA derived product. In some embodiments, the non-naturally occurring microbial organism further comprises a 3HB to 3HBAld conversion step or an R-3HB to R-3HAld conversion step. In some embodiments, the 3HB to 3HBAld conversion step or the R-3HB to R-3HAld conversion step comprises an exogenous nucleic acid encoding a CAR or a variant or a homolog thereof, or comprises a CAR or a variant or a homolog thereof, and wherein the CAR or variant or homolog thereof is heterologous to the non-naturally occurring microbial organism and is expressed or overexpressed, or the CAR or variant or homolog thereof is native to the non-naturally occurring microbial organism and is overexpressed. In some embodiments, the non-naturally occurring microbial organism produces reduced amounts of 3HB or R-3HB, and/or has increased or enhanced carbon flux through 3HBAld or R-3HBAld, and/or has increased production, titer, yield, and/or productivity of an acetyl-CoA derived product, compared to a microbial organism without a 3HB to 3HBAld or R-3HB to R-3HBAld conversion step. In some embodiments, the acetyl-CoA derived product is 1,3-BDO, optionally an R-1,3 BDO, (3R)-hydroxybutyl (3R)-hydroxybutyrate, butyrate, butanol, propane, formate, isopropanol, MAA, an MAA ester, HMD, CPL, adipate, 6ACA, or HDO. In some embodiments, non-naturally occurring microbial organism further comprises a 1,3-BDO, optionally an R-1,3 BDO, an (3R)-hydroxybutyl (3R)-hydroxybutyrate, a butyrate, a butanol, a propane, a formate, an isopropanol, an MAA, an MAA ester, an HMD, a CPL, an adipate, a 6ACA, or an HDO pathway. In some embodiments, said microbial organism comprises a 1,3-BDO, optionally an R-1,3 BDO, pathway. In some embodiments, said 1,3-BDO pathway comprises: (a) AcAc—CoA reductase (a CoA-dependent, aldehyde forming oxidoreductase); 3-oxobutyraldehyde reductase (a ketone reducing/alcohol forming oxidoreductase); 3HB-Ald reductase (an aldehyde reducing/alcohol forming oxidoreductase), acetaldehyde-alcohol dehydrogenase, 1,3-BDO or 1,3-butylene glycol dehydrogenase, or ADH; AcAc—COA reductase (a CoA-dependent, alcohol forming oxidoreductase); 3-oxobutyraldehyde reductase (an aldehyde reducing/alcohol forming oxidoreductase); 4-hydroxy-2-butanone reductase (a ketone reducing/alcohol forming oxidoreductase); AcAc—COA reductase (ketone reducing/alcohol forming oxidoreductase); 3HB—CoA reductase (a CoA-dependent, aldehyde forming oxidoreductase), ALD, or acetaldehyde-alcohol dehydrogenase; 3HB—CoA reductase (an alcohol forming, CoA dependent oxidoreductase); 3HB—CoA transferase or a 3HB—CoA hydrolase; 3HB dehydrogenase (a ketone reducing, alcohol forming oxidoreductase); AcAc—CoA transferase, AcAc—COA hydrolase, AcAc—COA synthetase; acetoacetate reductase (an aldehyde forming oxidoreductase); and a Ac-COA thiolase; (b) AcAc—COA thiolase; HBD, optionally a R—HBD, or AcAc—COA reductase (ketone reducing/alcohol forming oxidoreductase); butyraldehyde dehydrogenase or acetaldehyde-alcohol dehydrogenase; acetaldehyde-alcohol dehydrogenase; PTB; and BUK; (c) AcAc—COA thiolase; HBD, optionally R—HBD; (R)-3HB—CoA reductase; (R)-3HB-Ald reductase; Ac-COA carboxylase; AcAc—COA synthase; HBD, optionally S—HBD; and 3HB—CoA epimerase; (d) 4HB—CoA dehydratase (a hydro-lyase); CRT (a hydro-lyase); 3HB—CoA reductase (a Co-A dependent, aldehyde forming oxidoreductase); 3HB-Ald reductase (an alcohol forming oxidoreductase); and 3HB—CoA reductase (an alcohol forming oxidoreductase); and/or (e) a SucCoA transferase, SucCoA hydrolase, and/or a SucCoA synthetase (or SucCoA ligase); a SucCoA reductase (aldehyde forming); a 4HB dehydrogenase; a 4HB kinase; a phosphotrans-4-hydroxybutyrylase; a Succ reductase; a SucCoA reductase (alcohol forming); a 4HB—CoA transferase, or a 4HB—CoA synthetase, or a 4HB—CoA ligase; an AKG decarboxylase; a 4HB—CoA dehydratase; a CRT; a 3HB—CoA reductase (aldehyde forming); a 3-hydroxybutanal reductase; a 3HB—CoA reductase (alcohol forming); a 3HB—CoA hydrolase and/or a 3HB—CoA transferase; an alcohol forming reductase; a glutamate dehydrogenase and/or a glutamate transaminase; a glutamate decarboxylase; a 4-aminobutyrate dehydrogenase and/or a 4-aminobutyrate transaminase; and an AKGD. In some embodiments, said microbial organism comprises an (3R)-hydroxybutyl (3R)-hydroxybutyrate pathway. In some embodiments, said (3R)-hydroxybutyl (3R)-hydroxybutyrate pathway comprises: (a) AcAc—COA thiolase; HBD, optionally R—HBD; (R)-3HB—CoA reductase; (R)-3HB-Ald reductase; Ac-COA carboxylase; AcAc—COA synthase; HBD, optionally S—HBD; 3HB—CoA epimerase; and (3R)-hydroxybutyryl-CoA:(R)-1,3-butanediol alcohol transferase; and/or (b) AcAc—COA thiolase; HBD, optionally a R—HBD, or AcAc—COA reductase (ketone reducing/alcohol forming oxidoreductase); butyraldehyde dehydrogenase or acetaldehyde-alcohol dehydrogenase; acetaldehyde-alcohol dehydrogenase; PTB; BUK; and (3R)-hydroxybutyryl-CoA:(R)-1,3-butanediol alcohol transferase. In some embodiments, said microbial organism comprises a butyrate, butanol, propane, and/or formate pathway. In some embodiments, said butyrate, butanol, propane, and/or formate pathway comprises: AcAc—COA thiolase; HBD, optionally, S—HBD; CRT; BCD; ALDH; AHR; PTB; BUK; and ADO. In some embodiments, said microbial organism comprises an isopropanol pathway. In some embodiments, said isopropanol pathway comprises: CRT; HBD; acetoacetyl-CoA synthetase, acetyl-CoA:acetoacetate-CoA transferase, and/or acetoacetyl-CoA hydrolase; acetoacetate decarboxylase; and acetone reductase. In some embodiments, said microbial organism comprises an MAA pathway. In some embodiments, said MAA pathway comprises: (a) AcAc—CoA thiolase; AcAc—COA reductase (ketone reducing/alcohol forming oxidoreductase); 3HB—CoA mutase; 2HIB—CoA dehydratase; methacrylyl-CoA transferase, methacrylyl-CoA hydrolase and/or methacrylyl-CoA synthetase; 4HB—CoA dehydratase; vinylacetyl-CoA A-isomerase; and/CRT; and/or (b) AcAc—COA thiolase; AcAc—COA reductase (ketone reducing/alcohol forming oxidoreductase); methacrylyl-CoA transferase, methacrylyl-CoA hydrolase and/or methacrylyl-CoA synthetase; CRT; butyryl-CoA dehydrogenase; isobutyryl-CoA mutase; and isobutyryl-CoA dehydrogenase. In some embodiments, said microbial organism comprises a MAA ester pathway. The non-naturally occurring microbial organism of claim 66, wherein said MAA ester pathway comprises: (a) a methacrylyl-CoA transferase or a methacrylyl-CoA synthetase; and an alcohol transferase; and/or (b) or a methacrylate ester-forming enzyme.

Also provided herein is a non-naturally occurring microbial organism having eliminated, substantially eliminated, or decreased 3-hydroxybutyrate (3HB) by-product, wherein the non-naturally occurring microbial organism comprises (i) at least one an acetyl Coenzyme A (acetyl-CoA) derived product pathway; (ii) an enhanced or increased carbon flux through 3-hydroxybutyraldehyde (3HBAld); and (iii) a 3HB to 3HBAld conversion step. In some embodiments, the 3HB to 3HBAld conversion step comprises an exogenous nucleic acid encoding a CAR or a variant or a homolog thereof, or comprises a CAR or a variant or a homolog thereof. In some embodiments, the CAR or variant or homolog thereof has activity characterized by EC 1.2.1.- and/or EC 1.2.1.30. In some embodiments, the CAR or variant or homolog thereof has activity to convert 3HB to 3HBAld and/or has activity to convert (R)-3HB to (R)-3HBAld. In some embodiments, the CAR or variant or homolog thereof is heterologous to the microbial organism and is expressed or overexpressed in the microbial organism, or wherein the CAR or variant or homolog thereof is native to the microbial organism and is overexpressed in the microbial organism. In some embodiments, the CAR comprises the amino acid sequence set forth in SEQ ID NO: 2 or SEQ ID NO: 3, or is a variant or homolog thereof, or wherein the CAR is a variant or homolog comprising at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%, sequence identity to SEQ ID NO: 2 or SEQ ID NO: 3. In some embodiments, the 3HB to 3HBAld conversion step reduces the amount, production, titer, yield, and/or productivity of 3HB by-product; and/or increases or enhances the carbon flux through 3HBAld; and/or increases the amount, production, titer, yield, and/or productivity of an acetyl-CoA derived product. In some embodiments, the acetyl-CoA derived product is 1,3-BDO. In some embodiments, the non-naturally occurring microbial organism has eliminated, substantially eliminated, or decreased production of 3HB by-product, and/or has increased production, titer, yield and/or productivity of at least one acetyl-CoA derived product, and/or has increased or enhanced carbon flux through 3HBAld, compared to a microbial organism that lacks a 3HB to 3HBAld conversion step. In some embodiments, the non-naturally occurring microbial organism has eliminated, substantially eliminated, or decreased production of 3HB, and/or has increased production, titer, yield and/or productivity of at least one acetyl-CoA derived product, and/or has increased or enhanced carbon flux through 3HBAld, compared to a microbial organism that does not express a CAR or a homolog or variant thereof, and/or compared to a microbial organism with wild-type expression of a CAR or a homolog or a variant thereof. In some embodiments, the at least one acetyl-CoA derived product pathway comprises a 3HBAld intermediate or precursor. In some embodiments, the 3HB is R-3HB and the 3HBAld is R-3HBAld. In some embodiments, the at least one acetyl-CoA derived product pathway is one or more of a 1,3-BDO, an R-1,3 BDO, an (3R)-hydroxybutyl (3R)-hydroxybutyrate, a butyrate, a butanol, a propane, a formate, an isopropanol, an MAA, or an MAA ester product pathway. In some embodiments, the acetyl-CoA derived product pathway is a 1,3-BDO, optionally an R-1,3-BDO, product pathway. In some embodiments, the 1,3-BDO or R-1,3-BDO product pathway comprises: (a) AcAc—COA reductase (a CoA-dependent, aldehyde forming oxidoreductase); 3-oxobutyraldehyde reductase (a ketone reducing/alcohol forming oxidoreductase); 3HB-Ald reductase (an aldehyde reducing/alcohol forming oxidoreductase), acetaldehyde-alcohol dehydrogenase, 1,3-BDO or 1,3-butylene glycol dehydrogenase, or ADH; AcAc—COA reductase (a CoA-dependent, alcohol forming oxidoreductase); 3-oxobutyraldehyde reductase (an aldehyde reducing/alcohol forming oxidoreductase); 4-hydroxy-2-butanone reductase (a ketone reducing/alcohol forming oxidoreductase); AcAc—COA reductase (ketone reducing/alcohol forming oxidoreductase); 3HB—CoA reductase (a CoA-dependent, aldehyde forming oxidoreductase), ALD, or acetaldehyde-alcohol dehydrogenase; 3HB—CoA reductase (an alcohol forming, CoA dependent oxidoreductase); 3HB—CoA transferase or a 3HB—CoA hydrolase; 3HB dehydrogenase (a ketone reducing, alcohol forming oxidoreductase); AcAc—CoA transferase, AcAc—COA hydrolase, AcAc—COA synthetase; acetoacetate reductase (an aldehyde forming oxidoreductase); and a Ac-COA thiolase; (b) AcAc—COA thiolase; HBD, optionally a R—HBD, or AcAc—COA reductase (ketone reducing/alcohol forming oxidoreductase); butyraldehyde dehydrogenase or acetaldehyde-alcohol dehydrogenase; acetaldehyde-alcohol dehydrogenase; PTB; and BUK; (c) AcAc—COA thiolase; HBD, optionally R—HBD; (R)-3HB—CoA reductase; (R)-3HB-Ald reductase; Ac-COA carboxylase; AcAc—COA synthase; HBD, optionally S—HBD; and 3HB—CoA epimerase; (d) 4HB—CoA dehydratase (a hydro-lyase); CRT (a hydro-lyase); 3HB—CoA reductase (a Co-A dependent, aldehyde forming oxidoreductase); 3HB-Ald reductase (an alcohol forming oxidoreductase); and 3HB—CoA reductase (an alcohol forming oxidoreductase); and/or (e) a SucCoA transferase, SucCoA hydrolase, and/or a SucCoA synthetase (or SucCoA ligase); a SucCoA reductase (aldehyde forming); a 4HB dehydrogenase; a 4HB kinase; a phosphotrans-4-hydroxybutyrylase; a Succ reductase; a SucCoA reductase (alcohol forming); a 4HB—CoA transferase, or a 4HB—CoA synthetase, or a 4HB—CoA ligase; an AKG decarboxylase; a 4HB—CoA dehydratase; a CRT; a 3HB—CoA reductase (aldehyde forming); a 3-hydroxybutanal reductase; a 3HB—CoA reductase (alcohol forming); a 3HB—CoA hydrolase and/or a 3HB—CoA transferase; an alcohol forming reductase; a glutamate dehydrogenase and/or a glutamate transaminase; a glutamate decarboxylase; a 4-aminobutyrate dehydrogenase and/or a 4-aminobutyrate transaminase; and an AKGD. In some embodiments, the non-naturally occurring microbial organism is a species of bacteria, yeast, or fungus. In some embodiments, said non-naturally occurring microbial organism is in a substantially anaerobic culture medium, or is in a microaerobic or substantially microaerobic culture medium.

Also provided herein is a method for increasing the production of 1,3-BDO, the method comprising culturing the non-naturally occurring microbial organism of any one of claims 1-84, under conditions and for a sufficient period of time to increase the availability or production or titer or yield of 1,3-BDO. In some embodiments, the 1,3-BDO is (R)-1,3-BDO.

Also provided herein a method for increasing the production of 3R-hydroxybutyric acid-3R-hydroxybutryrate, the method comprising culturing the non-naturally occurring microbial organism of any one of claims 1-84, under conditions and for a sufficient period of time to increase the availability or production or titer or yield of 3R-hydroxybutyric acid-3R-hydroxybutryrate.

Also provided herein is a method for increasing the production of butyrate, butanol, propane, and/or formate, the method comprising culturing any one of the non-naturally occurring microbial organism provided herein, under conditions and for a sufficient period of time to increase the availability of butyrate, butanol, propane, and/or formate.

Also provided herein is a method for increasing the production of isopropanol, the method comprising culturing any one of the non-naturally occurring microbial organism provided herein, under conditions and for a sufficient period of time to increase the availability or production or titer or yield of isopropanol.

Also provided herein is a method for increasing the production of MAA, the method comprising culturing any one of the non-naturally occurring microbial organism provided herein, under conditions and for a sufficient period of time to increase the availability or production or titer or yield of MAA.

Also provided herein is a method for increasing the production of an MAA ester, the method comprising culturing any one of the non-naturally occurring microbial organism provided herein, under conditions and for a sufficient period of time to increase the availability or production or titer or yield of an MAA ester.

Also provided herein is a method for enhancing or increasing the carbon flux through 3HB—CoA in a non-naturally occurring microbial organism to increase the production, titer and/or yield of an acetyl-CoA derived product and to eliminate, substantially eliminate, or reduce the production of 3HB by-product, the method comprising culturing any one of the non-naturally occurring microbial organism provided herein under conditions and for a sufficient period of time to produce the acetyl-CoA derived product. In some embodiments, the acetyl-CoA derived product is 1,3-BDO, R-1,3 BDO, S-1,3-BDO, (3R)-hydroxybutyl (3R)-hydroxybutyrate, butyrate, butanol, propane, formate, MAA, MAA ester, HMD, CPL, adipate, 6ACA, or HDO. In some embodiments, the acetyl-CoA derived product comprises 1,3-BDO, optionally R-1,3-BDO. In some embodiments, the acetyl-CoA derived product comprises 3R-hydroxybutyric acid-3R-hydroxybutryrate. In some embodiments, the acetyl-CoA derived product comprises butyrate, butanol, propane, and/or formate. In some embodiments, the acetyl-CoA derived product comprises isopropanol. In some embodiments, the acetyl-CoA derived product comprises MAA. In some embodiments, the acetyl-CoA derived product comprises an MAA ester. In some embodiments, the acetyl-CoA derived product comprises HMD. In some embodiments, the acetyl-CoA derived product comprises CPL. In some embodiments, the acetyl-CoA derived product comprises adipate. In some embodiments, the acetyl-CoA derived product comprises 6ACA. In some embodiments, the acetyl-CoA derived product comprises HDO.

Also provided herein is a method for increasing the production, titer, yield, and/or productivity of an acetyl-CoA derived product, the method comprising culturing any one of the non-naturally occurring microbial organism provided herein.

Also provided herein is a non-naturally occurring microbial organism, comprising: an acetyl Coenzyme A (acetyl-CoA) derived product pathway; a 3HB recycling loop; and a 3HB to 3HBAld conversion step. In some embodiments: the acetyl-CoA derived product pathway is one or more of a 1,3-BDO, an R-1,3 BDO, an S-1,3-BDO, a (3R)-hydroxybutyl (3R)-hydroxybutyrate, a butyrate, a butanol, a propane, a formate, an MAA, or an MAA ester product pathway; the 3HB recycling loop comprises at least one exogenous nucleic acid encoding an acyl-CoA synthetase (ACS) or homolog thereof, or comprises an acyl-CoA synthetase or homolog thereof; and the 3HB to 3HBAld conversion step comprises a carboxylic acid reductase (CAR), or a variant or a homolog thereof, or comprises at least one exogenous nucleic acid encoding a CAR or a variant or a homolog thereof. In some embodiments: the non-naturally occurring microbial organism has eliminated, substantially eliminated, or decreased 3HB by-product; and/or the non-naturally occurring microbial organism has increased or enhanced carbon flux through 3HB—CoA and/or 3HB Ald; and/or the non-naturally occurring microbial organism has increased production, titer, yield, and/or productivity of an acetyl-CoA derived product; compared to a microbial organism that does not have a 3HB recycling loop and/or a 3HB to 3HBAld conversion step. In some embodiments, non-naturally occurring microbial organism further comprises an acetate recycling loop, wherein the acetate recycling loop comprises at least one exogenous nucleic acid encoding an acyl-CoA synthetase (ACS) or homolog thereof, or comprises an acyl-CoA synthetase or homolog thereof. In some embodiments, the non-naturally occurring microbial organism further has eliminated, substantially eliminated, or decreased acetate by-product; and/or further has increased carbon flux through acetyl-CoA; and/or further has increased production, titer, yield, and/or productivity of an acetyl-CoA derived product, compared to microbial organism that does not have any one or more of: a 3HB recycling loop, a 3HB to 3HBAld conversion step, and/or an acetate recycling loop. In some embodiments, the acetyl-CoA derived product pathway is a 1,3-BDO product pathway, or an R-1,3-BDO product pathway. In some embodiments, the 1,3-BDO or R-1,3-BDO product pathway comprises: (a) AcAc—COA reductase (a CoA-dependent, aldehyde forming oxidoreductase); 3-oxobutyraldehyde reductase (a ketone reducing/alcohol forming oxidoreductase); 3HB-Ald reductase (an aldehyde reducing/alcohol forming oxidoreductase), acetaldehyde-alcohol dehydrogenase, 1,3-BDO or 1,3-butylene glycol dehydrogenase, or ADH; AcAc—COA reductase (a CoA-dependent, alcohol forming oxidoreductase); 3-oxobutyraldehyde reductase (an aldehyde reducing/alcohol forming oxidoreductase); 4-hydroxy-2-butanone reductase (a ketone reducing/alcohol forming oxidoreductase); AcAc—COA reductase (ketone reducing/alcohol forming oxidoreductase); 3HB—CoA reductase (a CoA-dependent, aldehyde forming oxidoreductase), ALD, or acetaldehyde-alcohol dehydrogenase; 3HB—CoA reductase (an alcohol forming, CoA dependent oxidoreductase); 3HB—CoA transferase or a 3HB—CoA hydrolase; 3HB dehydrogenase (a ketone reducing, alcohol forming oxidoreductase); AcAc—COA transferase, AcAc—COA hydrolase, AcAc—COA synthetase; acetoacetate reductase (an aldehyde forming oxidoreductase); and a Ac-COA thiolase; (b) AcAc—COA thiolase; HBD, optionally a R—HBD, or AcAc—COA reductase (ketone reducing/alcohol forming oxidoreductase); butyraldehyde dehydrogenase or acetaldehyde-alcohol dehydrogenase; acetaldehyde-alcohol dehydrogenase; PTB; and BUK; (c) AcAc—COA thiolase; HBD, optionally R—HBD; (R)-3HB—CoA reductase; (R)-3HB-Ald reductase; Ac-COA carboxylase; AcAc—COA synthase; HBD, optionally S—HBD; and 3HB—CoA epimerase; (d) 4HB—CoA dehydratase (a hydro-lyase); CRT (a hydro-lyase); 3HB—CoA reductase (a Co-A dependent, aldehyde forming oxidoreductase); 3HB-Ald reductase (an alcohol forming oxidoreductase); and 3HB—CoA reductase (an alcohol forming oxidoreductase); and/or (e) a SucCoA transferase, SucCoA hydrolase, and/or a SucCoA synthetase (or SucCoA ligase); a SucCoA reductase (aldehyde forming); a 4HB dehydrogenase; a 4HB kinase; a phosphotrans-4-hydroxybutyrylase; a Succ reductase; a SucCoA reductase (alcohol forming); a 4HB—CoA transferase, or a 4HB—CoA synthetase, or a 4HB—CoA ligase; an AKG decarboxylase; a 4HB—CoA dehydratase; a CRT; a 3HB—CoA reductase (aldehyde forming); a 3-hydroxybutanal reductase; a 3HB—CoA reductase (alcohol forming); a 3HB—CoA hydrolase or a 3HB—CoA transferase; an alcohol forming reductase; a glutamate dehydrogenase and/or a glutamate transaminase; a glutamate decarboxylase; a 4-aminobutyrate dehydrogenase and/or a 4-aminobutyrate transaminase; and an AKGD. In some embodiments, the expression of the 3HB recycling loop, the 3HB to 3HBAld conversion step, and the acetate recycling loop is additive and/or synergistic and can result in the increased production, titer, yield, and/or productivity of an acetyl-CoA derived product compared to microbial organism that does not have any one or more of: a 3HB recycling loop, a 3HB to 3HBAld conversion step, and an acetate recycling loop. In some embodiments, the expression of the 3HB recycling loop, the 3HB to 3HBAld conversion step, and the acetate recycling loop is additive and/or synergistic compared to microbial organism that does not have any two or more, or all of: a 3HB recycling loop, a 3HB to 3HBAld conversion step, and an acetate recycling loop. In some embodiments: the 3HB recycling loop and the acetate recycling loop are additive and/or synergistic; the 3HB recycling loop and the 3HB to 3HBAld conversion step are additive and/or synergistic; and/or the 3HB recycling loop, the acetate recycling loop, and the 3HB to 3HBAld conversion step are additive and/or synergistic. The non-naturally occurring microbial organism of claim 106, wherein the 3HB recycling loop and the 3HB to 3HB Ald conversion step are additive and/or synergistic, and result in a greater decrease in 3HB by-product, and/or a greater increase in carbon flux through 3HB—CoA and/or 3HBAld, and/or a greater increase in the production of an acetyl-CoA derived product, compared to a microbial organism that only comprises a 3HB recycling loop or that only comprises a 3HB to 3HBAld conversion step.

### BRIEF DESCRIPTION OF THE DRAWINGS

FIG. 1 depicts a biosynthetic pathway comprising 3HB—CoA and 3HB-Ald intermediates, and depicts the generation of 3HB side product from 3HB—CoA, as well as the reintroduction or recycling of 3HB back into the biosynthetic pathway by converting it back into 3HB—CoA and/or by converting it to 3HB-Ald. As shown in the figure, 3HB—CoA is converted into 3HB-Ald in the main biosynthetic pathway by an (A) aldehyde dehydrogenase (ALD); while 3HB side product (by-product) can be generated from 3HB—CoA by: (B) spontaneous hydrolysis; the activity of (C) a thioesterase (e.g., a CoA thiosesterase); the activity of (E) a 3-hydroxybutyryl-CoA transferase; or the activity of (F) a 3-hydroxybutyryl-CoA hydrolase. 3HB can be converted back into 3HB—CoA by (D) the activity of an acyl-CoA synthetase (ACS, also referred to herein as a CoA-ligase) or a variant or homolog thereof. 3HB also can be converted to 3HB-Ald by (G) the activity of a carboxylic acid reductase (CAR) or a variant or homolog thereof.

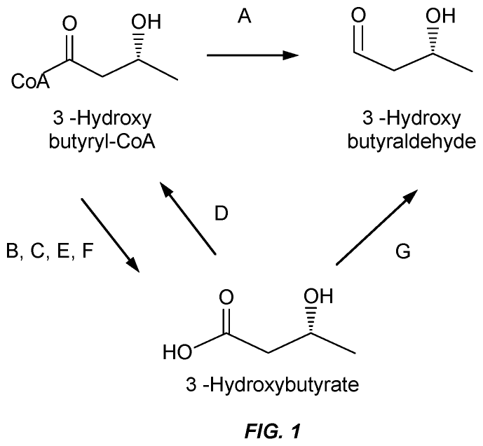

FIG. 2 depicts exemplary pathways from acetoacetyl-CoA (AcAc—COA) to 1,3-butanediol (1,3-BDO). The enzymatic steps are: (A) conversion of AcAc—COA to 3-oxobutyraldehyde by an acetoacetyl-CoA reductase (a CoA-dependent, aldehyde forming oxidoreductase); (B) conversion of 3-oxobutyraldehyde to 3-hydroxybutyraldehyde by a 3-oxobutyraldehyde reductase (a ketone reducing/alcohol forming oxidoreductase); (C) conversion of 3-hydroxybutyraldehyde to 1,3-butanediol by a 3-hydroxybutyraldehyde reductase (an aldehyde reducing/alcohol forming oxidoreductase; also known as an alcohol dehydrogenase); (D) conversion of acetoacetyl-CoA to 4-hydroxy-2-butanone by an acetoacetyl-CoA reductase (a CoA-dependent, alcohol forming oxidoreductase); (E) conversion of 3-oxobutyraldehyde to 4-hydroxy-2-butanone by a 3-oxobutyraldehyde reductase (an aldehyde reducing/alcohol forming oxidoreductase); (F) conversion of 4-hydroxy-2-butanone to 1,3-butanediol by a 4-hydroxy-2-butanone reductase (a ketone reducing/alcohol forming oxidoreductase); (G) conversion of acetoacetyl-CoA to 3-hydroxybutyryl-CoA by an acetoacetyl-CoA reductase (ketone reducing/alcohol forming oxidoreductase); (H) conversion of 3-hydroxybutyryl-CoA to 3-hydroxybutyraldehyde by a 3-hydroxybutyryl-CoA reductase (a CoA-dependent, aldehyde forming oxidoreductase); (I) conversion of 3-hydroxybutyryl-CoA to 1,3-butanediol by a 3-hydroxybutyryl-CoA reductase (a CoA-dependent/alcohol forming oxidoreductase); (J) conversion of 3-hydroxybutyryl-CoA to 3-hydroxybutyrate by a 3-hydroxybutyryl-CoA transferase, or a 3-hydroxybutyryl-CoA hydrolase; (K) conversion of acetoacetate to 3-hydroxybutyrate by a 3-hydroxybutyrate dehydrogenase (a ketone reducing, alcohol forming oxidoreductase); (L) conversion of 3-hydroxybutyrate to 3-hydroxybutyraldehyde by a 3-hydroxybutyrate reductase; (M) conversion of acetoacetyl-CoA to acetoacetate by an acetoacetyl-CoA transferase, an acetoacetyl-CoA hydrolase, or an acetoacetyl-CoA synthetase; and (N) conversion of acetoacetate to 3-oxobutyraldehyde by an acetoacetate reductase (an aldehyde forming oxidoreductase). The pathways can also include the recycling of acetoacetate by-product back to acetoacetyl-CoA, and/or the recycling of 3HB by-product back to 3HB—CoA, by an ACS or variant or homolog thereof, as described herein.

FIG. 3 shows exemplary pathways from 4-hydroxybutyryl-CoA to 1,3-butanediol. The enzymatic steps are: A) 4-hydroxybutyryl-CoA dehydratase (a hydro-lyase); B) crotonase (a hydro-lyase); C) 3-hydroxybutyryl-CoA reductase (a CoA-dependent, aldehyde forming oxidoreductase); D) 3-hydroxybutyraldehyde reductase (an alcohol forming oxidoreductase; also referred to as an alcohol dehydrogenase); and E) 3-hydroxybutyryl-CoA reductase (an alcohol forming oxidoreductase). The pathways can also include recycling of crotonate by-product back to crotonyl-CoA, and/or recycling of 3HB by-product back to 3HB—CoA, by an ACS or variant or homolog thereof, as described herein.

FIG. 4 shows exemplary pathways for the biosynthesis of 1,3 BDO from alpha-ketoglutarate (AKG), succinate (Succ) and succinyl-CoA (SucCoA). The enzymatic steps are: (A) conversion of Succ to SucCoA by a SucCoA transferase, SucCoA synthetase, or SucCoA ligase; (B) conversion of SucCoA to succinate semialdehyde (SSA) by a SucCoA reductase (aldehyde forming); (C) conversion of a SSA to 4-hydroxybutyrate (4HB) by a 4-hydroxybutyrate dehydrogenase (4HB dehydrogenase); (D) conversion of 4HB to 4-hydroxybutyryl phosphate (4HB—P) by a 4-hydroxybutyrate kinase (4HB kinase); (E) conversion of 4HB—P to 4-hydroxybutyryl-CoA (4HB—CoA) by a phosphotrans-4-hydroxybutyrylase; (F) conversion of Succ to SSA by a Succ reductase; (G) conversion of SucCoA to 4HB by a SucCoA reductase (alcohol forming); (H) conversion of 4HB to 4HB—CoA by a 4HB—CoA transferase, 4HB—CoA synthetase, or 4HB—CoA ligase; (I) conversion of AKG to SSA by an AKG decarboxylase; (J) conversion of a 4HB—CoA to a crotonyl-CoA by a 4HB—CoA dehydratase; (K) conversion of crotonyl-CoA to 3HB—CoA by a CRT; (L) conversion of 3HB—CoA to 3-hydroxybutanal (3-hydroxybutyraldehyde) by a 3-hydroxybutyryl-CoA reductase (aldehyde forming); (M) conversion of a 3-hydroxybutanal to 1,3 BDO by a 3-hydroxybutanal reductase (an alcohol forming oxidoreductase; also referred to as an alcohol dehydrogenase); (N) conversion of 3HB—CoA to 1,3 BDO by a 3-hydroxybutyryl-CoA reductase (alcohol forming); conversion of AKG to glutamate by (O) a glutamate dehydrogenase and/or (P) a glutamate transaminase; (Q) conversion of glutamate to 4-aminobutyrate by a glutamate decarboxylase; conversion of 4-aminobutyrate to SSA by (R) a 4-aminobutyrate dehydrogenase and/or(S) a 4-aminobutyrate transaminase; (T) conversion of AKG to SucCoA by an AKG dehydrogenase; (U) loss of 3HB—CoA to 3HB as described herein; (V) conversion of 3HB to 1,3 BDO by a 3-hydroxybutyrate reductase (alcohol forming reductase); (W) recycling of 3HB into 3HB—CoA as described herein; and (X) conversion of 3HB to 3HBAld (also referred to herein as 3-hydroxybutanal) by a 3HB to 3HBAld conversion step (i.e., by a CAR or variant or homolog thereof), as described herein. The pathways can also include the recycling of succinate by-product back to succinyl-CoA, and/or the recycling of crotonate by-product back to crotonyl-CoA, by an ACS or variant or homolog thereof, as described herein.

FIG. 5 shows exemplary pathways for the biosynthesis of (R)-1,3 BDO from Ac-COA and AcAc—COA. The enzymatic steps are: (A) the conversion of Ac-COA to AcAc—COA by AcAc—COA thiolase; (B) conversion of AcAc—COA to (R)-3-hydroxybutyryl-CoA (R-3HB—CoA) by a hydroxybutyryl-CoA dehydrogenase (HBD); (C) conversion of R-3HB—CoA to (R)-3-hydroxybutyraldehyde (R-3HB-Ald; also referred to as (R)-3-hydroxybutanal) by a butyraldehyde dehydrogenase or acetaldehyde-alcohol dehydrogenase (also referred to as an aldehyde dehydrogenase); (D) conversion of R-3HB-Ald to a R-1,3 BDO by an acetaldehyde-alcohol dehydrogenase (an alcohol forming oxidoreductase; also referred to as an alcohol dehydrogenase); (E) conversion of (R)-3HB—CoA to (R)-3-hydroxybutyrl-phosphotase (R-3HB—P) by a phosphotransbutyrylase (PTB); (F) conversion of R-3HB—P to (R)-3-hydroxybutyrate (R-3HB) by a butyrate kinase (BUK); (G) hydrolysis of (R)-3HB—CoA to (R)-3HB spontaneously or by the activity of nonspecific enzymes; (H) recycling of R-3HB back into R-3HB—CoA by an ACS as described herein; and (I) conversion of (R)-3HB to (R)-3HB—Ald by a CAR or variant or homolog thereof as described herein. The pathway can also include the recycling of acetate and/or acetoacetate by-product(s) back to acetyl-CoA and/or acetoacetyl-CoA, by an ACS or variant or homolog thereof, as described herein.

FIG. 6 depicts exemplary pathways for the production of butyrate, and butanol. The enzymatic steps are: (A) conversion of Ac-COA to AcAc—COA by an acetoacetyl-CoA thiolase; (B) conversion of AcAc—COA to(S)-3-hydroxybutyryl-CoA (S-3HB—CoA) by a 3-hydroxybutyryl-CoA dehydrogenase (HBD), optionally, an (S)-3-hydroxybutyryl-CoA dehydrogenase; (C) conversion of S-3HB—CoA to crotonyl-CoA by a crotonase (CRT); (D) conversion of crotonyl-CoA to butyryl-CoA by a butyryl-CoA dehydrogenase (BCD); (E) conversion of butyryl-CoA to butyraldehyde by a CoA acetylating aldehyde dehydrogenase (ALDH); (F) conversion of butyraldehyde to butanol by an aldehyde reductase (AHR); (G) conversion of butyryl-CoA to butyryl-phosphatase (butyryl-P) by a phosphotransbutyrylase (PTB); (H) conversion of butyryl-P to butyrate by a butyrate kinase (BUK); (I) conversion of butyraldehyde to propane and/or formate by an aldehyde deformulating dehydrogenase (ADO); (J) loss of S-3HB—CoA to S-3HB as described herein; and (K) recycling of S-3HB back to S-3HB—CoA as described herein. The pathway can also include the recycling of any one or more of acetate, acetoacetate, crotonate and/or butyrate by-product(s) back to their CoA thioesters/acyl-CoAs, i.e., acetyl-CoA, acetoacetyl-CoA, crotonyl-CoA, and/or butyryl-CoA, by an ACS or a variant or a homolog thereof, as described herein.

FIG. 7 shows exemplary pathways for the biosynthesis of (3R)-hydroxybutyl (3R)-hydroxybutyrate precursors (R)-1,3-butanediol, (3R)-hydroxybutyrate, (3R)-hydroxybutyryl-CoA, and acetoacetyl-CoA, and the biosynthesis of (3R)-hydroxybutyl (3R) hydroxybutyrate from (R)-1,3-butanediol and (3R)-hydroxybutyryl-CoA. The enzymatic steps are: (A) conversion of Ac-COA to AcAc—COA by an AcAc—COA thiolase; (B) conversion of AcAc—COA to (R)-3HB—CoA by a (3R)-hydroxybutyryl-CoA dehydrogenase; (C) conversion of (R)-3HB—CoA to (R)-3HBAld by a (3R)-hydroxybutyryl-CoA reductase (an aldehyde dehydrogenase); (D) conversion of (R)-3HBAld to (R)-1,3 BDO by a (3R) hydroxybutyraldehyde reductase (an alcohol forming oxidoreductase; also referred to as an alcohol dehydrogenase); (E) conversion of (R)-3HB—CoA and (R)-1,3 BDO to (3R)-hydroxybutyl (3R)-hydroxybutyrate by an enzyme or polypeptide with (3R)-hydroxybutyryl-CoA:(R)-1,3-butanediol alcohol transferase activity; (F) conversion of Ac—CoA to malonyl-CoA by an acetyl-CoA carboxylase; (G) conversion of a malonyl-CoA to AcAc—COA by an acetoacetyl-CoA synthase; (H) conversion of AcAc—COA to(S)-3HB—CoA by a (3S)-hydroxybutyryl-CoA dehydrogenase; (I) conversion of a(S)-3HB—CoA to (R)-3HB—CoA by a 3-hydroxybutyryl-CoA epimerase; (J) loss of (R)-3HB—CoA to (R)-3HB as described herein; (K) recycling of (R)-3HB back to (R)-3HB—CoA by a acyl-CoA synthethase (ACS) or a variant or homolog thereof, as described herein; and (L) conversion of (R)-3HB to (R)-3HBAld by a CAR or variant or homolog thereof as described herein. The pathway can also include the recycling of any one or more of acetate, acetoacetate, malonate, and/or(S)-3HB by-product(s) back to their respective or corresponding acyl-CoAs (or CoA thioesters), i.e., acetyl-CoA, acetoacetyl-CoA, malonyl-CoA, and/or(S)-3HB—CoA, by an ACS or variant or homolog thereof, as described herein.

FIG. 8 shows exemplary metabolic pathways for the conversion of Ac-COA, AcAc—CoA or 4-hydroxybutyryl-CoA into MAA. The enzymatic steps are: (A) conversion of Ac—CoA to AcAc—COA by an acetoacetyl-CoA thiolase; B) conversion of AcAc—COA to 3HB—CoA by an acetoacetyl-CoA reductase (ketone reducing/alcohol forming oxidoreductase); C) conversion of 3HB—CoA to 2-hydroxyisobutyryl-CoA by a 3-hydroxybutyryl-CoA mutase; D) conversion of 2-hydroxyisobutyryl-CoA to methacrylyl-CoA by a 2-hydroxyisobutyryl-CoA dehydratase; conversion of methacrylyl-CoA to methacrylic acid by E) a methacrylyl-CoA transferase, F) a methacrylyl-CoA hydrolase or G) a methacrylyl-CoA synthetase; H) conversion of 4-hydroxybutyryl-CoA to a vinylacetyl-CoA by a 4-hydroxybutyryl-CoA dehydratase; I) conversion of vinylacetyl-CoA to crotonyl-CoA by a vinylacetyl-CoA A-isomerase; and J) conversion of crotonyl-CoA to 3HB—CoA by a CRT. Methacrylate can be converted to methacrylyl-CoA by a methacrylyl-CoA transferase or a methacrylyl-CoA synthetase, which can be converted by an alcohol transferase in combination with an R—OH to an MAA (or methacrylate) ester. R denotes any organic functional group including, but not limited to, a methyl, ethyl, n-propyl, n-butyl, isopropyl, sec-butyl, tert-butyl, pentyl, or hexyl functional group. For example, if R denotes a methyl-group, R—OH denotes methanol and the product of the pathway is methylmethacrylate (MMA). Alternatively, MAA can be directly converted into a methacrylate ester directly by a methacrylate ester-forming enzyme or by chemical conversion (for example, heating in the presence of a dehydrating agent such as an acid). The pathways can also include the recycling of one or more of acetate, acetoacetate, and/or crotonate by-products back to acetyl-CoA, acetoacetyl-CoA, and/or crotonyl-CoA, by an ACS or variant or homolog thereof, as described herein.

FIG. 9 shows exemplary metabolic pathways for the conversion of acetyl-CoA or acetoacetyl-CoA into MAA. The enzymatic steps are: A) Ac-COA is converted to AcAc—COA by an acetoacetyl-CoA thiolase; B) AcAc—COA is converted to a 3HB—CoA by an acetoacetyl-CoA reductase; C) 3HB—CoA is converted to a crotonyl-CoA by a CRT; D) crotonyl-CoA is converted to a butyryl-CoA by a butyryl-CoA dehydrogenase; E) butyryl-CoA is converted to a isobutyryl-CoA by an isobutyryl-CoA mutase; F) isobutyryl-CoA is converted to a methacrylyl-CoA by an isobutyryl-CoA dehydrogenase; and methacrylyl-CoA is converted to methacrylic acid by a G) methacrylyl-CoA synthetase, H) methacrylyl-CoA hydrolase, or I) methacrylyl-CoA transferase. MAA can be esterified enzymatically or chemically (i.e., in vivo or ex vivo) to methacrylate esters (e.g., MMA or EMA). Methacrylate can be converted to a methacrylate ester as described in FIG. 8. The pathways can also include the recycling of one or more of acetate, acetoacetate, 3HB, crotonate, butyrate, and/or isobutyrate by-products back to acetyl-CoA, acetoacetyl-CoA, 3HB—CoA, crotonyl-CoA, butyryl-CoA, and/or isobutyryl-CoA, by an ACS or variant or homolog thereof, as described herein.

FIG. 10 shows exemplary metabolic pathways for the conversion of crotonyl-CoA or 3HB—CoA into isopropanol. The enzymatic steps are: A) crotonyl-CoA is converted to 3HB—CoA by CRT; B) 3HB—CoA is converted to AcAc—COA by HBD; C) AcAc—COA is converted to Acetoacetate by acetoacetyl-CoA synthetase, acetyl-CoA:acetoacetate-CoA transferase, and/or acetoacetyl-CoA hydrolase; F) acetoacetate is converted to acetone by acetoacetate decarboxylase; and G) acetone is converted to isopropanol by acetone reductase. The pathway can also include the recycling of one or more of crotonate, 3HB, and/or acetoacetate by-products back to crotonyl-CoA, 3HB—CoA, and/or acetoacetyl-CoA, by an ACS or variant or homolog thereof, as described herein.

FIG. 11 depicts the concentration of 1,3-BDO in mM, produced over time, by a strain not expressing acyl-CoA synthetase (control strain L20952), a strain weakly expressing acyl-CoA synthetase (L20929), a strain moderately expressing acyl-CoA synthetase (L20928), and a strain strongly expressing acyl-CoA synthetase (L20990). As shown in FIG. 11, the three strains expressing acyl-CoA synthetase (ACS) (L20929, L20928, and L20990) produced higher amounts of 1,3-BDO than the strain not expressing acyl-CoA synthetase (control strain L20952).

FIG. 12 depicts the concentration of 3HB in mM, produced over time, by a strain not expressing acyl-CoA synthetase (control strain L20952), a strain weakly expressing acyl-CoA synthetase (L20929), a strain moderately expressing acyl-CoA synthetase (L20928), and a strain strongly expressing acyl-CoA synthetase (L20990). As shown in FIG. 12, the control strain not expressing acyl-CoA synthetase (L20952) produced the largest amount of 3HB, with decreasing amounts of 3HB being produced by the strains as the expression level of acyl-CoA synthetase is increased.

FIGS. 13A-13B depict the concentrations of 1,3-BDO and 3HB, respectively, produced over time, by a control strain (L29031) not expressing a carboxylic acid reductase (CAR), and a strain overexpressing CAR (L29309). FIG. 13A depicts the concentration of 1,3-BDO in mM, produced over time, by a control strain (L29031) not expressing a CAR, and a strain overexpressing CAR (L29309). As shown in FIG. 13A, the strain overexpressing CAR (L29309) produced higher amounts of 1,3-BDO than the strain not expressing CAR (control strain L29031). FIG. 13B depicts the concentration of 3HB in mM, produced over time, by a control strain (L29031) not expressing a CAR, and a strain overexpressing CAR (L29309). As shown in FIG. 13B, the control strain not expressing CAR (L29031) produced larger amounts of 3HB than the strain overexpressing CAR.

FIG. 14 depicts the concentration of acetate in mM, produced over time, by a control strain not expressing acyl-CoA synthetase (ACS), and a strain expressing ACS. As shown in FIG. 14, the control strain not expressing ACS produced significantly more acetate over time, compared to the strain expressing ACS.

FIG. 15 depicts exemplary pathways from succinyl-CoA and acetyl-CoA to HDO. The enzymes are designated as follows: A) thiolase (Thl), B) a hydroxyadipyl-CoA dehydrogenase (Hbd), C) a crotonase (Crt), D) trans-enoyl CoA reductase (Ter), E) a 6ACA-aldehyde dehydrogenase (6ACA-ALD), F) an alcohol dehydrogenase, G) acyl-coenzyme A ligase (ACL) or acyl-CoA transferase, H) acetylating aldehyde dehydrogenase (ALDH) or acyl-CoA reductase, I) an alcohol dehydrogenase (ADH), and K) carboxylic acid reductase (CAR) or CAR and phosphopantetheinyl transferase (PPTase). The pathways can also include the recycling of acetate and/or succinate by-products back to acetyl-CoA or succinyl-CoA, respectively, by an ACS or variant or homolog thereof, as described herein.

FIG. 16 shows exemplary pathways from succinyl-CoA and acetyl-CoA to adipate, 6-aminocaproate (6ACA), caprolactam (CPL), and hexamethylenediamine (HMD). The enzymes are designated as follows: A) succinyl-CoA:acetyl-CoA transferase or a thiolase, B) 3-hydroxyacyl-CoA dehydrogenase, C) 3-hydroxyadipyl-CoA dehydratase, D) 5-carboxy-2-pentenoyl-CoA reductase, E) 3-oxoadipyl-CoA/acyl-CoA transferase, F) 3-oxoadipyl-CoA synthase, G) 3-oxoadipyl-CoA hydrolase, H) 3-oxoadipate reductase, I) 3-hydroxyadipate dehydratase, J) 5-carboxy-2-pentenoate reductase, K) adipyl-CoA/acyl-CoA transferase, L) adipyl-CoA synthase, M) adipyl-CoA hydrolase, N) adipyl-CoA reductase (aldehyde forming) or 6-ACA-aldehyde dehydrogenase (ALD), O) 6-aminocaproate (6ACA) transaminase, P) 6-aminocaproate dehydrogenase, Q) 6-aminocaproyl-CoA/acyl-CoA transferase, R) 6-aminocaproyl-CoA synthase or ACS, S) amidohydrolase, T) spontaneous and/or chemical cyclization, U) 6-aminocaproyl-CoA reductase (aldehyde forming) or ALDH, V) HMDA transaminase, W) HMDA dehydrogenase, X) adipate reductase, Y) adipate kinase, and Z) adipylphosphate reductase. The pathways can also include the recycling of acetate and/or succinate by-products back to acetyl-CoA or succinyl-CoA, respectively, by an ACS or variant or homolog thereof, as described herein.

### DETAILED DESCRIPTION

This invention is directed, in part, to engineered biosynthetic routes, engineered microorganisms, and methods to decrease by-products, such as 3-hydroxybutyrate (3HB), for enhancing or increasing carbon flux through 3-hydroxybutyryl-CoA (3HB—CoA) and/or 3-hydroxybutyraldehyde (3HB-Ald), and subsequently, through downstream intermediates of 3HB—CoA and/or 3HB-Ald, which thereby increases the titer, yield, and/or productivity of target products derived from acetyl-CoA. Exemplary target products include, without limitation, 1,3-butanediol (1,3-BDO), (R)-1,3-BDO, methacrylic acid (MAA) and its esters (e.g., methyl methacrylate (MMA) and ethyl methacrylate (EMA)), (3R)-hydroxybutyl (3R)-hydroxybutyrate, butyrate, butanol, isopropanol, propane and/or formate. However, it will be recognized by one skilled in the art that any biosynthetic pathway that includes 3HB—CoA, 3HB-Ald, and/or 3HB as an intermediate can exhibit enhanced product titers or yields by decreasing or eliminating by-product formation. The present invention provides non-naturally occurring microbial organisms having one or more exogenous genes encoding one or more enzymes, proteins or polypeptides whose activity or activities result in the decreased production of by-products, such as 3HB. In some embodiments, these non-naturally occurring microbial organisms also have one or more exogenous genes encoding enzymes that can catalyze the production of a desired product, such as, for example, 1,3-BDO, (R)-1,3-BDO, (3R)-hydroxybutyl (3R)-hydroxybutyrate, MAA or an ester thereof, butyrate, butanol, isopropanol, propane, and/or or formate.

The invention also is directed to engineered or modified biosynthetic routes, engineered (or recombinant or non-naturally occurring) microorganisms, and methods, to decrease, eliminate, or substantially eliminate by-products, such as, for example, carboxylic acid or carboxylate by-products, particularly short-chain (e.g., C2-C4, C2-C6, or C2-C8) carboxylic acids or carboxylate by-products of acetyl-CoA derived product pathways, which thereby increases the carbon flux through the acetyl-CoA derived product pathway intermediates, and increases the production, titer, yield, and/or productivity (rate of production) of acetyl-CoA derived products. Such by-products, include, but are not limited to, for example, acetate, acetoacetate, succinate, crotonate, butyrate, isobutyrate, 3-hydroxybutryate (3-HB), (S)-3HB, (R)-3HB, malonate, and/or hexanoate. The carboxylate by-products are converted to their corresponding CoA thioesters (also referred to herein as acyl-CoAs), i.e., acetyl-CoA, acetoacetyl-CoA, succinyl-CoA, crotonyl-CoA, butyryl-CoA, isobutyryl-CoA, 3HB—CoA, (S)-3HB—CoA, (R)-3HB—CoA, malonyl-CoA, and/or hexanoyl-CoA. The acetyl-CoA derived products can include, but are not limited to, for example, 1,3-butanediol (1,3-BDO), (R)-1,3-BDO, (S)-1,3-BDO, methacrylic acid (MAA), methacrylic acid esters (e.g., methyl methacrylate (MMA) and ethyl methacrylate (EMA)), (3R)-hydroxybutyl (3R)-hydroxybutyrate, butyrate, butanol, propane, formate, 3-hydroxybutyryl-coenzyme A (3HB—CoA), hexamethylenediamine (HMDA or HMD), caprolactam (CPL), adipate (or adipic acid), 6-aminocaproic acid (6-ACA), and/or 1,6-hexanediol (HDO), and related products derived therefrom. In some embodiments, these non-naturally occurring microbial organisms also have one or more genes or nucleic acids, such as one or more exogenous genes or nucleic acids, encoding enzymes that can catalyze the production of the target acetyl-CoA derived product(s).

In numerous engineered pathways, realization of maximum product titers or yields based on renewable (e.g., carbohydrate) feedstocks is hampered by the production of unwanted by-products. In accordance with some embodiments, the yields of acetyl-CoA derived products are increased by recycling unwanted 3HB by-product back into 3HB—CoA thereby enhancing or increasing carbon flux through 3HB—CoA. In some embodiments, the production of acetyl-CoA derived products is increased by converting unwanted 3HB to 3HB-Ald, thereby enhancing or increasing carbon flux through 3HB-Ald. In some embodiments, the production of acetyl-CoA derived products is increased by recycling one or more short-chain (e.g., C2-C4, C2-C6, or C2-C8) carboxylate by-products, including, for example, one or more of acetate, acetoacetate, succinate, crotonate, butyrate, isobutyrate, 3-HB, (S)-3HB, (R)-3HB, malonate, and/or hexanoate, back to their CoA thioesters or acyl-CoAs, i.e., acetyl-CoA, acetoacetyl-CoA, succinyl-CoA, crotonyl-CoA, butyryl-CoA, isobutyryl-CoA, 3HB—CoA, (S)-3HB—CoA, (R)-3HB—CoA, malonyl-CoA, and/or hexanoyl-CoA, respectively. This increases the carbon flux through the acyl-CoAs, which are precursors and/or intermediates of acetyl-CoA derived product pathways, and increases the production of acetyl-CoA derived products. Products that can be produced by the non-naturally occurring or engineered microorganisms, by-product recycling loops, biosynthetic pathways, and methods described herein include, by way of example, but without limitation, 1,3-BDO, (R)-1,3-BDO, (S)-1,3-BDO, MAA and its esters (e.g., MMA, EMA, and others), (3R)-hydroxybutyl (3R)-hydroxybutyrate, butyrate, butanol, isopropanol, propane, formate, hexamethylenediamine (HMDA or HMD), caprolactam (CPL), adipate (or adipic acid), 6-aminocaproic acid (6-ACA), and/or 1,6-hexanediol (HDO).

Abbreviations and Conventions:

| Abbreviation | Convention |
|---|---|
| Ala; A | Alanine |
| Arg; R | Arginine |
| Asn; N | Asparagine |
| Asp; D | Aspartic acid |
| Cys; C | Cysteine |
| Glu; E | Glutamic acid |
| Gln; Q | Glutamine |
| Gly; G | Glycine |
| His; H | Histidine |
| Ile; I | Isoleucine |
| Leu; L | Leucine |
| Lys; K | Lysine |
| Met; M | Methionine |
| Phe; F | Phenylalanine |
| Pro; P | Proline |
| Ser; S | Serine |
| Thr; T | Threonine |
| Trp; W | Tryptophan |
| Tyr; Y | Tyrosine |
| Val; V | Valine |
| 1,3-BDO | 1,3-butanediol |
| 3-HB, 3HB | 3-hydroxybutyrate |
| 3HB-CoA | 3-hydroxybutryl-CoA |
| 3HB-P | 3-hydroxybutryl-phosphate |
| 3HBAld | 3-hydroxybutyraldehyde or |
| | 3-hydroxybutanal |
| 4HB | 4-hydroxybutyrate |
| 4HB-CoA | 4-hydroxybutryl-CoA |
| 4HB-P | 4-hydroxybutryl-phosphate |
| 6-ACA | 6-aminocaproic acid or |
| | 6-aminocaproate |
| ACS | acyl-CoA synthetase |
| AcCoA | acetyl-CoA |
| AcAcCoA | acetoacetyl-CoA |
| ADH | alcohol dehydrogenase |
| ALD | aldehyde dehydrogenase |
| ADO | aldehyde deformulating |
| | dehydrogenase |
| AKG | alpha-ketoglutarate |
| AKGD | alpha-ketoglutarate |
| | dehydrogenase |
| ALD | aldehyde dehydrogenase |
| AHR | Aldehyde reductase |
| BCD | butyryl-CoA dehydrogenase |
| butyryl-P | butyryl-phosphatase |
| BUK | butyrate kinase |
| CAR | Carboxylic acid reductase |
| CoA | Coenzyme-A |
| CPL | Caprolactam |
| CRT | Crotonase or 3-oxoadipyl-CoA |
| | dehydratase |
| Hbd | hydroxyadipyl-CoA |
| | dehydrogenase or 3-oxoadipyl- |
| | CoA dehydrogenase |
| HDO | 1,6-hexanediol |
| HMDA or HMD | Hexamethylenediamine |
| MAA | methacrylic acid |
| MMA | methyl methacrylate |
| PTB | phosphotransbutyrylase |
| (R)-1,3 BDO, R-1,3 BDO | (R)-1,3-butanediol |
| (R)-3HB, R-3HB | (3R)-hydroxybutyrate |
| (R)-3HB-CoA, R-3HB- | (3R)-hydroxybutryl-CoA |
| CoA | |
| (R)-3HB-Ald, R-3HB-Ald | (3R)-hydroxybutyraldehyde or |
| | (3R)-hydroxybutanal |
| (S)-1,3 BDO, S-1,3 BDO | (S)-1,3-butanediol |
| (S)-3HB, S-3HB | (3S)-hydroxybutyrate |
| (S)-3HB-CoA, S-3HB- | (3S)-hydroxybutryl-CoA |
| CoA | |
| (S)-3HB-Ald, S-3HB-Ald | (3S)-hydroxybutyraldehyde or |
| | (3S)-hydroxybutanal |
| Succ | succinate |
| SucCoA | succinyl-CoA |
| SSA | succinate semialdehyde |
| TER | trans-enoyl CoA reductase or |
| | 5-carboxy-2-pentenoyl-CoA |
| | reductase |
| Thl | Thiolase, 3-oxoadipyl-CoA |
| | thiolase, or Beta-ketoadipyl- |
| | CoA thiolase |

As used herein, the term “1,3-butanediol,” or “1,3-BDO” is intended to mean one of four stable isomers of butanediol, having the chemical formula C₄H₁₀O₂ and a molecular mass of approximately 90.12 g/mol. The chemical compound 1,3-butanediol also is known in the art as 1,3-butylene glycol (1,3-BG) and is a chemical intermediate or precursor for a family of compounds commonly referred to as the BDO family of compounds.

As used herein, the term “1-butanol,” “butanol,” or “n-butanol” is intended to mean a molecule with the chemical formula C₄H₁₀O and a molecular mass of approximately 74.12 g/mol. The chemical compound butanol is also a chemical intermediate or precursor for a family of compounds commonly referred to as the BDO family of compounds.

As used herein, the term “3-hydroxybutyrate” or “3-HB” or “3HB” is intended to mean a molecule with the chemical formula C₄H₇O₃ and a molecular mass of approximately 103.10 g/mol. The chemical compound 3-hydroxybutyrate can be an undesirable by-product during the production of chemical compounds and intermediates thereof that are derived from acetyl-CoA. A reaction or pathway having “eliminated, substantially eliminated, or reduced” 3HB refers to the decreased concentration of 3HB comparatively to

As used herein, the term “3-hydroxybutyryl CoA ligase” or “3HB—CoA ligase” refers to an enzyme that catalyzes the ligation of 3HB with CoA, to form 3HB—CoA.

As used herein, the term “(3R)-hydroxybutyl (3R)-hydroxybutyrate” refers to a compound of formula (I):

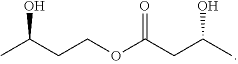

The term (3R)-hydroxybutyl (3R)-hydroxybutyrate is used interchangeably throughout with the terms (R)-(R)-3-hydroxybutyl 3-hydroxybutanoate, (3R)-hydroxybutyl (3R)-hydroxybutyrate, and (R)-3-hydroxybutyl (R)-3-hydroxybutanoate.

As used herein, the term “acetyl-CoA derived compound” or “acetyl-CoA derived product” as used interchangeably herein, refers to any compound or chemical entity (i.e., intermediate or end/target/desired product) that is made via a biochemical or biosynthetic pathway, wherein acetyl-CoA functions as an intermediate and/or is produced upstream of the compound or chemical entity in the pathway. For example, acetyl-CoA derived compounds can include, but are not limited to, for example, 1,3-BDO, (R)-1,3-BDO, (S)-1,3-BDO, MAA and its esters (e.g., MMA), (3R)-hydroxybutyl (3R)-hydroxybutyrate, butyrate, butanol, isopropanol, propane, formate, hexamethylenediamine (HMDA or HMD), caprolactam (CPL), adipate (or adipic acid), 6-aminocaproic acid (6-ACA), and 1,6-hexanediol (HDO).

In some instances, an acetyl-CoA derived product, contains a hydroxyl (OH) group and/or carboxyl (COOH) group, which can occur in various ionized forms, including fully protonated, partially protonated, and fully deprotonated forms. Accordingly, for intermediates and/or products containing a carboxyl group, the suffix “-ate,” or the acid form, can be used interchangeably to describe both the free acid form as well as any deprotonated form, in particular, since the ionization state of a compound is known to depend on the pH in which the compound is found. It is understood that carboxylate products or intermediates include ester forms of carboxylate products or pathway intermediates, including thioesters.

As used herein “acyl-CoA” refers to an acyl thioester formed between the carbonyl carbon of alkyl chain and the sulfhydryl group of the 4′-phosphopantethionyl moiety of coenzyme A (CoA), which has the formula R—C(O)S-CoA, where R is any alkyl group having at least 1 carbon atom. In some embodiments, the alkyl group has at least 2, at least 3, at least 4, at least 5, at least 6, or at least 7 carbon atoms. For example, where the acyl-CoA is 3HB—CoA, the acyl thioester is formed between the carbonyl carbon of 3HB and the sulfhydryl group of the 4′-phosphopantethionyl moiety of coenzyme A.

As used herein, an acyl-CoA synthetase (ACS) is an enzyme with an EC number of 6.2.1.-, 6.2.1.1, and/or 6.2.2.2, which catalyzes the ligation of a carboxylate substrate (such as, for example, 3-hydroxybutyrate (3HB), acetate, acetoacetate, etc.), with coenzyme A (CoA), to form an acyl-CoA (such as, for example, 3-hydroxybutyryl-CoA (or 3HB—CoA), acetyl-CoA, acetoacetyl-CoA, etc.). The acyl-CoA is a CoA thioester of the carboxylate substrate. The ACS with activity corresponding to EC 6.2.1.-, EC 6.2.1.1, and/or EC 6.2.2.2 catalyzes the conversion of short-chain (e.g., C2-C4, C2-C6, or C2-C8) carboxylic acids or carboxylates to their corresponding CoA thioesters or acyl-CoAs. This enzymatic/catalytic reaction requires the presence of ATP, and results in the formation of AMP. The term “acyl-CoA synthetase” is used interchangeably herein with “CoA ligase.”

The ACS can be heterologous to the non-naturally occurring microbial organisms provided herein, and can be expressed or overexpressed, or the ACS can be native to the non-naturally occurring microbial organism and is overexpressed.

As used herein, a polypeptide with acyl-CoA synthetase activity, or a polypeptide with CoA ligase activity, refers to a polypeptide, such as an enzyme or a catalytically active portion thereof, which is capable of catalyzing the ligation of a carboxylate substrate (such as, for example, 3-hydroxybutyrate (3HB)), with coenzyme A (CoA), to form an acyl-CoA (or a CoA thioester of the carboxylate substrate) (such as, for example, 3-hydroxybutyryl-CoA or 3HB—CoA).

As used herein, the term “butyrate”, “butanoate”, “butyric acid”, or “butanoic acid” is intended to mean a molecule with the chemical formula C₄H₇O₂— and a molecular mass of approximately 87.10 g/mol, or the chemical formula C₄H₇OOH and a molecular mass of 88.11 g/mol. The chemical compound butyrate can also be a chemical intermediate or precursor for a family of compounds commonly referred to as the BDO family of compounds.

As used herein, a carboxylic acid reductase (CAR) is an enzyme with an EC number of 1.2.1.- or 1.2.1.30, which catalyzes the ATP and NADPH-dependent reduction of carboxylic acids or carboxylates (such as 3HB) to their corresponding aldehydes (such as 3HBAld). The CARs with activity corresponding to EC 1.2.1.- or 1.2.1.30 catalyze the conversion of carboxylic acids or carboxylates (e.g., 3HB) to their corresponding aldehydes. This enzymatic/catalytic reaction requires the presence of ATP and NADPH.

The CAR can be heterologous to the non-naturally occurring microbial organisms provided herein, and can be expressed or overexpressed, or the CAR can be native to the non-naturally occurring microbial organism and is overexpressed.

As used herein, the term “CoA” or “coenzyme A” is intended to mean an organic cofactor or prosthetic group (non-protein portion of an enzyme) whose presence is required for the activity of certain enzymes to form an active enzyme system. An inactive enzyme without the cofactor, such as CoA, is referred to as an “apoenzyme.” Coenzyme A functions in certain condensing enzymes, acts in acetyl or other acyl group transfers, and in fatty acid synthesis and oxidation, in pyruvate oxidation, and in other acetylation reactions.

As used herein, the term “formate” is intended to mean a molecule with the chemical formula CHO₂ and a molecular mass of approximately 45.017 g/mol.

As used herein, the term “isopropanol” or “2-propanol” or “isopropyl alcohol” is intended to mean a molecule with the molecular formula (CH₃)₂CHOH and a molecular mass of approximately 60.10 g/mol.

As used herein, “methacrylic acid” (MAA) refers to a molecule having the chemical formula CH₂═C(CH₃)CO₂H or C₄H₆O₂ (IUPAC name 2-methyl-2-propenoic acid), which is the acid form of methacrylate, and it is understood that methacrylic acid and methacrylate can be used interchangeably throughout to refer to the compound in any of its neutral or ionized forms, including any salt forms thereof. It is understood by those skilled in the art that the specific form will depend on the pH. The chemical structure of MAA is shown below.

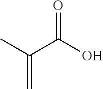

As used herein, “methyl methacrylate,” or “MMA,” having the chemical formula CH₂═C(CH₃)CO₂CH₃ and a molecular mass of 100.12 g/mol, is the methyl ester of methacrylic acid (MAA). MMA is used as the monomer for the production of the transparent plastic polymethyl methacrylate (PMMA).

As used herein, the term “propanol” is intended to mean a molecule with the molecular formula C₃H₈O and a molecular mass of approximately 60.10 g/mol.

As used herein the term “about” means±10% of the stated value. The term “about” can mean rounded to the nearest significant digit. Thus, about 5% means 4.5% to 5.5%. Additionally, about in reference to a specific number also includes that exact number. For example, about 5% also includes exact 5%.

As used herein, the term “biomass” refers to material obtained from biological sources, such as from an agricultural, plant, bacterial, or animal source. Examples of biomass include sugars or carbohydrates. As described herein, often biomass is processed to produce a product, such as 1,3-BDO, MAA (and its esters, e.g., MMA)), (3R)-hydroxybutyl (3R)-hydroxybutyrate, butyrate, butanol, isopropanol, propane, formate, hexamethylenediamine (HMDA or HMD), caprolactam (CPL), adipate (or adipic acid), 6-aminocaproic acid (6-ACA), and 1,6-hexanediol (HDO), through a biosynthetic pathway.

As used herein, the term “bioderived” means derived from or synthesized by a biological organism and can be considered a renewable resource since it can be generated by a biological organism. Such a biological organism, in particular the microbial organisms and cells of the invention disclosed herein, can utilize feedstock or biomass, such as, sugars (e.g., cellobiose, glucose, fructose, xylose, galactose (e.g., galactose from marine plant biomass), and sucrose), carbohydrates obtained from an agricultural, plant, bacterial, or animal source, and glycerol (e.g., crude glycerol by-product from biodiesel manufacturing) for synthesis of a desired bioderived compound. Alternatively, the biological organism can utilize atmospheric carbon.

As used herein, the term “biobased” means a product that is composed, in whole or in part, of a bioderived compound of the invention. A biobased or bioderived product is in contrast to a petroleum derived product, wherein such a product is derived from or synthesized from petroleum or a petrochemical feedstock.

As used herein, the term “by-product” refers to an undesired product that is produced during the production of a desired or target product. This includes, by way of example, as provided herein, the production of, for example, 3HB from 3HB—CoA, where 3HB—CoA is an intermediate in a pathway towards a desired or target product, such as, for example, 1,3-BDO, MAA (and its esters, e.g., MMA)), (3R)-hydroxybutyl (3R)-hydroxybutyrate, butyrate, butanol, isopropanol, propane, formate, or any other acetyl-CoA derived product having 3HB—CoA as an intermediate. The 3HB by-product can be (R)-3HB and/or(S)-3HB, and the 3HB—CoA can be (R)-3HB—CoA and/or(S)-3HB—CoA. Other examples of by-products include, for example, acetate, acetoacetate, succinate, crotonate, butyrate, isobutyrate, malonate, and hexanoate, which are generated from the respective acyl-CoA (or CoA thioester) precursors and/or intermediates of acetyl-CoA derived product pathways, i.e., acetyl-CoA, acetoacetyl-CoA, succinyl-CoA, crotonyl-CoA, butyryl-CoA, isobutyryl-CoA, malonyl-CoA, and hexanoyl-CoA, respectively. The term “carboxylate by-product” or “short-chain carboxylate by-product” can be used to refer to any of the by-products described herein. A short-chain carboxylate by-product, as described herein, typically has a carbon chain length of C8 or less, for example, C2 to C4, C2 to C6, or C2 to C8. Thus, any one or more of 3HB, (R)-3HB, (S)-3HB, acetate, acetoacetate, succinate, crotonate, butyrate, isobutyrate, malonate, and/or hexanoate, can be described herein as a “by-product”, as a “carboxylate by-product”, or as a “short-chain carboxylate by-product.” The terms “carboxylate by-product” and “short-chain carboxylate by-product” can thus be used to describe 3HB. For example, 3HB can be described herein as a “by-product”, as a “3HB by-product”, as a “carboxylate by-product”, or as a “short-chain carboxylate by-product.” The terms “CoA thioester”, “short-chain CoA thioester”, “acyl-CoA”, or “short-chain acyl-CoA” can be used interchangeably to describe the CoA thioesters/acyl-CoAs formed by the ligation of CoA with any one of the by-products (i.e., the carboxylate or short-chain carboxylate by-products) described herein.

As used herein, the term “carbon source” refers to any substrate that supports carbon, wherein the carbon can be used to support the growth of a cell, including a microbial organism, such as the microbial organism described herein. Exemplary carbon sources include: glucose, sucrose, xylose, arabinose, galactose, mannose, fructose, and starch. Another carbon sources useful herein includes methanol.

As used herein, the term “culture medium,” “medium,” “growth medium” or grammatical equivalents thereof refers to a liquid or solid (e.g., gelatinous) substance containing nutrients that support the growth of a cell, including a microbial organism, such as the microbial organism described herein. Nutrients that support growth include, but are not limited to, the following: a substrate that supplies carbon, such as, but are not limited to, cellobiose, galactose, glucose, sucrose, xylose, ethanol, acetate, arabinose, arabitol, sorbitol and glycerol; salts that provide essential elements including magnesium, nitrogen, phosphorus, and sulfur; a source for amino acids, such as peptone or tryptone; and a source for vitamin content, such as yeast extract. Culture medium can be a defined medium, in which quantities of all ingredients are known, or an undefined medium, in which the quantities of all ingredients are not known. Culture medium can also include substances other than nutrients needed for growth, such as a substance that only allows select cells to grow (e.g., antibiotic or antifungal), which are generally found in selective medium, or a substance that allows for differentiation of one microbial organism over another when grown on the same medium, which are generally found in differential or indicator medium. Such substances are well known to a person skilled in the art.

As used herein, the terms “eliminated, substantially eliminated, or reduced” in the context of the production of a by-product, is intended to mean the decrease the intracellular concentration of the by-product or the concentration of the by-product secreted into a culture medium by up to about 100%, up to about 90% or more, up to about 80% or more, up to about 70% or more, up to about 60% or more, up to about 50% or more, up to about 40% or more, up to about 30% or more, up to about 20% or more, up to about 10% or more, up to about 5% or more, up to about 4% or more, up to about 3% or more, up to about 2% or more, up to about 1% or more, or a decrease by any numerical value, and all integers in between as compared to a baseline.

As used herein, the phrases “enhanced or increased carbon flux” or “enhanced or increased carbon flow”, or grammatical equivalents thereof, are intended to mean to intensify, increase, or further improve the extent or flow of metabolic carbon through or to a desired pathway, pathway product, intermediate, or bioderived compound. The intensity, increase, or improvement can be relative to a predetermined baseline for a pathway product, intermediate, or bioderived compound. For example, an increased yield of acetyl-CoA derived product can be achieved by expressing one or more enzymes (or polypeptides) of a 3HB recycling loop pathway, as compared to in the absence of an enzyme (or polypeptide) of a 3HB recycling loop pathway. It is understood that, since an increased yield of 3HB—CoA can be achieved, a higher yield of any acetyl-CoA derived compound having a biosynthetic pathway that includes 3HB—CoA as an intermediate, such as 1,3-BDO, MAA (and its esters, e.g., MMA), (3R)-hydroxybutyl (3R)-hydroxybutyrate, butyrate, butanol, isopropanol, propane, or formate, can also be achieved.

The term “enzyme classification (EC) number” refers to a number that denotes a specific polypeptide sequence or enzyme. EC numbers classify enzymes according to the reaction they catalyze. EC numbers are established by the nomenclature committee of the international union of biochemistry and molecular biology (IUBMB), a description of which is available on the IUBMB enzyme nomenclature website on the world wide web.

The term “exogenous” as it is used herein is intended to mean that the referenced molecule (for example, nucleic acid, gene, polypeptide, protein, or enzyme), or the referenced activity is introduced into or added to the host microbial organism or cell. This can be achieved, for example, by introduction of an encoding nucleic acid into the host genetic material, such as by integration into a host chromosome, or by introduction of an encoding nucleic acid into the host organism or cell as non-chromosomal genetic material, such as on a plasmid. Therefore, the term “exogenous” as it is used in reference to expression of an encoding nucleic acid refers to introduction of the encoding nucleic acid in an expressible form into the microbial organism or cell. When used in reference to a biosynthetic activity, the term “exogenous” refers to an activity that is introduced into the reference host organism or cell. The source of the exogenous activity can be, for example, a homologous or heterologous encoding nucleic acid that, when expressed following introduction into the host organism or cell, results in the referenced activity.

Therefore, the term “endogenous” refers to a referenced molecule or activity that is present in the host organism or cell. Similarly, the term “endogenous,” when used in reference to expression of an encoding nucleic acid, refers to expression of an encoding nucleic acid contained or produced within the microbial organism or cell.

The term “heterologous” refers to a molecule (such as a nucleic acid, gene, polypeptide, protein, or enzyme, for example) or activity derived from a source other than the referenced species, whereas the term “homologous” refers to a molecule or activity derived from the host microbial organism or cell. Accordingly, exogenous expression of an encoding nucleic acid of the invention can utilize either or both of a heterologous or a homologous encoding nucleic acid.

It is understood that, when more than one exogenous nucleic acid is included in a microbial organism or cell, that the more than one exogenous nucleic acids refer to the referenced encoding nucleic acid or biosynthetic activity, as discussed above. It is further understood, as disclosed herein, that such more than one exogenous nucleic acids can be introduced into the host microbial organism or cell as separate nucleic acid molecules, as polycistronic nucleic acid molecules, or a combination thereof, and can still be considered as more than one exogenous nucleic acid. For example, as disclosed herein, a microbial organism or cell can be engineered to express two or more exogenous nucleic acids encoding a desired enzyme or protein required for a pathway, reaction, or series of reactions. In the case where two exogenous nucleic acids encoding a desired activity are introduced into a host microbial organism or cell, it is understood that the two exogenous nucleic acids can be introduced as a single nucleic acid, for example, on a single plasmid, on separate plasmids, or can be integrated into the host chromosome at a single site or at multiple sites, and still can be considered as two exogenous nucleic acids. Similarly, it is understood that more than two exogenous nucleic acids can be introduced into a host organism or cell in any desired combination, for example, on a single plasmid, or on separate plasmids, or can be integrated into the host chromosome at a single site or at multiple sites, and still can be considered as two or more exogenous nucleic acids, for example three exogenous nucleic acids. Thus, the number of referenced exogenous nucleic acids, polypeptides, proteins, enzymes, or biosynthetic activities refers to the number of encoding nucleic acids, polypeptides, proteins, enzymes, or biosynthetic activities, not to the number of separate nucleic acids introduced into the host organism or cell.

As used herein, the term “functional fragment” when used in reference to a peptide, polypeptide or protein is intended to refer to a portion of the peptide, polypeptide or protein that retains some or all of the activity (e.g., catalyzing the conversion of acetoacetyl-CoA to 3HB—CoA or 3HB—CoA to 3HBAld) of the original peptide, polypeptide or protein from which the fragment was derived. In the context of an enzyme or polypeptide with enzymatic activity, the functional fragment may be the catalytically active portion of the enzyme or the polypeptide with enzymatic activity. Such functional fragments include amino acid sequences that are about 200 to about 460, about 200 to about 450, about 200 to about 440, about 200 to about 430, about 200 to about 420, about 200 to about 410, about 200 to about 400, about 200 to about 390, about 200 to about 380, about 200 to about 370, about 200 to about 360, about 200 to about 350, about 300 to about 460, about 300 to about 450, about 300 to about 440, about 300 to about 430, about 300 to about 420, about 300 to about 410, about 300 to about 400, about 300 to about 390, about 300 to about 380, about 300 to about 370, about 300 to about 350, about 300 to about 340, about 300 to about 330, about 300 to about 320, about 300 to about 310, about 400 to about 460, about 400 to about 450, about 400 to about 440, about 400 to about 430, about 400 to about 420, about 400 to about 410, about 450 to about 460 amino acids in length. These functional fragments can, for example, be truncations (e.g., C-terminal or N-terminal truncations) of a peptide, polypeptide, or protein.

A “metabolic modification” refers to a biochemical reaction that is altered from its naturally occurring state. Therefore, non-naturally occurring microorganisms or cells can have genetic modifications to nucleic acids encoding metabolic polypeptides, or functional fragments thereof. Exemplary metabolic modifications are disclosed herein.

Those skilled in the art will understand that the genetic alterations, including the metabolic modifications exemplified herein, are described with reference to a suitable exemplary host organism, such as E. coli, and their corresponding metabolic reactions, or to a suitable source organism for desired genetic material, such as genes for a desired metabolic pathway, reaction, or series of reactions. However, given the complete genome sequencing of a wide variety of organisms, and the high level of skill in the area of genomics, those skilled in the art will readily be able to apply the teachings and guidance provided herein to essentially all other organisms and cells. For example, the E. coli genetic and/or metabolic alterations exemplified herein can readily be applied to other bacterial and non-bacterial species by incorporating the same or analogous encoding nucleic acid from species other than the referenced species. Such genetic alterations include, for example, genetic alterations of species homologs, in general, and in particular, orthologs, paralogs, or nonorthologous gene displacements.

An ortholog is a gene or genes that are related by vertical descent and are responsible for substantially the same or identical functions in different organisms. For example, mouse epoxide hydrolase and human epoxide hydrolase can be considered orthologs for the biological function of hydrolysis of epoxides. Genes are related by vertical descent when, for example, they share sequence similarity of sufficient amount to indicate they are homologous, or they are related by evolution from a common ancestor. Genes can also be considered orthologs if they share three-dimensional structure, but not necessarily sequence similarity, of a sufficient amount to indicate that they have evolved from a common ancestor to the extent that the primary sequence similarity is not identifiable. Genes that are orthologous can encode proteins with sequence similarity of about 25% to 100% amino acid sequence identity. Genes encoding proteins sharing an amino acid similarity of less than 25% can also be considered to have arisen by vertical descent if their three-dimensional structures also show similarities. Members of the serine protease family of enzymes, including tissue plasminogen activator and elastase, are considered to have arisen by vertical descent from a common ancestor.

Orthologs include genes or their encoded gene products that through, for example, evolution, have diverged in structure or overall activity. For example, where one species encodes a gene product exhibiting two functions and where such functions have been separated into distinct genes/gene products in a second species, the three genes and their corresponding products are considered to be orthologs. For the production of a biochemical product, those skilled in the art will understand that the orthologous gene harboring the metabolic activity to be introduced or disrupted is to be chosen for construction of the non-naturally occurring microorganism or cell. An example of orthologs exhibiting separable activities is where distinct activities have been separated into distinct gene products between two or more species or within a single species. A specific example is the separation of elastase proteolysis and plasminogen proteolysis, two types of serine protease activities, into distinct molecules as plasminogen activator and elastase. A second example is the separation of mycoplasma 5′-3′ exonuclease and Drosophila DNA polymerase III activity. The DNA polymerase from the first species can be considered an ortholog to either or both of the exonuclease or the polymerase from the second species and vice versa.

In contrast, paralogs are homologs are related by, for example, duplication followed by evolutionary divergence, and have similar or common, but not identical, functions. Paralogs can originate or derive from, for example, the same species or from a different species. For example, microsomal epoxide hydrolase (epoxide hydrolase I) and soluble epoxide hydrolase (epoxide hydrolase II) can be considered paralogs because they represent two distinct enzymes, co-evolved from a common ancestor, that catalyze distinct reactions and have distinct functions in the same species. Paralogs are proteins from the same species with significant sequence similarity to each other, suggesting that they are homologous, or are related through co-evolution from a common ancestor. Groups of paralogous protein families include HipA homologs, luciferase genes, peptidases, and others.

A nonorthologous gene displacement is a nonorthologous gene from one species that can substitute for a referenced gene function in a different species. Substitution includes, for example, being able to perform substantially the same or a similar function in the species of origin compared to the referenced function in the different species. Although generally, a nonorthologous gene displacement will be identifiable as structurally related to a known gene encoding the referenced function, less structurally related but functionally similar genes and their corresponding gene products nevertheless will still fall within the meaning of the term as it is used herein. Functional similarity requires, for example, at least some structural similarity in the active site or binding region of a nonorthologous gene product compared to a gene encoding the function sought to be substituted. Therefore, a nonorthologous gene includes, for example, a paralog or an unrelated gene.

Therefore, in identifying and constructing the non-naturally occurring microbial organisms or cells of the invention having biosynthetic pathways with decreased levels of (or eliminated) by-products, such as 3HB, those skilled in the art will understand, with applying the teachings and guidance provided herein to a particular species, that the identification of metabolic modifications can include identification and inclusion or inactivation of orthologs. To the extent that paralogs and/or nonorthologous gene displacements are present in the referenced microorganism that encode an enzyme catalyzing a similar or substantially similar metabolic reaction, those skilled in the art also can utilize these evolutionarily related genes. Similarly, for a gene disruption, evolutionarily related genes can also be disrupted or deleted in a host microbial organism to reduce or eliminate functional redundancy of enzymatic activities targeted for disruption.

Orthologs, paralogs, and nonorthologous gene displacements can be determined by methods well known to those skilled in the art. For example, inspection of nucleic acid or amino acid sequences for two polypeptides will reveal sequence identity and similarities between the compared sequences. Based on such similarities, one skilled in the art can determine if the similarity is sufficiently high to indicate that the proteins are related through evolution from a common ancestor. Algorithms well known to those skilled in the art, such as Align, BLAST, Clustal W and others compare and determine a raw sequence similarity or identity, and also determine the presence or significance of gaps in the sequence which can be assigned a weight or score. Such algorithms also are known in the art and are similarly applicable for determining nucleotide sequence similarity or identity. Parameters for sufficient similarity to determine relatedness are computed based on well-known methods for calculating statistical similarity, or the chance of finding a similar match in a random polypeptide, and the significance of the match is determined. A computed comparison of two or more sequences can, if desired, also be optimized visually by those skilled in the art. Related gene products or proteins can be expected to have a high similarity, for example, 25% to 100% sequence identity. Proteins that are unrelated can have an identity which is essentially the same as would be expected to occur by chance, if a database of sufficient size is scanned (about 5%). Sequences with identities of between 5% and 24% may or may not represent sufficient homology to conclude that the compared sequences are related. Additional statistical analysis to determine the significance of such matches given the size of the data set can be carried out to determine the relevance of these sequences.

When referring to two nucleic acid/polynucleotide or polypeptide/protein sequences, the “percentage of sequence identity” between the two sequences is determined by comparing the two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The “percentage of sequence identity” is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

Thus, the expression “percent identity,” or “percent sequence identity” in the context of two or more nucleic acid sequences or peptides or polypeptides, refers to two or more sequences or subsequences that are the same or have a specified percentage of nucleotides or amino acids that are the same (e.g., about 50%, 55%, 60%, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured e.g., using a BLAST or BLAST 2.0 sequence comparison algorithm with default parameters (see, e.g., Altschul et al. (1990) J. Mol. Biol. 215 (3): 403-410 and/or the NCBI web site at ncbi.nlm.nih.gov/BLAST/) or by manual alignment and visual inspection. Percent sequence identity between two nucleic acid or amino acid sequences also can be determined using e.g., the Needleman and Wunsch algorithm that has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6 (Needleman and Wunsch (1970) J. Mol. Biol. 48:444-453). The percent sequence identity between two nucleotide sequences also can be determined using the GAP program in the GCG software package, using a NWSgapdna.CMP matrix and a gap weight of 40, 50, 60, 70, or 80 and a length weight of 1, 2, 3, 4, 5, or 6. One of ordinary skill in the art can perform initial sequence identity calculations and adjust the algorithm parameters accordingly. A set of parameters that may be used if a practitioner is uncertain about which parameters should be applied to determine if a molecule is within a sequence identity limitation of the claims, are a Blossum 62 scoring matrix with a gap penalty of 12, a gap extend penalty of 4, and a frameshift gap penalty of 5. Additional methods of sequence alignment are known in the biotechnology arts (see, e.g., Rosenberg (2005) BMC Bioinformatics 6:278; Altschul et al. (2005) FEBS J. 272(20):5101-5109).

Exemplary parameters for determining relatedness of two or more sequences using the BLAST algorithm, for example, can be as set forth below. Briefly, amino acid sequence alignments can be performed using BLASTP version 2.0.8 (Jan. 5, 1999) and the following parameters: Matrix: 0 BLOSUM62; gap open: 11; gap extension: 1; x_dropoff: 50; expect: 10.0; wordsize: 3; filter: on. Nucleic acid sequence alignments can be performed using BLASTN version 2.0.6 (Sep. 16, 1998) and the following parameters: Match: 1; mismatch:-2; gap open: 5; gap extension: 2; x_dropoff: 50; expect: 10.0; wordsize: 11; filter: off. Those skilled in the art will know what modifications can be made to the above parameters to either increase or decrease the stringency of the comparison, for example, and determine the relatedness of two or more sequences.

As used herein, the terms “polypeptide” and “protein” are used interchangeably to refer to a polymer of amino acid residues that is typically 12 or more amino acids in length.

In certain aspects, a polypeptide may be described in reference to a sequence accession number, which is an identifier for a sequence record. Sequence Accession numbers throughout this description were obtained from databases provided by the NCBI (National Center for Biotechnology Information) maintained by the National Institutes of Health, U.S.A. (which are identified herein as “NCBI Accession Numbers” or alternatively as “GenBank Accession Numbers” or alternatively as “Accession Numbers”), and from the UniProt Knowledgebase (UniProtKB) and Swiss-Prot databases provided by the Swiss Institute of Bioinformatics (which are identified herein as “UniProtKB Accession Numbers”). Likewise, Sequence Record numbers throughout this description were obtained from databases provided by the NCBI (National Center for Biotechnology Information) maintained by the National Institutes of Health, U.S.A. (which are identified herein as “GenInfo Identifier Number” or alternatively as “GI Number”)

As used herein, the term “pathway” when used in reference to production of a desired product (e.g., 1,3-BDO, MAA and its esters (e.g., MMA, EMA, and others), (3R)-hydroxybutyl (3R)-hydroxybutyrate, butyrate, butanol, isopropanol, propane, and formate) refers to one or more polypeptides (e.g., proteins or enzymes) that catalyze the conversion of a substrate compound to a product compound and/or produce a co-substrate for the conversion of a substrate compound to a product compound. Such a product compound can be one of the bioderived compounds described herein, or an intermediate compound that can lead to the bioderived compound upon further conversion by other proteins or enzymes of the metabolic pathway. Accordingly, a metabolic pathway can be comprised of a series of metabolic polypeptides (e.g., two, three, four, five, six, seven, eight, nine, ten or more) that act upon a substrate compound to convert it to a given product compound through a series of intermediate compounds. The metabolic polypeptides of a metabolic pathway can be encoded by an exogenous nucleic acid as described herein or produced naturally by the host microbial organism. Likewise, the term “biosynthesis pathway” or “biosynthetic pathway” refers to one or more polypeptides that catalyze the conversion of a substrate compound to a product compound and/or produce a co-substrate for the conversion of a substrate compound to a product compound that leads to a bioderived or biobased product as described herein.

As used herein, the term “promoter” refers to a nucleotide sequence where transcription of a linked open reading frame (e.g., a nucleotide sequence encoding an engineered aldehyde dehydrogenase) by an RNA polymerase begins. A promoter sequence can be located directly upstream or at the 5′ end of the transcription initiation site. RNA polymerase and the necessary transcription factors bind to a promoter sequence and initiate transcription. Promoter sequences define the direction of transcription and indicate which DNA strand will be transcribed, i.e. the sense strand.

A “constitutive” promoter is a promoter that is active under most environmental and developmental conditions. An “inducible” promoter is a promoter that is active under environmental or developmental regulation.

A promoter that could be used to achieve expression of a nucleotide sequence coding a protein, may be not native to the nucleotide sequence coding for the protein to be expressed, i.e. a promoter that is heterologous to the nucleotide sequence (coding sequence) to which it is operably linked. In some instances, the promoter is homologous, i.e., endogenous to the host cell.

Promoter strength is a factor in determining how much mRNA is transcribed from a gene, which in turn, which may effect how much of the corresponding gene product (e.g., an enzyme as described herein) is produced. A promoter may be a “weak”, “moderate” or “strong” depending on the amount or frequency of RNA polymerase binding to the promoter, where little (or infrequent) binding indicates a weak promoter, significant (or frequent) binding indicates a strong promotor, and medium binding indicates a moderate promoter. Examples of weak promoters include: STE1, CYC1, and HIS3. Examples of moderate promoters include: ADH1. Examples of strong promoters include: TEF1, TDH1, TDH3, PGK1, ENO1, ENO2, TPI1, GAL10, GAL1, and LEU2. Other suitable promoters include: GAL7, PH05, ADC1, ACT1, TRP1, URA3, PDC1, and GPD1.

As used herein, the term “productivity” refers to the quantity of a product or intermediate (such as an acetyl-CoA derived product or intermediate thereof) that is produced per unit volume of host cell culture per unit time. The units for productivity can be, for example, mg/L/hour or g/L/hour. The term “rate” can be used interchangeably with “productivity.”

As used herein, the terms “microbial,” “microbial organism” or “microorganism” are intended to mean any organism that exists as a microscopic cell that is included within the domains of archaea, bacteria, or eukarya. Therefore, the term is intended to encompass prokaryotic or eukaryotic cells, or organisms having a microscopic size, and includes bacteria, archaea and eubacteria of all species, as well as eukaryotic microorganisms, such as yeast and fungi. The term also includes cell cultures of any species that can be cultured for the production of a biochemical or a bio-derived product.

As used herein, the term “non-naturally occurring” when used in reference to a microbial organism or microorganism or cell of the invention is intended to mean that the microbial organism has at least one genetic alteration not normally found in a naturally occurring strain of the referenced species, including wild-type strains of the referenced species. Genetic alterations include, for example, modifications introducing expressible nucleic acids encoding polypeptides (e.g., metabolic polypeptides), as well as other nucleic acid additions, nucleic acid deletions and/or other functional disruptions of the microbial organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon. Exemplary metabolic polypeptides include enzymes or proteins that result in an increase or decrease of a metabolic intermediate, for example. Exemplary metabolic intermediates include 3HB, 3HB-Ald, 3HB—CoA, acetyl-CoA, and acetoacetyl-CoA. Exemplary metabolic polypeptides include, for example, acyl-CoA synthetase (ACS).

A “non-naturally occurring” microbial organism, microorganism, or cell also can be referred to herein as an “engineered” or “recombinant” microbial organism, microorganism, or cell. As used herein the term “product” in reference to a “substrate-product” pair can refer to an intermediate in a product pathway, which in turn can serve as a substrate for the next or downstream substrate-product pair in the pathway. For example, in a pathway that includes the conversion of acetoacetyl-CoA to 3HB—CoA, and the conversion of 3HB—CoA to 3HBAld, 3HB—CoA can be considered a “product” when the “substrate-product” pair is acetoacetyl-CoA and 3HB—CoA, and 3HB—CoA can also be considered a “substrate” when the “substrate-product” pair is 3HB—CoA and 3HB Ald.

As used herein, the term “recycling loop,” as used interchangeably herein with “recycling pathway” or “recycling loop pathway,” or “by-product recycling loop” or “by-product recycling pathway,” refers to one or more reactions that convert a by-product back to a substrate or pathway intermediate that can then be metabolized and redirected to the desired product. For example, an exemplary recycling loop provided herein includes a 3HB recycling loop which, for example, converts the by-product 3HB, which is produced from 3HB—CoA, back to 3HB—CoA, and increases the titer, yield, and/or productivity of the desired acetyl-CoA derived product, such as 1,3-BDO, MAA and its esters (e.g., MMA, EMA, and others) (3R)-hydroxybutyl (3R)-hydroxybutyrate, butyrate, butanol, isopropanol, propane, or formate, or any other product produced via a pathway that includes 3HB—CoA as an intermediate. A by-product recycling loop, as used herein, generally refers to the conversion of a carboxylate by-product, particularly a short-chain (e.g., C2-C4, C2-C6, or C2-C8) carboxylate by-product, back to its corresponding acyl-CoA (also referred to herein as a CoA thioester or a CoA thioester of a carboxylate (or short-chain carboxylate) by-product). As used herein, a short-chain carboxylate by-product includes, but is not limited to, for example, acetate, acetoacetate, succinate, crotonate, butyrate, isobutyrate, 3HB, (S)-3HB, (R)-3HB, malonate, and/or hexanoate. Thus, another exemplary recycling loop is the acetate recycling loop, which, for example, converts the by-product acetate, which is produced from acetyl-CoA, back to acetyl-CoA, and increases the titer, yield, and/or productivity of the desired acetyl-CoA derived product. A by-product recycling loop or pathway can thus be referred to herein as a carboxylate by-product recycling loop or pathway, or as a short-chain carboxylate by-product recycling loop or pathway.

As used herein, a “3HB to 3HBAld conversion step”, a “3HB to 3HB-Ald conversion step” or a “3-hydroxybutyrate to 3-hydroxybutyraldehyde conversion step” refers to the catalytic conversion of 3HB to 3HBAld (used interchangeably herein with 3HB-Ald) by a carboxylic acid reductase (CAR) or a catalytically active portion thereof, or by an enzyme or a polypeptide with carboxylic acid reductase activity. The CAR or catalytically active portion thereof, or the enzyme or polypeptide with CAR activity has activity corresponding to an EC number of 1.2.1.- or 1.2.1.30, and catalyzes the ATP- and NADPH-dependent reduction of a carboxylic acid to an aldehyde.

As used herein, the term “substantially anaerobic,” when used in reference to culture or growth conditions, refers to conditions in which the amount of oxygen is less than about 10% of the saturated amount of dissolved oxygen in the liquid medium used for growth or culturing. This term is maintained in an atmosphere of oxygen of less than about 1%, the sealing chamber of a liquid or solid medium, is also included.

As used herein, the term “titer” refers to the quantity of the target (e.g., acetyl-CoA derived) product, or to the quantity of a pathway intermediate, that is produced per unit volume of host cell culture. The titer can be expressed in the units of millimolar (mM), molar (M), mg/L, g/L, or kg/L.

As used herein, the term “variant” is intended to mean a form or version of a nucleic acid, gene, polypeptide, or protein (e.g., an enzyme) that differs from the corresponding wild-type form thereof, or that differs from a template nucleic acid, gene, polypeptide, or protein. In some embodiments, the template is a variant of a wild-type nucleic acid, gene, polypeptide, or protein. An exemplary variant is a mutant version of an enzyme where the amino acid sequence of the variant enzyme differs from the amino acid sequence at one or more of the homologous or corresponding amino acid positions. A variant may have a different or altered function or activity relative to the wild-type enzyme. For example, a variant enzyme can have increased or decreased activity, and/or can have increased or decreased levels of expression, and/or can have increased or decreased specificity and/or selectivity for a substrate, in comparison to the wild-type or template enzyme. A variant gene/nucleic acid or polypeptide/protein can have one or more modifications (or alterations or mutations) in the nucleic acid or amino acid sequence, respectively, where a modification (or alteration or mutation), as used herein, can refer to an addition, deletion, or substitution (or replacement) of one or more nucleotides or amino acids. However, a variant need not be a mutant, and can encompass polymorphisms, paralogs, or orthologs. A variant can have at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, or more, modifications (or alterations or mutations), e.g., additions, deletions, and/or substitutions of nucleotides or amino acids, compared to template or wild-type sequence.

As used herein, the term “yield” refers to the efficiency by which an input carbon source is converted to product or intermediate, such as an acetyl-CoA derived product or intermediate thereof, in a host cell, cell culture, or microorganism. The yield is typically provided as a percentage.

Non-naturally occurring microbial organisms or cells, having pathways for the increased production of acetyl-CoA (Ac-COA) derived products, such as, for example, 1,3-BDO (including (R)- and (S)-isomers thereof), MAA and its esters (e.g., MMA, EMA, and others), butyrate, butanol, propane, formate, isopropanol, (3R)-hydroxybutyl (3R)-hydroxybutyrate, adipate, hexamethylenediamine (HMDA or HMD), 6-aminocaproic acid (6ACA or 6-ACA), caprolactam (CPL), and/or 1,6-hexanediol (HDO), and their use in methods and processes for the production of the Ac-COA derived products, previously have been described, for example, in International Application Publication Nos. WO 2005/068643, WO 2009/113853, WO 2009/113855, WO 2009/135074, WO 2009/151728, WO 2010/127319, WO 2010/129936, WO 2011/071682, WO 2011/078668, WO 2012/135789, WO 2012/177619, WO 2012/177721, WO 2013/036764, WO 2013/071226, WO 2014/071289, WO 2014/099725, WO 2014/190251, WO 2016/209883, and WO 2021/231271, in U.S. Pat. Nos. 9,017,983, 9,982,281, and in US 2013/0066035, US 2013/0065279, and US 2016/0108442, which are incorporated in their entirety by reference herein. As discussed above and elsewhere herein, the titer, rate, productivity, and/or yield of production of chemical compounds, such as 1,3-BDO, butyrate, butanol, propane, formate, isopropanol, MAA and its esters (e.g., MMA, EMA, and others), (3R)-hydroxybutyl (3R)-hydroxybutyrate, HMD, adipate, 6ACA, CPL, and/or HDO, by such non-naturally occurring microbial organisms or cells can be limited by the generation of unwanted by-products, such as 3-HB, acetate, acetoacetate, succinate, crotonate, butyrate, isobutyrate, (S)-3HB, (R)-3HB, malonate, and/or hexanoate.

The non-naturally occurring organisms and cells of the present invention can also have a glycolysis pathway which results in the production of Ac-COA. Briefly, pyruvate is produced in high quantities via glycolysis, which can then be converted to Ac-COA, for example, by the enzyme pyruvate formate lyase (e.g., in bacteria), or by the pyruvate dehydrogenase (PDH) complex (e.g., in yeast). Ac-COA also can be produced by metabolic pathways other than glycolysis, for example, by methanol metabolic pathways, formaldehyde assimilation pathways, or formate reutilization pathways, such as those described in, for example, WO 2014/099725 and WO 2014/035925, which are incorporated in their entirety by reference herein. Additionally, Ac-COA can be produced by the Wood-Ljungdahl pathway (see, e.g., Drake, H. L., J Bacteriol. 150:702-709 (1982); Drake and Daniel, Res Microbiol 155:869-883 (2004); Kellum and Drake, J Bacteriol. 160:466-469 (1984)).

Acetyl-CoA (Ac-COA) can be used to build precursors of target products, or product pathway intermediates, such as acetoacetyl-CoA (AcAc—COA), and is an important precursor for downstream products such as, for example, 1,3-BDO, MAA and its esters (e.g., MMA, EMA, and others), butyrate, butanol, propane, formate, isopropanol, (3R)-hydroxybutyl (3R)-hydroxybutyrate, HMD, adipate, 6ACA, CPL, and/or HDO. Accordingly, the present invention additionally provides a microorganism or cell expressing nucleic acids/genes encoding polypeptides/enzymes that catalyze reactions associated with the production of Ac-COA (e.g., via glycolysis and non-glycolysis pathways, such as the RuMP pathway described in WO 2014/099725, among other pathways set forth above), in addition to nucleic acids/genes for the production of one or more products derived from Ac-COA. Such an organism or cell is capable of generating Ac-COA and, further, of converting it into an Ac-COA derived product, including, for example, 1,3-BDO, MAA and its esters (e.g., MMA, EMA, and others), butyrate, butanol, propane, formate, isopropanol, (3R)-hydroxybutyl (3R)-hydroxybutyrate, HMD, adipate, 6ACA, CPL, and/or HDO. In some embodiments, the Ac—CoA derived product pathways in the non-naturally occurring organisms and cells provided herein include a 3HB—CoA and/or 3HB-Ald intermediate.

3HB—CoA is an unstable intermediate that can be converted to 3HB spontaneously (via hydrolysis), or through the action of nonspecific enzymes, such as, for example, CoA-thioesterase, 3-hydroxybutyryl-CoA transferase, or 3-hydroxybutyryl-CoA hydrolase. The conversion of 3HB—CoA to 3HB by-product reduces the amount of 3HB—CoA intermediate that is available and/or reduces the carbon flux through 3HB—CoA and/or 3HB-Ald, thus reducing the titer, yield, and/or productivity of Ac-COA derived products whose biosynthetic pathways include a 3HB—CoA and/or 3HB-Ald intermediate. Accordingly, in certain embodiments, provided herein is a 3HB recycling pathway that redirects or recycles 3HB back into 3HB—CoA to reduce or eliminate the production of 3HB by-product and/or to enhance or increase the carbon flux through 3HB—CoA, thereby increasing the yield, titer, and/or productivity of Ac—CoA derived products, such as, for example, 1,3-BDO, MAA or its esters (e.g., MMA, EMA, and others), butyrate, butanol, propane, formate, isopropanol, and (3R)-hydroxybutyl (3R)-hydroxybutyrate. Also provided herein is a 3HB to 3HB-Ald conversion step, which redirects 3HB back to 3HB-Ald, to reduce or eliminate the production of 3HB by-product and/or to enhance or increase the carbon flux through 3HB-Ald, thereby increasing the yield, titer, and/or productivity of Ac-COA derived products, such as, for example, 1,3-BDO and (3R)-hydroxybutyl (3R)-hydroxybutyrate. Accordingly, in certain embodiments, provided herein is a non-naturally occurring microbial organism or cell having reduced, eliminated, or substantially eliminated by-products, including a microbial organism or cell having an Ac-COA derived product pathway and an enhanced or increased carbon flux through 3HB—CoA. In some embodiments, the microbial organism includes a 3-HB recycling pathway.

Similarly, the CoA thioesters (acyl-CoAs) of other short-chain carboxylate by-products, e.g., acetyl-CoA, acetoacetyl-CoA, succinyl-CoA, crotonyl-CoA, butyryl-CoA, isobutyryl-CoA, malonyl-CoA, and hexanoyl-CoA, are unstable intermediates that can spontaneously be converted to the corresponding carboxylate by-products via hydrolysis, or by the action of nonspecific enzymes. This reduces the amount of acyl-CoA precursors and/or intermediates that are available and/or reduces the carbon flux through the acyl-CoA precursors and/or intermediates, thus reducing (or decreasing) the production, titer, yield, and/or productivity of Ac-COA derived products whose biosynthetic pathways include such intermediates. Accordingly, in certain embodiments, provided herein are carboxylate (e.g., short-chain carboxylate) by-product recycling pathways that redirect or recycle the carboxylates back into their corresponding CoA thioesters/acyl-CoAs, to reduce or eliminate or substantially eliminate the production of carboxylate by-products and/or to enhance or increase the carbon flux through the CoA thioesters of the carboxylates (i.e., the acyl-CoAs), thereby increasing the production, yield, titer, and/or productivity of acetyl-CoA derived products, such as, for example, 1,3-BDO, MAA or its esters (e.g., MMA, EMA, and others), butyrate, butanol, propane, formate, isopropanol, (3R)-hydroxybutyl (3R)-hydroxybutyrate, HMD, adipate, 6ACA, CPL, and/or HDO. As used herein, the term “short-chain carboxylate by-product” generally refers to C2-C4, C2-C6, or C2-C8 carboxylates, such as, for example, 3HB, (R)-3HB, (S)-3HB, acetate, acetoacetate, succinate, crotonate, butyrate, isobutyrate, malonate, and hexanoate, which are derived from their corresponding CoA thioesters or acyl-CoAs, i.e., 3HB—CoA, (R)-3HB—CoA, (S)-3HB—CoA, acetyl-CoA, acetoacetyl-CoA, succinyl-CoA, crotonyl-CoA, butyryl-CoA, isobutyryl-CoA, malonyl-CoA, and hexanoyl-CoA, respectively. One or more of such acyl-CoAs can be precursors and/or intermediates of the acetyl-CoA derived product pathway(s).

Accordingly, in certain embodiments, provided herein is a non-naturally occurring microbial organism or cell having reduced (decreased), eliminated, or substantially eliminated carboxylate (e.g., short-chain carboxylate) by-product(s), including a microbial organism or cell having at least one acetyl-CoA derived product pathway and an enhanced or increased carbon flux through one or more CoA thioesters of the carboxylate by-products (i.e., acyl-CoAs). In some embodiments, the microbial organism includes at least one carboxylate (e.g., short-chain carboxylate) recycling pathway. In some embodiments, the microbial organism comprises at least two, at least three, at least four, at least five, at least six, at least seven, at least eight, at least nine, at least ten, or more, carboxylate by-product recycling pathways. In some embodiments, the microbial organism comprises two carboxylate by-product recycling pathways.

As provided herein, a non-naturally occurring microbial organism or cell with a 3HB recycling loop or pathway is able to redirect carbons that were converted from 3HB—CoA into 3HB, back into 3HB—CoA. In one aspect, the non-naturally occurring microbial organism or cell described herein, having a 3HB recycling loop or pathway, includes an Ac-COA derived product pathway having a 3HB—CoA intermediate. Accordingly, in some embodiments, the non-naturally occurring microbial organism or cell having a 3HB recycling loop has enhanced or increased carbon flux through 3HB—CoA, reduced production of 3HB by-product, and/or increased titer, yield, and/or productivity of an Ac-COA derived product.

As described above, a microbial organism or cell, having a 3HB—CoA pathway or a product pathway that includes a 3HB—CoA intermediate, can also have one or more enzymes or activities that result in the loss of 3HB—CoA to 3HB, for example, by spontaneous hydrolysis, or by the activity of one or more of a CoA-thioesterase, a 3-hydroxybutyryl-CoA transferase, or a 3-hydroxybutyryl-CoA hydrolase. The recycling of 3HB back to 3HB—CoA can be catalyzed by an enzyme with acyl-CoA synthetase (ACS) activity or CoA ligase activity. Hence, in certain embodiments, the non-naturally occurring microbial organism or cell, having a 3HB recycling loop or pathway, has enhanced or increased carbon flux through 3HB—CoA, reduced, eliminated, or substantially eliminated production of 3HB by-product, and/or increased titer, yield, and/or productivity of an Ac-COA derived product, and includes one or more exogenous nucleic acids encoding an ACS, or a CoA ligase, or homologs, variants, or catalytically active portions thereof, or encoding a polypeptide having acyl-CoA synthetase or CoA ligase activity.

In certain embodiments, the non-naturally occurring microbial organism or cell expresses or overexpresses one or more exogenous nucleic acids encoding an ACS, or a CoA ligase, or homologs, variants, or catalytically active portions thereof, or encoding a polypeptide with ACS or CoA ligase activity. In some embodiments, the level of overexpression can be weak (or low), moderate (or medium), or strong (or high). The level of expression or overexpression of the nucleic acid(s) can be controlled by methods known in the art, such as, for example, by utilizing promoters that induce weak (e.g., p115 promoter), moderate (e.g., p108 promoter), or strong (e.g., p100 promoter) levels of expression. Alternatively, or additionally, the copy number of the encoding nucleic acid(s) or gene(s) is increased. For example, multiple copies of the nucleic acid encoding an ACS/CoA ligase (or the homolog, variant, or catalytically active portion thereof, or polypeptide having ACS/CoA ligase activity) are integrated into the genome/chromosome of the non-naturally occurring microbial organism or cell. Where the encoding nucleic acid or gene is introduced into the microbial organism or cell on a plasmid(s), multiple copies of the encoding nucleic acid or gene can be introduced into the plasmid(s), and/or the plasmid(s) containing the encoding nucleic acid(s) can be altered to increase expression. For example, a high copy number plasmid(s) can be used, or an origin of replication that results in high copy number can be introduced into the plasmid(s). As is known in the art, the higher the copy the number of the nucleic acid(s) and/or plasmid(s), the higher the level of expression of the encoded polypeptide. Alternatively, or additionally, the non-naturally occurring microbial organism can contain nucleic acid(s) encoding a variant of an ACS, or a CoA ligase, or a variant of a polypeptide with ACS or CoA ligase activity, wherein the nucleic acid(s) encoding the variant(s) contain(s) one or more modifications that result in increased expression. Alternatively or additionally, the nucleic acid encoding the ACS/CoA ligase (or the homolog, variant, or catalytically active portion thereof, or polypeptide having ACS/CoA ligase activity) can be codon optimized, by methods known in the art, to improve or increase expression. Alternatively or additionally, one or more variants of the ACS/CoA ligase (or the homolog, or catalytically active portion thereof, or polypeptide having ACS/CoA ligase activity) can be generated by mutagenesis, whereby the variants have increased activity in catalyzing the conversion of 3HB to 3HB—CoA, and/or have increased specificity or selectivity for the 3HB substrate. In some embodiments, the ACS/CoA ligase variants have increased activity in catalyzing the conversion of a short-chain carboxylate to its corresponding CoA thioester or acyl-CoA, and/or have increased specificity and/or selectivity for the short-chain carboxylate substrate, wherein the short-chain carboxylate substrate includes, for example, one or more of 3HB, (R)-3HB, (S)-3HB, acetate, acetoacetate, succinate, crotonate, butyrate, isobutyrate, malonate, and/or hexanoate.

In some embodiments, the non-naturally occurring microbial organism or cell, having a 3HB recycling loop, has enhanced or increased carbon flux through 3HB—CoA. In certain embodiments, the non-naturally occurring microbial organism having a 3HB recycling loop has enhanced or increased carbon flux through 3HB—CoA as compared to (i) a microbial organism without a 3HB recycling loop, or as compared to (ii) a microbial organism without a one or more exogenous nucleic acid(s) encoding an ACS, or a CoA ligase, or a homolog, variant, or catalytically active portion thereof, or encoding a polypeptide with ACS or CoA ligase activity, as described herein, or as compared to both (i) and (ii).

In some embodiments, the non-naturally occurring microbial organism or cell, having at least one carboxylate by-product recycling loop, has enhanced or increased carbon flux through the CoA thioester(s) of the carboxylate(s) (or the acyl-CoA(s)). In certain embodiments, the non-naturally occurring microbial organism having at least one carboxylate by-product recycling loop has enhanced or increased carbon flux through the CoA thioester(s) (or acyl-CoA(s)) of the carboxylate by-product(s) as compared to (i) a microbial organism without a carboxylate by-product recycling loop, or as compared to (ii) a microbial organism without a one or more exogenous nucleic acid(s) encoding an ACS, or a CoA ligase, or a homolog, variant, or catalytically active portion thereof, or encoding a polypeptide with ACS or CoA ligase activity, as described herein, or as compared to both (i) and (ii).

In certain embodiments, the non-naturally occurring microorganism or cell having a 3HB recycling loop has enhanced or increased carbon flux through 3HB—CoA, as compared to (i) a microbial organism without a 3HB recycling loop, or as compared to (ii) a microbial organism without a one or more exogenous nucleic acid(s) encoding an ACS, or a CoA ligase, or a homolog, variant, or catalytically active portion thereof, or encoding a polypeptide with ACS or CoA ligase activity, as described herein, or as compared to both (i) and (ii), wherein the enhancement or increase or carbon flux through 3HB—CoA is about a 5% to about a 100% enhancement or increase. In some embodiments, the enhancement or increase of carbon flux through 3HB—CoA is about a 20% to about a 95% enhancement or increase. In some embodiments, the enhancement or increase of carbon flux through 3HB—CoA is about a 30% to about a 90% enhancement or increase. In some embodiments, the enhancement or increase of carbon flux through 3HB—CoA is about a 40% to about an 85% enhancement or increase. In some embodiments, the enhancement or increase of carbon flux through 3HB—CoA is about a 50% to about an 80% enhancement or increase.

In some embodiments, the enhancement or increase of carbon flux through 3HB—CoA is about a 5% enhancement or increase. In some embodiments, the enhancement or increase of carbon flux through 3HB—CoA is about a 10% enhancement or increase. In some embodiments, the enhancement or increase of carbon flux through 3HB—CoA is about a 15% enhancement or increase. In some embodiments, the enhancement or increase of carbon flux through 3HB—CoA is about a 20% enhancement or increase. In some embodiments, the enhancement or increase of carbon flux through 3HB—CoA is about a 25% enhancement or increase. In some embodiments, the enhancement or increase of carbon flux through 3HB—CoA is about a 30% enhancement or increase. In some embodiments, the enhancement or increase of carbon flux through 3HB—CoA is about a 35% enhancement or increase. In some embodiments, the enhancement or increase of carbon flux through 3HB—CoA is about a 40% enhancement or increase. In some embodiments, the enhancement or increase of carbon flux through 3HB—CoA is about a 45% enhancement or increase. In some embodiments, the enhancement or increase of carbon flux through 3HB—CoA is about a 50% enhancement or increase. In some embodiments, the enhancement or increase of carbon flux through 3HB—CoA is about a 55% enhancement or increase. In some embodiments, the enhancement or increase of carbon flux through 3HB—CoA is about a 60% enhancement or increase. In some embodiments, the enhancement or increase of carbon flux through 3HB—CoA is about a 65% enhancement or increase. In some embodiments, the enhancement or increase of carbon flux through 3HB—CoA is about a 70% enhancement or increase. In some embodiments, the enhancement or increase of carbon flux through 3HB—CoA is about a 75% enhancement or increase. In some embodiments, the enhancement or increase of carbon flux through 3HB—CoA is about an 80% enhancement or increase. In some embodiments, the enhancement or increase of carbon flux through 3HB—CoA is about an 85% enhancement or increase. In some embodiments, the enhancement or increase of carbon flux through 3HB—CoA is about a 90% enhancement or increase. In some embodiments, the enhancement or increase of carbon flux through 3HB—CoA is about a 95% enhancement or increase. In some embodiments, the enhancement or increase of carbon flux through 3HB—CoA is about a 96% enhancement or increase. In some embodiments, the enhancement or increase of carbon flux through 3HB—CoA is about a 97% enhancement or increase. In some embodiments, the enhancement or increase of carbon flux through 3HB—CoA is about a 98% enhancement or increase. In some embodiments, the enhancement or increase of carbon flux through 3HB—CoA is about a 99% enhancement or increase. In some embodiments, the enhancement or increase of carbon flux through 3HB—CoA is or about a 100% enhancement or increase.

In some embodiments, the non-naturally occurring microbial organism or cell, having a 3HB recycling loop, produces a reduced titer, amount, yield, and/or concentration of 3HB by-product. In some embodiments, the non-naturally occurring microbial organism or cell, having a 3HB recycling loop, produces a reduced titer, amount, yield, and/or concentration of 3HB by-product, as compared to (i) a microbial organism without a 3HB recycling loop, or as compared to (ii) a microbial organism without a one or more exogenous nucleic acid(s) encoding an ACS, or a CoA ligase, or a homolog, variant, or catalytically active portion thereof, or encoding a polypeptide with ACS or CoA ligase activity, as described herein, or as compared to both (i) and (ii).

In some embodiments, the non-naturally occurring microbial organism or cell, having a carboxylate by-product recycling loop, produces a reduced titer, amount, yield, and/or concentration of the carboxylate by-product. In some embodiments, the non-naturally occurring microbial organism or cell, having a carboxylate by-product recycling loop, produces a reduced titer, amount, yield, and/or concentration of a carboxylate by-product, as compared to (i) a microbial organism without a carboxylate by-product recycling loop, or as compared to (ii) a microbial organism without a one or more exogenous nucleic acid(s) encoding an ACS, or a CoA ligase, or a homolog, variant, or catalytically active portion thereof, or encoding a polypeptide with ACS or CoA ligase activity, as described herein, or as compared to both (i) and (ii).

In certain embodiments, the non-naturally occurring microorganism or cell having a 3HB recycling loop produces reduced 3HB titer, amount, yield, and/or concentration, as compared to (i) a microbial organism without a 3HB recycling loop, or as compared to (ii) a microbial organism without a one or more exogenous nucleic acid(s) encoding an ACS, or a CoA ligase, or a homolog, variant, or catalytically active portion thereof, or encoding a polypeptide with ACS or CoA ligase activity, as described herein, or as compared to both (i) and (ii), wherein the reduction of 3HB titer, amount, yield, and/or concentration is about a 5% to about a 100% reduction. In some embodiments, the reduction in 3HB titer, amount, yield, and/or concentration is about a 20% to 95% reduction, about a 30% to 90% reduction, about a 40% to 85% reduction, or about a 50% to 80% reduction.

In some embodiments, the reduction in 3HB titer, amount, yield, and/or concentration is about a 5% reduction. In some embodiments, the reduction in 3HB titer, amount, yield, and/or concentration is about a 10% reduction. In some embodiments, the reduction in 3HB titer, amount, yield, and/or concentration is about a 15% reduction. In some embodiments, the reduction in 3HB titer, amount, yield, and/or concentration is about a 20% reduction. In some embodiments, the reduction in 3HB titer, amount, yield, and/or concentration is about a 25% reduction. In some embodiments, the reduction in 3HB titer, amount, yield, and/or concentration is about a 30% reduction. In some embodiments, the reduction in 3HB titer, amount, yield, and/or concentration is about a 35% reduction. In some embodiments, the reduction in 3HB titer, amount, yield, and/or concentration is about a 40% reduction. In some embodiments, the reduction in 3HB titer, amount, yield, and/or concentration is about a 45% reduction. In some embodiments, the reduction in 3HB titer, amount, yield, and/or concentration is about a 50% reduction. In some embodiments, the reduction in 3HB titer, amount, yield, and/or concentration is about a 55% reduction. In some embodiments, the reduction in 3HB titer, amount, yield, and/or concentration is about a 60% reduction. In some embodiments, the reduction in 3HB titer, amount, yield, and/or concentration is about a 65% reduction. In some embodiments, the reduction in 3HB titer, amount, yield, and/or concentration is about a 70% reduction. In some embodiments, the reduction in 3HB titer, amount, yield, and/or concentration is about a 75% reduction. In some embodiments, the reduction in 3HB titer, amount, yield, and/or concentration is about an 80% reduction. In some embodiments, the reduction in 3HB titer, amount, yield, and/or concentration is about an 85% reduction. In some embodiments, the reduction in 3HB titer, amount, yield, and/or concentration is about a 90% reduction. In some embodiments, the reduction in 3HB titer, amount, yield, and/or concentration is about a 95% reduction. In some embodiments, the reduction in 3HB titer, amount, yield, and/or concentration is about a 96% reduction. In some embodiments, the reduction in 3HB titer, amount, yield, and/or concentration is about a 97% reduction. In some embodiments, the reduction in 3HB titer, amount, yield, and/or concentration is about a 98% reduction. In some embodiments, the reduction in 3HB titer, amount, yield, and/or concentration is about a 99% reduction. In some embodiments, the reduction in 3HB titer, amount, yield, and/or concentration is about a 100% reduction.

In certain embodiments, the non-naturally occurring microorganism or cell having a carboxylate by-product recycling loop produces reduced carboxylate by-product titer, amount, yield, and/or concentration, as compared to (i) a microbial organism without a carboxylate by-product recycling loop, or as compared to (ii) a microbial organism without a one or more exogenous nucleic acid(s) encoding an ACS, or a CoA ligase, or a homolog, variant, or catalytically active portion thereof, or encoding a polypeptide with ACS or CoA ligase activity, as described herein, or as compared to both (i) and (ii), wherein the reduction of carboxylate by-product titer, amount, yield, and/or concentration is about a 5% to about a 100% reduction. In some embodiments, the reduction in carboxylate by-product titer, amount, yield, and/or concentration is about a 10% to 90% reduction, about a 20% to 95% reduction, about a 30% to 90% reduction, about a 40% to 85% reduction, or about a 50% to 80% reduction.

In some embodiments, the reduction in carboxylate by-product titer, amount, yield, productivity, rate and/or concentration is about a 5%, about a 10%, about a 15%, about a 20%, about a 25%, about a 30%, about a 35%, about a 40%, about a 45%, about a 50%, about a 55%, about a 60%, about a 65%, about a 70%, about a 75%, about an 80%, about an 85%, about a 90%, about a 95%, about a 96%, about a 97%, about a 98%, about a 99%, or about a 100% reduction.

As described above, decreasing the titer, amount, yield, and/or concentration of 3HB and/or one or more other carboxylate by-products, such as by having or expressing a 3HB and/or other carboxylate by-product recycling loop(s), can increase carbon flux through 3HB—CoA, and/or the CoA thioester(s) (or acyl-CoA(s)) of the one or more other carboxylate by-products, and therefore, can increase the production of Ac-COA derived products with a 3HB—CoA and/or one or more other CoA thioester or acyl-CoA intermediate or precursor, such as, for example, 1,3-BDO, (S)-1,3-BDO, (R)-1,3-BDO, MAA or its esters (e.g., MMA, EMA, and others), (3R)-hydroxybutyl (3R)-hydroxybutyrate, HMD, adipate, 6ACA, CPL, and/or HDO. Accordingly, in some embodiments, the microorganism or cell, having a 3HB and/or one or more other carboxylate by-product recycling loops, can have enhanced or increased carbon flux through 3HB—CoA and/or one or more CoA thioesters or acyl-CoAs of the one or more other carboxylate by-products, and increased production, titer, and/or yield of one or more Ac—CoA derived products, such as, for example, 1,3-BDO, (S)-1,3-BDO, (R)-1,3-BDO, (3R)-hydroxybutyl (3R)-hydroxybutyrate, MAA or its esters (e.g., MMA, EMA, and others), HMD, adipate, 6ACA, CPL, and/or HDO. In some embodiments, the microorganism or cell, having a 3HB and/or one or more other carboxylate by-product recycling loops, can have reduced, eliminated, or substantially eliminated 3HB and/or one or more carboxylate by-product production, titer, and/or yield, and increased production, titer, and/or yield of one or more Ac—CoA derived products, such as, for example, 1,3-BDO, (S)-1,3-BDO, (R)-1,3-BDO, (3R)-hydroxybutyl (3R)-hydroxybutyrate, MAA or its esters (e.g., MMA, EMA, and others), HMD, adipate, 6ACA, CPL, and/or HDO. For example, since acetyl-CoA is a precursor or intermediate for the production of all acetyl-CoA derived products, an engineered microorganism having an acetate by-product recycling loop (or acetate recycling loop) can have reduced, eliminated, or substantially eliminated acetate by-product formation, and increased production, titer, yield, and/or productivity of any one or more acetyl-CoA derived products described herein or known in the art.

In certain embodiments, the non-naturally occurring microbial organism or cell having a 3HB or carboxylate by-product recycling loop has increased production, titer, and/or yield of one or more Ac-COA derived product, as compared to (i) a microbial organism or cell without a 3HB or carboxylate by-product recycling loop, or as compared to (ii) a microbial organism without a one or more exogenous nucleic acid(s) encoding an ACS, a CoA ligase, or homologs, variants, or catalytically active portions thereof, or encoding a polypeptide with ACS or CoA ligase activity, as described herein, or as compared to both (i) and (ii).

In certain embodiments, the non-naturally occurring microorganism or cell having a 3HB or carboxylate by-product recycling loop has increased production, titer, and/or yield of an Ac-COA derived product or products, as compared to (i) a microbial organism without a 3HB or carboxylate by-product recycling loop, or as compared to (ii) a microbial organism without a one or more exogenous nucleic acid(s) encoding an ACS, or a CoA ligase, or a homolog, variant, or catalytically active portion thereof, or encoding a polypeptide with ACS or CoA ligase activity, as described herein, or as compared to both (i) and (ii), wherein the increase in production, titer, and/or yield of an Ac-COA derived product or products is about a 1% to about a 50% increase, or more. In some embodiments, the increase in production, titer, and/or yield of an Ac-COA derived product or products is about a 2% to about a 40% increase, about a 3% to about a 30% increase, about a 4% to about a 20% increase, or about a 5% to about a 15% increase.

In some embodiments, the increase in production, titer, and/or yield of an Ac-COA derived product or products is about or at least about a 1% increase. In some embodiments, the increase in production, titer, and/or yield of an Ac-COA derived product or products is about or at least about a 2% increase. In some embodiments, the increase in production, titer, and/or yield of an Ac-COA derived product or products is about or at least about a 3% increase. In some embodiments, the increase in production, titer, and/or yield of an Ac-COA derived product or products is about or at least about a 4% increase. In some embodiments, the increase in production, titer, and/or yield of an Ac-COA derived product or products is about or at least about a 5% increase. In some embodiments, the increase in production, titer, and/or yield of an Ac-COA derived product or products is about or at least about a 6% increase. In some embodiments, the increase in production, titer, and/or yield of an Ac-COA derived product or products is about or at least about a 7% increase. In some embodiments, the increase in production, titer, and/or yield of an Ac-COA derived product or products is about or at least about an 8% increase. In some embodiments, the increase in production, titer, and/or yield of an Ac-COA derived product or products is about or at least about a 9% increase. In some embodiments, the increase in production, titer, and/or yield of an Ac-COA derived product or products is about or at least about a 10% increase. In some embodiments, the increase in production, titer, and/or yield of an Ac-COA derived product or products is about or at least about a 11% increase. In some embodiments, the increase in production, titer, and/or yield of an Ac-COA derived product or products is about or at least about a 12% increase. In some embodiments, the increase in production, titer, and/or yield of an Ac—CoA derived product or products is about or at least about a 13% increase. In some embodiments, the increase in production, titer, and/or yield of an Ac-COA derived product or products is about or at least about a 14% increase. In some embodiments, the increase in production, titer, and/or yield of an Ac-COA derived product or products is about or at least about a 15% increase. In some embodiments, the increase in production, titer, and/or yield of an Ac-COA derived product or products is about or at least about a 16% increase. In some embodiments, the increase in production, titer, and/or yield of an Ac-COA derived product or products is about or at least about a 17% increase. In some embodiments, the increase in production, titer, and/or yield of an Ac-COA derived product or products is about or at least about a 18% increase. In some embodiments, the increase in production, titer, and/or yield of an Ac-COA derived product or products is about or at least about a 19% increase. In some embodiments, the increase in production, titer, and/or yield of an Ac-COA derived product or products is about or at least about a 20% increase. In some embodiments, the increase in production, titer, and/or yield of an Ac-COA derived product or products is about or at least about a 21% increase. In some embodiments, the increase in production, titer, and/or yield of an Ac—CoA derived product or products is about or at least about a 22% increase. In some embodiments, the increase in production, titer, and/or yield of an Ac-COA derived product or products is about or at least about a 23% increase. In some embodiments, the increase in production, titer, and/or yield of an Ac-COA derived product or products is about or at least about a 24% increase. In some embodiments, the increase in production, titer, and/or yield of an Ac-COA derived product or products is about or at least about a 25% increase. In some embodiments, the increase in production, titer, and/or yield of an Ac-COA derived product or products is about or at least about a 26% increase. In some embodiments, the increase in production, titer, and/or yield of an Ac-COA derived product or products is about or at least about a 27% increase. In some embodiments, the increase in production, titer, and/or yield of an Ac-COA derived product or products is about or at least about a 28% increase. In some embodiments, the increase in production, titer, and/or yield of an Ac-COA derived product or products is about or at least about a 29% increase. In some embodiments, the increase in production, titer, and/or yield of an Ac-COA derived product or products is about or at least about a 30% increase. In some embodiments, the increase in production, titer, and/or yield of an Ac-COA derived product or products is about or at least about a 31% increase. In some embodiments, the increase in production, titer, and/or yield of an Ac-COA derived product or products is about or at least about a 32% increase. In some embodiments, the increase in production, titer, and/or yield of an Ac-COA derived product or products is about or at least about a 33% increase. In some embodiments, the increase in production, titer, and/or yield of an Ac-COA derived product or products is about or at least about a 34% increase. In some embodiments, the increase in production, titer, and/or yield of an Ac-COA derived product or products is about or at least about a 35% increase. In some embodiments, the increase in production, titer, and/or yield of an Ac-COA derived product or products is about or at least about a 36% increase. In some embodiments, the increase in production, titer, and/or yield of an Ac-COA derived product or products is about or at least about a 37% increase. In some embodiments, the increase in production, titer, and/or yield of an Ac-COA derived product or products is about or at least about a 38% increase. In some embodiments, the increase in production, titer, and/or yield of an Ac-COA derived product or products is about or at least about a 39% increase. In some embodiments, the increase in production, titer, and/or yield of an Ac-COA derived product or products is about or at least about a 40% increase. In some embodiments, the increase in production, titer, and/or yield of an Ac-COA derived product or products is about or at least about a 41% increase. In some embodiments, the increase in production, titer, and/or yield of an Ac-COA derived product or products is about or at least about a 42% increase. In some embodiments, the increase in production, titer, and/or yield of an Ac-COA derived product or products is about or at least about a 43% increase. In some embodiments, the increase in production, titer, and/or yield of an Ac—CoA derived product or products is about or at least about a 44% increase. In some embodiments, the increase in production, titer, and/or yield of an Ac-COA derived product or products is about or at least about a 45% increase. In some embodiments, the increase in production, titer, and/or yield of an Ac-COA derived product or products is about or at least about a 46% increase. In some embodiments, the increase in production, titer, and/or yield of an Ac-COA derived product or products is about or at least about a 47% increase. In some embodiments, the increase in production, titer, and/or yield of an Ac-COA derived product or products is about or at least about a 48% increase. In some embodiments, the increase in production, titer, and/or yield of an Ac-COA derived product or products is about or at least about a 49% increase. In some embodiments, the increase in production, titer, and/or yield of an Ac-COA derived product or products is about or at least about a 50% increase or more.

In some embodiments, the reduction, elimination, or substantial elimination of the 3HB by-product or the carboxylate by-product results in about a 1.1-fold to about a 10-fold increase in the production, titer, and/or yield of an Ac-COA derived product or products, such as, for example, 1,3-BDO, (S)-1,3-BDO, (R)-1,3-BDO, butyrate, butanol, propane, formate, MAA or its esters (e.g., MMA, EMA, and others), (3R)-hydroxybutyl (3R)-hydroxybutyrate, HMD, adipate, 6ACA, CPL, or HDO, as compared to a microbial organism or cell without (i) a 3HB or carboxylate by-product recycling loop, or as compared to (ii) a microbial organism or cell without a one or more exogenous nucleic acids encoding an ACS, or a CoA ligase, or homologs, variants, or catalytically active portions thereof, or encoding a polypeptide with ACS or CoA ligase activity, as described herein, or as compared to both (i) and (ii).

In some embodiments, the reduction, elimination, or substantial elimination in the 3HB by-product or the carboxylate by-product results in about a 1.1-fold increase, or greater, in the production, titer, and/or yield of one or more Ac-COA derived products, such as, for example, 1,3-BDO, (S)-1,3-BDO, (R)-1,3-BDO, (3R)-hydroxybutyl (3R)-hydroxybutyrate, butyrate, butanol, propane, formate, MAA or its esters (e.g., MMA, EMA, and others), HMD, adipate, 6ACA, CPL, and/or HDO. In some embodiments, the reduction, elimination, or substantial elimination in the 3HB by-product or the carboxylate by-product results in about a 1.5-fold increase, a 2-fold increase, a 2.5-fold increase, a 3-fold increase, a 3.5-fold increase, a 4-fold increase, a 4.5-fold increase, a 5-fold increase, a 5.5-fold increase, a 6-fold increase, a 6.5-fold increase, a 7-fold increase, a 7.5-fold increase, an 8-fold increase, an 8.5-fold increase, a 9-fold increase, a 9.5-fold increase, a 10-fold or more increase, or greater, in the production, titer, and/or yield of one or more Ac-COA derived products, such as, for example, 1,3-BDO, (S)-1,3-BDO, (R)-1,3-BDO, (3R)-hydroxybutyl (3R)-hydroxybutyrate, butyrate, butanol, propane, formate, isopropanol, MAA or its esters (e.g., MMA, EMA, and others), HMD, adipate, 6ACA, CPL, and/or HDO.

The conversion of 3HB to 3HB—CoA, or the conversion of a carboxylate by-product (e.g., acetate, acetoacetate, succinate, crotonate, butyrate, isobutyrate, malonate, and/or hexanoate) to its corresponding acyl-CoA (e.g., acetyl-CoA, acetoacetyl-CoA, succinyl-CoA, crotonyl-CoA, butyryl-CoA, isobutyryl-CoA, malonyl-CoA, and/or hexanoyl-CoA), as described herein, can be catalyzed by an enzyme, protein or polypeptide with acyl-CoA synthetase or CoA ligase activity. This reaction results in the production of AMP, and is described by EC 6.2.1.-, EC 6.2.1.1, and/or EC 6.2.2.2. The amino acid sequence of an exemplary ACS is set forth in SEQ ID NO: 1 and is shown in the table below.

TABLE 1
Exemplary Acyl-CoA Synthetase (ACS)

| SEQ | | |
|---|---|---|
| ID | | |
| NO: | Sequence | Description |
| 1 | Acyl-CoA Synthetase | MTGSTIYDQHLDRTPANFTPLSPLSYIERTAAVYPDYPSV |
| | (ACS) from Ruegeria | VYGDRRYTWAETYTRCRRLASALAGRGLGKGDTVSIIA |
| | pomeroyi (Accession | ANIPEMYEAHFGVPMAGAVLNAINTRLDAPIIAFILSHAE |
| | No. | ARVLIVDPEFSEVVRDALAQIDRPDLLVVDIEDPSFAGGA |
| | WP_011046428.1) | PVGTLSYDALLAEGDPDFDWSLPGDEWDAIALNYTSGT |
| | | TGNPKGVVYHHRGAALNATSNILTWGMPQHAVYLWTL |
| | | PMFHCNGWCFPWTMAANAGVSVCLRAVRDEPIYRAFR |
| | | EEKVTHFCGAPIVLNMLANAPDHMKDFDHQIKVMTAGA |
| | | PPPAAVIEKMEAMGVDVTHVYGLTETYGPSVVCAWKEE |
| | | WDGRPGAERAALKVRQGVRYVALSGLMVADPETLEPV |
| | | PADGETMGEIFMQGNIVMKGYLKNPDATDRAFRGGWF |
| | | ASGDLGVMHPDGYIALKDRSKDIIISGGENISSVEVEDVL |
| | | YKHPAVMEAAVVARPDEKWGETPCAFVELKPGQSVEA |
| | | ADLIAHCRANMAHFKAPKTVVFGELPKTSTGKIQKFLLR |
| | | ERARAL |

Further exemplary ACS homologs, that can be used in the 3HB recycling loop or carboxylate by-product recycling loops described herein, are shown in the table below. The percent identity of each homolog, relative to the ACS of Ruegeria pomeroyi (SEQ ID NO: 1), also is listed in the table below.

TABLE 2
Exemplary Acyl-CoA Synthetase (ACS) Homologs

| | | | % |
|---|---|---|---|
| Enzyme | Organism | Accession No. | Identity |
| acyl-CoA | Ruegeria | WP_011046428.1 | 100 |
| synthetase | pomeroyi | | |
| acyl-CoA | Ruegeria | WP_164130245.1 | 94.10 |
| synthetase | sp. PrR005 | | |
| acyl-CoA | Roseibium | WP_155149127.1 | 94.10 |
| synthetase | sp. RKSG952 | | |
| acyl-CoA | Ruegeria | WP_093035935.1 | 93.91 |
| synthetase | marina | | |
| acyl-CoA | Ruegeria | WP_138845291.1 | 93.17 |
| synthetase | sediminis | | |
| acyl-CoA | Ruegeria | WP_068344751.1 | 93.17 |
| synthetase | marisrubri | | |
| acyl-CoA | Rhodobacteraceae | WP_121998088.1 | 92.62 |
| synthetase | bacterium EL53 | | |
| acyl-CoA | Roseobacteraceae | WP_113825616.1 | 92.62 |
| synthetase | | | |
| acyl-CoA | Cribrihabitans | WP_092368979.1 | 91.70 |
| synthetase | marinus | | |
| acyl-CoA | Rhodobacteraceae | WP_164621800.1 | 92.25 |
| synthetase | bacterium | | |
| | Alg241-R94 | | |
| acyl-CoA | Falsiruegeria | WP_108789882.1 | 90.77 |
| synthetase | mediterranea | | |
| acyl-CoA | Rhodobacteraceae | WP_008755673.1 | 91.88 |
| synthetase | bacterium KLH11 | | |
| acyl-CoA | Ruegeria | WP_039541522.1 | 91.14 |
| synthetase | sp. ANG-R | | |
| acyl-CoA | Ruegeria | WP_010437551.1 | 91.70 |
| synthetase | conchae | | |
| acyl-CoA | Ruegeria | WP_120634479.1 | 91.88 |
| synthetase | sp. EL01 | | |
| acyl-CoA | Tropicibacter | WP_164661710.1 | 91.51 |
| synthetase | sp. Alg240-R139 | | |
| acyl-CoA | Ruegeria | WP_068339543.1 | 92.44 |
| synthetase | profundi | | |
| acyl-CoA | Ruegeria | WP_108861336.1 | 91.88 |
| synthetase | sp. Alg231-54 | | |
| acyl-CoA | Falsiruegeria | WP_085794254.1 | 90.96 |
| synthetase | litorea | | |
| acyl-CoA | Ruegeria | WP_152457004.1 | 92.07 |
| synthetase | sp. THAF33 | | |
| acyl-CoA | Ruegeria | WP_142639092.1 | 92.44 |
| synthetase | faecimaris | | |
| acyl-CoA | Ruegeria | WP_109312224.1 | 91.88 |
| synthetase | sp. AU67 | | |
| acyl-CoA | Ruegeria | WP_074737858.1 | 90.59 |
| synthetase | halocynthiae | | |
| acyl-CoA | Ruegeria | WP_058273344.1 | 91.70 |
| synthetase | atlantica | | |
| acyl-CoA | Ruegeria | WP_037311307.1 | 91.70 |
| synthetase | halocynthiae | | |
| acyl-CoA | Ruegeria | WP_149776329.1 | 92.07 |
| synthetase | intermedia | | |
| acyl-CoA | Ruegeria | WP_005981195.1 | 92.07 |
| synthetase | lacuscaerulensis | | |
| acyl-CoA | Ruegeria | WP_113791374.1 | 91.70 |
| synthetase | sp. A3M17 | | |
| acyl-CoA | Ruegeria | WP_058279001.1 | 91.70 |
| synthetase | atlantica | | |
| acyl-CoA | Ruegeria | WP_085824387.1 | 90.77 |
| synthetase | meonggei | | |
| acyl-CoA | Ruegeria | WP_093965160.1 | 91.88 |
| synthetase | arenilitoris | | |
| acyl-CoA | Ruegeria | WP_058283544.1 | 90.77 |
| synthetase | denitrificans | | |
| acyl-CoA | Ruegeria | WP_039532659.1 | 89.67 |
| synthetase | sp. ANG-S4 | | |
| acyl-CoA | Ruegeria | WP_117869934.1 | 91.14 |
| synthetase | sp. AD91A | | |
| acyl-CoA | Ruegeria | WP_050605291.1 | 90.96 |
| synthetase | sp. 6PALISEP08 | | |
| acyl-CoA | Thioclava | WP_096433911.1 | 85.95 |
| synthetase | arenosa | | |
| acyl-CoA | Paracoccus | WP_095638535.1 | 85.98 |
| synthetase | salipaludis | | |
| acyl-CoA | Rhodobacteraceae | TNE69928.1 | 85.79 |
| synthetase | bacterium | | |
| acyl-CoA | Celeribacter | WP_096807141.1 | 85.79 |
| synthetase | ethanolicus | | |
| acyl-CoA | Paracoccus | WP_090756690.1 | 86.72 |
| synthetase | chinensis | | |
| acyl-CoA | Celeribacter | WP_066706782.1 | 85.42 |
| synthetase | ethanolicus | | |
| acyl-CoA | Gemmobacter | WP_091302351.1 | 84.69 |
| synthetase | aquatilis | | |
| acyl-CoA | Paracoccus | RQP07028.1 | 86.35 |
| synthetase | sp. BP8 | | |
| acyl-CoA | Marivivens | WP_086450185.1 | 85.93 |
| synthetase | niveibacter | | |
| acyl-CoA | Paracoccus | WP_101461757.1 | 84.53 |
| synthetase | sp. BM15 | | |
| acyl-CoA | unclassified | WP_028712121.1 | 86.16 |
| synthetase | Paracoccus | | |
| acyl-CoA | Paracoccus | WP_147099428.1 | 85.40 |
| synthetase | aurantiacus | | |
| acyl-CoA | Actibacterium | WP_035252339.1 | 84.87 |
| synthetase | atlanticum | | |
| acyl-CoA | Paracoccus | WP_090613365.1 | 85.56 |
| synthetase | alcaliphilus | | |
| acyl-CoA | Paracoccus | WP_074966379.1 | 84.32 |
| synthetase | aminovorans | | |
| acyl-CoA | Salipiger | WP_076697957.1 | 84.87 |
| synthetase | abyssi | | |
| acyl-CoA | Paracoccus | WP_166330075.1 | 85.21 |
| synthetase | xiamenensis | | |
| acyl-CoA | Paracoccus | WP_062563701.1 | 84.92 |
| synthetase | aminovorans | | |
| acyl-CoA | Youngimonas | WP_136340373.1 | 85.24 |
| synthetase | vesicularis | | |
| acyl-CoA | Paracoccus | TFE37157.1 | 84.92 |
| synthetase | sp. DMF | | |
| acyl-CoA | Poseidonocella | WP_138467233.1 | 86.30 |
| synthetase | sp. HB161398 | | |
| acyl-CoA | Paracoccus | WP_142662930.1 | 84.16 |
| synthetase | laeviglucosivorans | | |
| acyl-CoA | Paracoccus | WP_119900706.1 | 84.6 |
| synthetase | siganidrum | | |
| acyl-CoA | Paracoccus | WP_134726281.1 | 85.00 |
| synthetase | luteus | | |
| acyl-CoA | Paracoccus | WP_018000194.1 | 84.73 |
| synthetase | sp. N5 | | |
| acyl-CoA | Paracoccus | WP_143793102.1 | 84.53 |
| synthetase | sp. M683 | | |
| acyl-CoA | Paracoccus | WP_101500069.1 | 83.61 |
| synthetase | jeotgali | | |
| acyl-CoA | Paracoccus | WP_036742235.1 | 84.69 |
| synthetase | halophilus | | |
| acyl-CoA | Paracoccus | WP_155065929.1 | 84.50 |
| synthetase | limosus | | |
| acyl-CoA | unclassified | WP_155043768.1 | 83.98 |
| synthetase | Paracoccus | | |
| acyl-CoA | Nitratireductor | WP_136660038.1 | 84.01 |
| synthetase | sp. XY-223 | | |
| acyl-CoA | Rhodobacteraceae | WP_111429653.1 | 84.79 |
| synthetase | bacterium | | |
| | DSL-40 | | |
| acyl-CoA | Paracoccus | WP_112873394.1 | 83.58 |
| synthetase | endophyticus | | |
| acyl-CoA | Paracoccus | WP_020948954.1 | 83.76 |
| synthetase | aminophilus | | |
| acyl-CoA | Pseudooceanicola | WP_085886331.1 | 84.42 |
| synthetase | marinus | | |
| acyl-CoA | Paracoccus | TKW64993.1 | 83.49 |
| synthetase | denitrificans | | |
| acyl-CoA | Marivivens sp. | NBX08360.1 | 83.99 |
| synthetase | | | |
| acyl-CoA | Rhizobium | WP_084363456.1 | 83.46 |
| synthetase | sp. RU36D | | |
| acyl-CoA | Aestuarium | WP_105235638.1 | 83.96 |
| synthetase | zhoushanense | | |
| acyl-CoA | Pseudodonghicola | WP_028091971.1 | 83.46 |
| synthetase | xiamenensis | | |
| acyl-CoA | Rhodobacterales | WP_067549780.1 | 83.77 |
| synthetase | | | |
| acyl-CoA | Ciceribacter | WP_115669588.1 | 83.03 |
| synthetase | selenitireducens | | |
| acyl-CoA | Thioclava | WP_096434540.1 | 82.37 |
| synthetase | arenosa | | |
| acyl-CoA | Ciceribacter | WP_154722873.1 | 82.47 |
| synthetase | naphthalenivorans | | |
| acyl-CoA | Polymorphum | WP_013651294.1 | 82.26 |
| synthetase | gilvum | | |
| acyl-CoA | Celeribacter | WP_066704804.1 | 82.56 |
| synthetase | ethanolicus | | |
| acyl-CoA | Rhizobiales | WP_113487523.1 | 82.99 |
| synthetase | bacterium | | |
| acyl-CoA | Rhodobacteraceae | WP_111431920.1 | 82.75 |
| synthetase | bacterium DSL-40 | | |
| acyl-CoA | Salipiger | WP_076697983.1 | 82.37 |
| synthetase | abyssi | | |
| acyl-CoA | Rhizobiales | WP_113442369.1 | 82.81 |
| synthetase | bacterium | | |
| acyl-CoA | Rhizobiales | WP_113340940.1 | 82.81 |
| synthetase | bacterium | | |
| acyl-CoA | Celeribacter | WP_107720993.1 | 82.93 |
| synthetase | baekdonensis | | |
| acyl-CoA | Rhizobiales | WP_113447564.1 | 82.81 |
| synthetase | bacterium | | |
| acyl-CoA | Rhizobiales | WP_112828800.1 | 82.81 |
| synthetase | bacterium | | |
| acyl-CoA | Rhizobiales | WP_113466554.1 | 82.62 |
| synthetase | bacterium | | |
| acyl-CoA | Celeribacter | WP_107817574.1 | 82.56 |
| synthetase | persicus | | |
| acyl-CoA | Rhizobiaceae | WP_051438881.1 | 82.26 |
| synthetase | | | |
| fatty-acyl-CoA | Celeribacter | PTQ68305.1 | 82.56 |
| synthase | persicus | | |
| acyl-CoA | Ciceribacter | WP_147180576.1 | 82.44 |
| synthetase | naphthalenivorans | | |
| acyl-CoA | Rhizobium sp. | PJI41333.1 | 81.89 |
| synthetase | | | |
| acyl-CoA | Celeribacter | WP_009574226.1 | 82.00 |
| synthetase | baekdonensis | | |
| acyl-CoA | Celeribacter | WP_074646446.1 | 82.19 |
| synthetase | baekdonensis | | |
| acyl-CoA | Roseobacter | WP_152003206.1 | 81.82 |
| synthetase | sp. TSBP12 | | |
| acyl-CoA | Rhizobium | WP_163905799.1 | 81.89 |
| synthetase | daejeonense | | |
| acyl-CoA | Paracoccus | WP_145396361.1 | 82.47 |
| synthetase | sulfuroxidans | | |

In some embodiments, the ACS is derived from a species or strain of Ruegeria, Roseibium, Rhodobacteraceae, Roseobacteraceae, Cribrihabitans, Falsiruegeria, Tropicibacter, Thioclava, Paracoccus, Celeribacter, Gemmobacter, Marivivens, unclassified, Actibacterium, Salipiger, Youngimonas, Poseidonocella, Nitratireductor, Pseudooceanicola, Rhizobium, Aestuarium, Pseudodonghicola, Rhodobacterales, Ciceribacter, Polymorphum, Rhizobiales, Rhizobiaceae, or Roseobacter. For example, the ACS can be derived from a strain or species of Ruegeria pomeroyi, Ruegeria sp. PrR005, Roseibium sp. RKSG952, Ruegeria marina, Ruegeria sediminis, Ruegeria marisrubri, Rhodobacteraceae bacterium EL53, Roseobacteraceae, Cribrihabitans marinus, Rhodobacteraceae bacterium Alg241-R94, Falsiruegeria mediterranea, Rhodobacteraceae bacterium KLH11, Ruegeria sp. ANG-R, Ruegeria conchae, Ruegeria sp. EL01, Tropicibacter sp. Alg240-R139, Ruegeria profundi, Ruegeria sp. Alg231-54, Falsiruegeria litorea, Ruegeria sp. THAF33, Ruegeria faecimaris, Ruegeria sp. AU67, Ruegeria halocynthiae, Ruegeria atlantica, Ruegeria halocynthiae, Ruegeria intermedia, Ruegeria lacuscaerulensis, Ruegeria sp. A3M17, Ruegeria atlantica, Ruegeria meonggei, Ruegeria arenilitoris, Ruegeria denitrificans, Ruegeria sp. ANG-S4, Ruegeria sp. AD91A, Ruegeria sp. 6PALISEP08, Thioclava arenosa, Paracoccus salipaludis, Rhodobacteraceae bacterium, Celeribacter ethanolicus, Paracoccus chinensis, Celeribacter ethanolicus, Gemmobacter aquatilis, Paracoccus sp. BP8, Marivivens niveibacter, Paracoccus sp. BM15, unclassified Paracoccus, Paracoccus aurantiacus, Actibacterium atlanticum, Paracoccus alcaliphilus, Paracoccus aminovorans, Salipiger abyssi, Paracoccus xiamenensis, Paracoccus aminovorans, Youngimonas vesicularis, Paracoccus sp. DMF, Poseidonocella sp. HB161398, Paracoccus laeviglucosivorans, Paracoccus siganidrum, Paracoccus luteus, Paracoccus sp. N5, Paracoccus sp. M683, Paracoccus jeotgali, Paracoccus halophilus, Paracoccus limosus, unclassified Paracoccus, Nitratireductor sp. XY-223, Rhodobacteraceae bacterium DSL-40, Paracoccus endophyticus, Paracoccus aminophilus, Pseudooceanicola marinus, Paracoccus denitrificans, Marivivens sp., Rhizobium sp. RU36D, Aestuarium zhoushanense, Pseudodonghicola xiamenensis, Rhodobacterales, Ciceribacter selenitireducens, Thioclava arenosa, Ciceribacter naphthalenivorans, Polymorphum gilvum, Celeribacter ethanolicus, Rhizobiales bacterium, Rhodobacteraceae bacterium DSL-40, Salipiger abyssi, Rhizobiales bacterium, Rhizobiales bacterium, Celeribacter baekdonensis, Rhizobiales bacterium, Rhizobiales bacterium, Rhizobiales bacterium, Celeribacter persicus, Rhizobiaceae, Celeribacter persicus, Ciceribacter naphthalenivorans, Rhizobium sp., Celeribacter baekdonensis, Celeribacter baekdonensis, Roseobacter sp. TSBP12, Rhizobium daejeonense, or Paracoccus sulfuroxidans.

In certain embodiments, the polypeptide or enzyme with acyl-CoA synthetase activity is a wild-type ACS. In some embodiments, the wild-type ACS comprises the sequence set forth in SEQ ID NO: 1. In certain embodiments, the polypeptide or enzyme with acyl-CoA synthetase activity is an ACS variant. In some embodiments, the ACS variant is variant of SEQ ID NO: 1. In some embodiments, the ACS variant can be an ACS enzyme that is overexpressed relative to the wild-type ACS, but retains ACS activity. In some embodiments, the ACS variant has higher activity relative to the WT ACS. In some embodiments, the term “activity” as used herein to describe enzymatic activity, refers to catalytic activity, substrate specificity, substrate affinity, substrate selectivity, or any combination thereof. In some embodiments, the ACS variant contains one or more amino acid modifications (e.g., amino acid replacements/substitutions (mutations), additions, and/or deletions) that enhance or increase expression of the ACS variant, relative to WT ACS. In some embodiments, the ACS variant has increased activity for a 3HB or carboxylate by-product recycling loop, and/or has increased selectivity, affinity and/or specificity for a carboxylate by-product substrate (e.g., 3HB and others described herein), compared to WT ACS. Accordingly, in some embodiments, an ACS variant has 1) higher activity for the conversion of a carboxylate by-product (e.g., 3HB) to their CoA thioesters or acyl-CoAs (e.g., 3HB—CoA); 2) higher specificity for the carboxylate by-product (e.g., 3HB); 3) higher affinity for the carboxylate by-product (e.g., 3HB); or any combination thereof, as compared to WT ACS (e.g., SEQ ID NO: 1). Hence, in certain embodiments, the non-naturally occurring microbial organism having a 3HB or carboxylate by-product recycling loop and at least one nucleic acid, e.g., at least one exogenous nucleic acid, encoding an ACS variant, has enhanced carbon flux through CoA thioesters or acyl-CoAs (e.g., 3HB—CoA), and/or eliminated, substantially eliminated, or reduced production of carboxylate by-product (e.g., 3HB), and/or increased productivity, titer, and/or yield of one or more acetyl-CoA derived products or intermediate(s) thereof, when compared to a non-naturally occurring microbial organism having a 3HB or carboxylate by-product recycling loop and at least one exogenous nucleic acid encoding a WT ACS.

In some embodiments, the ACS variant can exhibit an activity that is at least the same or higher than the WT ACS (e.g., SEQ ID NO: 1), that is, it has activity that is the same or higher than an ACS without the modification or mutation at the same or corresponding amino acid position(s). In some embodiments, the ACS variant can exhibit two or more activities (e.g., ability to catalyze a reaction described herein and selectivity for a carboxylate by-product (e.g., 3HB) substrate) that are at least the same or higher than the WT ACS (e.g., SEQ ID NO: 1), that is, it has two or more activities that are the same or higher than an ACS without the modification or mutation at the same or corresponding amino acid position(s). For example, the ACS variants provided here can have one or more activity that is at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, or at least 200% higher, over a WT ACS. In some embodiments, an ACS variant provided herein has an activity that is at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, or at least 200% higher than the activity of a WT ACS consisting of or comprising the amino acid sequence of SEQ ID NO: 1. In some embodiments, an ACS variant provided herein has an activity that is at least 10% higher. In some embodiments, an ACS variant provided herein has an activity that is at least 20% higher. In some embodiments, an ACS variant provided herein has an activity that is at least 30% higher. In some embodiments, an ACS variant provided herein has an activity that is at least 40% higher. In some embodiments, an ACS variant provided herein has an activity that is at least 50% higher. In some embodiments, an ACS variant provided herein has an activity that is at least 60% higher. In some embodiments, an ACS variant provided herein has an activity that is at least 70% higher. In some embodiments, an ACS variant provided herein has an activity that is at least 80% higher. In some embodiments, an ACS variant provided herein has an activity that is at least 90% higher. In some embodiments, an ACS variant provided herein has an activity that is at least 100% higher. In some embodiments, an ACS variant provided herein has an activity that is at least 110% higher. In some embodiments, an ACS variant provided herein has an activity that is at least 120% higher. In some embodiments, an ACS variant provided herein has an activity that is at least 130% higher. In some embodiments, an ACS variant provided herein has an activity that is at least 140% higher. In some embodiments, an ACS variant provided herein has an activity that is at least 150% higher. In some embodiments, an ACS variant provided herein has an activity that is at least 160% higher. In some embodiments, an ACS variant provided herein has an activity that is at least 170% higher. In some embodiments, an ACS variant provided herein has an activity that is at least 180% higher. In some embodiments, an ACS variant provided herein has an activity that is at least 190% higher. In some embodiments, an ACS variant provided herein has an activity that is at least 200% higher. It is understood that activity refers to the ability of an ACS variant described herein to convert a substrate to a product relative to a WT ACS (e.g., SEQ ID NO: 1) under the same assay conditions, such as those known in the art.

In some embodiments, an ACS variant provided herein is a variant of a reference (or template) polypeptide, wherein the reference (or template) polypeptide has an amino acid sequence of SEQ ID NO: 1, and the ACS variant has one or more amino acid alterations or modifications at one or more positions relative to SEQ ID NO: 1. In some embodiments, the one or more amino acid alterations or modifications comprise one or more amino acid additions, deletions, substitutions (replacements), or any combination thereof. In some embodiments, the one or more amino acid alterations or modifications are conservative amino acid substitutions. In some embodiments, the one or more amino acid alterations are non-conservative amino acid substitutions. In some embodiments, an ACS variant provided herein includes a conservative amino acid substitution and/or a non-conservative amino acid substitution in 1 to 100 amino acid positions. In some aspects, such a conservative amino acid substitution is a chemically conservative or an evolutionary conservative amino acid substitution. Methods of identifying conservative amino acids are well known to one of skill in the art, any one of which can be used to generate the variant ACSs described herein.

An ACS variant provided herein may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 amino acid alterations or modifications relative to a WT ACS (e.g., SEQ ID NO: 1). An ACS variant provided herein may comprise at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 31, at most 32, at most 33, at most 34, at most 35, at most 36, at most 37, at most 38, at most 39, at most 40, at most 41, at most 42, at most 43, at most 44, at most 45, at most 46, at most 47, at most 48, at most 49, at most 50, at most 51, at most 52, at most 53, at most 54, at most 55, at most 56, at most 57, at most 58, at most 59, at most 60, at most 61, at most 62, at most 63, at most 64, at most 65, at most 66, at most 67, at most 68, at most 69, at most 70, at most 71, at most 72, at most 73, at most 74, at most 75, at most 76, at most 77, at most 78, at most 79, at most 80, at most 81, at most 82, at most 83, at most 84, at most 85, at most 86, at most 87, at most 88, at most 89, at most 90, at most 91, at most 92, at most 93, at most 94, at most 95, at most 96, at most 97, at most 98, at most 99, at most 100, at most 101, at most 102, at most 103, at most 104, at most 105, at most 106, at most 107, at most 108, at most 109, at most 110, at most 111, at most 112, at most 113, at most 114, at most 115, at most 116, at most 117, at most 118, at most 119, at most 120, at most 121, at most 122, at most 123, at most 124, at most 125, at most 126, at most 127, at most 128, at most 129, at most 130, at most 131, at most 132, at most 133, at most 134, at most 135, at most 136, at most 137, at most 138, at most 139, at most 140, at most 141, at most 142, at most 143, at most 144, at most 145, at most 146, at most 147, at most 148, at most 149, at most 150, at most 151, at most 152, at most 153, at most 154, at most 155, at most 156, at most 157, at most 158, at most 159, at most 160, at most 161, at most 162, at most 163, at most 164, at most 165, at most 166, at most 167, at most 168, at most 169, at most 170, at most 171, at most 172, at most 173, at most 174, at most 175, at most 176, at most 177, at most 178, at most 179, at most 180, at most 181, at most 182, at most 183, at most 184, at most 185, at most 186, at most 187, at most 188, at most 189, at most 190, at most 191, at most 192, at most 193, at most 194, at most 195, at most 196, at most 197, at most 198, at most 199, at most 200, at most 201, at most 202, at most 203, at most 204, at most 205, at most 206, at most 207, at most 208, at most 209, at most 210, at most 211, at most 212, at most 213, at most 214, at most 215, at most 216, at most 217, at most 218, at most 219, at most 220, at most 221, at most 222, at most 223, at most 224, at most 225, at most 226, at most 227, at most 228, at most 229, at most 230, at most 231, at most 232, at most 233, at most 234, at most 235, at most 236, at most 237, at most 238, at most 239, at most 240, at most 241, at most 242, at most 243, at most 244, at most 245, at most 246, at most 247, at most 248, at most 249, or at most 250 amino acid alterations or modifications relative to a WT ACS (SEQ ID NO: 1). The one or more amino acid alterations or modifications may be located at one or more positions corresponding to one or more positions in SEQ ID NO: 1. In some embodiments, the ACS variant is a naturally occurring variant, such as an ACS (e.g., a homolog) from a different species or organism. In some embodiments, the ACS variant is a genetically engineered, non-naturally occurring variant.

In some embodiments, the ACS, the ACS variant, or the ACS homolog provided herein comprises an amino acid sequence that has at least 20-99% sequence identity with the wild-type or template sequence, for example the amino acid sequence set forth in SEQ ID NO: 1. For example, the ACS, ACS variant or ACS homolog comprises an amino acid sequence that has at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or higher, sequence identity with the template or wild-type sequence, such as, for example, with the sequence set forth in SEQ ID NO: 1. In some embodiments, the ACS or variant or homolog thereof comprises an amino acid sequence that has at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or higher, sequence identity with an ACS derived from a species or strain of Ruegeria. In some embodiments, the ACS or variant or homolog thereof comprises an amino acid sequence that has at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or higher, sequence identity to an ACS derived from a species or strain of Ruegeria pomeroyi. In some embodiments, the ACS or variant or homolog thereof comprises an amino acid sequence that has at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or higher, sequence identity to an ACS derived from a strain or species of Ruegeria, Roseibium, Rhodobacteraceae, Roseobacteraceae, Cribrihabitans, Falsiruegeria, Tropicibacter, Thioclava, Paracoccus, Celeribacter, Gemmobacter, Marivivens, unclassified, Actibacterium, Salipiger, Youngimonas, Poseidonocella, Nitratireductor, Pseudooceanicola, Rhizobium, Aestuarium, Pseudodonghicola, Rhodobacterales, Ciceribacter, Polymorphum, Rhizobiales, Rhizobiaceae, or Roseobacter. In some embodiments, the ACS or variant or homolog thereof is derived from a strain or species listed in TABLE 1 or 2, or the ACS or variant or homolog thereof is one listed in TABLE 1 or 2. In some embodiments, the ACS is a variant or homolog of any one or more of the ACSs listed in TABLE 1 or 2. In some embodiments, the ACS is a variant or homolog, comprising one or more amino acid alterations or modifications relative to a reference or template sequence, wherein other than the one or more amino acid alterations or modifications, the ACS comprises an amino acid sequence that has at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or higher, sequence identity to any one or more of the ACSs listed in TABLE 1 or 2.

In some embodiments, the ACS or variant or homolog thereof is heterologous to the non-naturally occurring microbial organisms provided herein. In some embodiments, the ACS or variant or homolog thereof is native to the non-naturally occurring microbial organisms provided herein. In some embodiments, the ACS is overexpressed. In some embodiments, the ACS is encoded by an exogenous nucleic acid sequence. In some embodiments, the ACS or variant or homolog thereof is heterologous to the non-naturally occurring microbial organism and is expressed or overexpressed. In other embodiments, the ACS or variant or homolog thereof is native to the non-naturally occurring microbial organism and is overexpressed.

In some embodiments, the ACS, CoA ligase, or homologs, variants, or catalytically active portions thereof, or the polypeptide with ACS activity or CoA ligase activity, is encoded by a one or more heterologous nucleic acid(s). In some embodiments, the ACS, CoA ligase, or homologs, variants, or catalytically active portions thereof, or the polypeptide with ACS activity or CoA ligase activity, is encoded by a one or more exogenous nucleic acid(s). In certain embodiments, the one or more exogenous nucleic acid(s) is/are a heterologous nucleic acid(s). In some embodiments, the ACS, CoA ligase, or homolog, variant, or catalytically active portion thereof, or the polypeptide with ACS or CoA ligase activity, is expressed in a sufficient amount to decrease or eliminate or substantially eliminate the production of 3HB by-product or carboxylate by-product. In some embodiments, the ACS or CoA ligase, or homolog, variant, or catalytically active portion thereof, or the polypeptide with ACS activity or CoA ligase activity, is expressed in a sufficient amount to increase the titer, production, and/or yield of an Ac—CoA derived product, such as, for example, 1,3-BDO, (R)-1,3-BDO, (S)-1,3-BDO, MAA or its esters (e.g., MMA, EMA, and others), (3R)-hydroxybutyl (3R)-hydroxybutyrate, HMD, CPL, adipate, 6ACA, and/or HDO.

As shown, for example, in Example 4, the ACS described herein has broad substrate specificity, and can catalyze the conversion of short-chain (e.g., C2-C4, C2-C6, or C2-C8) carboxylates to their corresponding CoA thioesters or acyl-CoAs. Thus, any of the non-naturally occurring microbial organisms described herein, expressing or overexpressing an ACS, can include more than one by-product recycling pathway. For example, expression or overexpression of ACS can result in a decrease in the production of any one or more short-chain carboxylate by-products formed in the cell or microorganism, such as, for example, any one or more of 3HB, (R)-3HB, (S)-3HB, acetate, acetoacetate, succinate, crotonate, butyrate, isobutyrate, malonate, and/or hexanoate. This can result in an increased carbon flux through the corresponding CoA thioesters (acyl-CoAs) of the carboxylate by-products, and an increased production, titer, yield, and/or productivity of one or more acetyl-CoA derived products. For example, as shown in Examples 1 and 5, the expression of ACS in a microorganism comprising a 1,3-BDO pathway results in the increased production of 1,3-BDO, and the decreased production of 3HB and acetate by-products. Thus, for example, the expression or overexpression of ACS can result in a decrease in both acetate and 3HB by-products, which can result in an increased or enhanced carbon flux through 3HB—CoA (and/or 3HBAld), and acetyl-CoA, and can result in an even greater increase in the production of an acetyl-CoA derived product (for example, as compared to the recycling of 3HB by-product alone). Accordingly, the combination of reducing 3HB by-product and acetate by-product can be additive and can allow for an even greater productivity, titer, and/or yield of acetyl-CoA derived products. In some embodiments, the combination of reducing 3HB by-product and acetate by-product can be synergistic and can allow for an even greater productivity, titer, and/or yield of acetyl-CoA derived products.

Additionally or alternatively, 3HB can be brought back into the main biosynthetic pathway (for the acetyl-CoA derived product or an intermediate thereof) by the expression of an enzyme, or a polypeptide, or a catalytically active portion thereof, that catalyzes the conversion of 3HB into 3HBAld and/or enhances carbon flux through 3HBAld, thereby increasing the yield of one or more acetyl-CoA derived products or intermediates, such as, for example, 1,3-BDO, (R)-1,3-BDO, (S)-1,3-BDO, MAA, MMA, or (3R)-hydroxybutyl (3R)-hydroxybutyrate. Accordingly, provided herein, is a non-naturally occurring microbial organism having a reduced amount of 3HB by-product or eliminated or substantially eliminated 3HB by-product, and/or having a biosynthetic pathway that includes a 3HB to 3HBAld conversion step.

In some embodiments, a non-naturally occurring microbial organism having reduced, or eliminated, or substantially eliminated 3HB by-product can additionally or alternatively have a 3HB to 3HBAld conversion step. In one aspect, the non-naturally occurring microbial organism described herein, having a 3HB to 3HBAld conversion step includes an acetyl-CoA derived product pathway having a 3HB—CoA pathway, or a pathway resulting in an 3HB—CoA intermediate, and a downstream 3HBAld intermediate. As described above, a microbial organism having a 3HB—CoA pathway or a pathway with a 3HB—CoA intermediate can also have one or more enzymes or activities that result in the loss of 3HB—CoA intermediate or in the conversion of 3HB—CoA intermediate to 3HB by-product, for example, by spontaneous hydrolysis, or by the activity of one or more nonspecific enzymes, such as, for example, CoA thioesterases or CoA hydrolases.

Accordingly, in some embodiments, the non-naturally occurring microbial organism having a 3HB to 3HB Ald conversion step has enhanced carbon flux through 3HB-Ald, reduced production of 3HB by-product, and/or increased production, titer, productivity, and/or yield of an acetyl-CoA derived product. The conversion of 3HB by-product to 3HBAld can be catalyzed by an enzyme or polypeptide with carboxylic acid reductase (CAR) activity. Hence, in certain embodiments, the non-naturally occurring microbial organism having a 3HB to 3HBAld conversion step has enhanced carbon flux through 3HBAld, reduced production of 3HB by-product, and/or increased production of an acetyl-CoA derived product, whereby the microorganism expresses one or more exogenous nucleic acids encoding a carboxylic acid reductase (CAR) or a homolog or variant thereof, or an enzyme or polypeptide with CAR activity. In some embodiments, the CAR can be heterologous to the non-naturally occurring microbial organism and can be expressed or overexpressed. In other embodiments, the CAR can be native to the non-naturally occurring microbial organism and is overexpressed.

In some embodiments, the non-naturally occurring microbial organism having a 3HB to 3HBAld conversion step has reduced, eliminated, or substantially eliminated 3HB by-product formation. In some embodiments, the non-naturally occurring microbial organism having a 3HB to 3HBAld conversion step has reduced, eliminated, or substantially eliminated 3HB by-product formation, as compared to (i) a microbial organism without a 3HB to 3HBAld conversion step, or as compared to (ii) a microbial organism without a one or more exogenous nucleic acid(s) encoding a CAR or a homolog or variant thereof, or an enzyme or polypeptide with CAR activity, as described herein, or as compared to both (i) and (ii). In certain embodiments, the reduction in 3HB formation, concentration, or titer is about a 5% to about a 100% reduction. In some embodiments, the reduction in 3HB formation, concentration, or titer is about a 20% to 95%, about a 30% to 90%, about a 40% to 85%, or about a 50% to 80% reduction. In some embodiments, the reduction in 3HB formation, concentration, or titer is about a 5%, about a 10%, about a 15%, about a 20%, about a 25%, about a 30%, about a 35%, about a 40%, about a 45%, about a 50%, about a 55%, about a 60%, about a 65%, about a 70%, about a 75%, about an 80%, about an 85%, about a 90%, about a 95%, about a 96%, about a 97%, about a 98%, about a 99%, or about a 100% reduction.

As provided herein, eliminating, substantially eliminating, or decreasing the amount, titer, or concentration of 3HB by-product, such as by having a 3HB to 3HBAld conversion step, can increase carbon flux through 3HBAld. Accordingly, in some embodiments, the non-naturally occurring microbial organism having a 3HB to 3HBAld conversion step has enhanced carbon flux through 3HBAld. In certain embodiments, the non-naturally occurring microbial organism having a 3HB to 3HB Ald conversion step has enhanced carbon flux through 3HBAld as compared to (i) a microbial organism without a 3HB to 3HBAld conversion step, or as compared to (ii) a microbial organism without a one or more exogenous nucleic acid(s) encoding CAR or a homolog or a variant thereof, or a polypeptide or enzyme with CAR activity, as described herein, or as compared to both (i) and (ii). In certain embodiments, the enhancement of carbon flux through 3HBAld is about a 5% to about a 100% enhancement, about a 20% to about a 95% enhancement, about a 30% to about a 90% enhancement, about a 40% to about an 85% enhancement, or about a 50% to about an 80% enhancement. In some embodiments, the enhancement of carbon flux through 3HBAld is about a 5%, about a 10%, about a 15%, about a 20%, about a 25%, about a 30%, about a 35%, about a 40%, about a 45%, about a 50%, about a 55%, about a 60%, about a 65%, about a 70%, about a 75%, about an 80%, about an 85%, about a 90%, about a 95%, about a 96%, about a 97%, about a 98%, about a 99%, or about a 100% enhancement.

As provided herein, eliminating, substantially eliminating, or decreasing the amount or concentration or titer of 3HB by-product, such as by having a 3HB to 3HBAld conversion step, can increase carbon flux through 3HBAld and, therefore, increase the titer, productivity and/or yield of acetyl-CoA derived products or intermediates thereof, such as, for example, 1,3-BDO, (R)-1,3-BDO, (S)-1,3-BDO, MAA, MMA, or (3R)-hydroxybutyl (3R)-hydroxybutyrate. Accordingly, in some embodiments, the non-naturally occurring microbial organism having a 3HB to 3HBAld conversion step has increased productivity, titer, and/or yield of an acetyl-CoA derived product (or an intermediate thereof). In certain embodiments, the non-naturally occurring microbial organism having a 3HB to 3HB Ald conversion step has increased yield of an acetyl-CoA derived product (or an intermediate thereof) as compared to (i) a microbial organism without a 3HB to 3HBAld conversion step, or as compared to (ii) a microbial organism without a one or more exogenous nucleic acid(s) encoding a CAR or a homolog or a variant thereof, or a polypeptide or enzyme with CAR activity, as described herein, or as compared to both (i) and (ii). In certain embodiments, the increased productivity, titer, and/or yield of an acetyl-CoA derived product (or an intermediate thereof) is about a 0.01% to about an 80% increase, or more. In some embodiments, the increased productivity, titer, and/or yield of an acetyl-CoA derived product (or an intermediate thereof) is about a 0.1% to about a 10% increase, about a 0.5% to about a 15% increase, about a 1% to about a 20% increase, about a 1% to about a 30% increase, or more. In some embodiments, the increased productivity, titer, and/or yield of an acetyl-CoA derived product is about a 0.01% increase, about a 0.05% increase, about a 0.1% increase, about a 0.2% increase, about a 0.3% increase, about a 0.4% increase, about a 0.5% increase, about a 0.6% increase, about a 0.7% increase, about a 0.8% increase, or about a 0.9% increase. In some embodiments, the increase in the productivity, titer, and/or yield of an acetyl-CoA derived product is about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, or about 80%, or more.

In some embodiments, the elimination, substantial elimination, or reduction in the amount or concentration of 3HB by-product results in about a 0.01-fold to about a 10-fold increase, or more, in the productivity, titer, and/or yield of one or more acetyl-CoA derived products (or intermediate(s) thereof), as compared to (i) a microbial organism without a 3HB to 3HBAld conversion step, or as compared to (ii) a microbial organism without a one or more exogenous nucleic acid(s) encoding a CAR or a homolog or a variant thereof, or a polypeptide or enzyme with CAR activity, as described herein, or as compared to both (i) and (ii). In some embodiments, the elimination, substantial elimination, or reduction in the amount, titer, or concentration of 3HB by-product results in about a 0.01-fold, about a 0.02-fold, about a 0.03-fold, about a 0.04-fold, about a 0.05-fold, about a 0.06-fold, about a 0.07-fold, about a 0.08-fold, about a 0.09-fold, about a 0.1-fold, about a 0.2-fold, about a 0.3-fold, about a 0.4-fold, about a 0.5-fold, about a 0.6-fold, about a 0.7-fold, about a 0.8-fold, about a 0.9-fold, about a 1.0-fold, about a 1.1-fold, about a 1.2-fold, about a 1.3-fold, about a 1.4-fold, about a 1.5-fold, about a 1.6-fold, about a 1.7-fold, about a 1.8-fold, about a 1.9-fold, about 2.0-fold, about a 2.5-fold, about a 3.0-fold, about a 3.5-fold, about a 4.0-fold, about a 4.5-fold, about a 5.0-fold, about a 5.5-fold, about a 6.0-fold, about a 6.5-fold, about a 7.0-fold, about a 7.5-fold, about an 8.0-fold, about an 8.5-fold, about a 9.0-fold, about a 9.5-fold, or about a 10-fold, or more, increase in the productivity, titer, and/or yield of one or more acetyl-CoA derived products (or intermediate(s) thereof).

The conversion of 3HB to 3HBAld can be catalyzed by a polypeptide or an enzyme with carboxylic acid reductase activity. In some embodiments, enzymes with carboxylic acid reductase activity include Carboxylic Acid Reductase (CAR), as well as homologs and variants thereof, and catalytically active portions thereof.

Exemplary enzymes with carboxylic acid reductase activity include enzymes shown in TABLE 3 below.

TABLE 3
Exemplary CARs

| | | SEQ ID |
|---|---|---|
| Description | Sequence | NO. |
| Wild-type CAR | MSTATHDERLDRRVHELIATDPQFAAAQPDPAITAAL | 2 |
| from | EQPGLRLPQIIRTVLDGYADRPALGQRVVEFVTDAKT | |
| Mycobacterium | GRTSAQLLPRFETITYSEVAQRVSALGRALSDDAVHP | |
| avium | GDRVCVLGFNSVDYATIDMALGAIGAVSVPLQTSAAI | |
| (Accession No. | SSLQPIVAETEPTLIASSVNQLSDAVQLITGAEQAPTR | |
| WP_003872682. | LVVFDYHPQVDDQREAVQDAAARLSSTGVAVQTLAEL | |
| 1) | LERGKDLPAVAEPPADEDSLALLIYTSGSTGAPKGAM | |
| | YPQSNVGKMWRRGSKNWFGESAASITLNFMPMSHV | |
| | MGRSILYGTLGNGGTAYFAARSDLSTLLEDLELVRPT | |
| | ELNFVPRIWETLYGEFQRQVERRLSEAGDAGERRAVE | |
| | AEVLAEQRQYLLGGRFTFAMTGSAPISPELRNWVESL | |
| | LEMHLMDGYGSTEAGMVLFDGEIQRPPVIDYKLVDV | |
| | PDLGYFSTDRPHPRGELLLRTENMFPGYYKRAETTAG | |
| | VFDEDGYYRTGDVFAEIAPDRLVYVDRRNNVLKLAQ | |
| | GEFVTLAKLEAVFGNSPLIRQIYVYGNSAQPYLLAVV | |
| | VPTEEALASGDPETLKPKIADSLQQVAKEAGLQSYEV | |
| | PRDFIIETTPFSLENGLLTGIRKLAWPKLKQHYGERLE | |
| | QMYADLAAGQANELAELRRNGAQAPVLQTVSRAAGA | |
| | MLGSAASDLSPDAHFTDLGGDSLSALTFGNLLREIFDV | |
| | DVPVGVIVSPANDLAAIASYIEAERQGSKRPTFASVHG | |
| | RDATVVRAADLTLDKFLDAETLAAAPNLPKPATEVRT | |
| | VLLTGATGFLGRYLALEWLERMDMVDGKVIALVRAR | |
| | SDEEARARLDKTFDSGDPKLLAHYQQLAADHLEVIAG | |
| | DKGEANLGLGQDVWQRLADTVDVIVDPAALVNHVL | |
| | PYSELFGPNALGTAELIRLALTSKQKPYTYVSTIGVGD | |
| | QIEPGKFVENADIRQMSATRAINDSYANGYGNSKWA | |
| | GEVLLREAHDLCGLPVAVFRCDMILADTTYAGQLNLP | |
| | DMFTRLMLSLVATGIAPGSFYELDADGNRQRAHYDG | |
| | LPVEFIAAAISTLGSQITDSDTGFQTYHVMNPYDDGVG | |
| | LDEYVDWLVDAGYSIERIADYSEWLRRFETSLRALPD | |
| | RQRQYSLLPLLHNYRTPEKPINGSIAPTDVFRAAVQEA | |
| | KIGPDKDIPHVSPPVIVKYITDLQLLGLL | |
| Variant 1 | MSTATHDERLDRRVHELIATDPQFAAAQPDPAITAAL | 3 |
| | EQPGLRLPQIIRTVLDGYADRPALGQRVVEFVTDAKT | |
| | GRTSAQLLPRFETITYSEVAQRVSALGRALSDDAVHP | |
| | GDRVCVLGFNSVDYATIDMALGAIGAVSVPLQTSAAI | |
| | SSLQPIVAETEPTLIASSVNQLSDAVQLITGAEQAPTR | |
| | LVVFDYHPQVDDQREAVQDAAARLSSTGVAVQTLAEL | |
| | LERGKDLPAVAEPPADEDSLALLIYTSGSTGAPKGAM | |
| | YPQSNVGKMWRRGSKNWFGESAASITLNFMPMSHV | |
| | MGRSILYGTLGNGGTAYFAARSDLSTLLEDLELVRPT | |
| | ELNFVPRIWETLYGEFQRQVERRLSEAGDAGERRAVE | |
| | AEVLAEQRQYLLGGRFTFAMTSSAPISPELRNWVESLL | |
| | EMHLMDGYGSTEAGMVLFDGEIQRPPVIDYKLVDVP | |
| | DLGYFSTDRPHPRGELLLRTENMFPGYYKRAETTAGV | |
| | FDEDGYYRTGDVFAEIAPDRLVYVDRRNNVLKLAQG | |
| | EFVTLAKLEAVFGNSPLIRQIYVYGNSAQPYLLAVVVP | |
| | TEEALASGDPETLKPKIADSLQQVAKEAGLQSYEVPR | |
| | DFIIETTPFSLENGLLTGIRKLAWPKLKQHYGERLEQM | |
| | YADLAAGQANELAELRRNGAQAPVLQTVSRAAGAML | |
| | GSAASDLSPDAHFTDLGGDSLSALTFGNLLREIFDVD | |
| | VPVGVIVSPANDLAAIASYIEAERQGSKRPTFASVHGR | |
| | DATVVRAADLTLDKFLDAETLAAAPNLPKPATEVRTV | |
| | LLTGATGFLGRYLALEWLERMDMVDGKVIALVRARS | |
| | DEEARARLDKTFDSGDPKLLAHYQQLAADHLEVIAG | |
| | DKGEANLGLGQDVWQRLADTVDVIVDPAALVNHVLP | |
| | YSELFGPNALGTAELIRLALTSKQKPYTYVSTIGVGD | |
| | QIEPGKFVENADIRQMSATRAINDSYANGYGNSKWA | |
| | GEVLLREAHDLCGLPVAVFRCDMILADTTYAGQLNLP | |
| | DMFTRLMLSLVATGIAPGSFYELDADGNRQRAHYDGL | |
| | PVEFIAAAISTLGSQITDSDTGFQTYHVMNPYDDGVG | |
| | LDEYVDWLVDAGYSIERIADYSEWLRRFETSLRALPD | |
| | RQRQYSLLPLLHNYRTPEKPINGSIAPTDVFRAAVQEA | |
| | KIGPDKDIPHVSPPVIVKYITDLQLLGLL | |

Further exemplary CAR homologs, that can be used in the 3HB to 3HBAld conversion step described herein, are shown in the table below. The percent sequence identity of each homolog, relative to the amino acid sequence of wild-type (WT) CAR of Mycobacterium avium (SEQ ID NO: 2), also is listed in the table below.

TABLE 3.1
Exemplary CAR Homologs

| | | | % |
|---|---|---|---|
| | | | Identity |
| | | | to WT |
| | | | CAR |
| | | | (SEQ |
| | | Accession No. | ID NO: |
| Enzyme/Gene | Organism | (UniProtKB) | 2) |
| Carboxylic acid | Mycolicibacterium | Q741P9 | 99.5 |
| reductase/fadD9 | paratuberculosis | | |
| | (Mycobacterium | | |
| | paratuberculosis) | | |
| Carboxylic acid | Mycobacterium | A0A051U3G5 | 89.6 |
| reductase/car | [tuberculosis] | | |
| Carboxylic acid | Mycobacterium | A0A557XZJ5 | 82 |
| reductase/car | helveticum | | |
| Carboxylic acid | Mycobacterium | A0A9P3UYS2 | 81.5 |
| reductase/fadD9 | kiyosense | | |
| Carboxylic acid | Mycobacterium | A0A1X1V443 | 81.1 |
| reductase/car | fragae | | |
| Carboxylic acid | Mycobacterium | A0A1A3N7A1 | 80.9 |
| reductase/car | asiaticum | | |
| Carboxylic acid | Mycobacterium | A0A1X0DT32 | 80.9 |
| reductase/fadD9 | heidelbergense | | |
| Carboxylic acid | Mycobacterium sp. | A0A1A0M3R1 | 79.9 |
| reductase/car | 1164966.3 | | |
| Carboxylic acid | Mycobacterium | A0A0I9Z3I8 | 79.8 |
| reductase/car | haemophilus | | |
| Carboxylic acid | Mycobacterium | A0A1S1MVI3 | 79.8 |
| reductase/car | talmoniae | | |
| Carboxylic acid | Mycobacterium | A0A0Q2RTZ0 | 79.2 |
| reductase/car | gordonae | | |
| Carboxylic acid | Mycobacterium | A0A498QQD7 | 79 |
| reductase/car | pseudokansasii | | |
| Carrier domain- | Mycobacterium | A0A7G1IFN8 | 79 |
| containing protein | kansasii | | |
| Carboxylic acid | Mycobacterium | A0A7I9Y2B3 | 78.2 |
| reductase/fadD9 | botniense | | |
| Carboxylic acid | Mycobacterium | A0A7I7KQ88 | 78.1 |
| reductase/fadD9 | cookii | | |
| Carboxylic acid | Mycobacterium | A0A1E3SN70 | 77 |
| reductase/car | sherrisii | | |
| Carboxylic acid | Mycobacterium | I0RXB9 | 77 |
| reductase/car | xenopi | | |
| | RIVM700367 | | |
| Carboxylic acid | Mycobacterium | Q9CCT4 | 76.8 |
| reductase/fadD9 | leprae | | |
| Carboxylic acid | Mycolicibacterium | A0A132PRD2 | 75.6 |
| reductase/car | wolinskyi | | |
| Carboxylic acid | Mycolicibacillus | A0A1X2EPB4 | 75.2 |
| reductase/car | trivialis | | |
| Carboxylic acid | Mycolicibacterium | A0A0N9XFV2 | 75.1 |
| reductase/car | fortuitum | | |
| | (Mycobacterium | | |
| | fortuitum) | | |
| Carboxylic acid | Mycobacterium | A0A1Y0CFN4 | 75 |
| reductase/car | dioxanotrophicus | | |
| Carboxylic acid | Mycolicibacter | F5YUX6 | 74.9 |
| reductase/fadD9 | sinensis | | |
| Carboxylic acid | Mycobacterium sp. | A0A1A0M2E4 | 73.6 |
| reductase/car | 1164966.3 | | |
| Carboxylic acid | Mycobacterium | A0A1E3S739 | 73.2 |
| reductase/car | intermedium | | |
| Carboxylic acid | Mycolicibacterium | A0A132PS78 | 73.2 |
| reductase/car | wolinskyi | | |
| Carboxylic acid | Mycolicibacterium | W9AZM3 | 72.8 |
| reductase/car | cosmeticum | | |
| Carboxylic acid | Mycobacterium | B2HN69 | 72.5 |
| reductase/car | marinum | | |
| Carboxylic acid | Mycolicibacterium | A0QWI7 | 72.3 |
| reductase/car | smegmatis | | |
| | (Mycobacterium | | |
| | smegmatis) | | |
| Carboxylic acid | Mycobacterium | A0A1X1YTK3 | 72.1 |
| reductase/fadD9 | lacus | | |
| Carboxylic acid | Mycolicibacterium | A0A0D1L7Y1 | 72.1 |
| reductase/car | llatzerense | | |
| Carboxylic acid | Mycobacterium | A0A7Z7ILT5 | 72 |
| reductase/car | simulans | | |
| Carboxylic acid | Mycobacterium sp. | A0A1A3T4S7 | 71.9 |
| reductase/car | 1274761.0 | | |
| Carboxylic acid | Mycolicibacterium | A0A0N9YG13 | 71.9 |
| reductase/car | fortuitum | | |
| | (Mycobacterium | | |
| | fortuitum) | | |
| Carboxylic acid | Mycolicibacterium | A0A0J6WR15 | 71.8 |
| reductase/car | chubuense | | |
| | (Mycobacterium | | |
| | chubuense) | | |
| Carboxylic acid | Mycolicibacterium | I4BIH7 | 71.8 |
| reductase/car | chubuense | | |
| | (Mycobacterium | | |
| | chubuense) | | |
| | (strain NBB4) | | |
| Carboxylic acid | Mycobacterium sp. | A0A101ABK2 | 71.7 |
| reductase/car | GA-2829 | | |
| Carboxylic acid | Mycobacterium | A0A1X2LPX9 | 71.3 |
| reductase/car | decipiens | | |
| Carboxylic acid | Mycobacterium | A0A1Y0C5T9 | 71.3 |
| reductase/car | dioxanotrophicus | | |
| Carboxylic acid | Mycolicibacterium | W9B0J1 | 71.3 |
| reductase/car | cosmeticum | | |
| Carboxylic acid | Mycolicibacterium | A0A2A7NAH8 | 71.1 |
| reductase/car | agri | | |
| | (Mycobacterium) | | |
| | agri | | |
| Carboxylic acid | Mycobacterium | A0A7I7MPF4 | 70.9 |
| reductase/fadD9 | shinjukuense | | |
| Carboxylic acid | Mycobacterium | A0A975JWU9 | 70.8 |
| reductase/car | spongiae | | |
| Carboxylic acid | Mycolicibacterium | A0A1X1TAZ5 | 70.6 |
| reductase/car | doricum | | |
| Carboxylic acid | Mycobacterium sp. | A0A132SEI8 | 70.5 |
| reductase/car | NAZ190054 | | |
| Carboxylic acid | Mycolicibacterium | A0A3S4RS92 | 70.3 |
| reductase/IgrD_1 | aurum | | |
| | (Mycobacterium | | |
| | aurum) | | |
| Carboxylic acid | Mycolicibacterium | A0A7I7SXJ5 | 70.3 |
| reductase/car | sarraceniae | | |
| Carboxylic acid | Mycolicibacterium sp. | A0A5A7WZ06 | 70.2 |
| reductase/car | P9-64 | | |
| Carboxylic acid | Mycolicibacterium | A0A1E3S0K4 | 70.2 |
| reductase/car | holsaticum | | |
| Carboxylic acid | Mycobacterium | Q7D6X4 | 70.1 |
| reductase/car | tuberculosis | | |
| Carboxylic acid | Mycobacterium | Q50631 | 70.1 |
| reductase/car | tuberculosis | | |
| Carboxylic acid | Mycobacterium | A0A0H3LC90 | 70.1 |
| reductase/fadD9 | tuberculosis | | |
| Carboxylic acid | Mycobacterium | A5U5U0 | 70.1 |
| reductase/fadD9 | tuberculosis | | |
| Carboxylic acid | Mycobacterium | A0A1R3Y1N0 | 70 |
| reductase/fadD9 | bovis | | |
| Carboxylic acid | Pseudofrankia | A0A1V2I487 | 69.9 |
| reductase/car | asymbiotica | | |
| Carboxylic acid | Mycolicibacterium | A0A378TAR8 | 69.9 |
| reductase/fadD9 | tokaiense | | |
| Carboxylic acid | Mycolicibacterium | A0A7I7X9S1 | 69.6 |
| reductase/car | madagascariense | | |
| Carboxylic acid | Mycolicibacterium | A0A7I7XXG0 | 69.6 |
| reductase/car | confluentis | | |
| Carboxylic acid | Mycolicibacterium | A0A5N5VEC4 | 69.6 |
| reductase/car | phlei | | |
| Carboxylic acid | Mycobacterium sp. | A0A2C9SQE5 | 69.5 |
| reductase/car | shizuoka-1 | | |
| Carboxylic acid | Mycobacterium | A0A172UHW8 | 69.4 |
| reductase/car | adipatum | | |
| Carboxylic acid | Mycolicibacterium | A0A7I7XP41 | 69.3 |
| reductase/car | madagascariense | | |
| Carboxylic acid | Mycolicibacterium | A0A7I7QL20 | 69.3 |
| reductase/car | sediminis | | |
| Carboxylic acid | Mycobacterium sp. | A0A1A1ZE81 | 69.1 |
| reductase/car | ACS1612 | | |
| Carboxylic acid | Mycobacterium | A0A1E8Q6I1 | 68.9 |
| reductase/car | grossiae | | |
| Carboxylic acid | Mycolicibacterium | V5X749 | 68.8 |
| reductase/car | neoaurum | | |
| Carboxylic acid | Mycolicibacterium | G8RIP0 | 68.7 |
| reductase/car | rhodesiae | | |
| | (Mycobacterium | | |
| | rhodesiae) (strain | | |
| | NBB3) | | |
| Carboxylic acid | Mycobacterium sp. | A0A1A0TM44 | 68.6 |
| reductase/car | 852013- | | |
| | 51886_SCH5428379 | | |
| Carboxylic acid | Mycolicibacterium | A0A6N4W3I8 | 68.2 |
| reductase/car | anyangense | | |
| Carboxylic acid | Mycolicibacterium sp. | A0A5A7X3S8 | 68.1 |
| reductase/car | P9-64 | | |
| Carboxylic acid | Mycolicibacterium | A0A1X0D333 | 68.1 |
| reductase/car | insubricum | | |
| Carboxylic acid | Mycolicibacterium | A0A7I7QP40 | 67.9 |
| reductase/car | sediminis | | |
| Carboxylic acid | Mycobacterium sp. | A0A0T1W5Z5 | 67.7 |
| reductase/car | Root135 | | |
| Carboxylic acid | Mycolicibacterium | A0A0D1LMF4 | 67.7 |
| reductase/car | llatzerense | | |
| Carboxylic acid | Mycobacterium sp. | A0A0T1WDJ1 | 67.6 |
| reductase/car | Root135 | | |
| Carboxylic acid | Mycobacterium | A0A1E8QAA5 | 67.5 |
| reductase/car | grossiae | | |
| Carboxylic acid | Mycolicibacterium | A0A1X1RGM6 | 66.4 |
| reductase/car | fallax | | |
| | (Mycobacterium | | |
| | fallax) | | |
| Carboxylic acid | Mycolicibacterium | A0A6N4VDZ9 | 66.1 |
| reductase/car | poriferae | | |
| Carboxylic acid | Nocardia | A0A917RUF6 | 66 |
| reductase/fadD9 | jinanensis | | |
| Carboxylic acid | Mycobacterium sp. | A0A1A1YDQ5 | 65.8 |
| reductase/car | ACS1612 | | |
| Carboxylic acid | Mycobacterium sp. | A0A124EWF0 | 65.7 |
| reductase/car | GA-2829 | | |
| Carboxylic acid | Nocardia | A0A846XPH2 | 64.4 |
| reductase/car | speluncae | | |
| Carboxylic acid | Nocardia | A0A0U1Z2H3 | 64.4 |
| reductase/car | terpenica | | |
| Carboxylic acid | Mycolicibacterium | A0A2G5PCS5 | 64 |
| reductase/car | brumae | | |
| Carboxylic acid | Pseudonocardiaceae | A0A419HNG9 | 63.1 |
| reductase/car | bacterium YIM PH | | |
| | 21723 | | |
| Carboxylic acid | Nocardia | K0EPZ9 | 61.8 |
| reductase/car | brasiliensis | | |
| Carboxylic acid | Nocardia | Q6RKB1 | 61.6 |
| reductase/car | iowensis | | |
| Carboxylic acid | Mycobacterium | B2HE95 | 61.1 |
| reductase/fadD9_1 | marinum | | |
| Carboxylic acid | Nocardia | A0A6G9YNU3 | 60.3 |
| reductase/car | arthritidis | | |
| Carboxylic acid | Longimycelium | A0A8J3FU84 | 59.9 |
| reductase/car | tulufanense | | |
| Carboxylic acid | Nocardia | A0A164L8T1 | 59.5 |
| reductase/car | terpenica | | |
| Carboxylic acid | Nocardia | K0EY54 | 59.5 |
| reductase/car | brasiliensis | | |
| Carboxylic acid | Nocardia | A0A3A4K4S5 | 59.5 |
| reductase/car | panacis | | |

In some embodiments, the CAR is encoded by a car or fadD9 gene. In some embodiments, the CAR is derived from a species or strain of Mycobacterium, Mycolicibacterium, Mycolicibacillus, Mycolicibacter, Pseudofrankia, Norcadia, Pseudonocardiaceae, or Longimycelium. For example, the CAR can be derived from a strain or species of Mycolicibacterium paratuberculosis (Mycobacterium paratuberculosis), Mycobacterium [tuberculosis], Mycobacterium helveticum, Mycobacterium kiyosense, Mycobacterium fragae, Mycobacterium asiaticum, Mycobacterium heidelbergense, Mycobacterium sp. 1164966.3, Mycobacterium haemophilusycobacterium talmoniae, Mycobacterium gordonae, Mycobacterium pseudokansasii, Mycobacterium kansasii, Mycobacterium botniense, Mycobacterium cookii, Mycobacterium sherrisii, Mycobacterium xenopi RIVM700367, Mycobacterium leprae, Mycolicibacterium wolinskyi, Mycolicibacillus trivialis, Mycolicibacterium fortuitum (Mycobacterium fortuitum), Mycobacterium dioxanotrophicus, Mycolicibacter sinensis, Mycobacterium sp. 1164966.3, Mycobacterium intermedium, Mycolicibacterium wolinskyi, Mycolicibacterium cosmeticum, Mycobacterium marinum, Mycolicibacterium smegmatis (Mycobacterium smegmatis), Mycobacterium lacus, Mycolicibacterium llatzerense, Mycobacterium simulans, Mycobacterium sp. 1274761.0, Mycolicibacterium fortuitum (Mycobacterium fortuitum), Mycolicibacterium chubuense (Mycobacterium chubuense), Mycolicibacterium chubuense (Mycobacterium chubuense) (strain NBB4), Mycobacterium sp. GA-2829, Mycobacterium decipiens, Mycobacterium dioxanotrophicus, Mycolicibacterium cosmeticum, Mycolicibacterium agri (Mycobacterium agri), Mycobacterium shinjukuense, Mycobacterium spongiae, Mycolicibacterium doricum, Mycobacterium sp. NAZ190054, Mycolicibacterium aurum (Mycobacterium aurum), Mycolicibacterium sarraceniae, Mycolicibacterium sp. P9-64, Mycolicibacterium holsaticum, Mycobacterium tuberculosis, Mycobacterium tuberculosis, Mycobacterium tuberculosis, Mycobacterium tuberculosis, Mycobacterium bovis, Pseudofrankia asymbiotica, Mycolicibacterium tokaiense, Mycolicibacterium madagascariense, Mycolicibacterium confluentis, Mycolicibacterium phlei, Mycobacterium sp. shizuoka-1, Mycobacterium adipatum, Mycolicibacterium madagascariense, Mycolicibacterium sediminis, Mycobacterium sp. ACS1612, Mycobacterium grossiae, Mycolicibacterium neoaurum, Mycolicibacterium rhodesiae (Mycobacterium rhodesiae) (strain NBB3), Mycobacterium sp. 852013-51886 SCH5428379, Mycolicibacterium anyangense, Mycolicibacterium sp. P9-64, Mycolicibacterium insubricum, Mycolicibacterium sediminis, Mycobacterium sp. Root135, Mycolicibacterium llatzerense, Mycobacterium sp. Root135, Mycobacterium grossiae, Mycolicibacterium fallax (Mycobacterium fallax), Mycolicibacterium poriferae, Nocardia jinanensis, Mycobacterium sp. ACS1612, Mycobacterium sp. GA-2829, Nocardia speluncae, Nocardia terpenica, Mycolicibacterium brumae, Pseudonocardiaceae bacterium YIM PH 21723, Nocardia brasiliensis, Nocardia iowensis, Mycobacterium marinum, Nocardia arthritidis, Longimycelium tulufanense, Nocardia terpenica, Nocardia brasiliensis, or Nocardia panacis.

In certain embodiments, the polypeptide or enzyme with carboxylic acid reductase activity is a wild-type CAR. In some embodiments, the wild-type CAR comprises the sequence set forth in SEQ ID NO: 2. In certain embodiments, the polypeptide or enzyme with carboxylic acid reductase activity is a CAR variant. In some embodiments, the CAR variant is variant of SEQ ID NO: 2. In some embodiments, the CAR variant can be a CAR enzyme that is overexpressed relative to the wild-type CAR, but retains CAR activity. In some embodiments, the CAR variant contains one or more amino acid alterations or modifications that enhance or increase expression of the CAR variant, relative to WT CAR. In some embodiments, the CAR variant has higher activity relative to the WT CAR. In some embodiments, the term “activity” as used herein to describe enzymatic activity, refers to catalytic activity, substrate specificity, substrate affinity, substrate selectivity, or any combination thereof. In some embodiments, the CAR variant contains one or more amino acid alterations or modifications, and the CAR variant has increased activity for the conversion of 3HB to 3HBAld, and/or has increased substrate selectivity, affinity and/or specificity, compared to WT CAR. Accordingly, in some embodiments, a CAR variant has 1) higher activity for the conversion of 3HB to 3HBAld; 2) higher specificity for 3HB, 3) higher affinity for 3HB, 4) higher selectivity for 3HB, or any combination thereof, as compared to WT CAR (e.g., SEQ ID NO: 2). Hence, in certain embodiments, the non-naturally occurring microbial organism having a 3HB to 3HBAld conversion step, and at least one nucleic acid (e.g., an exogenous nucleic acid) encoding a CAR variant, has enhanced carbon flux through 3HB-Ald, and/or eliminated, substantially eliminated, or reduced production of 3HB by-product, and/or increased productivity, titer, and/or yield of one or more acetyl-CoA derived products or intermediate(s) thereof, when compared to a non-naturally occurring microbial organism having a 3HB to 3HBAld conversion step and at least one nucleic acid (e.g., an exogenous nucleic acid) encoding a WT CAR.

In some embodiments, the CAR variant can exhibit an activity that is at least the same or higher than the WT CAR (e.g., SEQ ID NO: 2), that is, it has activity that is the same or higher than a CAR without the modification or mutation or alteration at the same or corresponding amino acid position(s). In some embodiments, the CAR variant can exhibit two or more activities (e.g., ability to catalyze a reaction described herein and selectivity for a 3HB substrate) that are at least the same or higher than the WT CAR (e.g., SEQ ID NO: 2), that is, it has two or more activities that are the same or higher than a CAR without the modification or mutation or alteration at the same or corresponding amino acid position(s). For example, the CAR variants provided here can have one or more activity that is at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, or at least 200% higher over (or compared to) a WT CAR. In some embodiments, a CAR variant provided herein has an activity that is at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, or at least 200% higher than the activity of a WT CAR consisting of or comprising the amino acid sequence of SEQ ID NO: 2. In some embodiments, a CAR variant provided herein has an activity that is at least 10% higher. In some embodiments, a CAR variant provided herein has an activity that is at least 20% higher. In some embodiments, a CAR variant provided herein has an activity that is at least 30% higher. In some embodiments, a CAR variant provided herein has an activity that is at least 40% higher. In some embodiments, a CAR variant provided herein has an activity that is at least 50% higher. In some embodiments, a CAR variant provided herein has an activity that is at least 60% higher. In some embodiments, a CAR variant provided herein has an activity that is at least 70% higher. In some embodiments, a CAR variant provided herein has an activity that is at least 80% higher. In some embodiments, a CAR variant provided herein has an activity that is at least 90% higher. In some embodiments, a CAR variant provided herein has an activity that is at least 100% higher. In some embodiments, a CAR variant provided herein has an activity that is at least 110% higher. In some embodiments, a CAR variant provided herein has an activity that is at least 120% higher. In some embodiments, a CAR variant provided herein has an activity that is at least 130% higher. In some embodiments, a CAR variant provided herein has an activity that is at least 140% higher. In some embodiments, a CAR variant provided herein has an activity that is at least 150% higher. In some embodiments, a CAR variant provided herein has an activity that is at least 160% higher. In some embodiments, a CAR variant provided herein has an activity that is at least 170% higher. In some embodiments, a CAR variant provided herein has an activity that is at least 180% higher. In some embodiments, a CAR variant provided herein has an activity that is at least 190% higher. In some embodiments, a CAR variant provided herein has an activity that is at least 200% higher. It is understood that activity refers to the ability of a CAR variant described herein to convert a substrate to a product relative to a WT CAR (e.g., SEQ ID NO: 2) under the same assay conditions, such as those known in the art.

In some embodiments, a CAR variant provided herein is a variant of a reference or template polypeptide, wherein the reference or template polypeptide has an amino acid sequence of SEQ ID NO: 2, and the CAR variant has one or more alterations or modifications or mutations at an amino acid position relative to SEQ ID NO: 2. Accordingly, in some embodiments, a CAR variant provided herein includes one or more amino acid alterations or modifications or mutations at a residue corresponding to position 391. In some embodiments, the one or more amino acid alterations, modifications, or mutations comprise one or more amino acid additions, deletions, substitutions (or replacements), or any combination thereof. In some embodiments, the one or more amino acid alterations, modifications or mutations are conservative amino acid substitutions. In some embodiments, the one or more amino acid alterations, modifications, or mutations are non-conservative amino acid substitutions. In some embodiments, a CAR variant provided herein includes a conservative amino acid substitution and/or non-conservative amino acid substitution in 1 to 100 amino acid positions. In some aspects, such a conservative amino acid substitution is a chemically conservative or an evolutionary conservative amino acid substitution. Methods of identifying conservative amino acids are well known to one of skill in the art, any one of which can be used to generate the variant CARs described herein.

A CAR variant provided herein may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 alterations (or modifications or mutations) relative to a WT CAR (SEQ ID NO: 2). A CAR variant provided herein may comprise at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 31, at most 32, at most 33, at most 34, at most 35, at most 36, at most 37, at most 38, at most 39, at most 40, at most 41, at most 42, at most 43, at most 44, at most 45, at most 46, at most 47, at most 48, at most 49, at most 50, at most 51, at most 52, at most 53, at most 54, at most 55, at most 56, at most 57, at most 58, at most 59, at most 60, at most 61, at most 62, at most 63, at most 64, at most 65, at most 66, at most 67, at most 68, at most 69, at most 70, at most 71, at most 72, at most 73, at most 74, at most 75, at most 76, at most 77, at most 78, at most 79, at most 80, at most 81, at most 82, at most 83, at most 84, at most 85, at most 86, at most 87, at most 88, at most 89, at most 90, at most 91, at most 92, at most 93, at most 94, at most 95, at most 96, at most 97, at most 98, at most 99, at most 100, at most 101, at most 102, at most 103, at most 104, at most 105, at most 106, at most 107, at most 108, at most 109, at most 110, at most 111, at most 112, at most 113, at most 114, at most 115, at most 116, at most 117, at most 118, at most 119, at most 120, at most 121, at most 122, at most 123, at most 124, at most 125, at most 126, at most 127, at most 128, at most 129, at most 130, at most 131, at most 132, at most 133, at most 134, at most 135, at most 136, at most 137, at most 138, at most 139, at most 140, at most 141, at most 142, at most 143, at most 144, at most 145, at most 146, at most 147, at most 148, at most 149, at most 150, at most 151, at most 152, at most 153, at most 154, at most 155, at most 156, at most 157, at most 158, at most 159, at most 160, at most 161, at most 162, at most 163, at most 164, at most 165, at most 166, at most 167, at most 168, at most 169, at most 170, at most 171, at most 172, at most 173, at most 174, at most 175, at most 176, at most 177, at most 178, at most 179, at most 180, at most 181, at most 182, at most 183, at most 184, at most 185, at most 186, at most 187, at most 188, at most 189, at most 190, at most 191, at most 192, at most 193, at most 194, at most 195, at most 196, at most 197, at most 198, at most 199, at most 200, at most 201, at most 202, at most 203, at most 204, at most 205, at most 206, at most 207, at most 208, at most 209, at most 210, at most 211, at most 212, at most 213, at most 214, at most 215, at most 216, at most 217, at most 218, at most 219, at most 220, at most 221, at most 222, at most 223, at most 224, at most 225, at most 226, at most 227, at most 228, at most 229, at most 230, at most 231, at most 232, at most 233, at most 234, at most 235, at most 236, at most 237, at most 238, at most 239, at most 240, at most 241, at most 242, at most 243, at most 244, at most 245, at most 246, at most 247, at most 248, at most 249, or at most 250 alterations (or modifications or mutations) relative to a WT CAR (SEQ ID NO: 2). The one or more alterations or modifications may be located at one or more positions corresponding to one or more positions in SEQ ID NO: 2. As used herein, the phrase “a residue corresponding to position X in SEQ ID NO: Y” refers to a residue at a corresponding position following an alignment of two sequences. For example, the residue in SEQ ID NO: 2 corresponding to position 391 in SEQ ID NO: 3 is the residue at position 391 in SEQ ID NO: 2. In some embodiments, a reference or template sequence is a CAR that is not SEQ ID NO: 2. In certain embodiments, the one or more amino acid alterations or modifications result in a CAR variant having a serine(S) at a residue corresponding to position 391 in SEQ ID NO: 2.

In some embodiments, the CAR, the CAR variant or the CAR homolog provided herein comprises an amino acid sequence that has at least 20-99% sequence identity with the wild-type or template (or reference) sequence, for example the amino acid sequence set forth in SEQ ID NO: 2 or SEQ ID NO: 3. For example, the CAR, CAR variant or homolog comprises an amino acid sequence that has at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99%, or higher, sequence identity with the template or wild-type sequence, such as, for example, with the sequence set forth in SEQ ID NO: 2 or SEQ ID NO: 3. In some embodiments, the CAR or variant or homolog thereof comprises an amino acid sequence that has at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99%, or higher, sequence identity with an CAR derived from a species or strain of Mycobacterium. In some embodiments, the CAR or variant or homolog thereof comprises an amino acid sequence that has at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99%, or higher, sequence identity with an CAR derived from a species or strain of Mycolicibacterium. In some embodiments, the CAR or variant or homolog thereof comprises an amino acid sequence that has at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99%, or higher, sequence identity to a CAR derived from Mycobacterium avium, or to a CAR derived from Mycobacterium smegmatis. In some embodiments, the CAR or variant or homolog thereof comprises an amino acid sequence that has at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99%, or higher, sequence identity to CAR derived from a strain or species of Mycolicibacterium, Mycolicibacillus, Mycolicibacter, Pseudofrankia, Norcadia, Pseudonocardiaceae, or Longimycelium. In some embodiments, the CAR or variant or homolog thereof is derived from a strain or species listed in TABLE 3 or TABLE 3.1, or the CAR or variant or homolog thereof is one listed in TABLE 3 or TABLE 3.1. In some embodiments, the CAR is a variant or homolog of any one or more of the CARs listed in TABLE 3 or TABLE 3.1. In some embodiments, the CAR is a variant or homolog, comprising one or more amino acid alterations (or modifications) relative to a reference or template sequence, wherein other than the one or more amino acid alterations or modifications, the CAR comprises an amino acid sequence that has at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99%, or higher, sequence identity to any one or more of the CARs listed in TABLE 3 or Table 3.1. In some embodiments, the CAR variant is a naturally occurring variant, such as a CAR (e.g., a homolog) from a different species or organism. In some embodiments, the CAR variant is a genetically engineered, non-naturally occurring variant.

Exemplary CAR variants include CAR Variant 1 (SEQ ID NO: 3), having the mutation G391S, relative to the WT CAR sequence set forth in SEQ ID NO: 2. In some embodiments, the CAR or variant or homolog thereof is heterologous to the non-naturally occurring microbial organisms provided herein. In some embodiments, the CAR or variant or homolog thereof is native to the non-naturally occurring microbial organisms provided herein. In some embodiments, the CAR is overexpressed. In some embodiments, the CAR is encoded by an exogenous nucleic acid sequence. In some embodiments, the CAR or variant or homolog thereof is heterologous to the non-naturally occurring microbial organism and is expressed or overexpressed. In other embodiments, the CAR or variant or homolog thereof is native to the non-naturally occurring microbial organism and is overexpressed.

In some embodiments, the CAR, or the polypeptide or enzyme with CAR activity, or a variant thereof, is encoded by one or more heterologous nucleic acid(s). In some embodiments, the CAR, or the polypeptide or enzyme with CAR activity, or a variant thereof, is encoded by one or more exogenous nucleic acid(s). In certain embodiments, the one or more exogenous nucleic acid(s) is/are a heterologous nucleic acid(s). In some embodiments, the CAR, or the polypeptide or enzyme with CAR activity, or a variant thereof, is expressed in a sufficient amount to eliminate or substantially eliminate, or to decrease the production, or amount, titer, or concentration, of 3HB by-product. In some embodiments, the CAR or variant thereof is expressed in a sufficient amount to enhance carbon flow through 3HBAld. In some embodiments, the CAR, or the polypeptide or enzyme with CAR activity, or a variant thereof, is expressed in a sufficient amount to increase the productivity, titer and/or yield of an acetyl-CoA derived product, such as, for example, 1,3-BDO and (R)-1,3-BDO.

In some embodiments, the microbial organism includes both: (a) a 3HB recycling loop; and (b) a 3HB to 3HBAld conversion step. In certain embodiments, where the microbial organism includes both the 3HB recycling pathway and the 3HB to 3HBAld conversion step, the carbon flux through 3HB—CoA and through 3HBAld can be enhanced or increased, thereby increasing the production, yield, titer, and/or productivity of acetyl-CoA derived products, such as, for example, 1,3-BDO, (R)-1,3-BDO, MAA, (3R)-hydroxybutyl (3R)-hydroxybutyrate, or MMA, compared to a microbial organism that includes only one of a 3HB recycling pathway or a 3HB to 3HBAld conversion step. Accordingly, the combination of both mechanisms for reducing 3HB by-product can be additive and can allow for an even greater productivity, titer, and/or yield of acetyl-CoA derived products. In some embodiments, the combination of both mechanisms for reducing 3HB by-product can be synergistic and can allow for an even greater productivity, titer, and/or yield of acetyl-CoA derived products. Thus, (i) the expression of ACS reduces the amount of 3HB by-product and increases the production of an acetyl-CoA derived product (e.g., 1,3-BDO) with a 3HB-derived intermediate (e.g., 3HB—CoA, and optionally, 3HBAld); (ii) the expression of CAR reduces the amount of 3HB by-product and increases the production of an acetyl-CoA derived product (e.g., 1,3-BDO) with a 3HB-derived intermediate (e.g., 3HB—CoA and/or a 3HBAld); and (iii) the expression of both ACS and CAR in a strain expressing an acetyl-CoA derived product (e.g., 1,3-BDO) pathway with 3HB-derived intermediates (e.g., 3HB—CoA and 3HBAld), results in a greater reduction in 3HB by-product and/or a greater increase in the production of an acetyl-CoA derived product, compared to a strain expressing only an ACS or only a CAR.

In some embodiments, the expression or overexpression of an ACS can result in a 3HB by-product recycling loop and an acetate by-product recycling loop, which can have an additive and/or synergistic effect, and can allow for an even greater productivity, titer, and/or yield of acetyl-CoA derived products. In some embodiments, the addition of a 3HB to 3HBAld conversion step further increases the productivity, titer, and/or yield of acetyl-CoA derived products. Accordingly, the combination of (i) reducing 3HB by-product by expressing a 3HB recycling loop (or by expressing or overexpressing ACS or a homolog or variant thereof); (ii) reducing acetate by-product by expressing an acetate recycling loop (or by expressing or overexpressing ACS or a homolog or variant thereof); and (iii) reducing 3HB by-product by expressing a 3HB to 3HBAld conversion step (or by expressing or overexpressing a CAR or a variant or homolog thereof), can be additive and/or synergistic, and can allow for an even greater productivity, titer, and/or yield of acetyl-CoA derived products, for example, when compared to expression of (i) alone, (ii) alone, (iii) alone, or any two of (i), (ii), and (iii). In some embodiments, the expression of ACS or a homolog or variant thereof reduces the formation and/or intracellular concentration of one or more carboxylate by-products, which can be additive and/or synergistic, and can result in an increased productivity, titer, and/or yield of acetyl-CoA derived products, compared to the reduction of only one carboxylate by-product. In some embodiments, the addition of a 3HB to 3HBAld conversion step further increases the productivity, titer, and/or yield of acetyl-CoA derived products, compared to the absence of a 3HB to 3HBAld conversion step. Thus, the expression of a carboxylate by-product recycling pathway and a 3HB to 3HBAld conversion step can be additive and/or synergistic; the expression of more than one carboxylate by-product recycling pathway (e.g., at least two by-product recycling pathways) can be additive and/or synergistic; and the expression of more than one carboxylate by-product recycling pathway (e.g., at least two), and the expression of a 3HB to 3HBAld conversion step can be additive and/or synergistic; and all of these additive and/or synergistic combinations/pathways result in an increased production (e.g., titer, yield, and/or productivity) of at least one acetyl-CoA derived product, compared to the absence or one or more additive and/or synergistic pathways.

In some embodiments, a non-naturally occurring microbial organism having a 3HB recycling loop and a 3HB to 3HBAld conversion step results in about a 0.01-fold to about a 10-fold increase, or more, in the productivity, titer, and/or yield of acetyl-CoA derived products, as compared to a microbial organism without the 3HB recycling loop and without the 3HB to 3HBAld conversion step, or as compared to a microbial organism without one or more exogenous nucleic acids encoding an ACS or CoA ligase, or a polypeptide or enzyme with ACS activity or CoA ligase activity, or a homolog or variant thereof, and without one or more exogenous nucleic acids encoding a CAR, or a polypeptide or enzyme with CAR activity, or a homolog or variant thereof. In some embodiments, a non-naturally occurring microbial organism having a 3HB recycling loop and a 3HB to 3HBAld conversion step results in about a 0.02-fold, about a 0.03-fold, about a 0.04-fold, about a 0.05-fold, about a 0.06-fold, about a 0.07-fold, about a 0.08-fold, about a 0.09-fold, about a 0.1-fold, about a 0.2 fold, about a 0.3-fold, about a 0.4-fold, about a 0.5-fold, about a 0.6-fold, about a 0.7-fold, about a 0.8-fold, about a 0.9-fold, about a 1-fold, about a 2-fold, about a 3-fold, about a 4-fold, about a 5-fold, about a 6-fold, about a 7-fold, about an 8-fold, about a 9-fold, or about a 10-fold increase in the productivity, titer, and/or yield of acetyl-CoA derived products, such as 1,3-BDO, (R)-1,3-BDO, MAA, (3R)-hydroxybutyl (3R)-hydroxybutyrate, and MMA.

As provided herein, the non-naturally occurring microbial organism having eliminated, substantially eliminated, or reduced 3HB by-product, and enhanced carbon flux through 3HB—CoA and/or 3HB-Ald, by having a 3HB recycling loop and/or a 3HB to 3HBAld conversion step, also includes a: 3HB—CoA pathway, or a pathway having a 3HB—CoA intermediate, optionally wherein the 3HB—CoA pathway also has a 3HBAld downstream intermediate. In some embodiments, the pathway having a 3HB—CoA and/or 3HB Ald intermediate is an acetyl-CoA derived product pathway, such as, for example, a 1,3-BDO pathway, a (3R)-hydroxybutyl (3R)-hydroxybutyrate pathway, a methyl methacrylate (MMA) pathway, and/or a methacrylic acid (MAA) pathway. Accordingly, such a non-naturally occurring microbial organism having eliminated, substantially eliminated, or reduced 3HB by-product, comprises a microbial organism having increased production (e.g., in terms of productivity, titer, and/or yield) of an acetyl-CoA derived product, such as 1,3-BDO, (R)-1,3-BDO, MAA, (3R)-hydroxybutyl (3R)-hydroxybutyrate and/or MMA.

In some embodiments, the non-naturally occurring microbial organism or cell of the present invention, having a glycolysis pathway and/or an Ac—CoA pathway, can include an Ac—CoA derived product pathway with one or more precursor and/or intermediate that is a CoA thioester of a carboxylate by-product (i.e., an acyl-CoA precursor or intermediate), particularly a CoA thioester of a short-chain (e.g., C2-C4, C2-C6, or C2-C8) carboxylate by-product (i.e., a short-chain acyl-CoA precursor or intermediate). Accordingly, in some embodiments, the non-naturally occurring microbial organism having eliminated, substantially eliminated, or reduced carboxylate by-product(s), and/or enhanced or increased carbon flux through the one or more acyl-CoA precursor(s) and/or intermediate(s), also includes an acetyl-CoA derived product pathway.

In some embodiments, the non-naturally occurring microbial organism or cell of the present invention having a glycolysis pathway and/or an Ac—COA pathway, can include an Ac—CoA derived product pathway with a 3HB—CoA intermediate. Accordingly, in some embodiments, the non-naturally occurring microbial organism having eliminated, substantially eliminated, or reduced 3HB by-product, and/or enhanced or increased carbon flux through 3HB—CoA and/or 3HBAld, also includes an Ac—COA derived product pathway.

In certain embodiments, the 3HB—CoA is an R isomer or is an S isomer, or is a mixture thereof, such as a racemic mixture. Accordingly, in some embodiments, the 3HB—CoA is a (3R)-hydroxybutyryl-CoA ((3R)—HB—CoA) isomer, and the 3HB by-product is (3R)-hydroxybutryrate ((3R)—HB). In other embodiments, the 3HB—CoA is a (3S)-hydroxybutyryl-CoA ((3S)—HB—CoA) isomer, and the 3HB by-product is (3S)-hydroxybutryrate ((3S)—HB). It is understood that certain enzymes have greater specificity, selectivity and/or affinity for certain isomers (i.e., the S or R isomer), and are not known to efficiently catalyze reactions in which the other isomer (i.e., the R or S isomer, respectively) is a substrate. Accordingly, the present invention envisions the selection of exogenous nucleic acids encoding stereospecific, or stereoselective, enzymes for certain metabolites that will redirect carbon flux through pathways that can utilize the isomeric substrate.

In certain embodiments, the non-naturally occurring microbial organism or cell, having eliminated, substantially eliminated, or reduced 3HB by-product, and/or enhanced or increased carbon flux through 3HB—CoA, wherein the non-naturally occurring microbial organism or cell includes at least one exogenous nucleic acid encoding one or more of an enzyme or polypeptide that converts a substrate to 3HB—CoA, or an isomeric form thereof, including, for example:
(i) an enzyme or polypeptide that converts AcAc—COA to 3HB—CoA;
(ii) an enzyme or polypeptide that converts AcAc—COA to (3S)—HB—CoA;
(iii) an enzyme or polypeptide that converts AcAc—COA to (3R)—HB—CoA;
(iv) an enzyme or polypeptide that converts crotonyl-CoA to 3HB—CoA; and/or
(v) an enzyme or polypeptide that converts (3S)—HB—CoA to (3R)—HB—CoA;

In some embodiments, AcAc—COA is converted to 3HB—CoA by an enzyme or polypeptide having an AcAc—COA reductase (ketone reducing/alcohol forming oxidoreductase) activity (EC 1.1.a). In some embodiments, AcAc—COA is converted to (3S)—HB—CoA by an enzyme or polypeptide having an 3-hydroxybutyryl-CoA dehydrogenase (HBD), optionally, (3S)-hydroxybutyryl-CoA dehydrogenase (S—HBD) (EC 1.1.1.157) activity. In some embodiments, AcAc—COA is converted to (3R)—HB—CoA by an enzyme or polypeptide having HBD, optionally, (3R)-hydroxybutyryl-CoA dehydrogenase (R—HBD) (EC 1.1.1.157) activity. In some embodiments, crotonyl-CoA is converted to 3HB—CoA by an enzyme or polypeptide having a crotonase (CRT) activity (EC 4.2.1.a). In some embodiments, (3S)—HB—CoA is converted to (3R)—HB—CoA by an enzyme or polypeptide having a 3HB—CoA epimerase activity (EC 5.1.2.3).

In certain embodiments, an Ac—COA derived product pathway with a 3HB—CoA intermediate, and/or the pathway of an isomer thereof, can include one or more of the following enzymes: AcAc—COA thiolase and/or AcAc—COA reductase (ketone reducing); CRT; HBD optionally S—HBD and/or or R—HBD; and/or 3HB—CoA epimerase activity; or any combination thereof.

The non-naturally occurring microbial organism can include at least one exogenous nucleic acid encoding an enzyme of an Ac—COA derived product pathway with a 3HB—CoA intermediate or an acyl-CoA intermediate or precursor. The non-naturally occurring microbial organism can include two exogenous nucleic acids each encoding an enzyme of an Ac—COA derived product pathway with a 3HB—CoA intermediate or an acyl-CoA intermediate or precursor, in other embodiments, three exogenous nucleic acids each encoding an enzyme of an Ac—COA derived product pathway with a 3HB—CoA intermediate or an acyl-CoA intermediate or precursor, in other embodiments, four exogenous nucleic acids each encoding an enzyme of an Ac—CoA derived product pathway with a 3HB—CoA intermediate or an acyl-CoA intermediate or precursor, or more exogenous nucleic acids each encoding an enzyme of an Ac—COA derived product pathway with a 3HB—CoA intermediate or an acyl-CoA intermediate or precursor.

Any number of nucleic acids encoding these polypeptides or enzymes can be further introduced into a host microbial organism or cell including one, two, three, four, or five nucleic acids that encode other polypeptides or enzymes included in any one or more of the Ac—COA derived product pathway(s) with a 3HB—CoA intermediate or one or more acyl-CoA intermediates (or precursors) described herein. Where more than one exogenous nucleic acids are introduced, such nucleic acids can be any permutation of the multiple nucleic acids (e.g., polycistronic or multiple nucleic acids) already included in the host organism or cell.

In other embodiments, the non-naturally occurring microbial organism having eliminated, substantially eliminated, or reduced 3HB by-product, and/or enhanced or increased carbon flux through 3HB—CoA and/or 3HBAld, includes a 3HB—CoA pathway having a downstream 3HBAld intermediate. In some embodiments, the downstream 3HBAld intermediate can be an R isomer, an S isomer, or a mixture thereof, such as a racemic mixture thereof. Accordingly, in some embodiments, the downstream 3HBAld intermediate is (3R)-hydroxybutyraldehyde ((3R)—HBAld), wherein the 3HB by-product is (3R)—HB. In some embodiments, the downstream 3HBAld intermediate is (3S)-hydroxybutyraldehyde ((3S)—HBAld), wherein the 3HB by-product is (3S)—HB.

One skilled in the art will understand that these Ac—COA derived product pathways having a 3HB—CoA intermediate or one or more acyl-CoA intermediates or precursors, and/or the pathways of an isomer thereof, substrate-product pairs, and encoded enzymes or polypeptides are merely exemplary, and that any of the substrate-product pairs and/or encoded enzymes or polypeptides disclosed herein that are suitable to produce a desired acetyl-CoA derived product, such as those described herein, and for which an appropriate activity is available for the conversion of the substrate to the product, can be readily determined by one skilled in the art based on the teachings herein.

Any of the at least one exogenous nucleic acids can be a heterologous nucleic acid, and the non-naturally occurring microbial organism can be constructed for growth or culturing under substantially anaerobic conditions, for example, in a substantially anaerobic culture medium. Other culture conditions, including feedstock, are described in further detail herein.

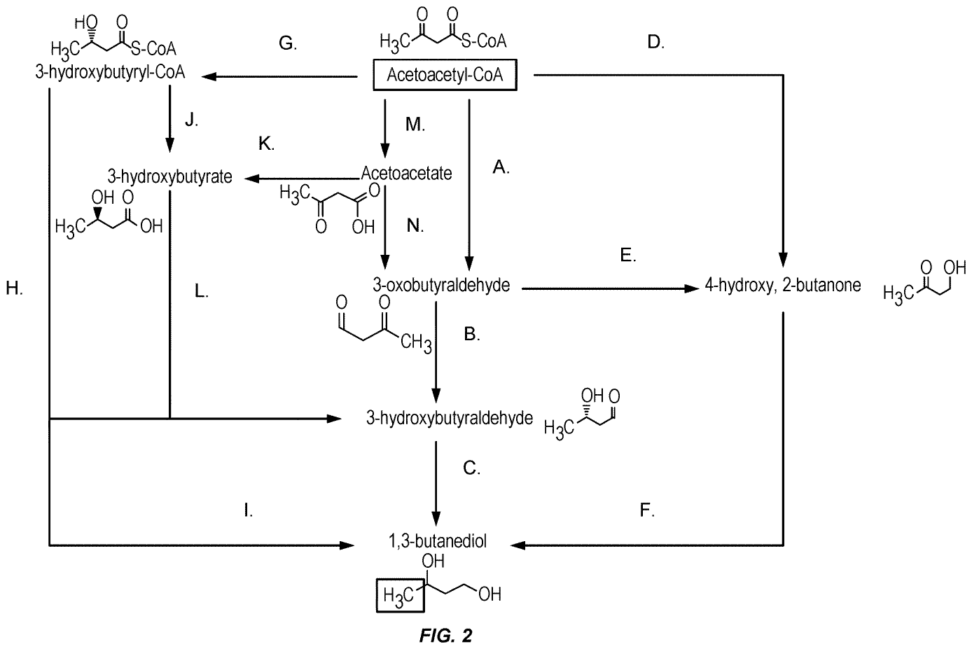

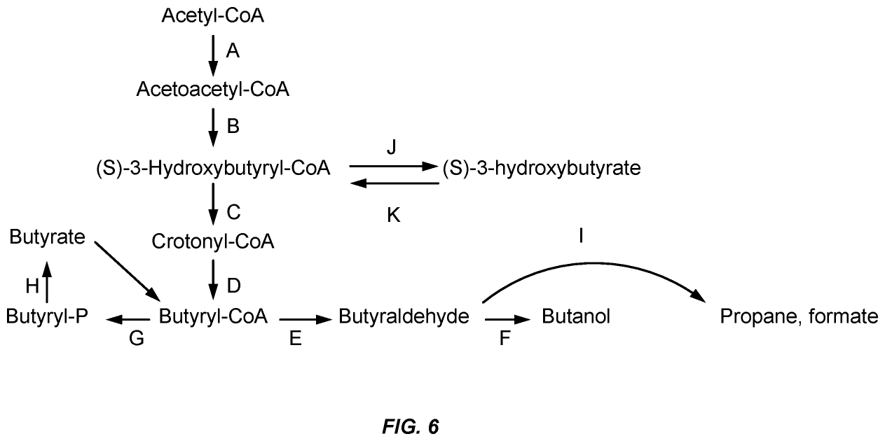

Thus, provided herein is a non-naturally occurring microbial organism or cell having eliminated, substantially eliminated, or reduced 3HB by-product, and/or enhanced or increased carbon flux through 3HB—CoA and/or 3HBAld, and containing at least one exogenous nucleic acid encoding one or more enzymes or polypeptides, where the one or more enzymes or polypeptides are part of a an Ac—CoA derived product pathway with a 3HB—CoA and/or a 3HB Ald intermediate, and/or the pathway of an isomer thereof, as exemplified by the pathways described herein and shown, for example in any one of FIG. 2 to FIG. 10.

The non-naturally occurring organism or cell of the present invention having a glycolysis pathway or an Ac—COA pathway can include an Ac—COA derived product pathway with a 3HB—CoA intermediate, and, in certain embodiments, a downstream 3HBAld intermediate, wherein the Ac—COA derived product is 1,3-butanediol (1,3-BDO). Accordingly, in certain embodiments, the non-naturally occurring microbial organism or cell, having eliminated, substantially eliminated, or reduced 3HB by-product, and/or enhanced or increased carbon flux through 3HB—CoA and/or 3HBAld, includes a 1,3-BDO pathway.

In certain embodiments, the 1,3-BDO is an R isomer, or is an S isomer, or is a mixture thereof, such as a racemic mixture thereof. Accordingly, in some embodiments, the 1,3-BDO is (R)-1,3-BDO, the 3HB—CoA is (3R)—HB—CoA, and the 3HB by-product is (3R)—HB. In other embodiments, the 1,3-BDO is (S)-1,3-BDO, the 3HB—CoA is (3S)—HB—CoA, and the 3HB by-product is (3S)—HB. It is understood that certain enzymes have greater specificity, selectivity, and/or affinity for certain isomers (i.e., the S or R isomer), and are not known to efficiently catalyze reactions in which the other isomer (i.e., the R or S isomer, respectively) is a substrate. Accordingly, the present invention envisions the selection of exogenous nucleic acids encoding stereospecific and/or stereoselective enzymes for certain metabolites that will redirect carbon flux through pathways that can utilize the isomeric substrate.

In certain embodiments, the non-naturally occurring microbial organism or cell, having eliminated, substantially eliminated, or reduced 3HB by-product or carboxylate by-product (e.g., acetate and/or acetoacetate), and/or enhanced or increased carbon flux through 3HB—CoA, and/or 3HBAld, and/or an acyl-CoA (particularly a short-chain acyl-CoA, e.g., acetyl-CoA and/or acetoacetyl-CoA), can further include a 1,3-BDO pathway, wherein the non-naturally occurring microbial organism or cell includes at least one exogenous nucleic acid encoding one or more of an enzyme or polypeptide that converts a substrate to a product, or an isomeric form thereof including, for example:
(i) an enzyme or polypeptide that converts Ac—COA to AcAc—COA;
(ii) an enzyme or polypeptide that converts AcAc—COA to 3HB—CoA;
(iii) an enzyme or polypeptide that converts 3HB—CoA to 3HBAld; and/or
(iv) an enzyme or polypeptide that converts 3HBAld to 1,3-BDO.

In certain embodiments, the non-naturally occurring microbial organism or cell, having eliminated, substantially eliminated, or reduced 3HB by-product or carboxylate by-product (e.g., acetate and/or acetoacetate), and/or enhanced or increased carbon flux through 3HB—CoA, and/or 3HB-Ald, and/or an acyl-CoA (particularly a short-chain acyl-CoA, e.g., acetyl-CoA and/or acetoacetyl-CoA), can further include a 1,3-BDO pathway, wherein the non-naturally occurring microbial organism or cell includes at least one exogenous nucleic acid encoding one or more of an enzyme or polypeptide that converts a substrate to a product, or an isomeric form thereof including, for example:
(i) an enzyme or polypeptide that converts Ac—COA to AcAc—COA;
(ii) an enzyme or polypeptide that converts AcAc—COA to 3HB—CoA; and/or
(iii) an enzyme or polypeptide that converts 3HB—CoA to 1,3-BDO.

In certain embodiments, the non-naturally occurring microbial organism or cell, having eliminated, substantially eliminated, or reduced 3HB by-product or carboxylate by-product (e.g., acetate and/or acetoacetate), and/or enhanced or increased carbon flux through 3HB—CoA, and/or 3HB-Ald, and/or an acyl-CoA (particularly a short-chain acyl-CoA, e.g., acetyl-CoA and/or acetoacetyl-CoA), can further include a 1,3-BDO pathway, wherein the non-naturally occurring microbial organism or cell includes at least one exogenous nucleic acid encoding one or more of an enzyme or a polypeptide that converts a substrate to a product, or an isomeric form thereof, including, for example:
(i) an enzyme or polypeptide that converts Ac—COA to AcAc—COA;
(ii) an enzyme or polypeptide that converts AcAc—COA to acetoacetate;
(iii) an enzyme or polypeptide that converts acetoacetate to 3HB;
   wherein 3HB gets recycled back into 3HB—CoA as described herein;
(iv) an enzyme or polypeptide that converts 3HB—CoA to 3HBAld; and/or
(v) an enzyme or polypeptide that converts 3HBAld to 1,3-BDO.

In certain embodiments, the non-naturally occurring microbial organism or cell, having eliminated, substantially eliminated, or reduced 3HB by-product or carboxylate by-product (e.g., acetate and/or acetoacetate), and/or enhanced or increased carbon flux through 3HB—CoA, and/or 3HB-Ald, and/or an acyl-CoA (particularly a short-chain acyl-CoA, e.g., acetyl-CoA and/or acetoacetyl-CoA), can further include a 1,3-BDO pathway, wherein the non-naturally occurring microbial organism or cell includes at least one exogenous nucleic acid encoding one or more of an enzyme or a polypeptide that converts a substrate to a product, or an isomeric form thereof including, for example:
(i) an enzyme or polypeptide that converts Ac—COA to AcAc—COA;
(ii) an enzyme or polypeptide that converts AcAc—COA to acetoacetate;
(iii) an enzyme or polypeptide that converts acetoacetate to 3HB; wherein 3HB gets recycled back into 3HB—CoA as described herein; and/or
(iv) an enzyme or polypeptide that converts 3HB—CoA to 1,3-BDO.

In some embodiments, Ac—COA is converted to AcAc—COA by an enzyme or polypeptide having an Ac—COA thiolase (EC 2.3.1) activity. In some embodiments, AcAc—COA is converted to 3HB—CoA by an enzyme or polypeptide having 3HB—CoA dehydrogenase (EC 1.1.1.a) activity or AcAc—COA reductase (ketone reducing) (EC 1.1.1.a) activity. In some embodiments, 3HB—CoA is converted to 1,3-BDO by an enzyme or polypeptide having 3HB—CoA reductase (an alcohol forming oxidoreductase) (CoA dependent) (EC 1.1.1.c) activity. In some embodiments, AcAc—COA is converted to acetoacetate by an enzyme or polypeptide having an AcAc—COA transferase (EC 2.8.3.a), an AcAc—COA hydrolase (EC 3.1.2.a), or an AcAc—COA synthetase (EC 6.2.1.a) activity. In some embodiments, acetoacetate is converted to 3HB by an enzyme or polypeptide having a 3HB dehydrogenase (a ketone reducing, alcohol forming oxidoreductase) (EC 1.1.1.a) activity, wherein 3HB gets recycled back into 3HB—CoA as described herein. In some embodiments, 3HB—CoA is converted to 3HBAld by an enzyme or polypeptide having 3HB—CoA reductase (aldehyde forming) (EC 1.2.1.b) activity, aldehyde dehydrogenase (ALD) (EC 1.2.1) activity, butyraldehyde dehydrogenase (EC 1.2.1) activity, or acetaldehyde-alcohol dehydrogenase (EC 1.2.1) activity. In some embodiments, 3HB is converted to 3HBAld by a 3HB to 3HBAld conversion step as described herein. In some embodiments, 3HBAld is converted to 1,3-BDO by an enzyme or polypeptide having 3HBAld reductase (EC 1.1.1.a) activity, acetaldehyde-alcohol dehydrogenase (EC 1.2.1) activity, 1,3-BDO or 1,3-butylene glycol dehydrogenase (EC 1.1.1) activity, or alcohol dehydrogenase (ADH) (EC 1.1.1) activity.

In certain embodiments, a 1,3-BDO pathway, and/or the pathway of an isomer thereof, can include one or more of the following enzymes: AcAc—COA reductase (a CoA-dependent, aldehyde forming oxidoreductase); 3-oxobutyraldehyde reductase (a ketone reducing/alcohol forming oxidoreductase); 3HB—Ald reductase (an aldehyde reducing/alcohol forming oxidoreductase), acetaldehyde-alcohol dehydrogenase, 1,3-BDO or 1,3-butylene glycol dehydrogenase, or ADH; AcAc—COA reductase (a CoA-dependent, alcohol forming oxidoreductase); 3-oxobutyraldehyde reductase (an aldehyde reducing/alcohol forming oxidoreductase); 4-hydroxy-2-butanone reductase (a ketone reducing/alcohol forming oxidoreductase); AcAc—COA reductase (ketone reducing/alcohol forming oxidoreductase); 3HB—CoA reductase (a CoA-dependent, aldehyde forming oxidoreductase), ALD, or acetaldehyde-alcohol dehydrogenase; 3HB—CoA reductase (an alcohol forming, CoA dependent oxidoreductase); 3HB—CoA transferase, or a 3HB—CoA hydrolase; 3HB dehydrogenase (a ketone reducing, alcohol forming oxidoreductase); AcAc—COA transferase, AcAc—COA hydrolase, AcAc—COA synthetase; acetoacetate reductase (an aldehyde forming oxidoreductase); and/or a Ac—COA thiolase.

The non-naturally occurring microbial organism can include at least one exogenous nucleic acid encoding a 1,3-BDO pathway enzyme. The non-naturally occurring microbial organism can include two exogenous nucleic acids each encoding a 1,3-BDO pathway enzyme, in other embodiments, three exogenous nucleic acids each encoding a 1,3-BDO pathway enzyme, in other embodiments, four exogenous nucleic acids each encoding a 1,3-BDO pathway enzyme, in other embodiments, five exogenous nucleic acids each encoding a 1,3-BDO pathway enzyme, in other embodiments, six exogenous nucleic acids each encoding a 1,3-BDO pathway enzyme, in other embodiments, seven exogenous nucleic acids each encoding a 1,3-BDO pathway enzyme, in other embodiments, eight exogenous nucleic acids each encoding a 1,3-BDO pathway enzyme, in other embodiments, nine exogenous nucleic acids each encoding a 1,3-BDO pathway enzyme, in other embodiments, ten exogenous nucleic acids each encoding a 1,3-BDO pathway enzyme, in other embodiments, eleven, in other embodiments, twelve exogenous nucleic acids each encoding a 1,3-BDO pathway enzyme, in other embodiments, exogenous nucleic acids each encoding a 1,3-BDO pathway enzyme, in other embodiments, thirteen exogenous nucleic acids each encoding a 1,3-BDO pathway enzyme, in other embodiments, fourteen exogenous nucleic acids each encoding a 1,3-BDO pathway enzyme, in other embodiments, fifteen or more exogenous nucleic acids each encoding a 1,3-BDO pathway enzyme.

Any number of nucleic acids encoding these enzymes or polypeptides can be further introduced into a host microbial organism or cell already including one, two, three, four, five, six, seven, eight, nine, ten, eleven, twelve, thirteen, fourteen, or fifteen nucleic acids that encode any one or more of the other enzymes or polypeptides in the 1,3-BDO pathway, and/or the pathway of an isomer thereof, as described herein. Where more than one exogenous nucleic acids are introduced, such nucleic acids can be any permutation of the multiple nucleic acids (e.g., polycistronic or multiple nucleic acids) already included in the host organism or cell.

One skilled in the art will understand that these 1,3-BDO pathways, and/or the pathways of an isomer thereof, substrate-product pairs, and encoded enzymes or polypeptides are merely exemplary, and that any of the substrate-product pairs and/or encoded enzymes or polypeptides disclosed herein that are suitable to produce a desired acetyl-CoA derived product, such as 1,3-BDO, and/or an isomer thereof, and for which an appropriate activity is available for the conversion of the substrate to the product, can be readily determined by one skilled in the art based on the teachings herein.

Any of the at least one exogenous nucleic acids can be a heterologous nucleic acid, and the non-naturally occurring microbial organism can be constructed for growth or culturing under substantially anaerobic conditions, for example, in a substantially anaerobic culture medium. Other culture conditions, including feedstock, are described in further detail herein.

Thus, provided herein is a non-naturally occurring microbial organism or cell having eliminated, substantially eliminated, or reduced 3HB by-product or carboxylate by-product (e.g., acetate and/or acetoacetate), and/or enhanced or increased carbon flux through 3HB—CoA, and/or 3HBAld, and/or an acyl-CoA (particularly a short-chain acyl-CoA, e.g., acetyl-CoA and/or acetoacetyl-CoA), and containing at least one exogenous nucleic acid encoding one or more enzymes or polypeptides, where the one or more enzymes or polypeptides are part of a 1,3-BDO pathway, and/or the pathway of an isomer thereof, converting acetyl-CoA or AcAc—COA to 1,3-BDO, and/or an isomer thereof, as exemplified by the pathways shown in FIG. 2.

In certain embodiments, the non-naturally occurring microbial organism or cell, having eliminated, substantially eliminated, or reduced (R)-3HB by-product, and/or enhanced or increased carbon flux through (R)-3HB—CoA and/or (R)-3HBAld, can further include a (R) 1,3-BDO pathway, wherein the non-naturally occurring microbial organism or cell includes at least one exogenous nucleic acid encoding one or more of an enzyme or polypeptide that converts a substrate to a product, or an isomeric form thereof including, for example:
(i) an enzyme or polypeptide that converts acetyl-CoA to AcAc—COA;
(ii) an enzyme or polypeptide that converts AcAc—COA, to (R)-3HB—CoA;
(iii) an enzyme or polypeptide that converts (R)-3HB—CoA to (R)-3HBAld; and/or
(iv) an enzyme or polypeptide that converts (R)-3HBAld to (R)-1,3 BDO.

In certain embodiments, the non-naturally occurring microbial organism or cell, having eliminated, substantially eliminated, or reduced 3HB by-product or carboxylate by-product (e.g., acetate and/or acetoacetate), and/or enhanced or increased carbon flux through 3HB—CoA, and/or 3HBAld, and/or an acyl-CoA (particularly a short-chain acyl-CoA, e.g., acetyl-CoA and/or acetoacetyl-CoA), can further include a 1,3-BDO pathway, wherein the non-naturally occurring microbial organism or cell includes at least one exogenous nucleic acid encoding one or more of an enzyme or polypeptide that converts a substrate to a product, or an isomeric form thereof including, for example:
(i) an enzyme or polypeptide that converts Ac—COA to AcAc—COA;
(ii) an enzyme or polypeptide that converts AcAc—COA, to (R)-3HB—CoA;
(iii) an enzyme or polypeptide that converts (R)-3HB—CoA to (R)-3-hydroxybutyryl-phosphate ((R)-3HB—P);
(iv) an enzyme or polypeptide that converts (R)-3HB—P to (R)-3HB,
   wherein 3HB gets recycled back into 3HB—CoA and/or is converted to 3HBAld by a 3HB to 3HBAld conversion step as described herein;
(v) an enzyme or polypeptide that converts (R)-3HB—CoA to (R)-3HBAld; and/or
(vi) an enzyme or polypeptide that converts (R)-3HBAld to (R)-1,3 BDO.

In some embodiments, Ac—COA is converted to AcAc—COA by an enzyme or polypeptide having an Ac—CoA thiolase (EC 2.3.1) activity. In some embodiments, AcAc—COA is converted to (R)-3HB—CoA by an enzyme or polypeptide having 3HB—CoA dehydrogenase (HBD), optionally (R)-3HB—CoA dehydrogenase (R—HBD) (EC 1.1.1.a) activity or AcAc—COA reductase (ketone reducing/alcohol forming oxidoreductase) (EC 1.1.1.a) activity. In some embodiments, (R)-3HB—CoA is converted to (R)-3HBAld by an enzyme or polypeptide having butyraldehyde dehydrogenase (EC 1.2.1.) activity or acetaldehyde-alcohol dehydrogenase (EC 1.2.1) activity. In some embodiments, (R)-3HBAld is converted to (R)-1,3-BDO by an enzyme or polypeptide having acetaldehyde-alcohol dehydrogenase (EC 1.2.1) activity. In some embodiments, (R)-3HB—CoA is converted to (R)-3HB—P by an enzyme or polypeptide having phosphotransbutyrylase (PTB) (EC 2.3.1) activity. In some embodiments, (R)-3HB—P is converted to (R)-3HB by an enzyme or polypeptide having butyrate kinase (BUK) (EC 2.7.2) activity, wherein 3HB gets recycled back into 3HB—CoA and/or is converted to 3HBAld by a 3HB to 3HBAld conversion step as described herein.

In certain embodiments, a 1,3-BDO pathway, and/or the pathway of an isomer thereof, can include one or more of the following enzymes: AcAc—COA thiolase; HBD, optionally a R—HBD, or AcAc—COA reductase (ketone reducing/alcohol forming oxidoreductase); butyraldehyde dehydrogenase or acetaldehyde-alcohol dehydrogenase; acetaldehyde-alcohol dehydrogenase; PTB; and/or BUK.

The non-naturally occurring microbial organism can include at least one exogenous nucleic acid encoding a 1,3-BDO pathway enzyme. The non-naturally occurring microbial organism can include two exogenous nucleic acids each encoding a 1,3-BDO pathway enzyme, in other embodiments, three exogenous nucleic acids each encoding a 1,3-BDO pathway enzyme, in other embodiments, four exogenous nucleic acids each encoding a 1,3-BDO pathway enzyme, in other embodiments, five exogenous nucleic acids each encoding a 1,3-BDO pathway enzyme, in other embodiments, six or more exogenous nucleic acids each encoding a 1,3-BDO pathway enzyme.

Any number of nucleic acids encoding these enzymes or polypeptides can be further introduced into a host microbial organism or cell already including one, two, three, four, five, or six nucleic acids that encode any one or more of the other enzymes or polypeptides in the 1,3-BDO pathway, and/or the pathway of an isomer thereof, as described herein. Where more than one exogenous nucleic acids are introduced, such nucleic acids can be any permutation of the multiple nucleic acids (e.g., polycistronic or multiple nucleic acids) already included in the host organism or cell.

One skilled in the art will understand that these 1,3-BDO pathways, and/or the pathways of an isomer thereof, substrate-product pairs, and encoded enzymes or polypeptides are merely exemplary, and that any of the substrate-product pairs and/or encoded enzymes or polypeptides disclosed herein that are suitable to produce a desired acetyl-CoA derived product, such as 1,3-BDO, and/or an isomer thereof, and for which an appropriate activity is available for the conversion of the substrate to the product, can be readily determined by one skilled in the art based on the teachings herein.

Any of the at least one exogenous nucleic acids can be a heterologous nucleic acid, and the non-naturally occurring microbial organism can be constructed for growth or culturing under substantially anaerobic conditions, for example, in a substantially anaerobic culture medium. Other culture conditions, including feedstock, are described in further detail herein.

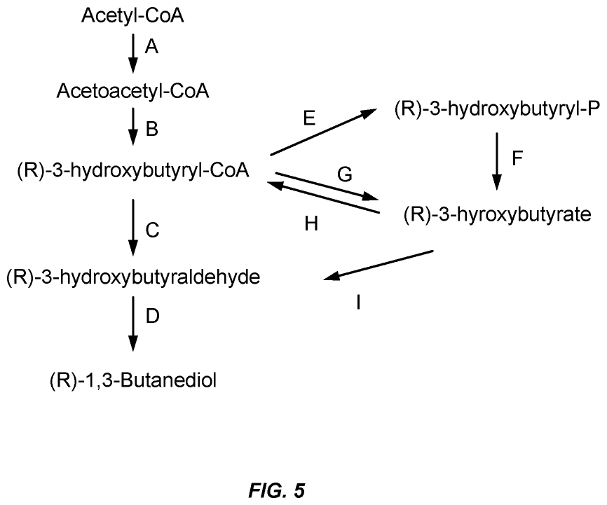

Thus, provided herein is a non-naturally occurring microbial organism or cell having eliminated, substantially eliminated, or reduced 3HB by-product or carboxylate by-product (e.g., acetate and/or acetoacetate), and/or enhanced or increased carbon flux through 3HB—CoA, and/or 3HBAld, and/or an acyl-CoA (particularly a short-chain acyl-CoA, e.g., acetyl-CoA and/or acetoacetyl-CoA), and containing at least one exogenous nucleic acid encoding one or more enzymes or polypeptides, where the one or more enzymes or polypeptides are part of a 1,3-BDO pathway, and/or the pathway of an isomer thereof, converting acetyl-CoA or AcAc—COA to 1,3-BDO, and/or an isomer thereof, as exemplified by the pathways shown in FIG. 5.

In certain embodiments, the non-naturally occurring microbial organism or cell, having eliminated, substantially eliminated, or reduced (R)-3HB by-product or carboxylate by-product (e.g., acetate and/or acetoacetate), and/or enhanced or increased carbon flux through (R)-3HB—CoA and/or (R)-3HBAld, and/or an acyl-CoA (particularly a short-chain acyl-CoA, e.g., acetyl-CoA and/or acetoacetyl-CoA), can further include a (R)-1,3-BDO pathway, wherein the non-naturally occurring microbial organism or cell includes at least one exogenous nucleic acid encoding one or more of an enzyme or polypeptide that converts a substrate to a product, or an isomeric form thereof including, for example:
(i) an enzyme or polypeptide that converts Ac—COA to AcAc—COA;
(ii) an enzyme or polypeptide that converts AcAc—COA to(S)-3HB—CoA;
(iii) an enzyme or polypeptide that converts (S)-3HB—CoA to (R)-3HB—CoA;
(iv) an enzyme or polypeptide that converts (R)-3HB—CoA to (R)-3HBAld; and/or
(v) an enzyme or polypeptide that converts (R)-3HBAld to (R)-1,3 BDO.

In certain embodiments, the non-naturally occurring microbial organism or cell, having eliminated, substantially eliminated, or reduced (R)-3HB by-product or carboxylate by-product (e.g., acetate, acetoacetate, and/or malonate), and/or enhanced or increased carbon flux through (R)-3HB—CoA, and/or (R)-3HBAld, and/or an acyl-CoA (particularly a short-chain acyl-CoA, e.g., acetyl-CoA, acetoacetyl-CoA, and/or malonyl-CoA), can further include a (R)-1,3-BDO pathway, wherein the non-naturally occurring microbial organism or cell includes at least one exogenous nucleic acid encoding one or more of an enzyme or polypeptide that converts a substrate to a product, or an isomeric form thereof including, for example:
(i) an enzyme or polypeptide that converts Ac—COA to malonyl-CoA
(ii) an enzyme or polypeptide that converts malonyl-CoA to AcAc—COA
(iii) an enzyme or polypeptide that converts AcAc—COA to (R)-3HB—CoA;
(iv) an enzyme or polypeptide that converts (R)-3HB—CoA to (R)-3HBAld; and/or
(v) an enzyme or polypeptide that converts (R)-3HBAld to (R)-1,3 BDO.

In certain embodiments, the non-naturally occurring microbial organism or cell, having eliminated, substantially eliminated, or reduced (R)-3HB by-product or carboxylate by-product (e.g., acetate, acetoacetate, and/or malonate), and/or enhanced or increased carbon flux through (R)-3HB—CoA, and/or (R)-3HBAld, and/or an acyl-CoA (particularly a short-chain acyl-CoA, e.g., acetyl-CoA, acetoacetyl-CoA, and/or malonyl-CoA), can further include an (R)-1,3-BDO pathway, wherein the non-naturally occurring microbial organism or cell includes at least one exogenous nucleic acid encoding one or more of an enzyme or polypeptide that converts a substrate to a product, or an isomeric form thereof including, for example:
(i) an enzyme or polypeptide that converts Ac—COA to malonyl-CoA
(ii) an enzyme or polypeptide that converts malonyl-CoA to AcAc—COA
(iii) an enzyme or polypeptide that converts AcAc—COA to(S)-3HB—CoA;
(iv) an enzyme or polypeptide that converts (S)-3HB—CoA to (R)-3HB—CoA;
(v) an enzyme or polypeptide that converts (R)-3HB—CoA to (R)-3HBAld; and/or
(vi) an enzyme or polypeptide that converts (R)-3HBAld to (R)-1,3 BDO.

In some embodiments, Ac—COA is converted to AcAc—COA by an enzyme or polypeptide having AcAc—COA thiolase (EC 2.3.1) activity. In some embodiments, AcAc—COA is converted to(S)-3HB—CoA by an enzyme or polypeptide having HBD, optionally (S)-3hydroxybutyryl-CoA dehydrogenase (S—HBD) (EC 1.1.1.157) activity. In some embodiments, (S)-3HB—CoA is converted to (R)-3HB—CoA by an enzyme or polypeptide having 3HB—CoA epimerase (EC 5.1.2.3) activity. In some embodiments, (R)-3HB—CoA is converted to (R)-3HBAld by an enzyme or polypeptide having (R)-3HB—CoA reductase (EC 1.2.1.b) activity. In some embodiments, (R)-3HBAld is converted to (R)-1,3 BDO by an enzyme or polypeptide having (R)-3HB—Ald reductase (EC 1.1.1.a) activity. In some embodiments, Ac—COA is converted to malonyl-CoA by an enzyme or polypeptide having Ac—CoA carboxylase (EC 6.4.12) activity. In some embodiments, malonyl-CoA is converted to AcAc—COA by an enzyme or polypeptide having AcAc—COA synthase (EC 2.3.1.194) activity. In some embodiments, AcAc—COA is converted to (R)-3HB—CoA by an enzyme or polypeptide having HBD, optionally, R—HBD, (EC1.1.157) activity.

In certain embodiments, a 1,3-BDO pathway, and/or the pathway of an isomer thereof, can include one or more of the following enzymes: AcAc—COA thiolase; HBD, optionally R—HBD; (R)-3HB—CoA reductase; (R)-3HB—Ald reductase; Ac—COA carboxylase; AcAc—COA synthase; HBD, optionally S—HBD; and/or 3HB—CoA epimerase.

The non-naturally occurring microbial organism can include at least one exogenous nucleic acid encoding a 1,3-BDO pathway enzyme. The non-naturally occurring microbial organism can include two exogenous nucleic acids each encoding a 1,3-BDO pathway enzyme, in other embodiments, three exogenous nucleic acids each encoding a 1,3-BDO pathway enzyme, in other embodiments, four exogenous nucleic acids each encoding a 1,3-BDO pathway enzyme, in other embodiments, five exogenous nucleic acids each encoding a 1,3-BDO pathway enzyme, in other embodiments, six exogenous nucleic acids each encoding a 1,3-BDO pathway enzyme, in other embodiments, seven exogenous nucleic acids each encoding a 1,3-BDO pathway enzyme, in other embodiments, eight or more exogenous nucleic acids each encoding a 1,3-BDO pathway enzyme.

Any number of nucleic acids encoding these enzymes or polypeptides can be further introduced into a host microbial organism or cell already including one, two, three, four, five, six, seven, or eight nucleic acids that encode any one or more of the other enzymes or polypeptides in the 1,3-BDO pathway, and/or the pathway of an isomer thereof, as described herein. Where one, two, three, four, five, six, seven, or eight exogenous nucleic acids are introduced, such nucleic acids can be any permutation of the additional eight nucleic acids already included in the host organism or cell.

One skilled in the art will understand that these 1,3-BDO pathways, and/or the pathways of an isomer thereof, substrate-product pairs, and encoded enzymes or polypeptides are merely exemplary, and that any of the substrate-product pairs and/or encoded enzymes or polypeptides disclosed herein that are suitable to produce a desired acetyl-CoA derived product, such as 1,3-BDO, and/or an isomer thereof, and for which an appropriate activity is available for the conversion of the substrate to the product, can be readily determined by one skilled in the art based on the teachings herein.

Any of the at least one exogenous nucleic acids can be a heterologous nucleic acid, and the non-naturally occurring microbial organism can be constructed for growth or culturing under substantially anaerobic conditions, for example, in a substantially anaerobic culture medium. Other culture conditions, including feedstock, are described in further detail herein.

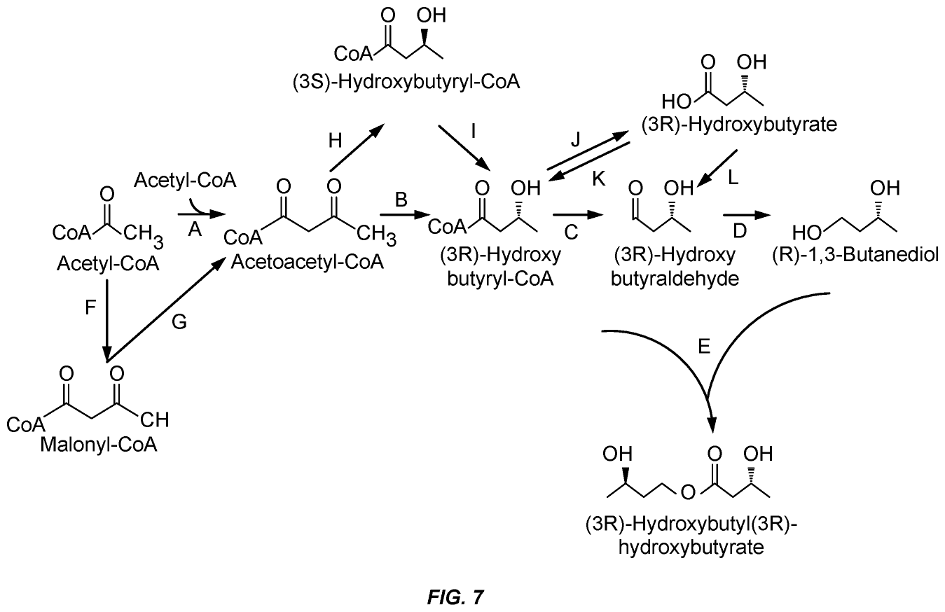

Thus, provided herein is a non-naturally occurring microbial organism or cell having eliminated, substantially eliminated, or reduced 3HB by-product or carboxylate by-product (e.g., acetate, acetoacetate, and/or malonate), and/or enhanced or increased carbon flux through 3HB—CoA, and/or 3HBAld, and/or an acyl-CoA (particularly a short-chain acyl-CoA, e.g., acetyl-CoA, acetoacetyl-CoA, and/or malonyl-CoA), and containing at least one exogenous nucleic acid encoding one or more enzymes or polypeptides, where the one or more enzymes or polypeptides are part of a 1,3-BDO pathway, and/or the pathway of an isomer thereof, converting Ac—COA or AcAc—COA to 1,3-BDO, and/or an isomer thereof, as exemplified by the pathways shown in FIG. 7.

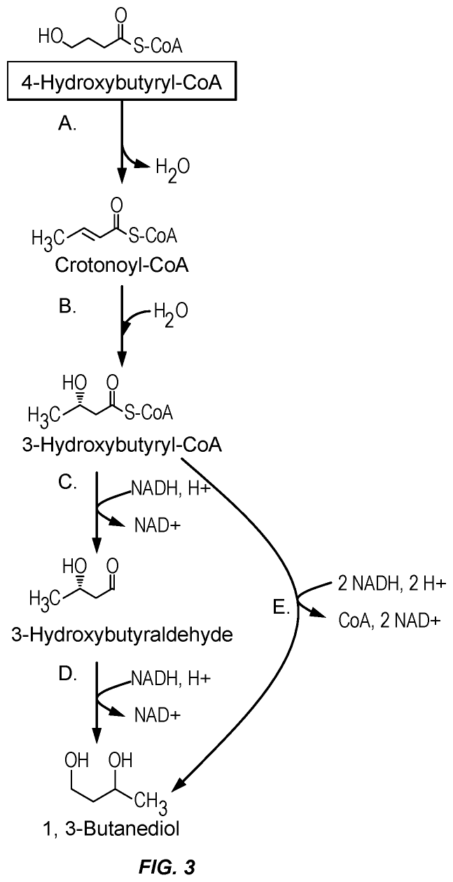

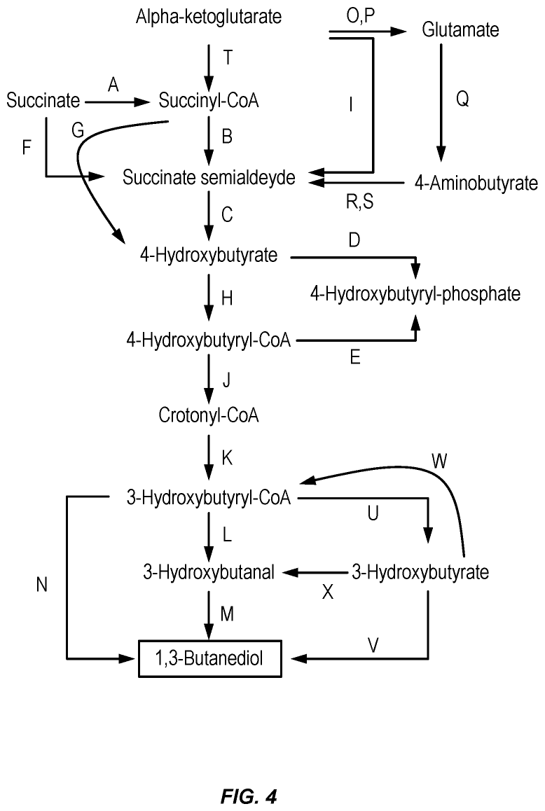

The non-naturally occurring organisms and cells of the present invention can also have one or more pathways which may produce precursors for pathways described herein. For example, 4HB—CoA may be produced as a by-product in one or more pathways in a non-naturally occurring organisms and cells of the present invention (see for example FIG. 4), wherein 4HB—CoA may be utilized as a precursor for pathways described herein, such as the pathway set forth in FIG. 3.

In an additional embodiment, the non-naturally occurring microbial organism or cell, having eliminated, substantially eliminated, or reduced 3HB by-product or carboxylate by-product (e.g., crotonate), and/or enhanced or increased carbon flux through 3HB—CoA, and/or 3HB—Ald, and/or an acyl-CoA (particularly a short-chain acyl-CoA, e.g., crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts 4-hydroxybutyryl-CoA (4HB—CoA) to crotonoyl-CoA;
(ii) an enzyme or polypeptide that converts crotonoyl-CoA to 3HB—CoA;
(iii) an enzyme or polypeptide that converts 3HB—CoA to 3-HBAld; and/or
(iv) an enzyme or polypeptide that converts 3-HBAld to 1,3-BDO.

In certain embodiments, the non-naturally occurring microbial organism or cell, having eliminated, substantially eliminated, or reduced 3HB by-product or carboxylate by-product (e.g., crotonate), and/or enhanced or increased carbon flux through 3HB—CoA, and/or 3HB-Ald, and/or an acyl-CoA (e.g., crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts 4-hydroxybutyryl-CoA (4HB—CoA) to crotonoyl-CoA;
(ii) an enzyme or polypeptide that converts crotonoyl-CoA to 3HB—CoA; and/or
(iii) an enzyme or polypeptide that converts 3HB—CoA to 1,3-BDO.

In some embodiments, 4HB—CoA is converted to crotonoyl-CoA by an enzyme or polypeptide having 4HB—CoA dehydratase (EC 4.2.1.a) activity. In some embodiments, crotonoyl-CoA is converted to 3HB—CoA by an enzyme or polypeptide having CRT (EC 4.2.1.a) activity. In some embodiments, the enzyme or polypeptide that converts 3HB—CoA to 1,3-BDO has 3HB—CoA reductase (an alcohol forming oxioreductase) (EC 1.1.1.c) activity. In some embodiments, 3HB—CoA is converted to 3HB—Ald by an enzyme or polypeptide having 3HB—CoA reductase (aldehyde forming) (EC 1.2.1.b) activity. In some embodiments, 3HBAld is converted to 1,3-BDO by an enzyme or polypeptide having 3HBAld reductase (EC 1.1.1.a) activity, acetaldehyde-alcohol dehydrogenase (EC 1.2.1) activity, 1,3-BDO or 1,3-butylene glycol dehydrogenase (EC 1.1.1) activity, or alcohol dehydrogenase (ADH) (EC 1.1.1) activity.

In certain embodiments, a 1,3-BDO pathway, and/or the pathway of an isomer thereof, can include one or more of the following enzymes: 4HB—CoA dehydratase (a hydro-lyase); CRT (a hydro-lyase); 3HB—CoA reductase (a Co-A dependent, aldehyde forming oxidoreductase); 3HB—Ald reductase (an alcohol forming oxidoreductase); and/or 3HB—CoA reductase (an alcohol forming oxidoreductase).

The non-naturally occurring microbial organism can include at least one exogenous nucleic acid encoding a 1,3-BDO pathway enzyme. The non-naturally occurring microbial organism can include two exogenous nucleic acids each encoding a 1,3-BDO pathway enzyme, in other embodiments, three exogenous nucleic acids each encoding a 1,3-BDO pathway enzyme, in other embodiments, four exogenous nucleic acids each encoding a 1,3-BDO pathway enzyme, in other embodiments, five or more exogenous nucleic acids each encoding a 1,3-BDO pathway enzyme.

Any number of nucleic acids encoding one or more of these enzymes or polypeptides can be introduced into a host microbial organism or cell that already includes one or more, for example, one, two, three, four, or five nucleic acids that encode other enzymes or polypeptides of the 1,3-BDO pathway(s) described above. Where more than one exogenous nucleic acids are introduced, such nucleic acids can be any permutation of the multiple nucleic acids (e.g., polycistronic or multiple nucleic acids) already included in the host organism or cell.

One skilled in the art will understand that these product pathways, substrate-product pairs, and/or enzymes or polypeptides are merely exemplary, and that any of the product pathways, substrate-product pairs, and/or enzymes or polypeptides disclosed herein, that are suitable to produce a desired acetyl-CoA derived product and/or for which an appropriate activity is available for the conversion of the substrate to the product can be readily determined by one skilled in the art based on the teachings herein.

Any of the at least one exogenous nucleic acids can be a heterologous nucleic acid, and the non-naturally occurring microbial organism can be constructed for growth or culturing under substantially anaerobic conditions, for example, in a substantially anaerobic culture medium. Other culture conditions, including feedstock, are described in further detail herein.

Thus, provided herein is a non-naturally occurring microbial organism or cell, having eliminated, substantially eliminated, or reduced 3HB by-product or carboxylate by-product (e.g., crotonate), and/or enhanced or increased carbon flux through 3HB—CoA and/or 3HBAld, and/or an acyl-CoA (e.g., crotonyl-CoA), and containing at least one exogenous nucleic acid encoding one or more of an enzyme or polypeptide, where the enzyme(s) or polypeptide(s) are included in or are a part of a 1,3-BDO pathway, and where the pathway includes converting 4HB—CoA to 1,3-BDO, as exemplified by the pathways shown in FIG. 3.

The non-naturally occurring organisms and cells of the present invention can also have a Citric Acid Cycle (TCA) which may produce precursors for pathways described herein. Briefly, Ac—COA, produced from pyruvate by glycolysis, enters into the TCA cycle and generates TCA intermediates, such as alpha-ketoglutarate (AKG) and succinate (Succ), which can be utilized as precursors for pathways described herein.

In an additional embodiment, the non-naturally occurring microbial organism or cell, having eliminated, substantially eliminated, or reduced 3HB by-product or carboxylate by-product (e.g., succinate and/or crotonate), and/or enhanced or increased carbon flux through 3HB—CoA and/or 3HBAld, and/or an acyl-CoA (e.g., succinyl-CoA and/or crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts AKG to SucCoA;
(ii) an enzyme or polypeptide that converts SucCoA to SSA;
(iii) an enzyme or polypeptide that converts SSA to 4-hydroxybutyrate (4HB);
(iv) an enzyme or polypeptide that converts 4-HB to 4HB—CoA;
(v) an enzyme or polypeptide that converts 4HB—CoA to crotonyl-CoA;
(vi) an enzyme or polypeptide that converts crotonyl-CoA to 3HB—CoA;
(vii) an enzyme or polypeptide that converts 3HB—CoA to 3-hydroxybutanal (3HBAld); and/or
(viii) an enzyme or polypeptide that converts 3-hydroxybutanal to 1,3 BDO.

In certain embodiments, the non-naturally occurring microbial organism or cell, having eliminated, substantially eliminated, or reduced 3HB by-product or carboxylate by-product (e.g., succinate and/or crotonate), and/or enhanced or increased carbon flux through 3HB—CoA, and/or an acyl-CoA (e.g., succinyl-CoA and/or crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts AKG to SucCoA;
(ii) an enzyme or polypeptide that converts SucCoA to SSA;
(iii) an enzyme or polypeptide that converts SSA to 4HB;
(iv) an enzyme or polypeptide that converts 4HB to 4HB—CoA;
(v) an enzyme or polypeptide that converts 4HB—CoA to crotonyl-CoA;
(vi) an enzyme or polypeptide that converts crotonyl-CoA to 3HB—CoA; and/or
(vii) an enzyme or polypeptide that converts 3HB—CoA to 1,3 BDO.

In certain embodiments, the non-naturally occurring microbial organism or cell, having eliminated, substantially eliminated, or reduced 3HB by-product or carboxylate by-product (e.g., succinate and/or crotonate), and/or enhanced or increased carbon flux through 3HB—CoA, and/or an acyl-CoA (e.g., succinyl-CoA and/or crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts AKG to SucCoA;
(ii) an enzyme or polypeptide that converts SucCoA to SSA;
(iii) an enzyme or polypeptide that converts SSA to 4HB;
(iv) an enzyme or polypeptide that converts 4HB to 4HB—CoA;
(v) an enzyme or polypeptide that converts 4HB—CoA to crotonyl-CoA;
(vi) an enzyme or polypeptide that converts crotonyl-CoA to 3HB—CoA;
(vii) an enzyme or polypeptide that converts 3HB—CoA to 3-hydroxybutyrate,
   wherein 3HB gets recycled back into 3HB—CoA as described herein or converted as described in the next step; and/or
(viii) an enzyme or polypeptide that converts 3-hydroxybutyrate to 1,3 BDO.

In an additional embodiment, the non-naturally occurring microbial organism or cell, having eliminated, substantially eliminated, or reduced 3HB by-product or carboxylate by-product (e.g., succinate and/or crotonate), and/or enhanced or increased carbon flux through 3HB—CoA, and/or 3HBAld, and/or an acyl-CoA (e.g., succinyl-CoA and/or crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts AKG to SucCoA;
(ii) an enzyme or polypeptide that converts SucCoA to 4HB;
(iii) an enzyme or polypeptide that converts 4HB to 4HB—CoA;
(iv) an enzyme or polypeptide that converts 4HB—CoA to crotonyl-CoA;
(v) an enzyme or polypeptide that converts crotonyl-CoA to 3HB—CoA;
(vi) an enzyme or polypeptide that converts 3HB—CoA to 3-hydroxybutanal (3HBAld); and/or
(vii) an enzyme or polypeptide that converts 3-hydroxybutanal (3HBAld) to 1,3 BDO.

In certain embodiments, the non-naturally occurring microbial organism or cell, having eliminated, substantially eliminated, or reduced 3HB by-product or carboxylate by-product (e.g., succinate and/or crotonate), and/or enhanced or increased carbon flux through 3HB—CoA, and/or an acyl-CoA (e.g., succinyl-CoA and/or crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts AKG to SucCoA;
(ii) an enzyme or polypeptide that converts SucCoA to 4HB;
(iii) an enzyme or polypeptide that converts 4HB to 4HB—CoA;
(iv) an enzyme or polypeptide that converts 4HB—CoA to crotonyl-CoA;
(v) an enzyme or polypeptide that converts crotonyl-CoA to 3HB—CoA; and/or
(vi) an enzyme or polypeptide that converts 3HB—CoA to 1,3 BDO.

In certain embodiments, the non-naturally occurring microbial organism or cell, having eliminated, substantially eliminated, or reduced 3HB by-product or carboxylate by-product (e.g., succinate and/or crotonate), and/or enhanced or increased carbon flux through 3HB—CoA, and/or an acyl-CoA (e.g., succinyl-CoA and/or crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts AKG to SucCoA;
(ii) an enzyme or polypeptide that converts SucCoA to 4HB;
(iii) an enzyme or polypeptide that converts 4HB to 4HB—CoA;
(iv) an enzyme or polypeptide that converts 4HB—CoA to crotonyl-CoA;
(v) an enzyme or polypeptide that converts crotonyl-CoA to 3HB—CoA;
(vi) an enzyme or polypeptide that converts 3HB—CoA to 3-hydroxybutyrate,
   wherein 3HB gets recycled back into 3HB—CoA as described herein or converted as described in the next step; and/or
(vii) an enzyme or polypeptide that converts 3-hydroxybutyrate to 1,3 BDO.

In certain embodiments, the non-naturally occurring microbial organism or cell, having eliminated, substantially eliminated, or reduced 3HB by-product or carboxylate by-product (e.g., succinate and/or crotonate), and/or enhanced or increased carbon flux through 3HB—CoA, and/or 3HBAld, and/or an acyl-CoA (e.g., succinyl-CoA and/or crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts AKG to SucCoA;
(ii) an enzyme or polypeptide that converts SucCoA to SSA;
(iii) an enzyme or polypeptide that converts SSA to 4HB;
(iv) an enzyme or polypeptide that converts 4HB to 4-hydroxybutyryl-phosphate (4HB—P);
(v) an enzyme or polypeptide that converts 4HB—P to 4HB—CoA;
(vi) an enzyme or polypeptide that converts 4HB—CoA to crotonyl-CoA;
(vii) an enzyme or polypeptide that converts crotonyl-CoA to 3HB—CoA;
(viii) an enzyme or polypeptide that converts 3HB—CoA to 3-hydroxybutanal (3HBAld); and/or
(ix) an enzyme or polypeptide that converts 3-hydroxybutanal to 1,3 BDO.

In certain embodiments, the non-naturally occurring microbial organism or cell, having eliminated, substantially eliminated, or reduced 3HB by-product or carboxylate by-product (e.g., succinate and/or crotonate), and/or enhanced or increased carbon flux through 3HB—CoA, and/or an acyl-CoA (e.g., succinyl-CoA and/or crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts AKG to SucCoA;
(ii) an enzyme or polypeptide that converts SucCoA to SSA;
(iii) an enzyme or polypeptide that converts SSA to 4HB;
(iv) an enzyme or polypeptide that converts 4HB to 4HB—P;
(v) an enzyme or polypeptide that converts 4HB—P to 4HB—CoA;
(vi) an enzyme or polypeptide that converts 4HB—CoA to crotonyl-CoA;
(vii) an enzyme or polypeptide that converts crotonyl-CoA to 3HB—CoA; and/or
(viii) an enzyme or polypeptide that converts 3HB—CoA to 1,3 BDO.

In certain embodiments, the non-naturally occurring microbial organism or cell, having eliminated, substantially eliminated, or reduced 3HB by-product or carboxylate by-product (e.g., succinate and/or crotonate), and/or enhanced or increased carbon flux through 3HB—CoA, and/or an acyl-CoA (e.g., succinyl-CoA and/or crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts AKG to SucCoA;
(ii) an enzyme or polypeptide that converts SucCoA to SSA;
(iii) an enzyme or polypeptide that converts SSA to 4HB;
(iv) an enzyme or polypeptide that converts 4HB to 4HB—P;
(v) an enzyme or polypeptide that converts 4HB—P to 4HB—CoA;
(vi) an enzyme or polypeptide that converts 4HB—CoA to crotonyl-CoA;
(vii) an enzyme or polypeptide that converts crotonyl-CoA to 3HB—CoA; and/or
(viii) an enzyme or polypeptide that converts 3HB—CoA to 3-hydroxybutyrate,
   wherein 3HB gets recycled back into 3HB—CoA as described herein or converted as described in the next step; and/or
(ix) an enzyme or polypeptide that converts 3-hydroxybutyrate to 1,3 BDO.

In certain embodiments, the non-naturally occurring microbial organism or cell, having eliminated, substantially eliminated, or reduced 3HB by-product or carboxylate by-product (e.g., succinate and/or crotonate), and/or enhanced or increased carbon flux through 3HB—CoA, and/or 3HBAld, and/or an acyl-CoA (e.g., succinyl-CoA and/or crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts AKG to SucCoA;
(ii) an enzyme or polypeptide that converts SucCoA to 4HB;
(iii) an enzyme or polypeptide that converts 4HB to 4HB—P;
(iv) an enzyme or polypeptide that converts 4HB—P to 4HB—CoA;
(v) an enzyme or polypeptide that converts 4HB—CoA to crotonyl-CoA;
(vi) an enzyme or polypeptide that converts crotonyl-CoA to 3HB—CoA;
(vii) an enzyme or polypeptide that converts 3HB—CoA to 3-hydroxybutanal (3HBAld); and/or
(viii) an enzyme or polypeptide that converts 3-hydroxybutanal (3HBAld) to 1,3 BDO.

In certain embodiments, the non-naturally occurring microbial organism or cell, having eliminated, substantially eliminated, or reduced 3HB by-product or carboxylate by-product (e.g., succinate and/or crotonate), and/or enhanced or increased carbon flux through 3HB—CoA, and/or an acyl-CoA (e.g., succinyl-CoA and/or crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts AKG to SucCoA;
(ii) an enzyme or polypeptide that converts SucCoA to 4HB;
(iii) an enzyme or polypeptide that converts 4HB to 4HB—P;
(iv) an enzyme or polypeptide that converts 4HB—P to 4HB—CoA;
(v) an enzyme or polypeptide that converts 4HB—CoA to crotonyl-CoA;
(vi) an enzyme or polypeptide that converts crotonyl-CoA to 3HB—CoA; and/or
(vii) an enzyme or polypeptide that converts 3HB—CoA to 1,3 BDO.

In certain embodiments, the non-naturally occurring microbial organism or cell, having eliminated, substantially eliminated, or reduced 3HB by-product or carboxylate by-product (e.g., succinate and/or crotonate), and/or enhanced or increased carbon flux through 3HB—CoA, and/or an acyl-CoA (e.g., succinyl-CoA and/or crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts AKG to SucCoA;
(ii) an enzyme or polypeptide that converts SucCoA to 4HB;
(iii) an enzyme or polypeptide that converts 4HB to 4HB—P;
(iv) an enzyme or polypeptide that converts 4HB—P to 4HB—CoA;
(v) an enzyme or polypeptide that converts 4HB—CoA to crotonyl-CoA;
(vi) an enzyme or polypeptide that converts crotonyl-CoA to 3HB—CoA; and/or
(vii) an enzyme or polypeptide that converts 3HB—CoA to 3-hydroxybutyrate,
   wherein 3HB gets recycled back into 3HB—CoA as described herein or converted as described in the next step; and/or
(viii) an enzyme or polypeptide that converts 3-hydroxybutyrate to 1,3 BDO.

In certain embodiments, the non-naturally occurring microbial organism or cell, having eliminated, substantially eliminated, or reduced 3HB by-product or carboxylate by-product (e.g., crotonate), and/or enhanced or increased carbon flux through 3HB—CoA, and/or 3HBAld, and/or an acyl-CoA (e.g., crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts AKG to glutamate;
(ii) an enzyme or polypeptide that converts glutamate to 4-aminobutyrate;
(iii) an enzyme or polypeptide that converts 4-aminobutyrate to SSA;
(iv) an enzyme or polypeptide that converts SSA to 4HB;
(v) an enzyme or polypeptide that converts 4HB to 4HB—CoA;
(vi) an enzyme or polypeptide that converts 4HB—CoA to crotonyl-CoA;
(vii) an enzyme or polypeptide that converts crotonyl-CoA to 3HB—CoA;
(viii) an enzyme or polypeptide that converts 3HB—CoA to 3-hydroxybutanal (3HBAld); and/or
(ix) an enzyme or polypeptide that converts 3-hydroxybutanal (3HBAld) to 1,3 BDO.

In certain embodiments, the non-naturally occurring microbial organism or cell, having eliminated, substantially eliminated, or reduced 3HB by-product or carboxylate by-product (e.g., crotonate), and/or enhanced or increased carbon flux through 3HB—CoA, and/or an acyl-CoA (e.g., crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts AKG to glutamate;
(ii) an enzyme or polypeptide that converts glutamate to 4-aminobutyrate;
(iii) an enzyme or polypeptide that converts 4-aminobutyrate to SSA;
(iv) an enzyme or polypeptide that converts SSA to 4HB;
(v) an enzyme or polypeptide that converts 4HB to 4HB—CoA;
(vi) an enzyme or polypeptide that converts 4HB—CoA to crotonyl-CoA;
(vii) an enzyme or polypeptide that converts crotonyl-CoA to 3HB—CoA; and/or
(viii) an enzyme or polypeptide that converts 3HB—CoA to 1,3 BDO.

In certain embodiments, the non-naturally occurring microbial organism or cell, having eliminated, substantially eliminated, or reduced 3HB by-product or carboxylate by-product (e.g., crotonate), and/or enhanced or increased carbon flux through 3HB—CoA, and/or an acyl-CoA (e.g., crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts AKG to glutamate;
(ii) an enzyme or polypeptide that converts glutamate to 4-aminobutyrate;
(iii) an enzyme or polypeptide that converts 4-aminobutyrate to SSA;
(iv) an enzyme or polypeptide that converts SSA to 4HB;
(v) an enzyme or polypeptide that converts 4HB to 4HB—CoA;
(vi) an enzyme or polypeptide that converts 4HB—CoA to crotonyl-CoA;
(vii) an enzyme or polypeptide that converts crotonyl-CoA to 3HB—CoA;
(viii) an enzyme or polypeptide that converts 3HB—CoA to 3-hydroxybutyrate,
   wherein 3HB gets recycled back into 3HB—CoA as described herein or converted as described in the next step; and/or
(ix) an enzyme or polypeptide that converts 3-hydroxybutyrate to 1,3 BDO.

In certain embodiments, the non-naturally occurring microbial organism or cell, having eliminated, substantially eliminated, or reduced 3HB by-product or carboxylate by-product (e.g., crotonate), and/or enhanced or increased carbon flux through 3HB—CoA, and/or 3HBAld, and/or an acyl-CoA (e.g., crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts AKG to glutamate;
(ii) an enzyme or polypeptide that converts glutamate to 4-aminobutyrate;
(iii) an enzyme or polypeptide that converts 4-aminobutyrate to SSA;
(iv) an enzyme or polypeptide that converts SSA to 4HB;
(v) an enzyme or polypeptide that converts 4HB to 4HB—P;
(vi) an enzyme or polypeptide that converts 4HB—P to 4HB—CoA;
(vii) an enzyme or polypeptide that converts 4HB—CoA to crotonyl-CoA;
(viii) an enzyme or polypeptide that converts crotonyl-CoA to 3HB—CoA;
(ix) an enzyme or polypeptide that converts 3HB—CoA to 3-hydroxybutanal (3HBAld); and/or
(x) an enzyme or polypeptide that converts 3-hydroxybutanal (3HBAld) to 1,3 BDO.

In certain embodiments, the non-naturally occurring microbial organism or cell, having eliminated, substantially eliminated, or reduced 3HB by-product or carboxylate by-product (e.g., crotonate), and/or enhanced or increased carbon flux through 3HB—CoA, and/or an acyl-CoA (e.g., crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts AKG to glutamate;
(ii) an enzyme or polypeptide that converts glutamate to 4-aminobutyrate;
(iii) an enzyme or polypeptide that converts 4-aminobutyrate to SSA;
(iv) an enzyme or polypeptide that converts SSA to 4HB;
(v) an enzyme or polypeptide that converts 4HB to 4HB—P;
(vi) an enzyme or polypeptide that converts 4HB—P to 4HB—CoA;
(vii) an enzyme or polypeptide that converts 4HB—CoA to crotonyl-CoA;
(viii) an enzyme or polypeptide that converts crotonyl-CoA to 3HB—CoA; and/or
(ix) an enzyme or polypeptide that converts 3HB—CoA to 1,3 BDO.

In certain embodiments, the non-naturally occurring microbial organism or cell, having eliminated, substantially eliminated, or reduced 3HB by-product or carboxylate by-product (e.g., crotonate), and/or enhanced or increased carbon flux through 3HB—CoA, and/or an acyl-CoA (e.g., crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts AKG to glutamate;
(ii) an enzyme or polypeptide that converts glutamate to 4-aminobutyrate;
(iii) an enzyme or polypeptide that converts 4-aminobutyrate to SSA;
(iv) an enzyme or polypeptide that converts SSA to 4HB;
(v) an enzyme or polypeptide that converts 4HB to 4HB—P;
(vi) an enzyme or polypeptide that converts 4HB—P to 4HB—CoA;
(vii) an enzyme or polypeptide that converts 4HB—CoA to crotonyl-CoA;
(viii) an enzyme or polypeptide that converts crotonyl-CoA to 3HB—CoA; and/or
(ix) an enzyme or polypeptide that converts 3HB—CoA to 3-hydroxybutyrate,
   wherein 3HB gets recycled back into 3HB—CoA as described herein or converted as described in the next step; and/or
(x) an enzyme or polypeptide that converts 3-hydroxybutyrate to 1,3 BDO.

In certain embodiments, the non-naturally occurring microbial organism or cell, having eliminated, substantially eliminated, or reduced 3HB by-product or carboxylate by-product (e.g., crotonate), and/or enhanced or increased carbon flux through 3HB—CoA, and/or 3HBAld, and/or an acyl-CoA (e.g., crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts AKG to SSA;
(ii) an enzyme or polypeptide that converts SSA to 4HB;
(iii) an enzyme or polypeptide that converts 4HB to 4HB—CoA;
(iv) an enzyme or polypeptide that converts 4HB—CoA to crotonyl-CoA;
(v) an enzyme or polypeptide that converts crotonyl-CoA to 3HB—CoA;
(vi) an enzyme or polypeptide that converts 3HB—CoA to 3-hydroxybutanal (3HBAld); and/or
(vii) an enzyme or polypeptide that converts 3-hydroxybutanal (3HBAld) to 1,3 BDO.

In certain embodiments, the non-naturally occurring microbial organism or cell, having eliminated, substantially eliminated, or reduced 3HB by-product, or carboxylate by-product (e.g., crotonate), and/or enhanced or increased carbon flux through 3HB—CoA, and/or an acyl-CoA (e.g., crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts AKG to SSA;
(ii) an enzyme or polypeptide that converts SSA to 4HB;
(iii) an enzyme or polypeptide that converts 4HB to 4HB—CoA;
(iv) an enzyme or polypeptide that converts 4HB—CoA to crotonyl-CoA;
(v) an enzyme or polypeptide that converts crotonyl-CoA to 3HB—CoA;
(vi) an enzyme or polypeptide that converts 3HB—CoA to 1,3 BDO.

In certain embodiments, the non-naturally occurring microbial organism or cell, having eliminated, substantially eliminated, or reduced 3HB by-product or carboxylate by-product (e.g., crotonate), and/or enhanced or increased carbon flux through 3HB—CoA, and/or an acyl-CoA (e.g., crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts AKG to SSA;
(ii) an enzyme or polypeptide that converts SSA to 4HB;
(iii) an enzyme or polypeptide that converts 4HB to 4HB—CoA;
(iv) an enzyme or polypeptide that converts 4HB—CoA to crotonyl-CoA;
(v) an enzyme or polypeptide that converts crotonyl-CoA to 3HB—CoA;
(vi) an enzyme or polypeptide that converts 3HB—CoA to 3-hydroxybutyrate,
   wherein 3HB gets recycled back into 3HB—CoA as described herein or converted as described in the next step; and/or
(vii) an enzyme or polypeptide that converts 3-hydroxybutyrate to 1,3 BDO.

In certain embodiments, the non-naturally occurring microbial organism or cell, having eliminated, substantially eliminated, or reduced 3HB by-product or carboxylate by-product (e.g., crotonate), and/or enhanced or increased carbon flux through 3HB—CoA, and/or 3HBAld, and/or an acyl-CoA (e.g., crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts AKG to SSA;
(ii) an enzyme or polypeptide that converts SSA to 4HB;
(iii) an enzyme or polypeptide that converts 4HB to 4HB—P;
(iv) an enzyme or polypeptide that converts 4HB—P to 4HB—CoA;
(v) an enzyme or polypeptide that converts 4HB—CoA to crotonyl-CoA;
(vi) an enzyme or polypeptide that converts crotonyl-CoA to 3HB—CoA;
(vii) an enzyme or polypeptide that converts 3HB—CoA to 3-hydroxybutanal (3HBAld); and/or
(viii) an enzyme or polypeptide that converts 3-hydroxybutanal (3HBAld) to 1,3 BDO.

In certain embodiments, the non-naturally occurring microbial organism or cell, having eliminated, substantially eliminated, or reduced 3HB by-product or carboxylate by-product (e.g., crotonate), and/or enhanced or increased carbon flux through 3HB—CoA, and/or an acyl-CoA (e.g., crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts AKG to SSA;
(ii) an enzyme or polypeptide that converts SSA to 4HB;
(iii) an enzyme or polypeptide that converts 4HB to 4HB—P;
(iv) an enzyme or polypeptide that converts 4HB—P to 4HB—CoA;
(v) an enzyme or polypeptide that converts 4HB—CoA to crotonyl-CoA;
(vi) an enzyme or polypeptide that converts crotonyl-CoA to 3HB—CoA;
(vii) an enzyme or polypeptide that converts 3HB—CoA to 1,3 BDO.

In certain embodiments, the non-naturally occurring microbial organism or cell, having eliminated, substantially eliminated, or reduced 3HB by-product or carboxylate by-product (e.g., crotonate), and/or enhanced or increased carbon flux through 3HB—CoA, and/or an acyl-CoA (e.g., crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts AKG to SSA;
(ii) an enzyme or polypeptide that converts SSA to 4HB;
(iii) an enzyme or polypeptide that converts 4HB to 4HB—P;
(iv) an enzyme or polypeptide that converts 4HB—P to 4HB—CoA;
(v) an enzyme or polypeptide that converts 4HB—CoA to crotonyl-CoA;
(vi) an enzyme or polypeptide that converts crotonyl-CoA to 3HB—CoA;
(vii) an enzyme or polypeptide that converts 3HB—CoA to 3-hydroxybutyrate,
   wherein 3HB gets recycled back into 3HB—CoA as described herein or converted as described in the next step; and/or
(viii) an enzyme or polypeptide that converts 3-hydroxybutyrate to 1,3 BDO.

In an additional embodiment, the non-naturally occurring microbial organism or cell, having eliminated, substantially eliminated, or reduced 3HB by-product or carboxylate by-product (e.g., succinate and/or crotonate), and/or enhanced or increased carbon flux through 3HB—CoA, and/or 3HBAld, and/or an acyl-CoA (e.g., succinyl-CoA and/or crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts Succ to SucCoA;
(ii) an enzyme or polypeptide that converts SucCoA to SSA;
(iii) an enzyme or polypeptide that converts SSA to 4HB;
(iv) an enzyme or polypeptide that converts 4HB to 4HB—CoA;
(v) an enzyme or polypeptide that converts 4HB—CoA to crotonyl-CoA;
(vi) an enzyme or polypeptide that converts crotonyl-CoA to 3HB—CoA;
(vii) an enzyme or polypeptide that converts 3HB—CoA to 3-hydroxybutanal (3HBAld); and/or
(viii) an enzyme or polypeptide that converts 3-hydroxybutanal (3HBAld) to 1,3 BDO.

In certain embodiments, the non-naturally occurring microbial organism or cell, having eliminated, substantially eliminated, or reduced 3HB by-product or carboxylate by-product (e.g., succinate and/or crotonate), and/or enhanced or increased carbon flux through 3HB—CoA, and/or an acyl-CoA (e.g., succinyl-CoA and/or crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts Succ to SucCoA;
(ii) an enzyme or polypeptide that converts SucCoA to SSA;
(iii) an enzyme or polypeptide that converts SSA to 4HB;
(iv) an enzyme or polypeptide that converts 4HB to 4HB—CoA;
(v) an enzyme or polypeptide that converts 4HB—CoA to crotonyl-CoA;
(vi) an enzyme or polypeptide that converts crotonyl-CoA to 3HB—CoA; and/or
(vii) an enzyme or polypeptide that converts 3HB—CoA to 1,3 BDO.

In certain embodiments, the non-naturally occurring microbial organism or cell, having eliminated, substantially eliminated, or reduced 3HB by-product or carboxylate by-product (e.g., succinate and/or crotonate), and/or enhanced or increased carbon flux through 3HB—CoA, and/or an acyl-CoA (e.g., succinyl-CoA and/or crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts Succ to SucCoA;
(ii) an enzyme or polypeptide that converts SucCoA to SSA;
(iii) an enzyme or polypeptide that converts SSA to 4HB;
(iv) an enzyme or polypeptide that converts 4HB to 4HB—CoA;
(v) an enzyme or polypeptide that converts 4HB—CoA to crotonyl-CoA;
(vi) an enzyme or polypeptide that converts crotonyl-CoA to 3HB—CoA;
(vii) an enzyme or polypeptide that converts 3HB—CoA to 3-hydroxybutyrate,
   wherein 3HB gets recycled back into 3HB—CoA as described herein or converted as described in the next step; and/or
(viii) an enzyme or polypeptide that converts 3-hydroxybutyrate to 1,3 BDO.

In certain embodiments, the non-naturally occurring microbial organism or cell, having eliminated, substantially eliminated, or reduced 3HB by-product or carboxylate by-product (e.g., succinate and/or crotonate), and/or enhanced or increased carbon flux through 3HB—CoA, and/or 3HBAld, and/or an acyl-CoA (e.g., succinyl-CoA and/or crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts Succ to SucCoA;
(ii) an enzyme or polypeptide that converts SucCoA to SSA;
(iii) an enzyme or polypeptide that converts SSA to 4HB;
(iv) an enzyme or polypeptide that converts 4HB to 4HB—P;
(v) an enzyme or polypeptide that converts 4HB—P to 4HB—CoA;
(vi) an enzyme or polypeptide that converts 4HB—CoA to crotonyl-CoA;
(vii) an enzyme or polypeptide that converts crotonyl-CoA to 3HB—CoA;
(viii) an enzyme or polypeptide that converts 3HB—CoA to 3-hydroxybutanal (3HBAld); and/or
(ix) an enzyme or polypeptide that converts 3-hydroxybutanal (3HBAld) to 1,3 BDO.

In certain embodiments, the non-naturally occurring microbial organism or cell, having eliminated, substantially eliminated, or reduced 3HB by-product or carboxylate by-product (e.g., succinate and/or crotonate), and/or enhanced or increased carbon flux through 3HB—CoA, and/or an acyl-CoA (e.g., succinyl-CoA and/or crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts Succ to SucCoA;
(ii) an enzyme or polypeptide that converts SucCoA to SSA;
(iii) an enzyme or polypeptide that converts SSA to 4HB;
(iv) an enzyme or polypeptide that converts 4HB to 4HB—P;
(v) an enzyme or polypeptide that converts 4HB—P to 4HB—CoA;
(vi) an enzyme or polypeptide that converts 4HB—CoA to crotonyl-CoA;
(vii) an enzyme or polypeptide that converts crotonyl-CoA to 3HB—CoA; and/or
(viii) an enzyme or polypeptide that converts 3HB—CoA to 1,3 BDO.

In certain embodiments, the non-naturally occurring microbial organism or cell, having eliminated, substantially eliminated, or reduced 3HB by-product or carboxylate by-product (e.g., succinate and/or crotonate), and/or enhanced or increased carbon flux through 3HB—CoA, and/or an acyl-CoA (e.g., succinyl-CoA and/or crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts Succ to SucCoA;
(ii) an enzyme or polypeptide that converts SucCoA to SSA;
(iii) an enzyme or polypeptide that converts SSA to 4HB;
(iv) an enzyme or polypeptide that converts 4HB to 4HB—P;
(v) an enzyme or polypeptide that converts 4HB—P to 4HB—CoA;
(vi) an enzyme or polypeptide that converts 4HB—CoA to crotonyl-CoA;
(vii) an enzyme or polypeptide that converts crotonyl-CoA to 3HB—CoA; and/or
(viii) an enzyme or polypeptide that converts 3HB—CoA to 3-hydroxybutyrate,
   wherein 3HB gets recycled back into 3HB—CoA as described herein or converted as described in the next step; and/or
(ix) an enzyme or polypeptide that converts 3-hydroxybutyrate to 1,3 BDO.

In an additional embodiment, the non-naturally occurring microbial organism or cell, having eliminated, substantially eliminated, or reduced 3HB by-product or carboxylate by-product (e.g., succinate and/or crotonate), and/or enhanced or increased carbon flux through 3HB—CoA, and/or 3HBAld, and/or an acyl-CoA (e.g., succinyl-CoA and/or crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts Succ to SSA;
(ii) an enzyme or polypeptide that converts SSA to 4HB;
(iii) an enzyme or polypeptide that converts 4HB to 4HB—CoA;
(iv) an enzyme or polypeptide that converts 4HB—CoA to crotonyl-CoA;
(v) an enzyme or polypeptide that converts crotonyl-CoA to 3HB—CoA;
(vi) an enzyme or polypeptide that converts 3HB—CoA to 3-hydroxybutanal (3HBAld); and/or
(vii) an enzyme or polypeptide that converts 3-hydroxybutanal (3HBAld) to 1,3 BDO.

In certain embodiments, the non-naturally occurring microbial organism or cell, having eliminated, substantially eliminated, or reduced 3HB by-product or carboxylate by-product (e.g., crotonate), and/or enhanced or increased carbon flux through 3HB—CoA, and/or acyl-CoA (e.g., crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts Succ to SSA;
(ii) an enzyme or polypeptide that converts SSA to 4HB;
(iii) an enzyme or polypeptide that converts 4HB to 4HB—CoA;
(iv) an enzyme or polypeptide that converts 4HB—CoA to crotonyl-CoA;
(v) an enzyme or polypeptide that converts crotonyl-CoA to 3HB—CoA; and/or
(vi) an enzyme or polypeptide that converts 3HB—CoA to 1,3 BDO.

In certain embodiments, the non-naturally occurring microbial organism or cell, having eliminated, substantially eliminated, or reduced 3HB by-product or carboxylate by-product (e.g., crotonate), and/or enhanced or increased carbon flux through 3HB—CoA, and/or acyl-CoA (e.g., crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts Succ to SSA;
(ii) an enzyme or polypeptide that converts SSA to 4HB;
(iii) an enzyme or polypeptide that converts 4HB to 4HB—CoA;
(iv) an enzyme or polypeptide that converts 4HB—CoA to crotonyl-CoA;
(v) an enzyme or polypeptide that converts crotonyl-CoA to 3HB—CoA;
(vi) an enzyme or polypeptide that converts 3HB—CoA to 3-hydroxybutyrate,
   wherein 3HB gets recycled back into 3HB—CoA as described herein or converted as described in the next step; and/or
(vii) an enzyme or polypeptide that converts 3-hydroxybutyrate to 1,3 BDO.

In certain embodiments, the non-naturally occurring microbial organism or cell, having eliminated, substantially eliminated, or reduced 3HB by-product or carboxylate by-product (e.g., crotonate), and/or enhanced or increased carbon flux through 3HB—CoA, and/or 3HBAld, and/or acyl-CoA (e.g., crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts Succ to SSA;
(ii) an enzyme or polypeptide that converts SSA to 4HB;
(iii) an enzyme or polypeptide that converts 4HB to 4HB—P;
(iv) an enzyme or polypeptide that converts 4HB—P to 4HB—CoA;
(v) an enzyme or polypeptide that converts 4HB—CoA to crotonyl-CoA;
(vi) an enzyme or polypeptide that converts crotonyl-CoA to 3HB—CoA;
(vii) an enzyme or polypeptide that converts 3HB—CoA to 3-hydroxybutanal (3HBAld); and/or
(viii) an enzyme or polypeptide that converts 3-hydroxybutanal (3HBAld) to 1,3 BDO.

In certain embodiments, the non-naturally occurring microbial organism or cell, having eliminated, substantially eliminated, or reduced 3HB by-product or carboxylate by-product (e.g., crotonate), and/or enhanced or increased carbon flux through 3HB—CoA, and/or acyl-CoA (e.g., crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts Succ to SSA;
(ii) an enzyme or polypeptide that converts SSA to 4HB;
(iii) an enzyme or polypeptide that converts 4HB to 4HB—P;
(iv) an enzyme or polypeptide that converts 4HB—P to 4HB—CoA;
(v) an enzyme or polypeptide that converts 4HB—CoA to crotonyl-CoA;
(vi) an enzyme or polypeptide that converts crotonyl-CoA to 3HB—CoA; and/or
(vii) an enzyme or polypeptide that converts 3HB—CoA to 1,3 BDO.

In certain embodiments, the non-naturally occurring microbial organism or cell, having eliminated, substantially eliminated, or reduced 3HB by-product or carboxylate by-product (e.g., crotonate), and/or enhanced or increased carbon flux through 3HB—CoA, and/or acyl-CoA (e.g., crotonyl-CoA), can further include a 1,3-BDO pathway, wherein the non-naturally occurring microbial organism comprises at least one exogenous nucleic acid encoding one or more of an enzyme or polypeptide that converts a substrate to a product, or an isomeric form thereof including, for example:
(i) an enzyme or polypeptide that converts Succ to SSA;
(ii) an enzyme or polypeptide that converts SSA to 4HB;
(iii) an enzyme or polypeptide that converts 4HB to 4HB—P;
(iv) an enzyme or polypeptide that converts 4HB—P to 4HB—CoA;
(v) an enzyme or polypeptide that converts 4HB—CoA to crotonyl-CoA;
(vi) an enzyme or polypeptide that converts crotonyl-CoA to 3HB—CoA; and/or
(vii) an enzyme or polypeptide that converts 3HB—CoA to 3-hydroxybutyrate,
   wherein 3HB gets recycled back into 3HB—CoA as described herein or converted as described in the next step; and/or
(viii) an enzyme or polypeptide that converts 3-hydroxybutyrate to 1,3 BDO.

In some embodiments, the enzyme or polypeptide that converts Succ to SucCoA has SucCoA transferase (EC 2.8.3.a) activity, SucCoA synthetase or ligase (EC 6.2.1.a) activity, or SucCoA hydrolase (EC 3.1.2.a) activity. In some embodiments, the enzyme or polypeptide that converts SucCoA to SSA has SucCoA reductase (aldehyde forming) (EC 1.2.1.b) activity. In some embodiments, the enzyme or polypeptide that converts SSA to 4HB has 4HB dehydrogenase (EC 1.1.1.a) activity. In some embodiments, the enzyme or polypeptide that converts 4HB to 4HB—P has 4HB kinase (EC 2.7.2.a) activity. In some embodiments, the enzyme or polypeptide that converts 4HB—P to 4HB—CoA has phosphotrans-4-hydroxybutyrylase (EC 2.3.1.a) activity. In some embodiments, the enzyme or polypeptide that converts Succ to SSA has Succ reductase (EC 1.2.1.e) activity. In some embodiments, the enzyme or polypeptide that converts SucCoA to 4HB has SucCoA reductase (alcohol forming) (EC 1.2.1) activity. In some embodiments, the enzyme or polypeptide that converts 4HB to 4HB—CoA has 4HB—CoA transferase, 4HB—CoA synthetase, or 4HB—CoA ligase (EC 2.8.3.a, 3.1.2.a, or 6.2.1.a) activity. In some embodiments, the enzyme or polypeptide that converts AKG to SSA has AKG decarboxylase (EC 4.1.1.a) activity. In some embodiments, the enzyme or polypeptide that converts 4HB—CoA to crotonyl-CoA has 4HB—CoA dehydratase (EC 4.2.1) activity. In some embodiments, the enzyme or polypeptide that converts crotonyl-CoA to 3HB—CoA has CRT (EC 4.2.1.a) activity. In some embodiments, the enzyme or polypeptide that converts 3HB—CoA to 3-hydroxybutanal has 3HB—CoA reductase (aldehyde forming) (EC 4.2.1) activity. In some embodiments, the enzyme or polypeptide that converts 3-hydroxybutanal to 1,3 BDO has 3-hydroxybutanal reductase (EC 1.1.1.a) activity. In some embodiments, the enzyme or polypeptide that converts 3HB—CoA to 1,3 BDO has 3HB—CoA reductase (a CoA dependent, an alcohol forming oxidoreductase) (EC 1.1.1.c) activity. In some embodiments, the enzyme or polypeptide that converts AKG to glutamate has glutamate dehydrogenase activity. In some embodiments, the enzyme or polypeptide that converts AKG to glutamate has glutamate transaminase or glutamate dehydrogenase (EC 1.4.1.a or 2.6.1.a) activity. In some embodiments, the enzyme or polypeptide that converts glutamate to 4-aminobutyrate has glutamate decarboxylase (EC 4.1.1.a) activity. In some embodiments, the enzyme or polypeptide that converts 4-aminobutyrate to SSA has 4-aminobutyrate dehydrogenase or 4-aminobutyrate transaminase (EC 1.4.1.a, or 2.6.1.a) activity. In some embodiments, the enzyme or polypeptide that converts AKG to SucCoA has AKGD (EC 1.2.4) activity. In some embodiments, 3HB—CoA is lost to 3HB as described herein, wherein 3HB gets recycled back into 3HB—CoA as described herein, or is converted to 3HB Ald by a 3HB to 3HBAld conversion step as described herein, or is converted to 1,3 BDO. In some embodiments, the enzyme or polypeptide that converts 3HB to 1,3 BDO has alcohol forming reductase (EC 1.1.1) activity.

In certain embodiments, the 1,3-BDO pathway comprises one or more of the following enzymes: a SucCoA transferase, SucCoA hydrolase, and/or a SucCoA synthetase (or SucCoA ligase); a SucCoA reductase (aldehyde forming); a 4HB dehydrogenase; a 4HB kinase; a phosphotrans-4-hydroxybutyrylase; a Succ reductase; a SucCoA reductase (alcohol forming); a 4HB—CoA transferase, or a 4HB—CoA synthetase, or a 4HB—CoA ligase; an AKG decarboxylase; a 4HB—CoA dehydratase; a CRT; a 3HB—CoA reductase (aldehyde forming); a 3-hydroxybutanal reductase; a 3HB—CoA reductase (alcohol forming); a 3HB—CoA hydrolase, and/or a 3HB—CoA transferase; an alcohol forming reductase; a glutamate dehydrogenase and/or a glutamate transaminase; a glutamate decarboxylase; a 4-aminobutyrate dehydrogenase and/or a 4-aminobutyrate transaminase; and/or an AKGD.

The non-naturally occurring microbial organism can include at least one exogenous nucleic acid encoding a 1,3-BDO pathway enzyme. The non-naturally occurring microbial organism can include two exogenous nucleic acids each encoding a 1,3-BDO pathway enzyme, in other embodiments, three exogenous nucleic acids each encoding a 1,3-BDO pathway enzyme, in other embodiments, four exogenous nucleic acids each encoding a 1,3-BDO pathway enzyme, in other embodiments, five exogenous nucleic acids each encoding a 1,3-BDO pathway enzyme, in other embodiments, six exogenous nucleic acids each encoding a 1,3-BDO pathway enzyme, in other embodiments, seven exogenous nucleic acids each encoding a 1,3-BDO pathway enzyme, in other embodiments, eight exogenous nucleic acids each encoding a 1,3-BDO pathway enzyme, in other embodiments, nine exogenous nucleic acids each encoding a 1,3-BDO pathway enzyme, in other embodiments, ten exogenous nucleic acids each encoding a 1,3-BDO pathway enzyme, in other embodiments, eleven, in other embodiments, twelve exogenous nucleic acids each encoding a 1,3-BDO pathway enzyme, in other embodiments, exogenous nucleic acids each encoding a 1,3-BDO pathway enzyme, in other embodiments, thirteen exogenous nucleic acids each encoding a 1,3-BDO pathway enzyme, in other embodiments, fourteen exogenous nucleic acids each encoding a 1,3-BDO pathway enzyme, in other embodiments, fifteen exogenous nucleic acids each encoding a 1,3-BDO pathway enzyme, in other embodiments, sixteen exogenous nucleic acids each encoding a 1,3-BDO pathway enzyme, in other embodiments, seventeen exogenous nucleic acids each encoding a 1,3-BDO pathway enzyme, in other embodiments, eighteen exogenous nucleic acids each encoding a 1,3-BDO pathway enzyme, in other embodiments, nineteen exogenous nucleic acids each encoding a 1,3-BDO pathway enzyme, in other embodiments, twenty or more exogenous nucleic acids each encoding a 1,3-BDO pathway enzyme.

Any number of nucleic acids encoding one or more of these enzymes or polypeptides can be introduced into a host microbial organism or cell that already includes one or more, for example, one, two, three, four, five, six, seven, eight, nine, ten, eleven, twelve, thirteen, fourteen, fifteen, sixteen, seventeen, eighteen, nineteen or twenty nucleic acids that encode other enzymes or polypeptides of the 1,3-BDO pathway(s) described above. Where more than one exogenous nucleic acids are introduced, such nucleic acids can be any permutation of the multiple nucleic acids (e.g., polycistronic or multiple nucleic acids) already included in the host organism or cell.

One skilled in the art will understand that these 1,3-BDO pathways, and/or the pathways of an isomer thereof, substrate-product pairs, and encoded enzymes or polypeptides are merely exemplary, and that any of the substrate-product pairs and/or encoded enzymes or polypeptides disclosed herein that are suitable to produce a desired acetyl-CoA derived product, such as 1,3-BDO, and/or an isomer thereof, and for which an appropriate activity is available for the conversion of the substrate to the product, can be readily determined by one skilled in the art based on the teachings herein.

Any of the at least one exogenous nucleic acids can be a heterologous nucleic acid, and the non-naturally occurring microbial organism can be constructed for growth or culturing under substantially anaerobic conditions, for example, in a substantially anaerobic culture medium. Other culture conditions, including feedstock, are described in further detail herein.

Thus, provided herein is a non-naturally occurring microbial organism or cell, having eliminated, substantially eliminated, or reduced 3HB by-product, and/or enhanced or increased carbon flux through 3HB—CoA, and containing at least one exogenous nucleic acid encoding one or more enzyme(s) or polypeptide(s), where the one or more enzymes or polypeptides are part of a 1,3-BDO pathway, and/or the pathway of an isomer thereof, converting AKG, Succ, and/or SucCoA to 1,3-BDO, and/or an isomer thereof, as exemplified by the pathways shown in FIG. 4.

In certain embodiments, the non-naturally occurring microbial organism or cell, having eliminated, substantially eliminated, or reduced 3HB by-product or carboxylate by-product (e.g., acetate, acetoacetate, crotonate and/or butyrate), and/or enhanced or increased carbon flux through 3HB—CoA, and/or acyl-CoA (e.g., Ac—COA, AcAc—COA, crotonyl-CoA and/or butyryl-CoA), can further include a butyrate, butanol, propane, and/or formate pathway, wherein the non-naturally occurring microbial organism or cell includes at least one exogenous nucleic acid encoding one or more of an enzyme or polypeptide that converts a substrate to a product, or an isomeric form thereof including, for example:
(i) an enzyme or polypeptide that converts Ac—COA to AcAc—CoA;
(ii) an enzyme or polypeptide that converts AcAc—CoA to (S)-3HB—CoA;
(iii) an enzyme or polypeptide that converts(S)-3HB—CoA to crotonyl-CoA;
(iv) an enzyme or polypeptide that converts crotonyl-CoA to butyryl-CoA;
(v) an enzyme or polypeptide that converts butyryl-CoA to butyryl-phosphate; and/or
(vi) an enzyme or polypeptide that converts butyryl-phosphate to butyrate.

In certain embodiments, the non-naturally occurring microbial organism or cell, having eliminated, substantially eliminated, or reduced 3HB by-product or carboxylate by-product (e.g., acetate, acetoacetate, crotonate and/or butyrate), and/or enhanced or increased carbon flux through 3HB—CoA, and/or acyl-CoA (e.g., Ac—COA, AcAc—COA, crotonyl-CoA and/or butyryl-CoA), can further include a butyrate, butanol, propane and/or formate pathway, wherein the non-naturally occurring microbial organism or cell includes at least one exogenous nucleic acid encoding one or more of an enzyme or polypeptide that converts a substrate to a product, or an isomeric form thereof including, for example:
(i) an enzyme or polypeptide that converts Ac—COA to AcAc—COA;
(ii) an enzyme or polypeptide that converts AcAc—COA to (S)-3HB—CoA;
(iii) an enzyme or polypeptide that converts (S)-3HB—CoA to crotonyl-CoA;
(iv) an enzyme or polypeptide that converts crotonyl-CoA to butyryl-CoA;
(v) an enzyme or polypeptide that converts butyryl-CoA to butyraldehyde; and/or
(vi) an enzyme or polypeptide that converts butyraldehyde to butanol.

In certain embodiments, the non-naturally occurring microbial organism or cell, having eliminated, substantially eliminated, or reduced 3HB by-product or carboxylate by-product (e.g., acetate, acetoacetate, crotonate and/or butyrate), and/or enhanced or increased carbon flux through 3HB—CoA, and/or acyl-CoA (e.g., Ac—COA, AcAc—COA, crotonyl-CoA and/or butyryl-CoA), can further include a butyrate, butanol, propane, and/or formate pathway, wherein the non-naturally occurring microbial organism or cell includes at least one exogenous nucleic acid encoding one or more of an enzyme or polypeptide that converts a substrate to a product, or an isomeric form thereof including, for example:
(i) an enzyme or polypeptide that converts Ac—COA to AcAc—COA;
(ii) an enzyme or polypeptide that converts AcAc—COA to (S)-3HB—CoA;
(iii) an enzyme or polypeptide that converts (S)-3HB—CoA to crotonyl-CoA;
(iv) an enzyme or polypeptide that converts crotonyl-CoA to butyryl-CoA;
(v) an enzyme or polypeptide that converts butyryl-CoA to butyraldehyde; and/or
(vi) an enzyme or polypeptide that converts butyraldehyde to propane and/or formate.

In some embodiments, the enzyme or polypeptide that converts Ac-Coa to AcAc—COA has AcAc—CoA thiolase (EC 2.3.1) activity. In some embodiments, the enzyme or polypeptide that converts AcAc—Coa to S-3HB—CoA has HBD, optionally, a S—HBD (EC 1.1.1.157) activity. In some embodiments, the enzyme or polypeptide that converts S-3HB—CoA to crotonyl-CoA has CRT (EC 4.2.1.a) activity. In some embodiments, the enzyme or polypeptide that converts crotonyl-CoA to butyryl-CoA has butyryl-CoA dehydrogenase (BCD) (EC 1.3.8) activity. In some embodiments, the enzyme or polypeptide that converts butyryl-CoA to butyraldehyde has CoA acetylating aldehyde dehydrogenase (ALDH) (EC 1.2.1) activity. In some embodiments, the enzyme or polypeptide that converts butyraldehyde to butanol has aldehyde reductase (AHR) (EC 1.1.1) activity. In some embodiments, the enzyme or polypeptide that converts butyryl-CoA to butyryl-phosphatase has phosphotransbutyrylase (PTB) (EC 2.3.1) activity. In some embodiments, the enzyme or polypeptide that converts butyryl-P to butyrate has butyrate kinase (BUK) (EC 2.7.2) activity. In some embodiments, the enzyme or polypeptide that converts butyraldehyde to propane and/or formate has aldehyde deformulating dehydrogenase (ADO) (EC 4.1.99) activity.

In some embodiments, the 1,3-BDO pathway includes at least one exogenous nucleic acid encoding one or more of the following enzymes: AcAc—COA thiolase; HBD, optionally, S—HBD; CRT; BCD; ALDH; AHR; PTB; BUK; and/or ADO.

The non-naturally occurring microbial organism can include at least one exogenous nucleic acid encoding a butyrate, butanol, propane, and/or formate pathway enzyme. The non-naturally occurring microbial organism can include two exogenous nucleic acids each encoding a butyrate, butanol, propane, and/or formate pathway enzyme, in other embodiments, three exogenous nucleic acids each encoding a butyrate, butanol, propane, and/or formate pathway enzyme, in other embodiments, four exogenous nucleic acids each encoding a butyrate, butanol, propane, and/or formate pathway enzyme, in other embodiments, five exogenous nucleic acids each encoding a butyrate, butanol, propane, and/or formate pathway enzyme, in other embodiments, six exogenous nucleic acids each encoding a butyrate, butanol, propane, and/or formate pathway enzyme, in other embodiments, seven exogenous nucleic acids each encoding a butyrate, butanol, propane, and/or formate pathway enzyme, in other embodiments, eight exogenous nucleic acids each encoding a butyrate, butanol, propane, and/or formate pathway enzyme, in other embodiments, nine or more exogenous nucleic acids each encoding a butyrate, butanol, propane, and/or formate pathway enzyme.

Any number of nucleic acids encoding these enzymes can be further introduced into a host microbial organism or cell including one, two, three, four, five, six, seven, eight, or nine nucleic acids that encode these enzymes. Where more than one exogenous nucleic acids are introduced, such nucleic acids can be any permutation of the multiple nucleic acids (e.g., polycistronic or multiple nucleic acids) already included in the host organism or cell.

One skilled in the art will understand that these butyrate, butanol, propane and/or formate pathways, substrate-product pairs, and encoded enzymes or polypeptides are merely exemplary, and that any of the substrate-product pairs and/or encoded enzymes or polypeptides disclosed herein that are suitable to produce a desired acetyl-CoA derived product, such as butyrate, butanol, propane and/or formate, and for which an appropriate activity is available for the conversion of the substrate to the product, can be readily determined by one skilled in the art based on the teachings herein.

Any of the at least one exogenous nucleic acids can be a heterologous nucleic acid, and the non-naturally occurring microbial organism can be constructed for growth or culturing under substantially anaerobic conditions, for example, in a substantially anaerobic culture medium. Other culture conditions, including feedstock, are described in further detail herein.

Thus, provided herein is a non-naturally occurring microbial organism having eliminated, substantially eliminated, or reduced 3HB by-product, and/or enhanced or increased carbon flux through 3HB—CoA, and containing at least one exogenous nucleic acid encoding one or more of an enzyme or polypeptide, where the enzyme(s) or polypeptide(s) are included in or are a part of a butyrate, butanol, propane and/or formate pathway, and includes converting acetyl-CoA or AcAc—COA to butyrate, butanol, propane and/or formate, as exemplified by the pathways shown in FIG. 5.

The non-naturally occurring microbial organism or cell of the present invention, having a glycolysis pathway or an acetyl-CoA pathway, can further include an acetyl-CoA derived product pathway and a 3HB—CoA (e.g., R-3HB—CoA) intermediate and a 1,3 BDO (e.g., R-1,3-BDO) intermediate wherein the acetyl-CoA derived product is (3R)-hydroxybutyl (3R)-hydroxybutyrate. Accordingly, in certain embodiments, the non-naturally occurring microbial organism or cell having eliminated, substantially eliminated, or reduced 3HB by-product, and/or enhanced or increased carbon flux through 3HB—CoA (e.g., R-3HB—CoA) and/or 3HBAld (e.g., R-3HBAld), further includes a (3R)-hydroxybutyl (3R)-hydroxybutyrate pathway. In some embodiments, a non-naturally occurring organism of the present invention having a (3R)-hydroxybutyl (3R)-hydroxybutyrate pathway can further include at least one exogenous nucleic acid encoding one or more of a 1,3 BDO (e.g., R-1,3-BDO) pathway enzyme, as described herein, for the production of (3R)-hydroxybutyl (3R)-hydroxybutyrate precursors and for the downstream production of (3R)-hydroxybutyl (3R)-hydroxybutyrate (see e.g., FIG. 7 and FIG. 5).

In certain embodiments, the non-naturally occurring microbial organism or cell, having eliminated, substantially eliminated, or reduced (R) 3HB by-product or carboxylate by-product (e.g., acetate and/or acetoacetate), and/or enhanced or increased carbon flux through (R) 3HB—CoA, and/or (R)-3HBAld, and/or acyl-CoA (e.g., acetyl-CoA and/or acetoacetyl-CoA), can further include a (3R)-hydroxybutyl (3R)-hydroxybutyrate pathway, wherein the non-naturally occurring microbial organism or cell includes at least one exogenous nucleic acid encoding one or more of an enzyme or polypeptide that converts a substrate to a product, or an isomeric form thereof including, for example:
(i) an enzyme or polypeptide that converts Ac—COA to AcAc—COA;
(ii) an enzyme or polypeptide that converts AcAc—COA to(S)-3HB—CoA;
(iii) an enzyme or polypeptide that converts(S)-3HB—CoA to (R)-3HB—CoA;
(iv) an enzyme or polypeptide that converts (R)-3HB—CoA to (R)-3HBAld;
(v) an enzyme or polypeptide that converts (R)-3HBAld to (R)-1,3 BDO; and/or
(vi) an enzyme or polypeptide that converts (R)-3HB—CoA and (R)-1,3 BDO to (3R)-hydroxybutyl (3R)-hydroxybutyrate.

In certain embodiments, the non-naturally occurring microbial organism or cell, having eliminated, substantially eliminated, or reduced (R) 3HB by-product or carboxylate by-product (e.g., acetate, malonate, and/or acetoacetate), and/or enhanced or increased carbon flux through (R) 3HB—CoA, and/or (R)-3HBAld, and/or acyl-CoA (e.g., acetyl-CoA, malonyl-CoA, and/or acetoacetyl-CoA), can further include a (3R)-hydroxybutyl (3R)-hydroxybutyrate pathway, wherein the non-naturally occurring microbial organism or cell includes at least one exogenous nucleic acid encoding one or more of an enzyme or polypeptide that converts a substrate to a product, or an isomeric form thereof including, for example:
(i) an enzyme or polypeptide that converts Ac—COA to malonyl-CoA
(ii) an enzyme or polypeptide that converts malonyl-CoA to AcAc—COA
(iii) an enzyme or polypeptide that converts AcAc—COA to (R)-3HB—CoA;
(iv) an enzyme or polypeptide that converts (R)-3HB—CoA to (R)-3HBAld;
(v) an enzyme or polypeptide that converts (R)-3HBAld to (R)-1,3 BDO and/or
(vi) an enzyme or polypeptide that converts (R)-3HB—CoA and (R)-1,3 BDO to (3R)-hydroxybutyl (3R)-hydroxybutyrate.

In certain embodiments, the non-naturally occurring microbial organism or cell, having eliminated, substantially eliminated, or reduced (R) 3HB by-product or carboxylate by-product (e.g., acetate, malonate, and/or acetoacetate), and/or enhanced or increased carbon flux through (R) 3HB—CoA, and/or (R)-3HBAld, and/or acyl-CoA (e.g., acetyl-CoA, malonyl-CoA, and/or acetoacetyl-CoA), can further include a (3R)-hydroxybutyl (3R)-hydroxybutyrate pathway, wherein the non-naturally occurring microbial organism or cell includes at least one exogenous nucleic acid encoding one or more of an enzyme or polypeptide that converts a substrate to a product, or an isomeric form thereof including, for example:
(i) an enzyme or polypeptide that converts Ac—COA to malonyl-CoA
(ii) an enzyme or polypeptide that converts malonyl-CoA to AcAc—COA
(iii) an enzyme or polypeptide that converts AcAc—COA to(S)-3HB—CoA;
(iv) an enzyme or polypeptide that converts(S)-3HB—CoA to (R)-3HB—CoA;
(v) an enzyme or polypeptide that converts (R)-3HB—CoA to (R)-3HBAld;
(vi) an enzyme or polypeptide that converts (R)-3HBAld to (R)-1,3 BDO; and/or
(vii) an enzyme or polypeptide that converts (R)-3HB—CoA and (R)-1,3 BDO to (3R)-hydroxybutyl (3R)-hydroxybutyrate.

In some embodiments, Ac—COA is converted to AcAc—COA by an enzyme or polypeptide having AcAc—COA thiolase (EC 2.3.1) activity. In some embodiments, AcAc—COA is converted to(S)-3HB—CoA by an enzyme or polypeptide having HBD, optionally(S)-3hydroxybutyryl-CoA dehydrogenase (S—HBD) (EC 1.1.1.157) activity. In some embodiments, (S)-3HB—CoA is converted to (R)-3HB—CoA by an enzyme or polypeptide having 3HB—CoA epimerase (EC 5.1.2.3) activity. In some embodiments, (R)-3HB—CoA is converted to (R)-3HBAld by an enzyme or polypeptide having (R)-3HB—CoA reductase (EC 1.2.1.b) activity. In some embodiments, (R)-3HBAld is converted to (R)-1,3 BDO by an enzyme or polypeptide having (R)-3HB—Ald reductase (EC 1.1.1.a) activity. In some embodiments, Ac—CoA is converted to malonyl-CoA by an enzyme or polypeptide having Ac—CoA carboxylase (EC 6.4.12) activity. In some embodiments, malonyl-CoA is converted to AcAc—COA by an enzyme or polypeptide having AcAc—COA synthase (EC 2.3.1.194) activity. In some embodiments, AcAc—COA is converted to (R)-3HB—CoA by an enzyme or polypeptide having HBD, optionally, R—HBD, (EC1.1.157) activity. In some embodiments, (R)-3HB—CoA and (R)-1,3 BDO are converted to (3R)-hydroxybutyl (3R)-hydroxybutyrate by an enzyme or polypeptide having (3R)-hydroxybutyryl-CoA:(R)-1,3-butanediol alcohol transferase (EC 2.3.1) activity.

In certain embodiments, a 1,3-BDO pathway, and/or the pathway of an isomer thereof, can include one or more of the following enzymes: AcAc—COA thiolase; HBD, optionally R—HBD; (R)-3HB—CoA reductase; (R)-3HB—Ald reductase; Ac—COA carboxylase; AcAc—COA synthase; HBD, optionally S—HBD; 3HB—CoA epimerase; and/or (3R)-hydroxybutyryl-CoA: (R)-1,3-butanediol alcohol transferase.

The non-naturally occurring microbial organism can include at least one exogenous nucleic acid encoding a (3R)-hydroxybutyl (3R)-hydroxybutyrate pathway enzyme. The non-naturally occurring microbial organism can include two exogenous nucleic acids each encoding a (3R)-hydroxybutyl (3R)-hydroxybutyrate pathway enzyme, in other embodiments, three exogenous nucleic acids each encoding a (3R)-hydroxybutyl (3R)-hydroxybutyrate pathway enzyme, in other embodiments, four exogenous nucleic acids each encoding a (3R)-hydroxybutyl (3R)-hydroxybutyrate pathway enzyme, in other embodiments, five exogenous nucleic acids each encoding a (3R)-hydroxybutyl (3R)-hydroxybutyrate pathway enzyme, in other embodiments, six exogenous nucleic acids each encoding a (3R)-hydroxybutyl (3R)-hydroxybutyrate pathway enzyme, in other embodiments, seven exogenous nucleic acids each encoding a (3R)-hydroxybutyl (3R)-hydroxybutyrate pathway enzyme, in other embodiments, eight exogenous nucleic acids each encoding a (3R)-hydroxybutyl (3R)-hydroxybutyrate pathway enzyme, in other embodiments, nine or more exogenous nucleic acids each encoding a (3R)-hydroxybutyl (3R)-hydroxybutyrate pathway enzyme.

Any number of nucleic acids encoding these enzymes or polypeptides can be further introduced into a host microbial organism or cell already including one, two, three, four, five, six, seven, eight, or nine nucleic acids that encode any one or more of the other enzymes or polypeptides in the (3R)-hydroxybutyl (3R)-hydroxybutyrate pathway, and/or the pathway of an isomer thereof, as described herein. Where more than one exogenous nucleic acids are introduced, such nucleic acids can be any permutation of the multiple nucleic acids (e.g., polycistronic or multiple nucleic acids) already included in the host organism or cell.

One skilled in the art will understand that these (3R)-hydroxybutyl (3R)-hydroxybutyrate pathways, and/or the pathways of an isomer thereof, substrate-product pairs, and encoded enzymes or polypeptides are merely exemplary, and that any of the substrate-product pairs and/or encoded enzymes or polypeptides disclosed herein that are suitable to produce a desired acetyl-CoA derived product, such as (3R)-hydroxybutyl (3R)-hydroxybutyrate, and/or an isomer thereof, and for which an appropriate activity is available for the conversion of the substrate to the product, can be readily determined by one skilled in the art based on the teachings herein.

Any of the at least one exogenous nucleic acids can be a heterologous nucleic acid, and the non-naturally occurring microbial organism can be constructed for growth or culturing under substantially anaerobic conditions, for example, in a substantially anaerobic culture medium. Other culture conditions, including feedstock, are described in further detail herein.

Thus, provided herein is a non-naturally occurring microbial organism or cell having eliminated, substantially eliminated, or reduced 3HB by-product and/or carboxylate by-product (e.g., acetate, malonate, and/or acetoacetate), and/or enhanced or increased carbon flux through 3HB—CoA, and/or (R)-3HBAld, and/or acyl-CoA (e.g., acetyl-CoA, malonyl-CoA, and/or acetoacetyl-CoA), and containing at least one exogenous nucleic acid encoding one or more enzymes or polypeptides, where the one or more enzymes or polypeptides are part of a (3R)-hydroxybutyl (3R)-hydroxybutyrate pathway, and/or the pathway of an isomer thereof, converting acetyl-CoA or AcAc—COA to (3R)-hydroxybutyl (3R)-hydroxybutyrate, and/or an isomer thereof, as exemplified by the pathways shown in FIG. 7, and such as those disclosed in U.S. application Ser. No. 14/893,510, published as U.S. 2016-0108442 A1, which is incorporated herein by reference in its entirety

In certain embodiments, the non-naturally occurring microbial organism or cell, having eliminated, substantially eliminated, or reduced (R) 3HB by-product or carboxylate by-product (e.g., acetate and/or acetoacetate), and/or enhanced or increased carbon flux through (R) 3HB—CoA, and/or (R)-3HBAld, and/or acyl-CoA (e.g., acetyl-CoA and/or acetoacetyl-CoA), can further include a (3R)-hydroxybutyl (3R)-hydroxybutyrate pathway, wherein the non-naturally occurring microbial organism or cell includes at least one exogenous nucleic acid encoding one or more of an enzyme or polypeptide that converts a substrate to a product, or an isomeric form thereof including, for example:
(i) an enzyme or polypeptide that converts Ac—COA to AcAc—COA;
(ii) an enzyme or polypeptide that converts AcAc—COA, to (R)-3HB—CoA;
(iii) an enzyme or polypeptide that converts (R)-3HB—CoA to (R)-3-hydroxybutyryl-phosphate ((R)-3HB—P);
(iv) an enzyme or polypeptide that converts (R)-3HB—P to (R)-3HB, wherein 3HB gets recycled back into 3HB—CoA as described herein;
(v) an enzyme or polypeptide that converts (R)-3HB—CoA to (R)-3HBAld;
(vi) an enzyme or polypeptide that converts (R)-3HBAld to (R)-1,3 BDO; and/or
(vii) an enzyme or polypeptide that converts (R)-3HB—CoA and (R)-1,3 BDO to (3R)-hydroxybutyl (3R)-hydroxybutyrate.

In some embodiments, Ac—COA is converted to AcAc—COA by an enzyme or polypeptide having an Ac—CoA thiolase (EC 2.3.1) activity. In some embodiments, AcAc—COA is converted to (R)-3HB—CoA by an enzyme or polypeptide having 3HB—CoA dehydrogenase (HBD), optionally (R)-3HB—CoA dehydrogenase (R—HBD) (EC 1.1.1.a) activity or AcAc—COA reductase (ketone reducing/alcohol forming oxidoreductase) (EC 1.1.1.a) activity. In some embodiments, (R)-3HB—CoA is converted to (R)-3HBAld by an enzyme or polypeptide having butyraldehyde dehydrogenase (EC 1.2.1.) activity or acetaldehyde-alcohol dehydrogenase (EC 1.2.1) activity. In some embodiments, (R)-3HBAld is converted to (R)-1,3-BDO by an enzyme or polypeptide having acetaldehyde-alcohol dehydrogenase (EC 1.2.1) activity. In some embodiments, (R)-3HB—CoA is converted to (R)-3HB—P by an enzyme or polypeptide having phosphotransbutyrylase (PTB) (EC 2.3.1) activity. In some embodiments, (R)-3HB—P is converted to (R)-3HB by an enzyme or polypeptide having butyrate kinase (BUK) (EC 2.7.2) activity, wherein 3HB gets recycled back into 3HB—CoA, or is converted to 3HBAld by a 3HB to 3HBAld conversion step, as described herein. In some embodiments, (R)-3HB—CoA and (R)-1,3 BDO are converted to (3R)-hydroxybutyl (3R)-hydroxybutyrate by an enzyme or polypeptide having (3R)-hydroxybutyryl-CoA:(R)-1,3-butanediol alcohol transferase (EC 2.3.1) activity.

In certain embodiments, a 1,3-BDO pathway, and/or the pathway of an isomer thereof, can include one or more of the following enzymes: AcAc—COA thiolase; HBD, optionally a R—HBD, or AcAc—COA reductase (ketone reducing/alcohol forming oxidoreductase); butyraldehyde dehydrogenase or acetaldehyde-alcohol dehydrogenase; acetaldehyde-alcohol dehydrogenase; PTB; BUK and/or (3R)-hydroxybutyryl-CoA:(R)-1,3-butanediol alcohol transferase.

The non-naturally occurring microbial organism can include at least one exogenous nucleic acid encoding a (3R)-hydroxybutyl (3R)-hydroxybutyrate pathway enzyme. The non-naturally occurring microbial organism can include two exogenous nucleic acids each encoding a (3R)-hydroxybutyl (3R)-hydroxybutyrate pathway enzyme, in other embodiments, three exogenous nucleic acids each encoding a (3R)-hydroxybutyl (3R)-hydroxybutyrate pathway enzyme, in other embodiments, four exogenous nucleic acids each encoding a (3R)-hydroxybutyl (3R)-hydroxybutyrate pathway enzyme, in other embodiments, five exogenous nucleic acids each encoding a (3R)-hydroxybutyl (3R)-hydroxybutyrate pathway enzyme, in other embodiments, six exogenous nucleic acids each encoding a (3R)-hydroxybutyl (3R)-hydroxybutyrate pathway enzyme, in other embodiments, seven or more exogenous nucleic acids each encoding a (3R)-hydroxybutyl (3R)-hydroxybutyrate pathway enzyme.

Any number of nucleic acids encoding these enzymes or polypeptides can be further introduced into a host microbial organism or cell already including one, two, three, four, five, six, or seven nucleic acids that encode any one or more of the other enzymes or polypeptides in the (3R)-hydroxybutyl (3R)-hydroxybutyrate pathway, and/or the pathway of an isomer thereof, as described herein. Where more than one exogenous nucleic acids are introduced, such nucleic acids can be any permutation of the multiple nucleic acids (e.g., polycistronic or multiple nucleic acids) already included in the host organism or cell.

One skilled in the art will understand that these (3R)-hydroxybutyl (3R)-hydroxybutyrate pathways, and/or the pathways of an isomer thereof, substrate-product pairs, and encoded enzymes or polypeptides are merely exemplary, and that any of the substrate-product pairs and/or encoded enzymes or polypeptides disclosed herein that are suitable to produce a desired acetyl-CoA derived product, such as (3R)-hydroxybutyl (3R)-hydroxybutyrate, and/or an isomer thereof, and for which an appropriate activity is available for the conversion of the substrate to the product, can be readily determined by one skilled in the art based on the teachings herein.

Any of the at least one exogenous nucleic acids can be a heterologous nucleic acid, and the non-naturally occurring microbial organism can be constructed for growth or culturing under substantially anaerobic conditions, for example, in a substantially anaerobic culture medium. Other culture conditions, including feedstock, are described in further detail herein.

Thus, provided herein is a non-naturally occurring microbial organism or cell having eliminated, substantially eliminated, or reduced 3HB by-product, and/or enhanced or increased carbon flux through 3HB—CoA, and containing at least one exogenous nucleic acid encoding one or more enzymes or polypeptides, where the one or more enzymes or polypeptides are part of a (3R)-hydroxybutyl (3R)-hydroxybutyrate pathway, and/or the pathway of an isomer thereof, converting acetyl-CoA or AcAc—COA to (3R)-hydroxybutyl (3R)-hydroxybutyrate, and/or an isomer thereof, as exemplified by the pathways shown in FIG. 5.

The non-naturally occurring microbial organism of the present invention, having a glycolysis pathway or an acetyl-CoA pathway, can further include an acetyl-CoA derived product pathway with a 3HB—CoA intermediate, wherein the acetyl-CoA derived product is methacrylic acid (MAA; also referred to as methacrylate). Accordingly, in certain embodiments, the non-naturally occurring microbial organism having eliminated, substantially eliminated, or reduced 3HB by-product, and/or enhanced or increased carbon flux through 3HB—CoA, further includes a MAA pathway.

In certain embodiments, the non-naturally occurring microbial organism or cell, having eliminated, substantially eliminated, or reduced 3HB by-product or carboxylate by-product (e.g., acetate and/or acetoacetate), and/or enhanced or increased carbon flux through 3HB—CoA, and/or acyl-CoA (e.g., acetyl-CoA and/or acetoacetyl-CoA), can further include a MAA pathway, wherein the non-naturally occurring microbial organism or cell includes at least one exogenous nucleic acid encoding one or more of an enzyme or polypeptide that converts a substrate to a product, or an isomeric form thereof including, for example:
(i) an enzyme or polypeptide that converts Ac—COA to AcAc—COA;
(ii) an enzyme or polypeptide that converts AcAc—COA to 3HB—CoA;
(iii) an enzyme or polypeptide that converts 3HB—CoA to 2-hydroxyisobutyryl-COA (2HIB—CoA);
(iv) an enzyme or polypeptide that converts 2HIB—CoA to methacrylyl-CoA; and/or
(v) an enzyme or polypeptide that converts methacrylyl-CoA to MAA.

In certain embodiments, the non-naturally occurring microbial organism or cell, having eliminated, substantially eliminated, or reduced 3HB by-product or carboxylate by-product (e.g., vinylacetate and/or crotonate), and/or enhanced or increased carbon flux through 3HB—CoA, and/or acyl-CoA (e.g., vinylacetyl-CoA and/or crotonyl-CoA), can further include a MAA pathway, wherein the non-naturally occurring microbial organism or cell includes at least one exogenous nucleic acid encoding one or more of an enzyme or polypeptide that converts a substrate to a product, or an isomeric form thereof including, for example:
(i) an enzyme or polypeptide that converts 4HB—CoA to vinylacetyl-CoA;
(ii) an enzyme or polypeptide that converts vinylacetyl-CoA to crotonyl-CoA;
(iii) an enzyme or polypeptide that converts crotonyl-CoA to 3HB—CoA;
(iv) an enzyme or polypeptide that converts 3HB—CoA to 2HIB—CoA;
(v) an enzyme or polypeptide that converts 2HIB—CoA to methacrylyl-CoA; and/or
(vi) an enzyme or polypeptide that converts methacrylyl-CoA to MAA.

In some embodiments, the enzyme or polypeptide that converts Ac—COA to AcAc—COA has AcAc—COA thiolase (EC 2.3.1) activity. In some embodiments, the enzyme or polypeptide that converts AcAc—COA to 3HB—CoA has AcAc—COA reductase (ketone reducing/alcohol forming oxidoreductase) (EC 1.1.1.a) activity. In some embodiments, the enzyme or polypeptide that converts 3HB—CoA to 2HIB—CoA has 3HB—CoA mutase (EC 5.4.99.-) activity. In some embodiments, the enzyme or polypeptide that converts 2HIB—CoA to methacrylyl-CoA has 2HIB—CoA dehydratase (EC 4.2.1.-) activity. In some embodiments, the enzyme or polypeptide that converts methacrylyl-CoA to MAA has methacrylyl-CoA transferase, methacrylyl-CoA hydrolase or methacrylyl-CoA synthetase (EC 2.8.3.-, EC 3.1.2.-, or EC 6.2.1.-) activity. In some embodiments, the enzyme or polypeptide that converts 4HB—CoA to vinylacetyl-CoA has 4HB—CoA dehydratase (EC 4.2.1.-) activity. In some embodiments, the enzyme or polypeptide that converts vinylacetyl-CoA to crotonyl-CoA has vinylacetyl-CoA A-isomerase (EC 5.3.3.3) activity. In some embodiments, the enzyme or polypeptide that converts crotonyl-CoA to 3HB—CoA has CRT (EC 4.2.1.a) activity.

In certain embodiments, a MAA pathway, and/or the pathway of an isomer thereof, can include one or more of the following enzymes: AcAc—COA thiolase; AcAc—COA reductase (ketone reducing/alcohol forming oxidoreductase); 3HB—CoA mutase; 2HIB—CoA dehydratase; methacrylyl-CoA transferase, methacrylyl-CoA hydrolase and/or methacrylyl-CoA synthetase; 4HB—CoA dehydratase; vinylacetyl-CoA A-isomerase; and/or CRT.

The non-naturally occurring microbial organism can include one exogenous nucleic acid encoding a MAA pathway enzyme, in other embodiments, two exogenous nucleic acids each encoding a MAA pathway enzyme, in other embodiments, three exogenous nucleic acids each encoding a MAA pathway enzyme, in other embodiments, four exogenous nucleic acids each encoding a MAA pathway enzyme, in other embodiments, five exogenous nucleic acids each encoding a MAA pathway enzyme, in other embodiments, six exogenous nucleic acids each encoding a MAA pathway enzyme, in other embodiments, seven exogenous nucleic acids each encoding a MAA pathway enzyme, in other embodiments, eight or more exogenous nucleic acids each encoding a MAA pathway enzyme.

Any number of nucleic acids encoding these enzymes or polypeptides can be further introduced into a host microbial organism or cell already including one, two, three, four, five, six, seven, or eight nucleic acids that encode any one or more of the other enzymes or polypeptides in the MAA pathway, and/or the pathway of an isomer thereof, as described herein. Where more than one exogenous nucleic acids are introduced, such nucleic acids can be any permutation of the multiple nucleic acids (e.g., polycistronic or multiple nucleic acids) already included in the host organism or cell.

One skilled in the art will understand that these MAA pathways, and/or the pathways of an isomer thereof, substrate-product pairs, and encoded enzymes or polypeptides are merely exemplary, and that any of the substrate-product pairs and/or encoded enzymes or polypeptides disclosed herein that are suitable to produce a desired acetyl-CoA derived product, such as MAA, and/or an isomer thereof, and for which an appropriate activity is available for the conversion of the substrate to the product, can be readily determined by one skilled in the art based on the teachings herein.

Any of the at least one exogenous nucleic acids can be a heterologous nucleic acid, and the non-naturally occurring microbial organism can be constructed for growth or culturing under substantially anaerobic conditions, for example, in a substantially anaerobic culture medium. Other culture conditions, including feedstock, are described in further detail herein.

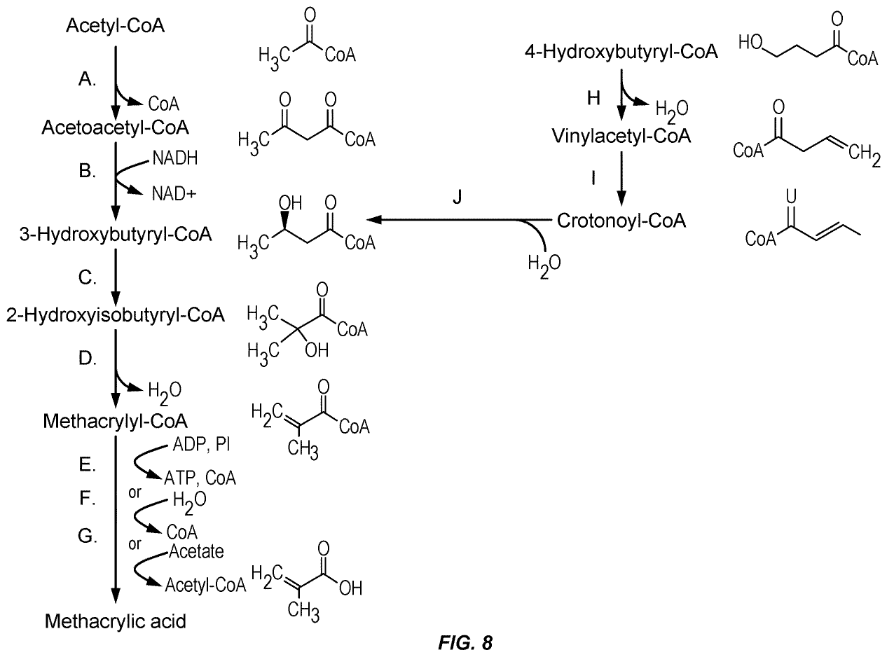

Thus, provided herein is a non-naturally occurring microbial organism or cell having eliminated, substantially eliminated, or reduced 3HB by-product or carboxylate by-product (e.g., acetate, acetoacetate, vinylacetate and/or crotonate), and/or enhanced or increased carbon flux through 3HB—CoA, and/or acyl-CoA (e.g., acetyl-CoA, acetoacetyl-CoA, vinylacetyl-CoA, and/or crotonyl-CoA), and containing at least one exogenous nucleic acid encoding one or more enzymes or polypeptides, where the one or more enzymes or polypeptides are part of a MAA pathway, and/or the pathway of an isomer thereof, converting acetyl-CoA, AcAc—COA, or 4HB—CoA to MAA, and/or an isomer thereof, as exemplified by the pathways shown in FIG. 8.

In certain embodiments, the non-naturally occurring microbial organism or cell, having eliminated, substantially eliminated, or reduced 3HB by-product or carboxylate by-product (e.g., acetate, acetoacetate, crotonate, butyrate, and/or isobutyrate), and/or enhanced or increased carbon flux through 3HB—CoA, and/or acyl-CoA (e.g., acetyl-CoA, acetoacetyl-CoA, crotonyl-CoA, butyryl-CoA, and/or isobutyryl-CoA), can further include a MAA pathway, wherein the non-naturally occurring microbial organism or cell includes at least one exogenous nucleic acid encoding one or more of an enzyme or polypeptide that converts a substrate to a product, or an isomeric form thereof including, for example:
(i) an enzyme or polypeptide that converts Ac—COA to AcAc—COA;
(ii) an enzyme or polypeptide that converts AcAc—COA to 3HB—CoA;
(iii) an enzyme or polypeptide that converts 3HB—CoA to crotonyl-CoA;
(iv) an enzyme or polypeptide that converts crotonyl-CoA to butyryl-CoA;
(v) an enzyme or polypeptide that converts butyryl-CoA to isobutyryl-CoA;
(vi) an enzyme or polypeptide that converts isobutyryl-CoA to methacrylyl-CoA; and/or
(vii) an enzyme or polypeptide that converts methacrylyl-CoA to MAA.

In some embodiments, the enzyme or polypeptide that converts Ac—COA to AcAc—COA has AcAc—COA thiolase (EC 2.3.1) activity. In some embodiments, the enzyme or polypeptide that converts AcAc—COA to 3HB—CoA has AcAc—COA reductase (ketone reducing/alcohol forming oxidoreductase) (EC 1.1.1.a) activity. In some embodiments, the enzyme or polypeptide that converts crotonyl-CoA to 3HB—CoA has CRT (EC 4.2.1.a) activity. In some embodiments, the enzyme or polypeptide that converts crotonyl-CoA to butyryl-CoA has butyryl-CoA dehydrogenase (EC 1.3.99.-) activity. In some embodiments, the enzyme or polypeptide that converts butyryl-CoA to isobutyryl-CoA has isobutyryl-CoA mutase (EC 5.4.99.13) activity. In some embodiments, the enzyme or polypeptide that converts isobutyryl-CoA to methacrylyl-CoA has isobutyryl-CoA dehydrogenase (EC 1.3.99.12) activity. In some embodiments, the enzyme or polypeptide that converts methacrylyl-CoA to MAA has methacrylyl-CoA transferase, methacrylyl-CoA hydrolase or methacrylyl-CoA synthetase (EC 2.8.3.-, EC 3.1.2.-, or EC 6.2.1.-) activity.

In certain embodiments, a MAA pathway, and/or the pathway of an isomer thereof, can include one or more of the following enzymes: AcAc—COA thiolase; AcAc—COA reductase (ketone reducing/alcohol forming oxidoreductase); methacrylyl-CoA transferase, methacrylyl-CoA hydrolase and/or methacrylyl-CoA synthetase; CRT; butyryl-CoA dehydrogenase; isobutyryl-CoA mutase; and/or isobutyryl-CoA dehydrogenase.

The non-naturally occurring microbial organism can include one exogenous nucleic acid encoding a MAA pathway enzyme, in other embodiments, two exogenous nucleic acids each encoding a MAA pathway enzyme, in other embodiments, three exogenous nucleic acids each encoding a MAA pathway enzyme, in other embodiments, four exogenous nucleic acids each encoding a MAA pathway enzyme, in other embodiments, five exogenous nucleic acids each encoding a MAA pathway enzyme, in other embodiments, six exogenous nucleic acids each encoding a MAA pathway enzyme, in other embodiments, or seven exogenous nucleic acids each encoding a MAA pathway enzyme.

Any number of nucleic acids encoding these enzymes or polypeptides can be further introduced into a host microbial organism or cell already including one, two, three, four, five, six, or seven nucleic acids that encode any one or more of the other enzymes or polypeptides in the MAA pathway, and/or the pathway of an isomer thereof, as described herein. Where more than one exogenous nucleic acids are introduced, such nucleic acids can be any permutation of the multiple nucleic acids (e.g., polycistronic or multiple nucleic acids) already included in the host organism or cell.

One skilled in the art will understand that these MAA pathways, and/or the pathways of an isomer thereof, substrate-product pairs, and encoded enzymes or polypeptides are merely exemplary, and that any of the substrate-product pairs and/or encoded enzymes or polypeptides disclosed herein that are suitable to produce a desired acetyl-CoA derived product, such as MAA, and/or an isomer thereof, and for which an appropriate activity is available for the conversion of the substrate to the product, can be readily determined by one skilled in the art based on the teachings herein.

Any of the at least one exogenous nucleic acids can be a heterologous nucleic acid, and the non-naturally occurring microbial organism can be constructed for growth or culturing under substantially anaerobic conditions, for example, in a substantially anaerobic culture medium. Other culture conditions, including feedstock, are described in further detail herein.

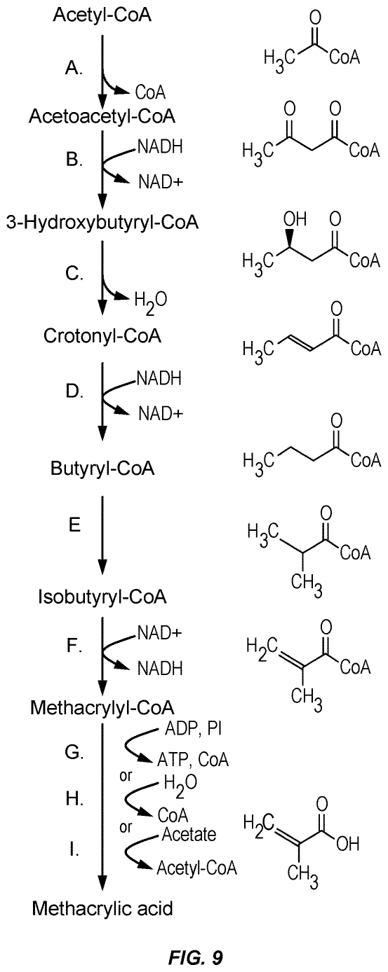

Thus, provided herein is a non-naturally occurring microbial organism or cell having eliminated, substantially eliminated, or reduced 3HB by-product or carboxylate by-product (e.g., acetate, acetoacetate, crotonate, butyrate, and/or isobutyrate), and/or enhanced or increased carbon flux through 3HB—CoA, and/or acyl-CoA (e.g., acetyl-CoA, acetoacetyl-CoA, crotonyl-CoA, butyryl-CoA, and/or isobutyryl-CoA), and containing at least one exogenous nucleic acid encoding one or more enzymes or polypeptides, where the one or more enzymes or polypeptides are part of a MAA pathway, and/or the pathway of an isomer thereof, converting acetyl-CoA, or AcAc—COA, to MAA, and/or an isomer thereof, as exemplified by the pathways shown in FIG. 8 and FIG. 9.

In some embodiments, the non-naturally occurring microbial organism of the present invention, having a glycolysis pathway or an acetyl-CoA pathway, has an acetyl-CoA derived product pathway with a 3HB—CoA intermediate, wherein the acetyl-CoA derived product is ester of MAA, such as methyl methacrylate (MMA). Accordingly, in an additional embodiment, the non-naturally occurring microbial organism having eliminated, substantially eliminated, or reduced 3HB by-product, and/or enhanced or increased carbon flux through 3HB—CoA, can include a MAA pathway and can further include an MAA ester pathway.

In some embodiments, a non-naturally occurring organism of the present invention having a MAA ester pathway can further include at least one exogenous nucleic acid encoding one or more MAA ester pathway enzymes, as described herein for the production of MMA precursors (see e.g., FIG. 8 and FIG. 9). In certain embodiments, the MAA ester pathway includes an MAA pathway, whereby the MAA product is then esterified with methanol to produce an MAA ester. Different methacrylate esters can be produced in such an organism by esterification of the MAA with different alcohols. For example, the MAA can be esterified with ethanol, n-propanol, isopropanol, n-butanol, sec-butanol, tert-butanol, pentanol, or hexanol, to produce ethyl methacrylate, n-propyl methacrylate, isopropyl methacrylate, n-butyl methacrylate, sec-butyl methacrylate, tert-butyl methacrylate, pentyl methacrylate, or hexyl methacrylate, respectively.

In certain embodiments, the non-naturally occurring microbial organism or cell, having eliminated, substantially eliminated, or reduced 3HB by-product or carboxylate by-product, and/or enhanced or increased carbon flux through 3HB—CoA, and/or acyl-CoA, can include a MAA pathway and can further include a MAA ester pathway, wherein the non-naturally occurring microbial organism or cell includes at least one exogenous nucleic acid encoding one or more of an enzyme or polypeptide that converts a substrate to a product, or an isomeric form thereof including, for example an enzyme or polypeptide that converts MAA and an alcohol (i.e., R—OH, wherein R denotes any organic functional group including, but not limited to, a methyl, ethyl, n-propyl, n-butyl, i-propyl, sec-butyl, and tert-butyl, pentyl, or hexyl functional group) to a MAA ester.

In certain embodiments, the non-naturally occurring microbial organism or cell, having eliminated, substantially eliminated, or reduced 3HB by-product or carboxylate by-product, and/or enhanced or increased carbon flux through 3HB—CoA, and/or acyl-CoA, can include a MAA pathway and can further include a MAA ester pathway, wherein the non-naturally occurring microbial organism or cell includes at least one exogenous nucleic acid encoding one or more of an enzyme or polypeptide that converts a substrate to a product, or an isomeric form thereof including, for example:
(i) an enzyme or polypeptide that converts MAA to methacrylyl-CoA; and/or
(ii) an enzyme or polypeptide that converts methacrylyl-CoA and an alcohol (i.e., R—OH, wherein R denotes any organic functional group including, but not limited to, a methyl, ethyl, n-propyl, n-butyl, i-propyl, sec-butyl, and tert-butyl, pentyl, or hexyl functional group) to a MAA ester.

In some embodiments, MAA and an alcohol is converted to a MAA ester by an enzyme or polypeptide having methacrylate ester-forming enzyme activity. In some embodiments, MAA and an alcohol (i.e., R—OH, wherein R denotes any organic functional group including, but not limited to, a methyl, ethyl, n-propyl, n-butyl, i-propyl, sec-butyl, and tert-butyl, pentyl, or hexyl functional group) is converted to a MAA ester by chemical conversion/esterification, by any suitable method, for example, heating in the presence of a dehydrating agent such as an acid. In some embodiments, MAA is converted to methacrylyl-CoA by an enzyme or polypeptide having methacrylyl-CoA synthetase or methacrylyl-CoA transferase activity. In some embodiments, methacrylyl-CoA and an alcohol (i.e., R—OH, wherein R denotes any organic functional group including, but not limited to, a methyl, ethyl, n-propyl, n-butyl, i-propyl, sec-butyl, and tert-butyl, pentyl, or hexyl functional group) is converted to an MAA ester by an enzyme or polypeptide having an alcohol transferase activity.

In certain embodiments, a MAA ester pathway, and/or the pathway of an isomer thereof, can include one or more of the following enzymes: a methacrylyl-CoA transferase or a methacrylyl-CoA synthetase; an alcohol transferase; and/or a methacrylate ester-forming enzyme.

The non-naturally occurring microbial organism can include one exogenous nucleic acid encoding a MAA ester pathway enzyme, in other embodiments, two exogenous nucleic acids each encoding a MAA ester pathway enzyme, in other embodiments, three exogenous nucleic acids each encoding a MAA ester pathway enzyme, or more exogenous nucleic acids each encoding a MAA pathway enzyme.

Any number of nucleic acids encoding these enzymes or polypeptides can be further introduced into a host microbial organism or cell already including one, two, or three nucleic acids that encode any one or more of the other enzymes or polypeptides in the MAA ester pathway, and/or the pathway of an isomer thereof, as described herein. Where more than one exogenous nucleic acids are introduced, such nucleic acids can be any permutation of the multiple nucleic acids (e.g., polycistronic or multiple nucleic acids) already included in the host organism or cell.

In certain embodiments, the MAA precursor is produced by the microbial organism, using any of the MAA pathways described herein, or described elsewhere, or known in the art. Production of a MAA ester, such as MMA, using microorganisms is known in the art, as exemplified in U.S. Pat. Nos. 9,133,487, and 9,346,902, each of which are incorporated herein by reference in their entirety.

Thus, provided herein is a non-naturally occurring microbial organism or cell having eliminated, substantially eliminated, or reduced 3HB by-product or carboxylate by-product, and/or enhanced or increased carbon flux through 3HB—CoA and/or acyl-CoA, and containing at least one exogenous nucleic acid encoding one or more enzymes or polypeptides, where the one or more enzymes or polypeptides are part of an MAA ester pathway, and/or the pathway of an isomer thereof, converting MAA to a MAA ester, and/or an isomer thereof, as exemplified by the pathways shown in FIGS. 8 and 9.

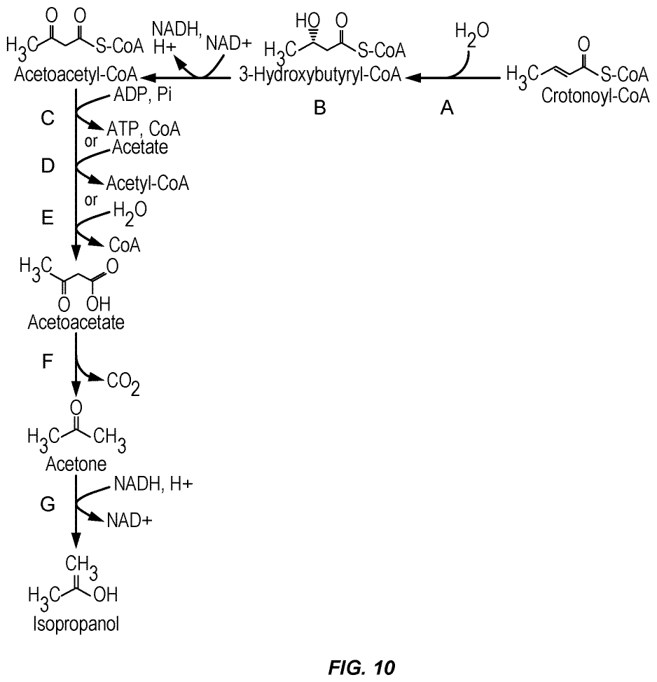

The non-naturally occurring organisms and cells of the present invention can also have one or more pathways which may produce precursors for pathways described herein. For example, crotonyl-CoA may be produced as a by-product in one or more pathways in a non-naturally occurring organisms and cells of the present invention (see for example FIGS. 3, 4, 6 and 8), wherein crotonyl-CoA may be utilized as a precursor for pathways described herein, such as the pathway set forth in FIG. 10.

In certain embodiments, the non-naturally occurring microbial organism or cell, having eliminated, substantially eliminated, or reduced 3HB by-product or carboxylate by-product (e.g., crotonate), and/or enhanced or increased carbon flux through 3HB—CoA and/or acyl-CoA (e.g., crotonyl-CoA), can further include a isopropanol pathway, wherein the non-naturally occurring microbial organism or cell includes at least one exogenous nucleic acid encoding one or more of an enzyme or polypeptide that converts a substrate to a product, or an isomeric form thereof including, for example:
(i) an enzyme or polypeptide that converts crotonyl-CoA to 3HB—CoA;
(ii) an enzyme or polypeptide that converts 3HB—CoA to AcAc—COA;
(iii) an enzyme or polypeptide that converts AcAc—COA to acetoacetate;
(iv) an enzyme or polypeptide that converts acetoacetate to acetone; and/or
(v) an enzyme or polypeptide that converts acetone to isopropanol.

In some embodiments, the enzyme or polypeptide that converts crotonyl-CoA to 3HB—CoA has CRT (EC 4.2.1.a) activity. In some embodiments, the enzyme or polypeptide that converts 3HB—CoA to AcAc—COA has HBD (EC 1.1.1.a) activity. In some embodiments, the enzyme or polypeptide that converts AcAc—COA to Acetoacetate has acetoacetyl-CoA synthetase (EC 6.2.1.a) activity, acetyl-CoA: acetoacetate-CoA transferase (EC 2.8.3.a) activity, and/or acetoacetyl-CoA hydrolase (EC 3.1.2.a) activity. In some embodiments, the enzyme or polypeptide that converts acetoacetate to acetone has acetoacetate decarboxylase (EC 4.1.1.a) activity. In some embodiments, the enzyme or polypeptide that converts acetone to isopropanol has acetone reductase (EC 1.1.1.a) activity.

In some embodiments, the 1,3-BDO pathway includes at least one exogenous nucleic acid encoding one or more of the following enzymes: CRT; HBD; acetoacetyl-CoA synthetase, acetyl-CoA: acetoacetate-CoA transferase, and/or acetoacetyl-CoA hydrolase; acetoacetate decarboxylase; and/or acetone reductase.

The non-naturally occurring microbial organism can include at least one exogenous nucleic acid encoding an isopropanol pathway enzyme. The non-naturally occurring microbial organism can include two exogenous nucleic acids each encoding an isopropanol pathway enzyme, in other embodiments, three exogenous nucleic acids each encoding an isopropanol pathway enzyme, in other embodiments, four exogenous nucleic acids each encoding an isopropanol pathway enzyme, in other embodiments, five or more exogenous nucleic acids each encoding an isopropanol pathway enzyme.

Any number of nucleic acids encoding these enzymes can be further introduced into a host microbial organism or cell including one, two, three, four, or five nucleic acids that encode these enzymes. Where more than one exogenous nucleic acids are introduced, such nucleic acids can be any permutation of the multiple nucleic acids (e.g., polycistronic or multiple nucleic acids) already included in the host organism or cell.

One skilled in the art will understand that these isopropanol pathways, substrate-product pairs, and encoded enzymes or polypeptides are merely exemplary, and that any of the substrate-product pairs and/or encoded enzymes or polypeptides disclosed herein that are suitable to produce a desired acetyl-CoA derived product, such as isopropanol, and for which an appropriate activity is available for the conversion of the substrate to the product, can be readily determined by one skilled in the art based on the teachings herein.

Any of the at least one exogenous nucleic acids can be a heterologous nucleic acid, and the non-naturally occurring microbial organism can be constructed for growth or culturing under substantially anaerobic conditions, for example, in a substantially anaerobic culture medium. Other culture conditions, including feedstock, are described in further detail herein.

Thus, provided herein is a non-naturally occurring microbial organism having eliminated, substantially eliminated, or reduced 3HB by-product or carboxylate by-product (e.g., crotonate), and/or enhanced or increased carbon flux through 3HB—CoA and/or acyl-CoA (e.g., crotonyl-CoA), and containing at least one exogenous nucleic acid encoding one or more of an enzyme or polypeptide, where the enzyme(s) or polypeptide(s) are included in or are a part of an isopropanol pathway, and includes converting crotonyl-CoA to isopropanol, as exemplified by the pathways shown in FIG. 10.

Also provided herein is a non-naturally occurring microbial organism, having eliminated, substantially eliminated, or reduced 3HB by-product or carboxylate by-product, and/or enhanced or increased carbon flux through 3HB—CoA and/or one or more other acyl-CoAs, and containing at least one exogenous nucleic acid encoding one or more of an enzyme or polypeptide, where the enzyme(s) or polypeptide(s) are included in or are a part of an HMD, adipate, 6ACA, CPL, or HDO pathway. Such pathways are known in the art and are described, for example, in WO 2010/129936, WO 2016/209883, WO 2009/151728, WO 2014/099725, WO 2012/177721, WO 2009/113853, WO 2005/068643, WO 2009/113855, and WO 2011/078668, which are incorporated herein by reference in their entirety.

In some embodiments, the non-naturally occurring microbial organism having eliminated, substantially eliminated, or reduced carboxylate by-products (e.g., acetate or succinate), and enhanced carbon flux through the corresponding acyl-CoA intermediate or precursor (e.g., acetyl-CoA or succinyl-CoA), by having a carboxylate by-product recycling loop, also includes at least one acetyl-CoA derived product pathway, a glycolysis pathway, and/or a pathway having an acyl-CoA intermediate or precursor (e.g., acetyl-CoA or succinyl-CoA). In some embodiments, the acetyl-CoA derived product pathway includes, for example, a hexamethylenediamine (HMDA or HMD) pathway, a caprolactam (CPL) pathway, an adipate (or adipic acid) pathway, a 6-aminocaproic acid (6-ACA) pathway, and/or a 1,6-hexanediol (HDO) pathway. Accordingly, such a non-naturally occurring microbial organism having eliminated, substantially eliminated, or reduced carboxylate by-product, comprises a microbial organism having increased production (e.g., in terms of productivity, titer, and/or yield) of an acetyl-CoA derived product, such as HMD, CPL, adipate, 6-ACA, and HDO.

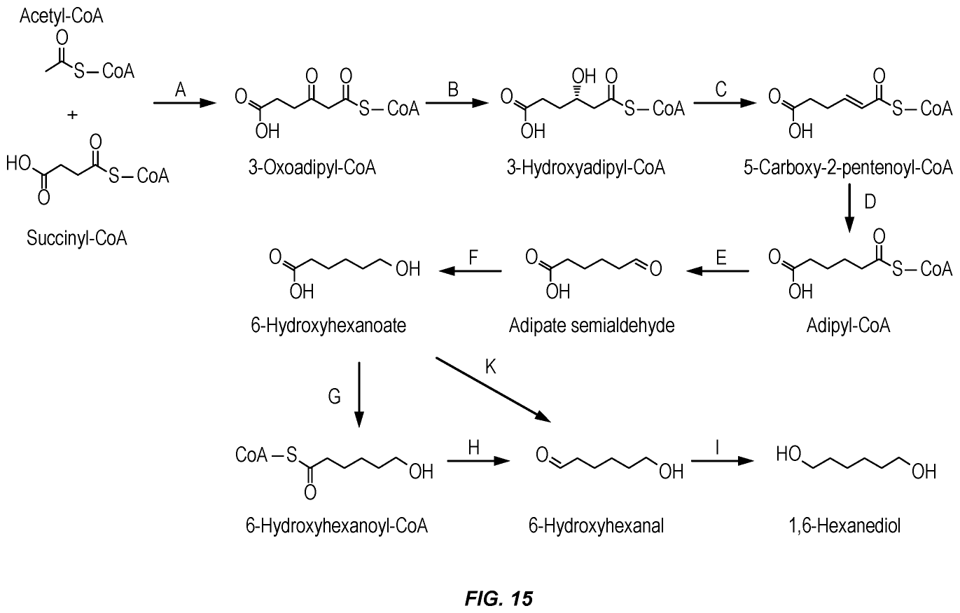

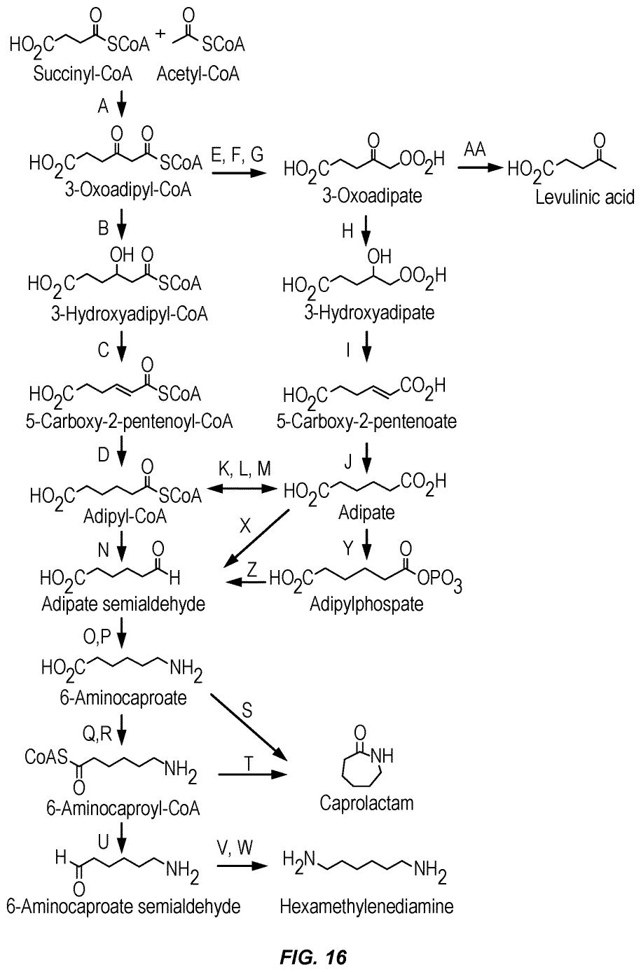

Thus, provided herein is a non-naturally occurring microbial organism or cell having one or more (or at least one) eliminated, substantially eliminated, or reduced carboxylate by-product(s) (e.g., acetate or succinate), and/or enhanced or increased carbon flux through the corresponding acyl-CoA intermediate or precursor (e.g., acetyl-CoA or succinyl-CoA), and containing at least one nucleic acid or at least one exogenous nucleic acid encoding one or more enzymes or polypeptides, where the one or more enzymes or polypeptides are part of an acetyl-CoA derived product pathway with an acyl-CoA intermediate or precursor (e.g., acetyl-CoA or succinyl-CoA), and/or the pathway of an isomer thereof, as exemplified by the pathways described herein and shown, for example, in any one of FIG. 15 and FIG. 16.

For example, provided herein is a non-naturally occurring microbial organism or cell of the present invention having at least one acetyl-CoA derived product pathway, a glycolysis pathway, and/or a pathway having an acyl-CoA intermediate or precursor (e.g., acetyl-CoA or succinyl-CoA), wherein the acetyl-CoA derived product is HDO. Accordingly, in certain embodiments, the non-naturally occurring microbial organism or cell, having one or more (or at least one) eliminated, substantially eliminated, or reduced carboxylate by-product(s) (e.g., acetate or succinate), and/or enhanced or increased carbon flux through one or more (or at least one) acyl-CoA intermediate or precursor (e.g., acetyl-CoA or succinyl-CoA), includes an HDO pathway.

In certain embodiments, the non-naturally occurring microbial organism or cell, having one or more (or at least one) eliminated, substantially eliminated, or reduced carboxylate by-product(s) (e.g., acetate and/or succinate), and/or enhanced or increased carbon flux through one or more (or at least one) acyl-CoA intermediate or precursor (e.g., acetyl-CoA or succinyl-CoA), can further include an HDO pathway, wherein the non-naturally occurring microbial organism or cell includes at least one nucleic acid or at least one exogenous nucleic acid encoding one or more of an enzyme or polypeptide that converts a substrate to a product, or an isomeric form thereof including, for example:
(i) an enzyme or polypeptide that converts succinyl-CoA and acetyl-CoA to 3-oxoadipyl-CoA, wherein the succinyl-CoA and/or the acetyl-CoA can get converted to succinate and/or acetate by-products respectively, and wherein the succinate and/or acetate gets recycled back into succinyl-CoA and/or the acetyl-CoA, respectively, (e.g., by a carboxylate by-product recycling loop) as described herein;
(ii) an enzyme or polypeptide that converts 3-oxoadipyl-CoA to 3-hydroxyadipyl-CoA;
(iii) an enzyme or polypeptide that converts 3-hydroxyadipyl-CoA to 5-carboxy-2-pentenoyl-CoA (also referred to as 2,3-dehydroadipyl-coA);
(iv) an enzyme or polypeptide that converts 5-carboxy-2-pentenoyl-CoA to adipyl-CoA;
(v) an enzyme or polypeptide that converts adipyl-CoA to adipate semialdehyde;
(vi) an enzyme or polypeptide that converts adipate semialdehyde to 6-hydroxyhexanoate;
(vii) an enzyme or polypeptide that converts 6-hydroxyhexanoate to 6-hydroxyhexanoyl-CoA;
(viii) an enzyme or polypeptide that converts 6-hydroxyhexanoyl-CoA to 6-hydroxyhexanal; and/or
(ix) an enzyme or polypeptide that converts 6-hydroxyhexanal to HDO.

In certain embodiments, the non-naturally occurring microbial organism or cell, having at least one (or one or more) eliminated, substantially eliminated, or reduced carboxylate by-product(s) (e.g., acetate and/or succinate), and/or enhanced or increased carbon flux through at least one (or one or more) acyl-CoA intermediate or precursor (e.g., acetyl-CoA or succinyl-CoA), can further include an HDO pathway, wherein the non-naturally occurring microbial organism or cell includes at least one nucleic acid or at least one exogenous nucleic acid encoding one or more of an enzyme or polypeptide that converts a substrate to a product, or an isomeric form thereof including, for example:
(i) an enzyme or polypeptide that converts succinyl-CoA and acetyl-CoA to 3-oxoadipyl-CoA, wherein the succinyl-CoA and/or the acetyl-CoA can get converted to succinate and/or acetate by-product(s) respectively, and wherein the succinate and/or acetate gets recycled back into succinyl-CoA and/or the acetyl-CoA, respectively, (e.g., by a carboxylate by-product recycling loop) as described herein;
(ii) an enzyme or polypeptide that converts 3-oxoadipyl-CoA to 3-hydroxyadipyl-CoA;
(iii) an enzyme or polypeptide that converts 3-hydroxyadipyl-CoA to 5-carboxy-2-pentenoyl-CoA (also referred to as 2,3-dehydroadipyl-coA);
(iv) an enzyme or polypeptide that converts 5-carboxy-2-pentenoyl-CoA to adipyl-CoA;
(v) an enzyme or polypeptide that converts adipyl-CoA to adipate semialdehyde;
(vi) an enzyme or polypeptide that converts adipate semialdehyde to 6-hydroxyhexanoate;
(vii) an enzyme or polypeptide that converts 6-hydroxyhexanoate to 6-hydroxyhexanal; and/or
(viii) an enzyme or polypeptide that converts 6-hydroxyhexanal to HDO.

In some embodiments, succinyl-CoA and acetyl-CoA is converted to 3-oxoadipyl-CoA by an enzyme or polypeptide having a thiolase (Thl) (EC 2.3.1.-) activity. In some embodiments, 3-oxoadipyl-CoA is converted to 3-hydroxyadipyl-CoA by an enzyme or polypeptide having a hydroxyadipyl-CoA dehydrogenase (Hbd) (EC 1.1.1.-) activity. In some embodiments, 3-hydroxyadipyl-CoA is converted to 5-carboxy-2-pentenoyl-CoA by an enzyme or polypeptide having a crotonase (Crt) (EC 4.2.1.-) activity. In some embodiments, 5-carboxy-2-pentenoyl-CoA is converted to adipyl-CoA by an enzyme or polypeptide having a trans-enoyl CoA reductase (Ter) (EC 1.3.1.-) activity. In some embodiments, adipyl CoA is converted to adipate semialdehyde by an enzyme or polypeptide having a 6ACA-aldehyde dehydrogenase (6ACA-ALD) (EC 1.2.1.-) activity. In some embodiments, adipate semialdehyde is converted to 6-hydroxyhexanoate by an enzyme or polypeptide having an alcohol dehydrogenase (EC 1.1.1.-) activity. In some embodiments, 6-hydroxyhexanoate is converted to 6-hydroxyhexanoyl-CoA by an enzyme or polypeptide having an acyl-coenzyme A ligase (ACL) (EC 6.2.1.-) or an acyl-CoA transferase activity. In some embodiments, 6-hydroxyhexanoyl-CoA is converted to 6-hydroxyhexanal by an enzyme or polypeptide having an acetylating aldehyde dehydrogenase (ALDH) or an acyl-CoA reductase/dehydrogenase activity. In some embodiments, 6-hydroxyhexanal is converted to HDO by an enzyme or polypeptide having an alcohol dehydrogenase (ADH) (EC 1.1.1.-) activity. In some embodiments, 6-hydroxyhexanoate is converted to 6-hydroxyhexanal by an enzyme or polypeptide having a carboxylic acid reductase (CAR) (EC 1.2.1.-) activity. In some embodiments, 6-hydroxyhexanoate is converted to 6-hydroxyhexanal by an enzyme or polypeptide having CAR activity and an enzyme or polypeptide having phosphopantetheinyl transferase (PPTase) activity.

In certain embodiments, a HDO pathway, and/or the pathway of an isomer thereof, can include one or more of the following enzymes: thiolase (Thl), hydroxyadipyl-CoA dehydrogenase (Hbd), crotonase (Crt), trans-enoyl CoA reductase (Ter), 6ACA-aldehyde dehydrogenase (6ACA-ALD), alcohol dehydrogenase, acyl-coenzyme A ligase (ACL) or acyl-CoA transferase, acetylating aldehyde dehydrogenase (ALDH) or an acyl-CoA reductase/dehydrogenase, an alcohol dehydrogenase (ADH), a carboxylic acid reductase (CAR) and/or a phosphopantetheinyl transferase (PPTase).

The non-naturally occurring microbial organism can include at least one exogenous nucleic acid encoding an HDO pathway enzyme. The non-naturally occurring microbial organism can include two exogenous nucleic acids each encoding an HDO pathway enzyme, in other embodiments, three exogenous nucleic acids each encoding an HDO pathway enzyme, in other embodiments, four exogenous nucleic acids each encoding an HDO pathway enzyme, in other embodiments, five exogenous nucleic acids each encoding an HDO pathway enzyme, in other embodiments, six exogenous nucleic acids each encoding an HDO pathway enzyme, in other embodiments, seven exogenous nucleic acids each encoding an HDO pathway enzyme, or in other embodiments, eight or more exogenous nucleic acids, each encoding an HDO pathway enzyme.

Any number of nucleic acids encoding these enzymes or polypeptides can be further introduced into a host microbial organism or cell already including one, two, three, four, five, six, seven, eight, or nine nucleic acids that encode any one or more of the other enzymes or polypeptides in the HDO pathway, and/or the pathway of an isomer thereof, as described herein. Where more than one exogenous nucleic acids are introduced, such nucleic acids can be any permutation of the multiple nucleic acids (e.g., polycistronic or multiple nucleic acids) already included in the host organism or cell.

One skilled in the art will understand that these HDO pathways, and/or the pathways of an isomer thereof, substrate-product pairs, and encoded enzymes or polypeptides are merely exemplary, and that any of the substrate-product pairs and/or encoded enzymes or polypeptides disclosed herein that are suitable to produce a desired acetyl-CoA derived product, such as HDO, and/or an isomer thereof, and for which an appropriate activity is available for the conversion of the substrate to the product, can be readily determined by one skilled in the art based on the teachings herein.

Any of the at least one exogenous nucleic acids can be a heterologous nucleic acid, and the non-naturally occurring microbial organism can be constructed for growth or culturing under substantially anaerobic conditions, for example, in a substantially anaerobic culture medium. Other culture conditions, including feedstock, are described in further detail herein.

Thus, provided herein is a non-naturally occurring microbial organism or cell having at least one (or one or more) eliminated, substantially eliminated, or reduced carboxylate by-product(s) (e.g., acetate and/or succinate), and/or enhanced or increased carbon flux through at least one (or one or more) acyl-CoA (particularly a short-chain acyl-CoA, e.g., acetyl-CoA and/or succinyl-CoA) intermediate or precursor, and containing at least one nucleic acid or at least one exogenous nucleic acid encoding one or more enzymes or polypeptides, where the one or more enzymes or polypeptides are part of an HDO pathway, and/or the pathway of an isomer thereof, converting acetyl-CoA or succinyl-CoA to HDO, and/or an isomer thereof, as exemplified by the pathways shown in FIG. 15.

Also provided herein is a non-naturally occurring organism or cell of the present invention having at least one acetyl-CoA derived product pathway, a glycolysis pathway, and/or a pathway having an acyl-CoA intermediate or precursor (e.g., acetyl-CoA or succinyl-CoA), wherein the acetyl-CoA derived product is adipate. Accordingly, in certain embodiments, the non-naturally occurring microbial organism or cell, having at least on (or one or more) eliminated, substantially eliminated, or reduced carboxylate by-products (e.g., acetate or succinate), and/or enhanced or increased carbon flux through at least one (or one or more) acyl-CoA intermediate or precursor (e.g., acetyl-CoA or succinyl-CoA), includes an adipate pathway, a 6ACA pathway, a CPL pathway, and/or an HMD pathway.

In certain embodiments, the non-naturally occurring microbial organism or cell, having at least one (or one or more) eliminated, substantially eliminated, or reduced carboxylate by-product (e.g., acetate and/or succinate), and/or enhanced or increased carbon flux through at least one (or one or more) acyl-CoA intermediate or precursor (e.g., acetyl-CoA or succinyl-CoA), can further include an adipate pathway, wherein the non-naturally occurring microbial organism or cell includes at least one exogenous nucleic acid encoding one or more of an enzyme or polypeptide that converts a substrate to a product, or an isomeric form thereof including, for example:
(i) an enzyme or polypeptide that converts succinyl-CoA and acetyl-CoA to 3-oxoadipyl-CoA, wherein the succinyl-CoA and/or the acetyl-CoA can get converted to succinate and/or acetate by-products, respectively, and wherein the succinate and/or acetate gets recycled back into succinyl-CoA and/or the acetyl-CoA, respectively, (e.g., by a carboxylate by-product recycling loop) as described herein;
(ii) an enzyme or polypeptide that converts 3-oxoadipyl-CoA to 3-hydroxyadipyl-CoA;
(iii) an enzyme or polypeptide that converts 3-hydroxyadipyl-CoA to 5-carboxy-2-pentenoyl-CoA;
(iv) an enzyme or polypeptide that converts 5-carboxy-2-pentenoyl-CoA to adipyl-CoA; and/or
(v) an enzyme or polypeptide that converts adipyl-CoA to adipate.

In certain embodiments, the non-naturally occurring microbial organism or cell, having at least one eliminated, substantially eliminated, or reduced carboxylate by-product (e.g., acetate and/or succinate), and/or enhanced or increased carbon flux through at least one acyl-CoA intermediate or precursor (e.g., acetyl-CoA or succinyl-CoA), can further include an adipate pathway, wherein the non-naturally occurring microbial organism or cell includes at least one exogenous nucleic acid encoding one or more of an enzyme or polypeptide that converts a substrate to a product, or an isomeric form thereof including, for example:
(i) an enzyme or polypeptide that converts succinyl-CoA and acetyl-CoA to 3-oxoadipyl-CoA, wherein the succinyl-CoA and/or the acetyl-CoA can get converted to succinate and/or acetate by-product, respectively, and wherein the succinate and/or acetate gets recycled back into succinyl-CoA and/or the acetyl-CoA, respectively, as described herein;
(ii) an enzyme or polypeptide that converts 3-oxoadipyl-CoA to 3-oxoadipate;
(iii) an enzyme or polypeptide that converts 3-oxoadipate to 3-hydroxyadipate;
(iv) an enzyme or polypeptide that converts 3-hydroxyadipate to 5-carboxy-2-pentenoate; and/or
(v) an enzyme or polypeptide that converts 5-carboxy-2-pentenoate to adipate.

In certain embodiments, the non-naturally occurring microbial organism or cell, having one or more eliminated, substantially eliminated, or reduced carboxylate by-product (e.g., acetate and/or succinate), and/or enhanced or increased carbon flux through one or mor acyl-CoA intermediate or precursor (e.g., acetyl-CoA or succinyl-CoA), can further include 6ACA pathway, wherein the non-naturally occurring microbial organism or cell includes at least one exogenous nucleic acid encoding one or more of an enzyme or polypeptide that converts a substrate to a product, or an isomeric form thereof including, for example:
(i) an enzyme or polypeptide that converts succinyl-CoA and acetyl-CoA to 3-oxoadipyl-CoA, wherein the succinyl-CoA and/or the acetyl-CoA can get converted to succinate and/or acetate by-product, respectively, and wherein the succinate and/or acetate gets recycled back into succinyl-CoA and/or the acetyl-CoA, respectively, as described herein;
(ii) an enzyme or polypeptide that converts 3-oxoadipyl-CoA to 3-hydroxyadipyl-CoA;
(iii) an enzyme or polypeptide that converts 3-hydroxyadipyl-CoA to 5-carboxy-2-pentenoyl-CoA;
(iv) an enzyme or polypeptide that converts 5-carboxy-2-pentenoyl-CoA to adipyl-CoA;
(v) an enzyme or polypeptide that converts adipyl-CoA to adipate semialdehyde; and/or
(vi) an enzyme or polypeptide that converts adipate semialdehyde to 6ACA.

In certain embodiments, the non-naturally occurring microbial organism or cell, having at least one eliminated, substantially eliminated, or reduced carboxylate by-product (e.g., acetate and/or succinate), and/or enhanced or increased carbon flux through at least one acyl-CoA intermediate or precursor (e.g., acetyl-CoA or succinyl-CoA), can further include a 6ACA pathway, wherein the non-naturally occurring microbial organism or cell includes at least one nucleic acid or at least one exogenous nucleic acid encoding one or more of an enzyme or polypeptide that converts a substrate to a product, or an isomeric form thereof including, for example:
(i) an enzyme or polypeptide that converts succinyl-CoA and acetyl-CoA to 3-oxoadipyl-CoA, wherein the succinyl-CoA and/or the acetyl-CoA can get converted to succinate and/or acetate by-product, respectively, and wherein the succinate and/or acetate gets recycled back into succinyl-CoA and/or the acetyl-CoA, respectively, (e.g., by a carboxylate by-product recycling loop) as described herein;
(ii) an enzyme or polypeptide that converts 3-oxoadipyl-CoA to 3-hydroxyadipyl-CoA;
(iii) an enzyme or polypeptide that converts 3-hydroxyadipyl-CoA to 5-carboxy-2-pentenoyl-CoA;
(iv) an enzyme or polypeptide that converts 5-carboxy-2-pentenoyl-CoA to adipyl-CoA;
(v) an enzyme or polypeptide that converts adipyl-CoA to adipate;
(vi) an enzyme or polypeptide that converts adipate to adipate semialdehyde; and/or
(vii) an enzyme or polypeptide that converts adipate semialdehyde to 6ACA.

In certain embodiments, the non-naturally occurring microbial organism or cell, having one or more eliminated, substantially eliminated, or reduced carboxylate by-product (e.g., acetate and/or succinate), and/or enhanced or increased carbon flux through one or more acyl-CoA intermediate or precursor (e.g., acetyl-CoA or succinyl-CoA), can further include a 6ACA pathway, wherein the non-naturally occurring microbial organism or cell includes at least one nucleic acid or at least one exogenous nucleic acid encoding one or more of an enzyme or polypeptide that converts a substrate to a product, or an isomeric form thereof including, for example:
(i) an enzyme or polypeptide that converts succinyl-CoA and acetyl-CoA to 3-oxoadipyl-CoA, wherein the succinyl-CoA and/or the acetyl-CoA can get converted to succinate and/or acetate by-product, respectively, and wherein the succinate and/or acetate gets recycled back into succinyl-CoA and/or the acetyl-CoA, respectively, as described herein;
(ii) an enzyme or polypeptide that converts 3-oxoadipyl-CoA to 3-hydroxyadipyl-CoA;
(iii) an enzyme or polypeptide that converts 3-hydroxyadipyl-CoA to 5-carboxy-2-pentenoyl-CoA;
(iv) an enzyme or polypeptide that converts 5-carboxy-2-pentenoyl-CoA to adipyl-CoA;
(v) an enzyme or polypeptide that converts adipyl-CoA to adipate;
(vi) an enzyme or polypeptide that converts adipate to adiplyphosphate;
(vii) an enzyme or polypeptide that converts adiplyphosphate to adipate semialdehyde; and/or
(viii) an enzyme or polypeptide that converts adipate semialdehyde to 6ACA.

In certain embodiments, the non-naturally occurring microbial organism or cell, having at least one eliminated, substantially eliminated, or reduced carboxylate by-product (e.g., acetate and/or succinate), and/or enhanced or increased carbon flux through at least one acyl-CoA intermediate or precursor (e.g., acetyl-CoA or succinyl-CoA), can further include an 6ACA pathway, wherein the non-naturally occurring microbial organism or cell includes at least one nucleic acid or at least one exogenous nucleic acid encoding one or more of an enzyme or polypeptide that converts a substrate to a product, or an isomeric form thereof including, for example:
(i) an enzyme or polypeptide that converts succinyl-CoA and acetyl-CoA to 3-oxoadipyl-CoA, wherein the succinyl-CoA and/or the acetyl-CoA can get converted to succinate and/or acetate by-product, respectively, and wherein the succinate and/or acetate gets recycled back into succinyl-CoA and/or the acetyl-CoA, respectively, as described herein;
(ii) an enzyme or polypeptide that converts 3-oxoadipyl-CoA to 3-oxoadipate;
(iii) an enzyme or polypeptide that converts 3-oxoadipate to 3-hydroxyadipate;
(iv) an enzyme or polypeptide that converts 3-hydroxyadipate to 5-carboxy-2-pentenoate;
(v) an enzyme or polypeptide that converts 5-carboxy-2-pentenoate to adipate;
(vi) an enzyme or polypeptide that converts adipate to adipate semialdehyde; and/or
(vii) an enzyme or polypeptide that converts adipate semialdehyde to 6ACA.

In certain embodiments, the non-naturally occurring microbial organism or cell, having one or more eliminated, substantially eliminated, or reduced carboxylate by-product (e.g., acetate and/or succinate), and/or enhanced or increased carbon flux through one or more acyl-CoA intermediate or precursor (e.g., acetyl-CoA or succinyl-CoA), can further include an 6ACA pathway, wherein the non-naturally occurring microbial organism or cell includes at least one exogenous nucleic acid encoding one or more of an enzyme or polypeptide that converts a substrate to a product, or an isomeric form thereof including, for example:
(i) an enzyme or polypeptide that converts succinyl-CoA and acetyl-CoA to 3-oxoadipyl-CoA, wherein the succinyl-CoA and/or the acetyl-CoA can get converted to succinate and/or acetate by-product, respectively, and wherein the succinate and/or acetate gets recycled back into succinyl-CoA and/or the acetyl-CoA, respectively, as described herein;
(ii) an enzyme or polypeptide that converts 3-oxoadipyl-CoA to 3-oxoadipate;
(iii) an enzyme or polypeptide that converts 3-oxoadipate to 3-hydroxyadipate;
(iv) an enzyme or polypeptide that converts 3-hydroxyadipate to 5-carboxy-2-pentenoate;
(v) an enzyme or polypeptide that converts 5-carboxy-2-pentenoate to adipate;
(vi) an enzyme or polypeptide that converts adipate to adipylphosphate;
(vii) an enzyme or polypeptide that converts adipylphosphate to adipate semialdehyde; and/or
(viii) an enzyme or polypeptide that converts adipate semialdehyde to 6ACA.

In certain embodiments, the non-naturally occurring microbial organism or cell, having at least one eliminated, substantially eliminated, or reduced carboxylate by-product (e.g., acetate and/or succinate), and/or enhanced or increased carbon flux through at least one acyl-CoA intermediate or precursor (e.g., acetyl-CoA or succinyl-CoA), can further include an 6ACA pathway, wherein the non-naturally occurring microbial organism or cell includes at least one exogenous nucleic acid encoding one or more of an enzyme or polypeptide that converts a substrate to a product, or an isomeric form thereof including, for example:
(i) an enzyme or polypeptide that converts succinyl-CoA and acetyl-CoA to 3-oxoadipyl-CoA, wherein the succinyl-CoA and/or the acetyl-CoA can get converted to succinate and/or acetate by-product, respectively, and wherein the succinate and/or acetate gets recycled back into succinyl-CoA and/or the acetyl-CoA, respectively, as described herein;
(ii) an enzyme or polypeptide that converts 3-oxoadipyl-CoA to 3-oxoadipate;
(iii) an enzyme or polypeptide that converts 3-oxoadipate to 3-hydroxyadipate;
(iv) an enzyme or polypeptide that converts 3-hydroxyadipate to 5-carboxy-2-pentenoate;
(v) an enzyme or polypeptide that converts 5-carboxy-2-pentenoate to adipate;
(vi) an enzyme or polypeptide that converts adipate to adipyl-CoA;
(vii) an enzyme or polypeptide that converts adipyl-CoA to adipate semialdehyde; and/or
(viii) an enzyme or polypeptide that converts adipate semialdehyde to 6ACA.

In certain embodiments, the non-naturally occurring microbial organism or cell, having at least one eliminated, substantially eliminated, or reduced carboxylate by-product (e.g., acetate and/or succinate), and/or enhanced or increased carbon flux through at least one acyl-CoA intermediate or precursor (e.g., acetyl-CoA or succinyl-CoA), can further include CPL pathway, wherein the non-naturally occurring microbial organism or cell includes at least one exogenous nucleic acid encoding one or more of an enzyme or polypeptide that converts a substrate to a product, or an isomeric form thereof including, for example:
(i) an enzyme or polypeptide that converts succinyl-CoA and acetyl-CoA to 3-oxoadipyl-CoA, wherein the succinyl-CoA and/or the acetyl-CoA can get converted to succinate and/or acetate by-product, respectively, and wherein the succinate and/or acetate gets recycled back into succinyl-CoA and/or the acetyl-CoA, respectively, as described herein;
(ii) an enzyme or polypeptide that converts 3-oxoadipyl-CoA to 3-hydroxyadipyl-CoA;
(iii) an enzyme or polypeptide that converts 3-hydroxyadipyl-CoA to 5-carboxy-2-pentenoyl-CoA;
(iv) an enzyme or polypeptide that converts 5-carboxy-2-pentenoyl-CoA to adipyl-CoA;
(v) an enzyme or polypeptide that converts adipyl-CoA to adipate semialdehyde;
(vi) an enzyme or polypeptide that converts adipate semialdehyde to 6ACA; and/or
(vii) an enzyme or polypeptide that converts 6ACA to caprolactam.

In certain embodiments, the non-naturally occurring microbial organism or cell, having at least one eliminated, substantially eliminated, or reduced carboxylate by-product (e.g., acetate and/or succinate), and/or enhanced or increased carbon flux through at least one acyl-CoA intermediate or precursor (e.g., acetyl-CoA or succinyl-CoA), can further include CPL pathway, wherein the non-naturally occurring microbial organism or cell includes at least one nucleic acid or at least one exogenous nucleic acid encoding one or more of an enzyme or polypeptide that converts a substrate to a product, or an isomeric form thereof including, for example:
(i) an enzyme or polypeptide that converts succinyl-CoA and acetyl-CoA to 3-oxoadipyl-CoA, wherein the succinyl-CoA and/or the acetyl-CoA can get converted to succinate and/or acetate by-product, respectively, and wherein the succinate and/or acetate gets recycled back into succinyl-CoA and/or the acetyl-CoA, respectively, (e.g., by a carboxylate by-product recycling loop) as described herein;
(ii) an enzyme or polypeptide that converts 3-oxoadipyl-CoA to 3-hydroxyadipyl-CoA;
(iii) an enzyme or polypeptide that converts 3-hydroxyadipyl-CoA to 5-carboxy-2-pentenoyl-CoA;
(iv) an enzyme or polypeptide that converts 5-carboxy-2-pentenoyl-CoA to adipyl-CoA;
(v) an enzyme or polypeptide that converts adipyl-CoA to adipate semialdehyde;
(vi) an enzyme or polypeptide that converts adipate semialdehyde to 6ACA;
(vii) an enzyme or polypeptide that converts 6ACA to 6-aminocaproyl-CoA; and/or
(viii) an enzyme or polypeptide that converts 6-aminocaproyl-CoA to CPL.

In certain embodiments, the non-naturally occurring microbial organism or cell, having one or more eliminated, substantially eliminated, or reduced carboxylate by-product (e.g., acetate and/or succinate), and/or enhanced or increased carbon flux through one or more acyl-CoA intermediate or precursor (e.g., acetyl-CoA or succinyl-CoA), can further include a CPL pathway, wherein the non-naturally occurring microbial organism or cell includes at least one exogenous nucleic acid encoding one or more of an enzyme or polypeptide that converts a substrate to a product, or an isomeric form thereof including, for example:
(i) an enzyme or polypeptide that converts succinyl-CoA and acetyl-CoA to 3-oxoadipyl-CoA, wherein the succinyl-CoA and/or the acetyl-CoA can get converted to succinate and/or acetate by-product, respectively, and wherein the succinate and/or acetate gets recycled back into succinyl-CoA and/or the acetyl-CoA, respectively, as described herein;
(ii) an enzyme or polypeptide that converts 3-oxoadipyl-CoA to 3-hydroxyadipyl-CoA;
(iii) an enzyme or polypeptide that converts 3-hydroxyadipyl-CoA to 5-carboxy-2-pentenoyl-CoA;
(iv) an enzyme or polypeptide that converts 5-carboxy-2-pentenoyl-CoA to adipyl-CoA;
(v) an enzyme or polypeptide that converts adipyl-CoA to adipate;
(vi) an enzyme or polypeptide that converts adipate to adipate semialdehyde;
(vii) an enzyme or polypeptide that converts adipate semialdehyde to 6ACA; and/or
(viii) an enzyme or polypeptide that converts 6ACA to caprolactam.

In certain embodiments, the non-naturally occurring microbial organism or cell, having one or more eliminated, substantially eliminated, or reduced carboxylate by-product (e.g., acetate and/or succinate), and/or enhanced or increased carbon flux through one or more acyl-CoA intermediate or precursor (e.g., acetyl-CoA or succinyl-CoA), can further include a CPL pathway, wherein the non-naturally occurring microbial organism or cell includes at least one nucleic acid or at least one exogenous nucleic acid encoding one or more of an enzyme or polypeptide that converts a substrate to a product, or an isomeric form thereof including, for example:

(i) an enzyme or polypeptide that converts succinyl-CoA and acetyl-CoA to 3-oxoadipyl-CoA, wherein the succinyl-CoA and/or the acetyl-CoA can get converted to succinate and/or acetate by-product, respectively, and wherein the succinate and/or acetate gets recycled back into succinyl-CoA and/or the acetyl-CoA, respectively, (e.g., by a carboxylate by-product recycling loop) as described herein;
(ii) an enzyme or polypeptide that converts 3-oxoadipyl-CoA to 3-hydroxyadipyl-CoA;
(iii) an enzyme or polypeptide that converts 3-hydroxyadipyl-CoA to 5-carboxy-2-pentenoyl-CoA;
(iv) an enzyme or polypeptide that converts 5-carboxy-2-pentenoyl-CoA to adipyl-CoA;
(v) an enzyme or polypeptide that converts adipyl-CoA to adipate;
(vi) an enzyme or polypeptide that converts adipate to adipate semialdehyde;
(vii) an enzyme or polypeptide that converts adipate semialdehyde to 6ACA;
(viii) an enzyme or polypeptide that converts 6ACA to 6-aminocaproyl-CoA; and/or
(ix) an enzyme or polypeptide that converts 6-aminocaproyl-CoA to CPL.

In certain embodiments, the non-naturally occurring microbial organism or cell, having one or more eliminated, substantially eliminated, or reduced carboxylate by-product (e.g., acetate and/or succinate), and/or enhanced or increased carbon flux through one or more acyl-CoA intermediate or precursor (e.g., acetyl-CoA or succinyl-CoA), can further include a CPL pathway, wherein the non-naturally occurring microbial organism or cell includes at least one nucleic acid or at least one exogenous nucleic acid encoding one or more of an enzyme or polypeptide that converts a substrate to a product, or an isomeric form thereof including, for example:
(i) an enzyme or polypeptide that converts succinyl-CoA and acetyl-CoA to 3-oxoadipyl-CoA, wherein the succinyl-CoA and/or the acetyl-CoA can get converted to succinate and/or acetate by-product, respectively, and wherein the succinate and/or acetate gets recycled back into succinyl-CoA and/or the acetyl-CoA, respectively, as described herein;
(ii) an enzyme or polypeptide that converts 3-oxoadipyl-CoA to 3-hydroxyadipyl-CoA;
(iii) an enzyme or polypeptide that converts 3-hydroxyadipyl-CoA to 5-carboxy-2-pentenoyl-CoA;
(iv) an enzyme or polypeptide that converts 5-carboxy-2-pentenoyl-CoA to adipyl-CoA;
(v) an enzyme or polypeptide that converts adipyl-CoA to adipate;
(vi) an enzyme or polypeptide that converts adipate to adiplyphosphate;
(vii) an enzyme or polypeptide that converts adiplyphosphate to adipate semialdehyde; and/or
(viii) an enzyme or polypeptide that converts adipate semialdehyde to 6ACA; and/or
(ix) an enzyme or polypeptide that converts 6ACA to CPL.

In certain embodiments, the non-naturally occurring microbial organism or cell, having one or more eliminated, substantially eliminated, or reduced carboxylate by-product (e.g., acetate and/or succinate), and/or enhanced or increased carbon flux through one or more acyl-CoA intermediate or precursor (e.g., acetyl-CoA or succinyl-CoA), can further include a CPL pathway, wherein the non-naturally occurring microbial organism or cell includes at least one nucleic acid or at least one exogenous nucleic acid encoding one or more of an enzyme or polypeptide that converts a substrate to a product, or an isomeric form thereof including, for example:
(i) an enzyme or polypeptide that converts succinyl-CoA and acetyl-CoA to 3-oxoadipyl-CoA, wherein the succinyl-CoA and/or the acetyl-CoA can get converted to succinate and/or acetate by-product, respectively, and wherein the succinate and/or acetate gets recycled back into succinyl-CoA and/or the acetyl-CoA, respectively, as described herein;
(ii) an enzyme or polypeptide that converts 3-oxoadipyl-CoA to 3-hydroxyadipyl-CoA;
(iii) an enzyme or polypeptide that converts 3-hydroxyadipyl-CoA to 5-carboxy-2-pentenoyl-CoA;
(iv) an enzyme or polypeptide that converts 5-carboxy-2-pentenoyl-CoA to adipyl-CoA;
(v) an enzyme or polypeptide that converts adipyl-CoA to adipate;
(vi) an enzyme or polypeptide that converts adipate to adiplyphosphate;
(vii) an enzyme or polypeptide that converts adiplyphosphate to adipate semialdehyde; and/or
(viii) an enzyme or polypeptide that converts adipate semialdehyde to 6ACA;
(ix) an enzyme or polypeptide that converts 6ACA to 6-aminocaproyl-CoA; and/or
(x) an enzyme or polypeptide that converts 6-aminocaproyl-CoA to CPL.

In certain embodiments, the non-naturally occurring microbial organism or cell, having one or more eliminated, substantially eliminated, or reduced carboxylate by-product (e.g., acetate and/or succinate), and/or enhanced or increased carbon flux through one or more acyl-CoA intermediate or precursor (e.g., acetyl-CoA or succinyl-CoA), can further include an CPL pathway, wherein the non-naturally occurring microbial organism or cell includes at least one nucleic acid or at least one exogenous nucleic acid encoding one or more of an enzyme or polypeptide that converts a substrate to a product, or an isomeric form thereof including, for example:
(i) an enzyme or polypeptide that converts succinyl-CoA and acetyl-CoA to 3-oxoadipyl-CoA, wherein the succinyl-CoA and/or the acetyl-CoA can get converted to succinate and/or acetate by-product, respectively, and wherein the succinate and/or acetate gets recycled back into succinyl-CoA and/or the acetyl-CoA, respectively, as described herein;
(ii) an enzyme or polypeptide that converts 3-oxoadipyl-CoA to 3-oxoadipate;
(iii) an enzyme or polypeptide that converts 3-oxoadipate to 3-hydroxyadipate;
(iv) an enzyme or polypeptide that converts 3-hydroxyadipate to 5-carboxy-2-pentenoate;
(v) an enzyme or polypeptide that converts 5-carboxy-2-pentenoate to adipate;
(vi) an enzyme or polypeptide that converts adipate to adipate semialdehyde;
(vii) an enzyme or polypeptide that converts adipate semialdehyde to 6ACA; and/or
(viii) an enzyme or polypeptide that converts 6ACA to CPL.

In certain embodiments, the non-naturally occurring microbial organism or cell, having one or more eliminated, substantially eliminated, or reduced carboxylate by-product (e.g., acetate and/or succinate), and/or enhanced or increased carbon flux through one or more acyl-CoA intermediate or precursor (e.g., acetyl-CoA or succinyl-CoA), can further include an CPL pathway, wherein the non-naturally occurring microbial organism or cell includes at least one nucleic acid or at least one exogenous nucleic acid encoding one or more of an enzyme or polypeptide that converts a substrate to a product, or an isomeric form thereof including, for example:
(i) an enzyme or polypeptide that converts succinyl-CoA and acetyl-CoA to 3-oxoadipyl-CoA, wherein the succinyl-CoA and/or the acetyl-CoA can get converted to succinate and/or acetate by-product, respectively, and wherein the succinate and/or acetate gets recycled back into succinyl-CoA and/or the acetyl-CoA, respectively, as described herein;
(ii) an enzyme or polypeptide that converts 3-oxoadipyl-CoA to 3-oxoadipate;
(iii) an enzyme or polypeptide that converts 3-oxoadipate to 3-hydroxyadipate;
(iv) an enzyme or polypeptide that converts 3-hydroxyadipate to 5-carboxy-2-pentenoate;
(v) an enzyme or polypeptide that converts 5-carboxy-2-pentenoate to adipate;
(vi) an enzyme or polypeptide that converts adipate to adipate semialdehyde;
(vii) an enzyme or polypeptide that converts adipate semialdehyde to 6ACA;
(viii) an enzyme or polypeptide that converts 6ACA to 6-aminocaproyl-CoA; and/or
(ix) an enzyme or polypeptide that converts 6-aminocaproyl-CoA to CPL.

In certain embodiments, the non-naturally occurring microbial organism or cell, having one or more eliminated, substantially eliminated, or reduced carboxylate by-product (e.g., acetate and/or succinate), and/or enhanced or increased carbon flux through one or more acyl-CoA intermediate or precursor (e.g., acetyl-CoA or succinyl-CoA), can further include an CPL pathway, wherein the non-naturally occurring microbial organism or cell includes at least one nucleic acid or at least one exogenous nucleic acid encoding one or more of an enzyme or polypeptide that converts a substrate to a product, or an isomeric form thereof including, for example:
(i) an enzyme or polypeptide that converts succinyl-CoA and acetyl-CoA to 3-oxoadipyl-CoA, wherein the succinyl-CoA and/or the acetyl-CoA can get converted to succinate and/or acetate by-product, respectively, and wherein the succinate and/or acetate gets recycled back into succinyl-CoA and/or the acetyl-CoA, respectively, as described herein;
(ii) an enzyme or polypeptide that converts 3-oxoadipyl-CoA to 3-oxoadipate;
(iii) an enzyme or polypeptide that converts 3-oxoadipate to 3-hydroxyadipate;
(iv) an enzyme or polypeptide that converts 3-hydroxyadipate to 5-carboxy-2-pentenoate;
(v) an enzyme or polypeptide that converts 5-carboxy-2-pentenoate to adipate;
(vi) an enzyme or polypeptide that converts adipate to adipylphosphate;
(vii) an enzyme or polypeptide that converts adipylphosphate to adipate semialdehyde;
(viii) an enzyme or polypeptide that converts adipate semialdehyde to 6ACA; and/or
(ix) an enzyme or polypeptide that converts 6ACA to CPL.

In certain embodiments, the non-naturally occurring microbial organism or cell, having one or more eliminated, substantially eliminated, or reduced carboxylate by-product (e.g., acetate and/or succinate), and/or enhanced or increased carbon flux through one or more acyl-CoA intermediate or precursor (e.g., acetyl-CoA or succinyl-CoA), can further include an CPL pathway, wherein the non-naturally occurring microbial organism or cell includes at least one exogenous nucleic acid encoding one or more of an enzyme or polypeptide that converts a substrate to a product, or an isomeric form thereof including, for example:
(i) an enzyme or polypeptide that converts succinyl-CoA and acetyl-CoA to 3-oxoadipyl-CoA, wherein the succinyl-CoA and/or the acetyl-CoA can get converted to succinate and/or acetate by-product, respectively, and wherein the succinate and/or acetate gets recycled back into succinyl-CoA and/or the acetyl-CoA, respectively, as described herein;
(ii) an enzyme or polypeptide that converts 3-oxoadipyl-CoA to 3-oxoadipate;
(iii) an enzyme or polypeptide that converts 3-oxoadipate to 3-hydroxyadipate;
(iv) an enzyme or polypeptide that converts 3-hydroxyadipate to 5-carboxy-2-pentenoate;
(v) an enzyme or polypeptide that converts 5-carboxy-2-pentenoate to adipate;
(vi) an enzyme or polypeptide that converts adipate to adipylphosphate;
(vii) an enzyme or polypeptide that converts adipylphosphate to adipate semialdehyde;
(viii) an enzyme or polypeptide that converts adipate semialdehyde to 6ACA;
(ix) an enzyme or polypeptide that converts 6ACA to 6-aminocaproyl-CoA; and/or
(x) an enzyme or polypeptide that converts 6-aminocaproyl-CoA to CPL.

In certain embodiments, the non-naturally occurring microbial organism or cell, having one or more eliminated, substantially eliminated, or reduced carboxylate by-product (e.g., acetate and/or succinate), and/or enhanced or increased carbon flux through one or more acyl-CoA intermediate or precursor (e.g., acetyl-CoA or succinyl-CoA), can further include an CPL pathway, wherein the non-naturally occurring microbial organism or cell includes at least one nucleic acid or at least one exogenous nucleic acid encoding one or more of an enzyme or polypeptide that converts a substrate to a product, or an isomeric form thereof including, for example:
(i) an enzyme or polypeptide that converts succinyl-CoA and acetyl-CoA to 3-oxoadipyl-CoA, wherein the succinyl-CoA and/or the acetyl-CoA can get converted to succinate and/or acetate by-product, respectively, and wherein the succinate and/or acetate gets recycled back into succinyl-CoA and/or the acetyl-CoA, respectively, as described herein;
(ii) an enzyme or polypeptide that converts 3-oxoadipyl-CoA to 3-oxoadipate;
(iii) an enzyme or polypeptide that converts 3-oxoadipate to 3-hydroxyadipate;
(iv) an enzyme or polypeptide that converts 3-hydroxyadipate to 5-carboxy-2-pentenoate;
(v) an enzyme or polypeptide that converts 5-carboxy-2-pentenoate to adipate;
(vi) an enzyme or polypeptide that converts adipate to adipyl-CoA;
(vii) an enzyme or polypeptide that converts adipyl-CoA to adipate semialdehyde;
(viii) an enzyme or polypeptide that converts adipate semialdehyde to 6ACA; and/or
(ix) an enzyme or polypeptide that converts 6ACA to CPL.

In certain embodiments, the non-naturally occurring microbial organism or cell, having one or more eliminated, substantially eliminated, or reduced carboxylate by-product (e.g., acetate and/or succinate), and/or enhanced or increased carbon flux through one or more acyl-CoA intermediate or precursor (e.g., acetyl-CoA or succinyl-CoA), can further include an CPL pathway, wherein the non-naturally occurring microbial organism or cell includes at least one nucleic acid or at least one exogenous nucleic acid encoding one or more of an enzyme or polypeptide that converts a substrate to a product, or an isomeric form thereof including, for example:
(i) an enzyme or polypeptide that converts succinyl-CoA and acetyl-CoA to 3-oxoadipyl-CoA, wherein the succinyl-CoA and/or the acetyl-CoA can get converted to succinate and/or acetate by-product, respectively, and wherein the succinate and/or acetate gets recycled back into succinyl-CoA and/or the acetyl-CoA, respectively, as described herein;
(ii) an enzyme or polypeptide that converts 3-oxoadipyl-CoA to 3-oxoadipate;
(iii) an enzyme or polypeptide that converts 3-oxoadipate to 3-hydroxyadipate;
(iv) an enzyme or polypeptide that converts 3-hydroxyadipate to 5-carboxy-2-pentenoate;
(v) an enzyme or polypeptide that converts 5-carboxy-2-pentenoate to adipate;
(vi) an enzyme or polypeptide that converts adipate to adipyl-CoA;
(vii) an enzyme or polypeptide that converts adipyl-CoA to adipate semialdehyde;
(viii) an enzyme or polypeptide that converts adipate semialdehyde to 6ACA;
(ix) an enzyme or polypeptide that converts 6ACA to 6-aminocaproyl-CoA; and/or
(x) an enzyme or polypeptide that converts 6-aminocaproyl-CoA to CPL.

In some embodiments, the 6ACA is produced by any of the pathways described herein (e.g., in FIG. 16), and then is isolated, collected, harvested, and/or purified from the culture medium or fermentation broth and/or from the non-naturally occurring microbial organism, and is converted to CPL by a chemical or thermal (e.g., heating) conversion step. Such conversion step is known and described in the art.

In certain embodiments, the non-naturally occurring microbial organism or cell, having one or more eliminated, substantially eliminated, or reduced carboxylate by-product (e.g., acetate and/or succinate), and/or enhanced or increased carbon flux through one or more acyl-CoA intermediate or precursor (e.g., acetyl-CoA or succinyl-CoA), can further include HMD pathway, wherein the non-naturally occurring microbial organism or cell includes at least one nucleic acid or at least one exogenous nucleic acid encoding one or more of an enzyme or polypeptide that converts a substrate to a product, or an isomeric form thereof including, for example:
(i) an enzyme or polypeptide that converts succinyl-CoA and acetyl-CoA to 3-oxoadipyl-CoA, wherein the succinyl-CoA and/or the acetyl-CoA can get converted to succinate and/or acetate by-product, respectively, and wherein the succinate and/or acetate gets recycled back into succinyl-CoA and/or the acetyl-CoA, respectively, as described herein;
(ii) an enzyme or polypeptide that converts 3-oxoadipyl-CoA to 3-hydroxyadipyl-CoA;
(iii) an enzyme or polypeptide that converts 3-hydroxyadipyl-CoA to 5-carboxy-2-pentenoyl-CoA;
(iv) an enzyme or polypeptide that converts 5-carboxy-2-pentenoyl-CoA to adipyl-CoA;
(v) an enzyme or polypeptide that converts adipyl-CoA to adipate semialdehyde;
(vi) an enzyme or polypeptide that converts adipate semialdehyde to 6ACA;
(vii) an enzyme or polypeptide that converts 6ACA to 6-aminocaproyl-CoA;
(viii) an enzyme or polypeptide that converts 6-aminocaproyl-CoA to 6-aminocaproate semialdehyde; and/or
(ix) an enzyme or polypeptide that converts 6-aminocaproate semialdehyde to HMD.

In certain embodiments, the non-naturally occurring microbial organism or cell, having one or more eliminated, substantially eliminated, or reduced carboxylate by-product (e.g., acetate and/or succinate), and/or enhanced or increased carbon flux through one or more acyl-CoA intermediate or precursor (e.g., acetyl-CoA or succinyl-CoA), can further include a HMD pathway, wherein the non-naturally occurring microbial organism or cell includes at least one nucleic acid or at least one exogenous nucleic acid encoding one or more of an enzyme or polypeptide that converts a substrate to a product, or an isomeric form thereof including, for example:
(i) an enzyme or polypeptide that converts succinyl-CoA and acetyl-CoA to 3-oxoadipyl-CoA, wherein the succinyl-CoA and/or the acetyl-CoA can get converted to succinate and/or acetate by-product, respectively, and wherein the succinate and/or acetate gets recycled back into succinyl-CoA and/or the acetyl-CoA, respectively, as described herein;
(ii) an enzyme or polypeptide that converts 3-oxoadipyl-CoA to 3-hydroxyadipyl-CoA;
(iii) an enzyme or polypeptide that converts 3-hydroxyadipyl-CoA to 5-carboxy-2-pentenoyl-CoA;
(iv) an enzyme or polypeptide that converts 5-carboxy-2-pentenoyl-CoA to adipyl-CoA;
(v) an enzyme or polypeptide that converts adipyl-CoA to adipate;
(vi) an enzyme or polypeptide that converts adipate to adipate semialdehyde;
(vii) an enzyme or polypeptide that converts adipate semialdehyde to 6ACA;
(viii) an enzyme or polypeptide that converts 6ACA to 6-aminocaproyl-CoA;
(ix) an enzyme or polypeptide that converts 6-aminocaproyl-CoA to 6-aminocaproate semialdehyde; and/or
(x) an enzyme or polypeptide that converts 6-aminocaproate semialdehyde to HMD.

In certain embodiments, the non-naturally occurring microbial organism or cell, having one or more eliminated, substantially eliminated, or reduced carboxylate by-product (e.g., acetate and/or succinate), and/or enhanced or increased carbon flux through one or more acyl-CoA intermediate or precursor (e.g., acetyl-CoA or succinyl-CoA), can further include a HMD pathway, wherein the non-naturally occurring microbial organism or cell includes at least one nucleic acid or at least one exogenous nucleic acid encoding one or more of an enzyme or polypeptide that converts a substrate to a product, or an isomeric form thereof including, for example:
(i) an enzyme or polypeptide that converts succinyl-CoA and acetyl-CoA to 3-oxoadipyl-CoA, wherein the succinyl-CoA and/or the acetyl-CoA can get converted to succinate and/or acetate by-product, respectively, and wherein the succinate and/or acetate gets recycled back into succinyl-CoA and/or the acetyl-CoA, respectively, as described herein;
(ii) an enzyme or polypeptide that converts 3-oxoadipyl-CoA to 3-hydroxyadipyl-CoA;
(iii) an enzyme or polypeptide that converts 3-hydroxyadipyl-CoA to 5-carboxy-2-pentenoyl-CoA;
(iv) an enzyme or polypeptide that converts 5-carboxy-2-pentenoyl-CoA to adipyl-CoA;
(v) an enzyme or polypeptide that converts adipyl-CoA to adipate;
(vi) an enzyme or polypeptide that converts adipate to adiplyphosphate;
(vii) an enzyme or polypeptide that converts adiplyphosphate to adipate semialdehyde;
(viii) an enzyme or polypeptide that converts adipate semialdehyde to 6ACA
(ix) an enzyme or polypeptide that converts 6ACA to 6-aminocaproyl-CoA;
(x) an enzyme or polypeptide that converts 6-aminocaproyl-CoA to 6-aminocaproate semialdehyde; and/or
(xi) an enzyme or polypeptide that converts 6-aminocaproate semialdehyde to HMD.

In certain embodiments, the non-naturally occurring microbial organism or cell, having one or more eliminated, substantially eliminated, or reduced carboxylate by-product (e.g., acetate and/or succinate), and/or enhanced or increased carbon flux through one or more acyl-CoA intermediate or precursor (e.g., acetyl-CoA or succinyl-CoA), can further include an HMD pathway, wherein the non-naturally occurring microbial organism or cell includes at least one nucleic acid or at least one exogenous nucleic acid encoding one or more of an enzyme or polypeptide that converts a substrate to a product, or an isomeric form thereof including, for example:
(i) an enzyme or polypeptide that converts succinyl-CoA and acetyl-CoA to 3-oxoadipyl-CoA, wherein the succinyl-CoA and/or the acetyl-CoA can get converted to succinate and/or acetate by-product, respectively, and wherein the succinate and/or acetate gets recycled back into succinyl-CoA and/or the acetyl-CoA, respectively, as described herein;
(ii) an enzyme or polypeptide that converts 3-oxoadipyl-CoA to 3-oxoadipate;
(iii) an enzyme or polypeptide that converts 3-oxoadipate to 3-hydroxyadipate;
(iv) an enzyme or polypeptide that converts 3-hydroxyadipate to 5-carboxy-2-pentenoate;
(v) an enzyme or polypeptide that converts 5-carboxy-2-pentenoate to adipate;
(vi) an enzyme or polypeptide that converts adipate to adipate semialdehyde;
(vii) an enzyme or polypeptide that converts adipate semialdehyde to 6ACA;
(viii) an enzyme or polypeptide that converts 6ACA to 6-aminocaproyl-CoA;
(ix) an enzyme or polypeptide that converts 6-aminocaproyl-CoA to 6-aminocaproate semialdehyde; and/or
(x) an enzyme or polypeptide that converts 6-aminocaproate semialdehyde to HMD.

In certain embodiments, the non-naturally occurring microbial organism or cell, having one or more eliminated, substantially eliminated, or reduced carboxylate by-product (e.g., acetate and/or succinate), and/or enhanced or increased carbon flux through one or more acyl-CoA intermediate or precursor (e.g., acetyl-CoA or succinyl-CoA), can further include an HMD pathway, wherein the non-naturally occurring microbial organism or cell includes at least one exogenous nucleic acid encoding one or more of an enzyme or polypeptide that converts a substrate to a product, or an isomeric form thereof including, for example:
(i) an enzyme or polypeptide that converts succinyl-CoA and acetyl-CoA to 3-oxoadipyl-CoA, wherein the succinyl-CoA and/or the acetyl-CoA can get converted to succinate and/or acetate by-product, respectively, and wherein the succinate and/or acetate gets recycled back into succinyl-CoA and/or the acetyl-CoA, respectively, as described herein;
(ii) an enzyme or polypeptide that converts 3-oxoadipyl-CoA to 3-oxoadipate;
(iii) an enzyme or polypeptide that converts 3-oxoadipate to 3-hydroxyadipate;
(iv) an enzyme or polypeptide that converts 3-hydroxyadipate to 5-carboxy-2-pentenoate;
(v) an enzyme or polypeptide that converts 5-carboxy-2-pentenoate to adipate;
(vi) an enzyme or polypeptide that converts adipate to adipylphosphate;
(vii) an enzyme or polypeptide that converts adipylphosphate to adipate semialdehyde;
(viii) an enzyme or polypeptide that converts adipate semialdehyde to 6ACA;
(ix) an enzyme or polypeptide that converts 6ACA to 6-aminocaproyl-CoA;
(x) an enzyme or polypeptide that converts 6-aminocaproyl-CoA to 6-aminocaproate semialdehyde; and/or
(xi) an enzyme or polypeptide that converts 6-aminocaproate semialdehyde to HMD.

In certain embodiments, the non-naturally occurring microbial organism or cell, having one or more eliminated, substantially eliminated, or reduced carboxylate by-product (e.g., acetate and/or succinate), and/or enhanced or increased carbon flux through one or more acyl-CoA intermediate or precursor (e.g., acetyl-CoA or succinyl-CoA), can further include an HMD pathway, wherein the non-naturally occurring microbial organism or cell includes at least one exogenous nucleic acid encoding one or more of an enzyme or polypeptide that converts a substrate to a product, or an isomeric form thereof including, for example:
(i) an enzyme or polypeptide that converts succinyl-CoA and acetyl-CoA to 3-oxoadipyl-CoA, wherein the succinyl-CoA and/or the acetyl-CoA can get converted to succinate and/or acetate by-product, respectively, and wherein the succinate and/or acetate gets recycled back into succinyl-CoA and/or the acetyl-CoA, respectively, as described herein;
(ii) an enzyme or polypeptide that converts 3-oxoadipyl-CoA to 3-oxoadipate;
(iii) an enzyme or polypeptide that converts 3-oxoadipate to 3-hydroxyadipate;
(iv) an enzyme or polypeptide that converts 3-hydroxyadipate to 5-carboxy-2-pentenoate;
(v) an enzyme or polypeptide that converts 5-carboxy-2-pentenoate to adipate;
(vi) an enzyme or polypeptide that converts adipate to adipyl-CoA;
(vii) an enzyme or polypeptide that converts adipyl-CoA to adipate semialdehyde;
(viii) an enzyme or polypeptide that converts adipate semialdehyde to 6ACA
(ix) an enzyme or polypeptide that converts 6ACA to 6-aminocaproyl-CoA;
(x) an enzyme or polypeptide that converts 6-aminocaproyl-CoA to 6-aminocaproate semialdehyde; and/or
(xi) an enzyme or polypeptide that converts 6-aminocaproate semialdehyde to HMD.

In some embodiments, succinyl-CoA and acetyl-CoA is converted to 3-oxoadipyl-CoA by an enzyme or polypeptide having a succinyl-CoA: acetyl-CoA transferase (EC 2.3.1.-) or thiolase activity. In some embodiments, 3-oxoadipyl-CoA is converted to 3-hydroxyadipyl-CoA by an enzyme or polypeptide having a 3-hydroxyacyl-CoA dehydrogenase (EC 1.1.3.-) activity. In some embodiments, 3-hydroxyadipyl-CoA is converted to 5-carboxy-2-pentenoyl-CoA by an enzyme or polypeptide having a 3-hydroxyadipyl-CoA dehydratase (EC 4.2.1.-) activity. In some embodiments, 5-carboxy-2-pentenoyl-CoA is converted to adipyl-CoA by an enzyme or polypeptide having a 5-carboxy-2-pentenoyl-CoA reductase (EC 1.3.1.-) activity. In some embodiments, adipyl-CoA is converted to adipate by an enzyme or polypeptide having a adipyl-CoA/acetyl-CoA transferase, adipyl-CoA synthase, adipyl-CoA hydrolase (EC 2.3.1.-, EC 6.2.1.-, EC 3.1.2.-) activity. In some embodiments, 3-oxoadipyl-CoA is converted to 3-oxoadipate by an enzyme or polypeptide having a 3-oxoadipyl-CoA/acetyl-CoA transferase, 3-oxoadipyl-CoA synthase, or 3-oxoadipyl-CoA hydrolase activity. In some embodiments, 3-oxoadipate is converted to 3-hydroxyadipate by an enzyme or polypeptide having a 3-oxoadipate reductase (EC 1.1.1-) activity. In some embodiments, 3-hydroxyadipate is converted to 5-carboxy-2-pentenoate by an enzyme or polypeptide having a 3-hydroxyadipate activity. In some embodiments, 5-carboxy-2-pentenoate is converted to adipate by an enzyme or polypeptide having a 5-carboxy-2-pentenoate reductase activity. In some embodiments, adipyl-CoA is converted to adipate semialdehyde by an enzyme or polypeptide having adipyl-CoA reductase (aldehyde forming) (EC 1.2.1.-) or 6-ACA-aldehyde dehydrogenase (ALD), activity. In some embodiments, adipate semialdehyde is converted to 6ACA by an enzyme or polypeptide having a 6-aminocaproate transaminase, 6-aminocaproate dehydrogenase activity. In some embodiments, 6ACA is converted to 6-aminocaproate by an enzyme or polypeptide having a 6-aminocaproyl-CoA/acyl-CoA transferase, 6-aminocaproyl-CoA synthase, or ACL (EC 6.2.1-) activity. In some embodiments, 6-aminocaproate is converted to CPL by an enzyme or polypeptide having a amidohydrolase (EC 3.5.1.-) activity. In some embodiments, 6-aminocaproyl-CoA is converted to CPL by an enzyme or polypeptide having a spontaneous cyclization activity. In some embodiments, 6-aminocaproyl-CoA is converted to 6-aminocaproate semialdehyde by an enzyme or polypeptide having a 6-aminocaproyl-CoA reductase (aldehyde forming) or ALDH (EC 1.1.1-) activity. In some embodiments, 6-aminocaproate semialdehyde is converted to HMD by an enzyme or polypeptide having a HMDA transaminase or HMDA dehydrogenase activity. In some embodiments, adipate is converted to adipate semialdehyde by an enzyme or polypeptide having an adipate reductase activity. In some embodiments, adipate is converted to adipylphosphate by an enzyme or polypeptide having an adipate kinase (EC 2.7.2.-) activity.

In certain embodiments, an HMD pathway, and/or the pathway of an isomer thereof, can include one or more of the following enzymes or activity: succinyl-CoA: acetyl-CoA transferase; thiolase, 3-hydroxyacyl-CoA dehydrogenase; 3-hydroxyadipyl-CoA dehydratase; 5-carboxy-2-pentenoyl-CoA reductase; adipyl-CoA/acetyl-CoA transferase, adipyl-CoA synthase, adipyl-CoA hydrolase; 3-oxoadipyl-CoA/acetyl-CoA transferase, 3-oxoadipyl-CoA synthase, 3-oxoadipyl-CoA hydrolase; 3-oxoadipate reductase; 3-hydroxyadipate dehydratase; 5-carboxy-2-pentenoate reductase; adipyl-CoA reductase (aldehyde forming); 6-aminocaproate transaminase, 6-aminocaproate dehydrogenase; 6-aminocaproyl-CoA/acyl-CoA transferase, 6-aminocaproyl-CoA synthase, or ACL; amidohydrolase; spontaneous cyclization (or chemical or thermal conversion); 6-aminocaproyl-CoA reductase (aldehyde forming) or ALDH; PPTase; HMDA transaminase or HMDA dehydrogenase; adipate reductase; adipate kinase; and adipylphosphate reductase.

The non-naturally occurring microbial organism can include at least one exogenous nucleic acid encoding an adipate, 6ACA, CPL, HMD, and/or HDO pathway enzyme. The non-naturally occurring microbial organism can include two exogenous nucleic acids each encoding an adipate, 6ACA, CPL, HMD, and/or HDO pathway enzyme, in other embodiments, three exogenous nucleic acids each encoding an adipate, 6ACA, CPL, HMD, and/or HDO pathway enzyme, in other embodiments, four exogenous nucleic acids each encoding an adipate, 6ACA, CPL, HMD, and/or HDO pathway enzyme, in other embodiments, five exogenous nucleic acids each encoding an adipate, 6ACA, CPL, HMD, and/or HDO pathway enzyme, in other embodiments, six exogenous nucleic acids each encoding an adipate, 6ACA, CPL, HMD, and/or HDO pathway enzyme, in other embodiments, seven exogenous nucleic acids each encoding an adipate, 6ACA, CPL, HMD, and/or HDO pathway enzyme, in other embodiments, eight or more exogenous nucleic acids each encoding an adipate, 6ACA, CPL, HMD, and/or HDO pathway enzyme, in other embodiments, 9 or more exogenous nucleic acids each encoding an adipate, 6ACA, CPL, HMD, and/or HDO pathway enzyme, in other embodiments, 10 or more exogenous nucleic acids each encoding an adipate, 6ACA, CPL, HMD, and/or HDO pathway enzyme, in other embodiments, 11 or more exogenous nucleic acids each encoding an adipate, 6ACA, CPL, HMD, and/or HDO pathway enzyme, in other embodiments, 12 or more exogenous nucleic acids each encoding an adipate, 6ACA, CPL, HMD, and/or HDO pathway enzyme, in other embodiments, 13 or more exogenous nucleic acids each encoding an adipate, 6ACA, CPL, HMD, and/or HDO pathway enzyme, in other embodiments, 14 or more exogenous nucleic acids each encoding an adipate, 6ACA, CPL, HMD, and/or HDO pathway enzyme, in other embodiments, 15 or more exogenous nucleic acids each encoding an adipate, 6ACA, CPL, and/or HDO pathway enzyme, in other embodiments, 16 or more exogenous nucleic acids each encoding an adipate, 6ACA, CPL, and/or HDO pathway enzyme, in other embodiments, 17 or more exogenous nucleic acids each encoding an adipate, 6ACA, CPL, HMD, and/or HDO pathway enzyme, in other embodiments, 18 or more exogenous nucleic acids each encoding an adipate, 6ACA, CPL, HMD, and/or HDO pathway enzyme.

Any number of nucleic acids encoding these enzymes or polypeptides can be further introduced into a host microbial organism or cell already including one, two, three, four, five, six, seven, eight, nine, 10, 11, 12, 13, 14, 15, 16, 17, 18, or 19 nucleic acids that encode any one or more of the other enzymes or polypeptides in the adipate, 6ACA, CPL, HMD, and/or HDO pathway, and/or the pathway of an isomer thereof, as described herein. Where more than one exogenous nucleic acids are introduced, such nucleic acids can be any permutation of the multiple nucleic acids (e.g., polycistronic or multiple nucleic acids) already included in the host organism or cell.

One skilled in the art will understand that these adipate, 6ACA, CPL, HMD, and/or HDO pathways, and/or the pathways of an isomer thereof, substrate-product pairs, and encoded enzymes or polypeptides are merely exemplary, and that any of the substrate-product pairs and/or encoded enzymes or polypeptides disclosed herein that are suitable to produce a desired acetyl-CoA derived product, such as adipate, 6ACA, CPL, HMD, and/or HDO, and/or an isomer thereof, and for which an appropriate activity is available for the conversion of the substrate to the product, can be readily determined by one skilled in the art based on the teachings herein.

Any of the at least one exogenous nucleic acids can be a heterologous nucleic acid, and the non-naturally occurring microbial organism can be constructed for growth or culturing under substantially anaerobic conditions, for example, in a substantially anaerobic culture medium or in microaerobic or substantially microaerobic conditions or culture mediums. Other culture conditions, including feedstock, are described in further detail herein.

Thus, provided herein is a non-naturally occurring microbial organism or cell having one or more eliminated, substantially eliminated, or reduced carboxylate by-product (e.g., acetate and/or succinate), and/or enhanced or increased carbon flux through one or more acyl-CoA (particularly a short-chain acyl-CoA, e.g., acetyl-CoA and/or succinyl-CoA), and containing at least one exogenous nucleic acid encoding one or more enzymes or polypeptides, where the one or more enzymes or polypeptides are part of an adipate, 6ACA, CPL, HMD, and/or HDO pathway, and/or the pathway of an isomer thereof, converting acetyl-CoA or succinyl-CoA to adipate, 6ACA, CPL, HMD, and/or HDO, and/or an isomer thereof, as exemplified by the pathways shown in FIG. 15 and/or FIG. 16. In any of the acetyl-CoA derived product pathways described herein, including the biosynthetic pathways for 1,3-BDO, (3R)-hydroxybutyl (3R)-hydroxybutyrate, MAA, methacrylic acid esters (e.g., MMA or EMA), butyrate, butanol, propane, formate, isopropanol, hexamethylenediamine (HMDA or HMD), caprolactam (CPL), adipate (or adipic acid), 6-aminocaproic acid (6-ACA), or 1,6-hexanediol (HDO), the pathways can include one or more acyl-CoA intermediates and/or precursors, including, for example, 3HB—CoA, (R)-3HB—CoA, (S)-3HB—CoA, acetyl-CoA, acetoacetyl-CoA, succinyl-CoA, crotonyl-CoA, butyryl-CoA, isobutyryl-CoA, malonyl-CoA, and/or hexanoyl-CoA. The spontaneous or enzyme catalyzed hydrolysis of such acyl-CoA intermediates or precursors results in the formation of the corresponding carboxylate by-products, i.e., 3HB, (R)-3HB, (S)-3HB, acetate, acetoacetate, succinate, crotonate, butyrate, isobutyrate, malonate, and/or hexanoate. As described herein, the ACS (e.g., the AMP-forming ACS) can catalyze the conversion or the recycling of the carboxylate by-products back to their corresponding acyl-CoA intermediates or precursors. Any one or more of the acetyl-CoA derived product pathways described herein, or known in the art, that contain acyl-CoAs of short-chain carboxylates as precursors or intermediates, can thus be modified or engineered to include one or more carboxylate by-product recycling loops, to convert any carboxylate by-products that are generated back to their corresponding acyl-CoAs, thus, reducing, eliminating, or substantially eliminating the production of carboxylate by-products, and/or increasing or enhancing the carbon flux through the acyl-CoA intermediates or precursors, and/or increasing the production, titer, yield, and/or productivity of one or more acetyl-CoA derived products. For example, any one or more of the acetyl-CoA derived product pathways described herein can include, or can be modified or engineered to include, the expression of an ACS or a homolog or variant thereof, and/or the expression of a 3HB to 3HBAld conversion step (i.e., the expression of a CAR or a variant or homolog thereof).

Thus, also provided herein is a non-naturally occurring microbial organism, having eliminated, substantially eliminated, or reduced carboxylate by-product(s), and/or enhanced or increased carbon flux through one or more acyl-CoA intermediates or precursors, and containing at least one exogenous nucleic acid encoding one or more of an enzyme or polypeptide, where the enzyme(s) or polypeptide(s) are included in or are a part of a 1,3-BDO, (R)-1,3-BDO, (S)-1,3-BDO, MAA, MAA ester (e.g., MMA, EMA, and others), butyrate, butanol, propane, formate, isopropanol, (3R)-hydroxybutyl (3R)-hydroxybutyrate, HMD, adipate, 6ACA, CPL, or HDO product pathway, wherein: the carboxylate by-product is one or more of 3HB, (R)-3HB, (S)-3HB, acetate, acetoacetate, succinate, crotonate, butyrate, isobutyrate, malonate, and/or hexanoate; the acyl-CoA intermediate or precursor is one or more of 3HB—CoA, (R)-3HB—CoA, (S)-3HB—CoA, acetyl-CoA, acetoacetyl-CoA, succinyl-CoA, crotonyl-CoA, butyryl-CoA, isobutyryl-CoA, malonyl-CoA, and/or hexanoyl-CoA; and the non-naturally occurring microbial organism expresses an ACS (or CoA ligase), such as an AMP-forming ACS characterized by EC 6.2.1.-, EC 6.2.1.1, and/or EC 6.2.2.2 that catalyzes the ligation of the carboxylate by-product with CoA, resulting in the recycling of the carboxylate by-product back to its respective/corresponding acyl-CoA. The non-naturally occurring microbial organism can further comprise a 3HB to 3HBAld conversion step, for example, by expressing an enzyme having carboxylic acid reductase activity characterized by EC 1.2.1.- and/or EC 1.2.1.30, where the microbial organism comprises an acetyl-CoA product pathway having a 3HB—CoA and/or a 3HBAld intermediate. Additionally, the microbial organism can comprise an acetyl-CoA derived product pathway, as described herein.

It is understood that the expression of the exogenous nucleic acids described herein can be regulated by various promoters, such as an endogenous promoter, an exogenous promoter, a synthetic promoter, a constitutive promoter, or an inducible promoter. A person of skill in the art would understand which type of promoter to use according to the level and duration of expression desired. For example, if the level of expression desired was the endogenous level, a person of average skill would understand that an endogenous promoter could be used to control the expression of the exogenous nucleic acid. Further, if the expression was desired to be, for example, temporary, or at a specific point during fermentation or growth of the microbial organism, an inducible promoter can be used to control the expression of the exogenous nucleic acid. However, if the expression was desired to be, for example, constant and robust, a constitutive promoter can be used to control the expression of the exogenous nucleic acid. Therefore, in some embodiments, the exogenous nucleic acid encoding an enzyme (or a polypeptide having the desired activity) is regulated by an endogenous promoter, a constitutive promoter, or an inducible promoter. Examples of inducible promoters are: lac, IPTG, spac, xyl, and acrAB. Examples of constitutive promoters are J23107, CMV, EF1A, and SV40.

Additionally, or alternatively, the promoter that is used to control the expression of the exogenous nucleic acid can be one that results in weak expression or low levels of expression; medium or moderate levels of expression; or strong expression or high levels of expression. Examples of promoters that result in weak or low levels of expression are STE1, CYC1, and HIS3. Examples of promoters that result in moderate or medium levels of expression include ADH1. Examples of promoters that result in strong or high levels of expression include TEF1, TDH1, TDH3, PGK1, ENO1, ENO2, TPI1, GAL10, GAL1, and LEU2.

While generally described herein as a microbial organism having eliminated, substantially eliminated, or reduced 3HB by-products or carboxylate by-product(s), and/or enhanced or increased carbon flux through 3HB—CoA, and/or 3HBAld, and/or one or more acyl-CoAs, it is understood that the invention additionally provides a non-naturally occurring microbial organism comprising at least one exogenous nucleic acid encoding at least one enzyme or polypeptide expressed in a sufficient amount to result in or increase the production of a desired Ac—COA derived product, such as 1,3-BDO and/or an R or S isomer thereof, (3R)-hydroxybutyl (3R)-hydroxybutyrate, butyrate, butanol, propane, formate, isopropanol, MAA and/or MAA esters thereof (e.g., MMA, EMA, and others), and/or HMD, CPL, adipate, 6ACA, and HDO, as well as intermediates or precursors thereof.

Accordingly, it is understood that the invention additionally provides, in some embodiments, a non-naturally occurring microbial organism that includes an intermediate of a 1,3-BDO and/or an R or S isomer thereof, (3R)-hydroxybutyl (3R)-hydroxybutyrate, butyrate, butanol, propane, formate, isopropanol, MAA, MAA ester (e.g., MMA, EMA, and others), HMD, adipate, CPL, 6ACA, and/or HDO biosynthesis pathway, wherein the pathway contains at least one enzyme or polypeptide that acts on an Ac—COA derived product intermediate or precursor, and the pathway enzyme or polypeptide is expressed in a sufficient amount to produce an intermediate or a precursor of a 1,3-BDO and/or an R or S isomer thereof, (3R)-hydroxybutyl (3R)-hydroxybutyrate, butyrate, butanol, propane, formate, isopropanol, MAA, MAA ester (e.g., MMA, EMA, and others), HMD, adipate, CPL, 6ACA, and/or HDO pathway. Therefore, in addition to a microbial organism containing a 1,3-BDO and/or an R or S isomer thereof, (3R)-hydroxybutyl (3R)-hydroxybutyrate, butyrate, butanol, propane, formate, isopropanol, MAA, MAA ester (e.g., MMA, EMA, and others), HMD, adipate, CPL, 6ACA, and/or HDO biosynthesis pathway that produces 1,3-BDO, R-1,3 BDO, S-1,3 BDO, (3R)-hydroxybutyl (3R)-hydroxybutyrate, butyrate, butanol, propane, formate, isopropanol, MAA, MAA ester, HMD, adipate, CPL, 6ACA, and HDO, respectively, the invention additionally provides a non-naturally occurring microbial organism, where the microbial organism produces an intermediate or precursor of an Ac—COA derived product, such as an intermediate or precursor of a 1,3-BDO and/or an R or S isomer thereof, (3R)-hydroxybutyl (3R)-hydroxybutyrate, butyrate, butanol, propane, formate, isopropanol, MAA, MAA ester (e.g., MMA, EMA, and others), HMD, adipate, CPL, 6ACA, and/or HDO biosynthesis pathway.

It is understood that any of the pathways disclosed herein, as described throughout and incorporated by reference in their entirety, can be utilized by the non-naturally occurring microbial organism having eliminated, substantially eliminated, or reduced 3HB by-product and/or carboxylate by-product(s), and/or enhanced or increased carbon flux through 3HB—CoA, and/or 3HBAld, and/or one or more acyl-CoAs (e.g., acyl-CoA intermediates or precursors), to generate a non-naturally occurring microbial organism that further produces any pathway intermediate, precursor, or product derived from Ac—COA, as desired. As disclosed herein, such a microbial organism that produces an intermediate or precursor can be used in combination with another microbial organism expressing downstream pathway enzymes or polypeptides to produce a desired product. However, it is understood that a non-naturally occurring microbial organism that produces an intermediate or precursor of an Ac—COA derived product, such as an intermediate or precursor of a 1,3-BDO and/or an R or S isomer thereof, (3R)-hydroxybutyl (3R)-hydroxybutyrate, butyrate, butanol, propane, formate, isopropanol, MAA, MAA ester (e.g., MMA, EMA, and others), HMD, adipate, CPL, 6ACA, and/or HDO biosynthesis pathway, can be utilized to produce the intermediate or precursor as a desired product.

The invention is described herein with general reference to eliminating, substantially eliminating, or reducing the by-product(s) of one or more metabolic reactions or steps in a pathway or pathways, or a reactant/substrate or product thereof, or, with specific reference to one or more nucleic acids or genes encoding an enzyme or polypeptide associated with or catalyzing the referenced metabolic reaction, pathway step, reactant/substrate, and/or product. Those skilled in the art will understand that reference to a reaction or pathway step also can include reference to the reactants/substrates and products of the reaction. Similarly, reference to a reactant/substrate or product also can refer to the reaction or pathway step, and reference to any of these metabolic constituents also can reference the gene or genes, or nucleic acid or acids, encoding the enzymes or polypeptides that catalyze and/or are involved in the referenced reaction or pathway step, reactant/substrate, and/or product. Likewise, given the well-known fields of metabolic biochemistry, enzymology and genomics, reference herein to a gene or encoding nucleic acid also includes reference to the corresponding encoded enzyme or polypeptide, and the reaction or pathway step it catalyzes, or a protein associated with the reaction or pathway step, as well as the reactants/substrates and products of the reaction.

The non-naturally occurring microbial organisms of the invention can be produced by introducing expressible nucleic acids encoding one or more of the enzymes or polypeptides described herein, including, for example, enzymes or polypeptides included in the 3HB recycling loop, the carboxylate by-product recycling loop(s), and/or the 3HB to 3HBAld conversion step. In addition, in some embodiments, the non-naturally occurring microbial organisms of the invention can be produced by further introducing one or more of the enzymes or polypeptides in, for example, one or more Ac—COA derived product biosynthesis pathways, including, for example, the pathways for 1,3-BDO and/or an R or S isomer thereof, (3R)-hydroxybutyl (3R)-hydroxybutyrate, butyrate, butanol, propane, formate, isopropanol, MAA, MAA esters (e.g., MMA, EMA, and others), HMD, adipate, CPL, 6ACA, or HDO, or any other Ac—COA derived product.

Depending on the host microbial organism chosen for product biosynthesis, nucleic acids encoding some or all of the enzymes, polypeptides, or proteins of a particular biosynthetic pathway that converts Ac—CoA into a desired product, such as 1,3-BDO and/or an R or S isomer thereof, (3R)-hydroxybutyl (3R)-hydroxybutyrate, butyrate, butanol, propane, formate, isopropanol, MAA, MAA esters (e.g., MMA, EMA, and others), HMD, adipate, CPL, 6ACA, and/or HDO, can be introduced. For example, if the host organism or cell is deficient in one or more enzymes, polypeptides, or proteins for a desired biosynthetic pathway, then expressible nucleic acids that encode the deficient enzyme(s), polypeptide(s), or protein(s) can be introduced into the host for subsequent exogenous expression. Alternatively, if the chosen host exhibits endogenous expression of one or more pathway genes, but is deficient in others, then an encoding nucleic acid can be introduced for the deficient enzyme(s), polypeptide(s), or protein(s), to achieve biosynthesis of the bioderived product of interest. Thus, a non-naturally occurring microbial organism of the invention can be produced by introducing exogenous enzyme, polypeptide, or protein activities, to obtain a desired biosynthetic pathway, or a desired biosynthetic pathway can be obtained by introducing one or more exogenous enzyme, polypeptide, or protein activities that, together with one or more endogenous enzymes, polypeptides, or proteins, result in the production of a desired bioderived product, such as, for example, 1,3-BDO and/or an R or S isomer thereof, (3R)-hydroxybutyl (3R)-hydroxybutyrate, butyrate, butanol, propane, formate, isopropanol, MAA, MAA esters (e.g., MMA, EMA, and others), HMD, adipate, CPL, 6ACA, and/or HDO.

Host microbial organisms can be selected from, and the non-naturally occurring microbial organisms can be generated or derived from, for example, bacteria (including cyanobacteria), yeast, fungi, or any of a variety of other microorganisms applicable to fermentation processes. Exemplary bacteria include Escherichia coli, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, and Pseudomonas putida. Other exemplary bacteria include species of Ruegeria, Rhodobacteraceae, Cribrihabitans, Falsiruegeria, Tropicibacter, Thioclava, Paracoccus, Celeribacter, Gemmobacter, Marivivens, Actibacterium, Salipiger, Youngimonas, Poseidonocella, Nitratireductor, Rhizobium, Aestuarium, Pseudodonghicola, Rhodobacterales, Ciceribacter, Polymorphum, Rhizobiales, Salipiger, Rhizobiaceae, or Roseobacter. Exemplary yeasts or fungi include Saccharomyces cerevisiae, Schizosaccharomyces pombe, Kluyveromyces lactis, Kluyveromyces marxianus, Aspergillus terreus, Aspergillus niger, Pichia pastoris, Rhizopus arrhizus, Rhizopus oryzae, and the like. E. coli is a particularly useful host organism since it is a well characterized microbial organism that is suitable for genetic engineering. Other particularly useful host organisms include yeast, such as Saccharomyces cerevisiae. It is understood that any suitable microbial host organism or cell can be used to introduce metabolic and/or genetic modifications to produce a desired product.

Given the teachings and guidance provided herein, those skilled in the art will understand that the number of encoding nucleic acids to introduce in an expressible form will, at least, parallel the pathway deficiencies of the selected host microbial organism having eliminated, substantially eliminated, or reduced by-products, such as carboxylate by-products, particularly short-chain (e.g., C2-C4, C2-C6, or C2-C8) carboxylate by-products. For example, a non-naturally occurring microbial organism of the invention can have one or two nucleic acids encoding the enzymes, polypeptides, or proteins constituting a 3HB recycling loop pathway, and/or a carboxylate by-product recycling loop pathway, as disclosed herein for reducing by-products, such as 3HB and/or one or more other carboxylate by-product(s), and/or for enhancing or increasing carbon flux through 3HB—CoA and/or one or more other acyl-CoA(s). Additionally, or alternatively, a non-naturally occurring microbial organism of the invention can have one or two nucleic acids encoding the enzymes, polypeptides, or proteins constituting a 3HB to 3HBAld conversion step, as disclosed herein for reducing 3HB by-product, and/or for enhancing or increasing carbon flux through 3HBAld. The enhanced or increased carbon flux through 3HB—CoA, and/or other carboxylate by-products (e.g., acetate, acetoacetate, succinate, crotonate, butyrate, isobutyrate, malonate, and/or hexanoate) and/or 3HBAld, can increase the productivity, yield, and/or titer of Ac—COA derived products, such as 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, adipate, CPL, 6ACA, and/or HDO. In some embodiments, the non-naturally occurring microbial organisms also can include other genetic modifications that facilitate or optimize the biosynthesis of Ac—COA derived products, such as 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, adipate, CPL, 6ACA, and/or HDO, and/or any other Ac—COA derived product, or that confer other useful functions or properties onto the host microbial organism. One such other functionality can include, for example, augmentation of the synthesis of one or more of the Ac—CoA derived product pathway precursors and/or intermediates, such as, for example, pyruvate, Ac—COA, AcAc—COA, malonyl-CoA, 3HB—CoA, 4HB—COA, AKG, succinate, Suc-CoA, glutamate, 4-aminobutyrate, methacrylyl-CoA, or a precursor and/or intermediate of any other Ac—COA derived product.

Therefore, a non-naturally occurring microbial organism of the invention, having eliminated, substantially eliminated, or reduced by-products, such as carboxylate by-products (including, e.g., 3HB, acetate, acetoacetate, succinate, crotonate, butyrate, isobutyrate, malonate, and/or hexanoate), and/or enhanced or increased carbon flux through 3HB—CoA, and/or one or more other acyl-CoAs, and/or 3HBAld, can have one or more nucleic acids encoding the enzymes, polypeptides, or proteins constituting a 3HB recycling loop, a carboxylate by-product recycling loop, and/or a 3HB to 3HBAld conversion step disclosed herein. In some embodiments, a non-naturally occurring microbial organism of the invention having eliminated, substantially eliminated, or reduced by-products (such as carboxylate by-products), and/or enhanced or increased carbon flux through 3HB—CoA, and/or one or more other acyl-CoAs, and/or 3HBAld, can include one, two, three, four, five, six, seven, or eight, or more, up to all the nucleic acids encoding the enzymes, polypeptides, or proteins constituting one or more biosynthetic pathway disclosed herein. In some embodiments, the non-naturally occurring microbial organisms also can include other genetic modifications that facilitate or optimize the biosynthesis of Ac—COA derived products, such as, for example, 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, adipate, CPL, 6ACA, HDO, or any other Ac—CoA derived product, or that confer other useful functions and/or properties onto the host microbial organism. By way of example, one such other functionality can include, for example, augmentation of the synthesis of one or more of the 1,3-BDO pathway precursors or intermediates, such as 3HB—CoA, acetyl-CoA, acetoacetyl-CoA, and/or 3HBAld.

Generally, a host microbial organism is selected such that it produces the precursor of an Ac—CoA dependent pathway, either as a naturally produced molecule or as an engineered product that either provides de novo production of a desired precursor or increased production of a precursor naturally produced by the host microbial organism. For example, Ac—COA is produced naturally in a host organism such as E. coli. A host organism can be engineered to increase production of a precursor, as disclosed herein. In addition, a microbial organism that has been engineered to produce a desired precursor can be used as a host organism, and can be further engineered to express one or more of the enzymes, polypeptides, or proteins of an Ac—CoA derived product pathway or pathways.

In some embodiments, a non-naturally occurring microbial organism of the invention, having eliminated, substantially eliminated, or reduced 3HB by-product and/or other carboxylate by-product, is generated from a host that contains the enzymatic capability to synthesize an Ac—COA derived product, such as 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, adipate, CPL, 6ACA, and/or HDO. In this specific embodiment, it can be useful to increase the synthesis or accumulation of an intermediate and/or precursor of an Ac—CoA derived product pathway, for example, to drive the Ac—COA derived product pathway reactions/steps toward production of an Ac—COA derived product. Increased synthesis or accumulation of a pathway intermediate or precursor can be accomplished by, for example, overexpression of one or more nucleic acids encoding one or more enzymes, polypeptides or proteins involved in pathways for producing Ac—COA derived products.

Overexpression of the enzyme(s), polypeptide(s), and/or protein(s) described herein, that are capable of eliminating, substantially eliminating, or decreasing 3HB by-product and/or other carboxylate by-product(s), and/or enhancing or increasing carbon flux through 3HB—CoA, and/or one or more other acyl-CoAs, and/or 3HBAld, and thereby increasing the productivity, yield, and/or titer of Ac—COA derived products, such as 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, adipate, CPL, 6ACA, or HDO, can occur, for example, through the introduction and expression of exogenous nucleic acid(s) or gene(s) encoding the same gene product(s) as the endogenous nucleic acid(s) or gene(s), or through the introduction and expression of a exogenous and/or heterologous nucleic acid(s) or gene(s). Thus, these enzyme(s), polypeptide(s), and/or protein(s) can be heterologous or can be native to the non-naturally occurring microorganisms described herein.

Therefore, naturally occurring organisms can be readily engineered into the non-naturally occurring microbial organisms provided herein, for example, the non-naturally occurring microbial organisms having eliminated, substantially eliminated, or reduced 3HB by-product and/or other carboxylate by-product(s), and/or enhanced or increased carbon flux through 3HB—CoA, and/or one or more acyl-CoAs, and/or 3HBAld, and/or increased productivity, titer, and/or yield of Ac—COA derived products, such as 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, adipate, CPL, 6ACA, and/or HDO, through the expression or overexpression of one, two, three, four, five, six, seven, or eight, or more, depending on the number of enzymes in the pathway, that is, up to all nucleic acids encoding the enzymes, polypeptides, or proteins described herein that can result in eliminated, substantially eliminated, or reduced by-products, as well as increased productivity, yield and/or titer of, for example, 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, adipate, CPL, 6ACA, and/or HDO, or any other Ac—COA derived product. In addition, a non-naturally occurring microbial organism can be generated by mutagenesis of an endogenous gene or genes that results in an increase in the expression levels or in the activity of an enzyme, polypeptide, or protein described herein that can result in eliminated, substantially eliminated, or decrease by-products, as well as increased productivity, titer and/or yield of, for example, 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, adipate, CPL, 6ACA, and/or HDO, or any other Ac—COA derived product described herein or known in the art.

In some embodiments, the encoding nucleic acid(s) that are expressed are exogenous, i.e., introduced into the host microorganism or cell, for example, on a plasmid(s), or via integration into the host chromosomal DNA. As described elsewhere herein, the exogenous nucleic acid(s) can be heterologous (e.g., not found naturally in the host), or the exogenous nucleic acid(s) can encode the same gene product(s) as an endogenous gene or genes. The introduction of exogenous nucleic acid(s) confers the ability to control or modify the expression level of the encoded product(s), as well as the ability to induce expression at a certain time point or points. This can be achieved, for example, by the introduction of various regulatory sequences and elements, such as, for example, promoters, enhancers, silencers, operators, or any regulatory sequences and elements known in the art. For example, various promoters that are known in the art can induce strong (or high levels of), medium or moderate levels of, or weak (or low levels of) expression, while other promoters can be induced or activated by the addition of an inducing agent or agents. Additionally, or alternatively, when the exogenous nucleic acid(s) are introduced into the host via a plasmid, the level of expression can be controlled by the plasmid's origin of replication, which determines the plasmid copy number. For example, the copy number of the plasmid can be low (e.g., ~5 or less copies per cell), medium (e.g., ~15-20 copies/cell), or high (e.g., >100 copies/cell).

However, as described herein, expression of endogenous nucleic acid(s)/gene(s) also can be utilized in other embodiments, such as by removing a negative regulatory element or by induction of the gene's promoter when linked to an inducible promoter or other regulatory element. Thus, an endogenous gene having a naturally occurring inducible promoter can be up-regulated or overexpressed by providing the appropriate inducing agent, or the regulatory region of an endogenous gene can be engineered to incorporate an inducible regulatory element, thereby allowing the regulation of increased expression of an endogenous gene at a desired time. The expression levels of endogenous nucleic acids/genes also can be controlled, for example, by the use of promoters that induce varying levels of expression, or by the introduction of regulatory elements, such as enhancers, or by the removal or silencing of repressors, or by any other methods known in the art.

It is understood that, in methods of the invention, any of the one or more exogenous nucleic acids can be introduced into a microbial organism to produce a non-naturally occurring microbial organism of the invention having eliminated, substantially eliminated, or reduced 3HB by-product, and/or carboxylate by-product (e.g., short-chain (e.g., C2-C4, C2-C6, or C2-C8) carboxylate by-product), and/or enhanced or increased carbon flux through 3HB—CoA, and/or one or more acyl-CoA(s) (e.g., short-chain (e.g., C2-C4, C2-C6, or C2-C8) acyl-CoA(s)), and/or 3HBAld. The one or more exogenous nucleic acids can be introduced so as to confer, for example, a microbial organism having a 3HB recycling loop and/or other carboxylate by-product recycling loop. For example, in come embodiments, the microbial organism can have a 3HB recycling loop and an acetate recycling loop. In some embodiments, the one or more exogenous nucleic acids can be introduced so as to confer, for example, a microbial organism having a 3HB recycling loop and/or other carboxylate by-product recycling loop, and/or a 3HB to 3HBAld conversion step. For example, in some embodiments, the microbial organism can comprise a 3HB recycling loop and a 3HB to 3HBAld conversion step, or the microbial organism can comprise a 3HB recycling loop, an acetate recycling loop, and/or a 3HB to 3HBAld conversion step. In some embodiments, the microbial organism having eliminated, substantially eliminated, or reduced by-products can further include one or more exogenous nucleic acids introduced so as to confer, for example, a microbial organism having a biosynthetic pathway for production of an Ac—COA derived product, such as, for example, 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, adipate, CPL, 6ACA, and/or HDO, or any other acetyl-CoA derived product. Alternatively, one or more exogenous nucleic acids can be introduced to produce a microbial organism having the biosynthetic capability to catalyze some of the required reactions to confer biosynthesis of 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, adipate, CPL, 6ACA, and/or HDO, or any other Ac—COA derived product described herein or known in the art. For example, a non-naturally occurring microbial organism having eliminated, substantially eliminated, or reduced 3HB by-product, and/or having eliminated, substantially eliminated, or reduced carboxylate by-product, and/or enhanced or increased carbon flux through 3HB—CoA, and/or one or more acyl-CoAs, and/or 3HBAld, and/or increased productivity, yield, and/or titer of one or more Ac—COA derived products, such as, for example, 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, adipate, CPL, 6ACA, and/or HDO, can include at least one or at least two exogenous nucleic acids encoding desired enzymes, polypeptides, or proteins, such as, for example: (i) an ACS or a CoA ligase, or a variant or homolog thereof, or a polypeptide or a protein with ACS or CoA ligase activity; (ii) an enzyme, polypeptide or protein with activity that converts a substrate to 3HB—CoA; (iii) an enzyme, polypeptide, or protein with activity that converts 3HB to 3HBAld, such as a carboxylic acid reductase; or (iv) any combination of any one of (i), (ii), and (iii). Thus, it is understood that any combination of two or more enzymes, polypeptides, or proteins of a 3HB recycling pathway, a carboxylate by-product recycling pathway, and/or a 3HB to 3HBAld conversion step, can be expressed in a non-naturally occurring microbial organism provided herein, whereby the microbial organism can additionally express any one or more enzymes, polypeptides, or proteins of an Ac—COA derived product pathway.

In addition to the biosynthesis of, for example, 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, adipate, CPL, 6ACA, and/or HDO, or any other Ac—COA derived product as described herein or known in the art, the non-naturally occurring microbial organisms having eliminated, substantially eliminated, or decreased 3HB by-product and/or carboxylate by-product, and/or enhanced or increased carbon flux through 3HB—CoA, and/or one or more acyl-CoAs, and/or 3HBAld, and the methods provided herein for eliminating, substantially eliminating, or decreasing such by-products, can also be utilized in various combinations with each other and with other microbial organisms and methods described in or well known in the art to achieve product biosynthesis by other routes. For example, one alternative to produce an Ac—COA derived product (e.g., 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, adipate, CPL, 6ACA, and/or HDO) in a non-naturally occurring microbial organism having eliminated, substantially eliminated, or decreased 3HB by-product and/or carboxylate by-product, and/or enhanced or increased carbon flux through 3HB—CoA, and/or one or more acyl-CoAs, and/or 3HBAld, is through the use of another microbial organism capable of converting an intermediate of the Ac—COA derived product pathway to the corresponding Ac—CoA derived product.

For example, a first non-naturally occurring microbial organism can be used to produce an intermediate of an Ac—COA derived product pathway, and the intermediate can then be used as a substrate for a second non-naturally occurring microbial organism, whereby the second organism converts the intermediate to the Ac—COA derived product. As described herein, the Ac—COA derived product can be, for example, 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, adipate, CPL, 6ACA, and/or HDO, or any other Ac—COA derived product described or known in the art. The intermediate of the Ac—COA derived product pathway, that is produced by the first non-naturally occurring microbial organism, can be added directly to another culture medium, containing the second non-naturally occurring microbial organism, or the original culture medium containing the intermediate can be depleted of the first microbial organism, and then the second non-naturally occurring microbial organism can be added to the original culture medium to produce the final Ac—COA derived product without intermediate purification steps. Depletion of the first microbial organism from the original culture medium can be achieved by, for example, cell separation techniques (e.g., centrifugation and/or filtration), or by heat killing, or by any other methods described or known in the art.

In other embodiments, the non-naturally occurring microbial organisms described herein, having eliminated, substantially eliminated, or reduced 3HB by-product and/or carboxylate by-product, and/or enhanced or increased carbon flux through 3HB—CoA, and/or one or more other acyl-CoA(s), and/or 3HBAld, and the methods described herein for eliminating, substantially eliminating, or reducing by-products, can be assembled to include a wide variety of subpathways to achieve biosynthesis of, for example, 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, adipate, CPL, 6ACA, and/or HDO, or any other acetyl-CoA derived product. In these embodiments, biosynthetic pathways for a desired product can be divided into two or more different parts, and each part can be encoded into a different microbial organism, whereby the different microbial organisms can be co-cultured to produce the final product. In such a biosynthetic scheme, the product of one microbial organism serves as the substrate for a second microbial organism, and so forth, until the final product is synthesized. One or more of the microbial organisms that each encodes a portion of the biosynthetic pathway can be engineered to have eliminated, substantially eliminated, or reduced 3HB by-product, and/or eliminated, substantially eliminated, or reduced carboxylate by-product(s), and/or enhanced or increased carbon flux through 3HB—CoA, and/or one or more acyl-CoA(s), and/or 3HBAld, thereby increasing the productivity, yield, and/or titer of the Ac—CoA derived product(s).

Given the teachings and guidance provided herein, those skilled in the art will understand that a wide variety of combinations and permutations exist for the non-naturally occurring microbial organisms and methods of the invention together with other microbial organisms. For example, i) the non-naturally occurring microbial organism can be engineered to have eliminated, substantially eliminated, or reduced 3HB by-product and/or carboxylate by-product(s) (e.g., short-chain (e.g., C2-C4, C2-C6, or C2-C8) carboxylate by-product(s)), and/or enhanced or increased carbon flux through 3HB—CoA, and/or one or more other acyl-CoA(s) (particularly short-chain (e.g., C2-C4, C2-C6, or C2-C8) acyl-CoA(s)), and/or 3HBAld, and to include one or more biosynthetic pathways for the production or one or more Ac—COA derived products; or ii) the non-naturally occurring microbial organism having eliminated, substantially eliminated, or reduced 3HB by-product and/or carboxylate by-product(s) (e.g., short-chain (e.g., C2-C4, C2-C6, or C2-C8) carboxylate by-product(s)), and/or enhanced or increased carbon flux through 3HB—CoA, and/or one or more other acyl-CoA(s) (particularly short-chain (e.g., C2-C4, C2-C6, or C2-C8) acyl-CoA(s)), and/or 3HBAld, can be co-cultured with another non-naturally occurring microbial organism having one or more Ac—COA derived biosynthetic pathways; or iii) the non-naturally occurring microbial organism having eliminated, substantially eliminated, or reduced 3HB by-product, and/or carboxylate by-product(s) (e.g., short-chain (e.g., C2-C4, C2-C6, or C2-C8) carboxylate by-product(s)), and/or enhanced or increased carbon flux through 3HB—CoA, and/or one or more other acyl-CoA(s) (particularly short-chain (e.g., C2-C4, C2-C6, or C2-C8) acyl-CoA(s)), and/or 3HBAld, can be co-cultured with two or more other non-naturally occurring microbial organisms, each having a subpathway, whereby the combination of the subpathways produces an Ac—CoA derived product or products; or iv) the non-naturally occurring microbial organisms having eliminated, substantially eliminated, or reduced 3HB by-product, and/or carboxylate by-product(s) (e.g., short-chain (e.g., C2-C4, C2-C6, or C2-C8) carboxylate by-product(s)), and/or enhanced or increased carbon flux through 3HB—CoA, and/or one or more other acyl-CoA(s) (particularly short-chain (e.g., C2-C4, C2-C6, or C2-C8) acyl-CoA(s)), and/or 3HBAld, can be engineered to include a subpathway, and can be co-cultured with one or more other non-naturally occurring microbial organisms, each having a different subpathway, whereby the combination of the subpathways produces an Ac—COA derived product or products.

Similarly, it is understood by those skilled in the art that a host organism can be selected based on desired characteristics for the introduction of one or more gene modifications to increase production (e.g., productivity, titer, and/or yield) of 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, adipate, CPL, 6ACA, and/or HDO, and/or any other acetyl-CoA derived product. Thus, it is understood that, if a genetic modification is to be introduced into a host organism, any homologs, orthologs, or paralogs that encode enzymes, polypeptides or proteins that catalyze similar, yet non-identical metabolic reactions, can similarly be modified to ensure that a desired metabolic reaction is sufficiently modified. Because certain differences exist among metabolic networks and pathways between different organisms, those skilled in the art will understand that the actual genes that are modified in a given organism may differ between organisms or species of organisms. However, given the teachings and guidance provided herein, those skilled in the art also will understand that the methods of the invention can be applied to any suitable host microorganism, and the cognate metabolic modifications needed to construct or engineer an organism in a species of interest, that can eliminate, substantially eliminate, or reduce by-products and/or increase biosynthesis of 11,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, adipate, CPL, 6ACA, and/or HDO, or any other Ac—COA derived product, readily can be identified.

Sources of encoding nucleic acids for a 3HB recycling loop, a carboxylate by-product recycling loop, a 3HB to 3HBAld conversion step, and/or a pathway enzyme, polypeptide, or protein that uses Ac—COA, or an Ac—COA derived molecule/intermediate, as a substrate can include, for example, any species where the encoded gene product is capable of catalyzing the reaction of interest. Such species include both prokaryotic and eukaryotic organisms and their cells, including, but not limited to, bacteria, including archaea and eubacteria, and eukaryotes, including fungi, yeasts, plant cells, insect cells, and animal cells, including mammalian cells, such as human cells. Exemplary species for such sources include, for example, Escherichia coli, as well as other exemplary species disclosed herein or available as source organisms for corresponding genes. Exemplary species for such sources include, for example, Escherichia coli; species of Ruegeria, including, e.g., Ruegeria pomeroyi; Escherichia fergusonii; Methanocaldococcus jannaschii; Leptospira interrrogans; Geobacter sulfurreducens; Chloroflexus aurantiacus; Roseiflexus sp. RS-1; Chloroflexus aggregans; Achromobacter xylosoxydans; Clostrdia species, including Clostridium kluyveri, Clostridium symbiosum, Clostridium acetobutylicum, Clostridium saccharoperbutylacetonicum, and Clostridium ljungdahlii, Trichomonas vaginalis G3; Trypanosoma brucei; Acidaminococcus fermentans; Fusobacterium species, including Fusobacterium nucleatum, and Fusobacterium mortiferum; Corynebacterium glutamicum, Saccharomyces species, including Saccharomyces cerevisiae; Apsergillus species, including Aspergillus terreus, Aspergillus oryzae, and Aspergillus niger, Gibberella zeae; Pichia stipitis; Mycobacterium species, including Mycobacterium smegmatis, and Mycobacterium avium, including subsp. Paratuberculosis, Salinispora arenicola; Pseudomonas species, including Pseudomonas sp. CF600, Pseudomonas putida, Pseudomonas fluorescens, and Pseudomonas aeruginosa; Ralstonia species, including Ralstonia eutropha, Ralstonia eutropha JMP134, Ralstonia eutropha H16, and Ralstonia pickettii, Lactobacillus plantarum; Klebsiella oxytoca; Bacillus species, including Bacillus methanolicus, Bacillus subtilis, Bacillus pumilus, and Bacillus megaterium; Pedicoccus pentosaceus; Chlorofexus species, including Chloroflexus aurantiacus and Chloroflexus aggregans; Rhodobacter sphaeroides; Methanocaldococcus jannaschii; Leptospira interrrogans; Candida maltosa, Salmonella species, including Salmonella enterica serovar Typhimurium; Shewanella species, including Shewanella oneidensis and Shewanella sp. MR-4; Alcaligenes faecalis; Geobacillus stearothermophilus; Serratia marcescens; Vibrio cholerae; Eubacterium barkeri, Bacteroides capillosus; Archaeoglobus fulgidus; Haloarcula marismortui; Pyrobaculum aerophilum str. IM2; Rhizobium species, including Rhizobium leguminosarum; Rattus norvegicus; species of Rhodobacteraceae, Cribrihabitans, Falsiruegeria, Tropicibacter, Thioclava, Paracoccus, Celeribacter, Gemmobacter, Marivivens, Actibacterium, Salipiger, Youngimonas, Poseidonocella, Nitratireductor, Rhizobium, Aestuarium, Pseudodonghicola, Rhodobacterales, Ciceribacter, Polymorphum, Rhizobiales, Salipiger, Rhizobiaceae, or Roseobacter; and Homo sapiens, as well as other exemplary species disclosed herein or available as source organisms for corresponding genes. However, with the complete genome sequence now available for more than 550 species (with more than half of these available on public databases, such as the NCBI), including the genomes of 395 microorganisms and of a variety of yeast, fungi, plant, and mammalian species, genes encoding polypeptides/proteins with the requisite activity to eliminate, substantially eliminate, or decrease by-products, such as 3HB by-product and/or one or more other carboxylate by-products, and/or to enhance or increase carbon flux through 3HB—CoA, and/or one or more other acyl-CoAs, and/or 3HBAld, along with genes encoding enzymes, polypeptides, and/or proteins that are part of the biosynthetic pathways for 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, adipate, CPL, 6ACA, and/or HDO, or other Ac—COA derived products, readily can be identified in related or distant species. For example, the identification of homologues, orthologues, paralogues, and nonorthologous gene displacements of known genes, and the interchange of genetic variations between organisms/species is routine and well known in the art. Accordingly, the metabolic modifications allowing for eliminated, substantially eliminated, or reduced 3HB by-product and/or one or more other carboxylate by-products, and/or enhanced or increased carbon flux through 3HB—CoA, and/or one or more other acyl-CoAs, and/or 3HBAld, and/or biosynthesis of one or more Ac—COA derived products, that are described herein with reference to a particular organism, such as, for example, E. coli, can be readily applied to other species of microorganisms, including other bacterial species, or other prokaryotic and eukaryotic species. Given the teachings and guidance provided herein, those skilled in the art will know that a metabolic modification exemplified in one species or organism can be applied equally to other species or organisms.

In some instances, a different enzyme (or polypeptide or protein) with the ability or activity to catalyze the same or a similar (but not identical) step in a biosynthetic pathway for an Ac—COA derived product can exist in a different (e.g., unrelated) species than the selected host species. In such instances, (an) exogenous nucleic acid(s), encoding the enzyme from the unrelated species that catalyzes a similar, but not identical metabolic reaction, can be introduced into the host species to achieve the metabolic reaction of interest.

Because certain differences among metabolic networks exist between different species, those skilled in the art will understand that the reactions catalyzed by the different gene products can differ amongst different species. However, given the teachings and guidance provided herein, those skilled in the art also will understand that the teachings and methods of the invention can be applied to all microbial organisms using the metabolic modifications corresponding to those exemplified herein, to construct a microbial organism in a species of interest that will synthesize 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, adipate, CPL, 6ACA, and/or HDO, and/or any other acetyl-CoA derived product(s).

Recombinant techniques and methods for the construction of a non-naturally occurring microbial organism having eliminated, substantially eliminated, or reduced 3HB by-product and/or one or more other carboxylate by-products, and/or enhanced or increased carbon flux through 3HB—CoA, and/or one or more other acyl-CoAs, and/or 3HBAld, as well as methods for determining the expression levels in such an organism, are well known in the art. Such methods are described in, for example, Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, MD (1999). Any such suitable method may be used to construct the non-naturally occurring microbial organism described herein.

Exogenous nucleic acid sequences involved in a pathway for eliminating, substantially eliminating, or reducing by-products, such as 3HB by-product and/or one or more other carboxylate by-products, and/or to enhance or increase carbon flux through Ac—COA and/or 3HB—CoA, and/or one or more other acyl-CoAs, and/or 3HBAld, thereby increasing Ac—COA derived products, such as 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, adipate, CPL, 6ACA, and/or HDO, and/or any other acetyl-CoA derived product(s), can be introduced stably or transiently into a host cell using techniques well known in the art, including, but not limited to, conjugation, electroporation, chemical transformation, transduction, transfection, and ultrasound transformation. Nucleic acids (e.g., genes or cDNAs) derived from eukaryotic species can encode targeting signals, such as an N-terminal mitochondrial or other targeting signal. Thus, for the expression of exogenous nucleic acids in E. coli or other prokaryotic cells, some portions or sequences in eukaryotic nucleic acids can be removed before transformation into prokaryotic host cells, if desired. For example, removal of a mitochondrial leader sequence has been shown to result in increased expression in E. coli (see, e.g., Hoffmeister et al., J. Biol. Chem. 280:4329-4338 (2005)). For expression in yeast or other eukaryotic cells, of exogenous nucleic acids that lack certain elements, such as a leader sequence or secretion signal, the nucleic acids can be expressed in the cytosol without the addition of leader sequences, or the nucleic acids or their encoded products can be targeted to the mitochondria or other organelles, or can be targeted for secretion, by the addition of a targeting sequence or secretion signal suitable for the host cell. Thus, it is understood that appropriate modifications to a nucleic acid sequence, for example, to remove or include a targeting or other sequence can be incorporated into an exogenous nucleic acid sequence to impart desirable properties. Furthermore, genes and nucleic acid sequences can be subjected to codon optimization with techniques well known in the art to achieve optimized expression of the encoded proteins.

An expression vector or vectors can be constructed to include one or more exogenous nucleic acids, each encoding an enzyme expressed in a sufficient amount to eliminate, substantially eliminate, or reduce by-products, and/or to increase or enhance carbon flux through 3HB—CoA, and/or one or more acyl-CoA(s), and/or 3HBAld, as exemplified herein, where the nucleic acids are operably linked to expression control sequences functional in the host organism. In some embodiments, an expression vector or vectors can be further constructed to include one or more exogenous nucleic acids, each encoding an enzyme expressed in a sufficient amount to increase production of an Ac—COA derived product such as, for example, 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, adipate, CPL, 6ACA, HDO, or other Ac—COA derived product, as exemplified herein, where the nucleic acids are operably linked to expression control sequences functional in the host organism. Expression vectors applicable for use in the microbial host organisms of the invention include, for example, plasmids, phage vectors, viral vectors, episomes and artificial chromosomes, including vectors and selection sequences or markers operable for stable integration into a host chromosome. Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein.

Suitable purification and/or assays to test for the production of by-products, such as 3HB by-product or any other carboxylate by-product as described herein, Ac—COA, or Ac—COA derived products, such as 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, CPL, adipate, 6ACA, and/or HDO, can be performed using well known methods. Suitable replicates such as triplicate cultures can be grown for each engineered strain to be tested. For example, product and by-product formation in the engineered production host can be monitored. The final product and intermediates, and other organic compounds, can be analyzed by methods such as HPLC (High Performance Liquid Chromatography), GC-MS (Gas Chromatography-Mass Spectroscopy) and LC-MS (Liquid Chromatography-Mass Spectroscopy) or other suitable analytical methods using routine procedures well known in the art. The release of product in the fermentation broth can also be tested with the culture supernatant. By-products and residual glucose can be quantified by HPLC using, for example, a refractive index detector for glucose and alcohols, and a UV detector for organic acids (Lin et al., Biotechnol. Bioeng. 90:775-779 (2005)), or other suitable assay and detection methods well known in the art. The individual enzyme or protein activities from the exogenous DNA sequences can also be assayed using methods well known in the art.

The production of Ac—COA derived compounds, such as for example, 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, CPL, adipate, 6ACA, and/or HDO, can be separated from other components in the culture using a variety of methods well known in the art. Such separation methods include, for example, extraction procedures as well as methods that include continuous liquid-liquid extraction, pervaporation, membrane filtration, membrane separation, reverse osmosis, electrodialysis, distillation, crystallization, centrifugation, extractive filtration, ion exchange chromatography, size exclusion chromatography, adsorption chromatography, and ultrafiltration. All of the above methods are well known in the art.

Any of the non-naturally occurring microbial organisms having eliminated, substantially eliminated, or reduced by-products and/or increased or enhanced carbon flux through 3HB—CoA, and/or one or more acyl-CoA(s), and/or 3HBAld, as described herein, can be cultured to produce and/or secrete the biosynthetic Ac—COA derived products of the invention. For example, the Ac—COA derived product producers can be cultured for the biosynthetic production of a desired Ac—COA derived product, such as 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, CPL, adipate, 6ACA, and/or HDO.

For the production of the desired Ac—COA derived product, the recombinant strains are cultured in a medium with carbon source and other essential nutrients. It is sometimes desirable and can be highly desirable to maintain anaerobic conditions in the fermenter to reduce the cost of the overall process. Such conditions can be obtained, for example, by first sparging the medium with nitrogen and then sealing the flasks with a septum and crimp-cap. For strains where growth is not observed anaerobically, microaerobic or substantially anaerobic conditions can be applied by perforating the septum with a small hole for limited aeration. Exemplary anaerobic conditions have been described previously and are well-known in the art. Exemplary aerobic and anaerobic conditions are described, for example, in US application publication No. 2009/0047719, filed Aug. 10, 2007. Fermentations can be performed in a batch, fed-batch or continuous manner, as disclosed herein.

If desired, the pH of the medium can be maintained at a desired pH, in particular neutral pH, such as a pH of around 7 by addition of a base, such as NaOH or other bases, or acid, as needed to maintain the culture medium at a desirable pH. The growth rate can be determined by measuring optical density using a spectrophotometer (600 nm), and the glucose uptake rate by monitoring carbon source depletion over time.

The growth medium can include, for example, any carbohydrate source or feedstock which can supply a source of carbon to the non-naturally occurring microorganism. Such sources include, for example, sugars such as glucose, xylose, arabinose, galactose, mannose, fructose, sucrose, starch, and combinations thereof. Other sources of carbohydrate include, for example, renewable feedstocks and biomass.

Such microbial organisms or cells can use a carbon feedstock, such as one or more carbohydrates. Exemplary carbohydrates include, but are not limited to, starch, glucose, sucrose, xylose, arabinose, galactose, mannose, fructose, and glycerol, as well as combinations thereof. Other sources of carbohydrates include, for example, renewable feedstocks and biomass. Exemplary types of biomasses that can be used as feedstocks herein include cellulosic biomass, hemicellulosic biomass, and lignin feedstocks or portions of feedstocks. Such biomass feedstocks contain, for example, carbohydrate substrates useful as carbon sources, such as glucose, xylose, arabinose, galactose, mannose, fructose, and starch. Given the teachings and guidance provided herein, those skilled in the art will understand that renewable feedstocks and biomass other than those exemplified above also can be used for culturing the microbial organisms and cells of the invention for the production of the desired or target Ac—CoA derived product(s). In addition to renewable feedstocks such as those exemplified above, the microbial organisms and cells of the invention also can be modified for growth on syngas as the source of carbon. In this specific embodiment, one or more proteins or enzymes are expressed in the Ac—COA derived product producing organisms or cells, to provide a metabolic pathway for utilization of syngas or other gaseous carbon source, such as methane, or carbon dioxide, or combinations thereof. Additionally, the microbial organisms and cells provided herein can be modified for growth on alcohols, such as methanol, as the carbon source. In such an embodiment, the organisms or cells are modified or engineered to include a methanol metabolic pathway (MMP) for the utilization of methanol as the carbon source. In other embodiments, the carbon source can be formate, isopropanol, and the organisms or cells are modified to include a formaldehyde assimilation pathway (FAP).

Synthesis gas, also known as syngas or producer gas, is the major product of gasification of coal and of carbonaceous materials such as biomass materials, including agricultural crops and residues. Syngas is a mixture primarily of H2 and CO and can be obtained from the gasification of any organic feedstock, including but not limited to coal, coal oil, natural gas, biomass, and waste organic matter. Gasification is generally carried out under a high fuel to oxygen ratio. Although largely H2 and CO, syngas can also include CO₂ and other gases in smaller quantities. Thus, synthesis gas provides a cost effective source of gaseous carbon such as CO and, additionally, CO₂.

The Wood-Ljungdahl pathway catalyzes the conversion of CO and H2 to Ac—COA and other products such as acetate. Organisms capable of utilizing CO and syngas also generally have the capability of utilizing CO₂ and CO₂/H2 mixtures through the same basic set of enzymes and transformations encompassed by the Wood-Ljungdahl pathway. H2-dependent conversion of CO₂ to acetate by microorganisms was recognized long before it was revealed that CO also could be used by the same organisms and that the same pathways were involved. Many acetogens have been shown to grow in the presence of CO₂ and produce compounds such as acetate as long as hydrogen is present to supply the necessary reducing equivalents (see for example, Drake, Acetogenesis, pp. 3-60 Chapman and Hall, New York, (1994)). This can be summarized by the following equation:

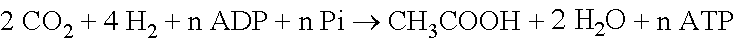

Hence, non-naturally occurring microorganisms possessing the Wood-Ljungdahl pathway can utilize CO₂ and H₂ mixtures as well for the production of Ac—COA and other desired products.

The Wood-Ljungdahl pathway is well known in the art and consists of 12 reactions which can be separated into two branches: (1) methyl branch and (2) carbonyl branch. The methyl branch converts syngas to methyl-tetrahydrofolate (methyl-THF) whereas the carbonyl branch converts methyl-THF to Ac—COA. The reactions in the methyl branch are catalyzed in order by the following enzymes or proteins: ferredoxin oxidoreductase, formate dehydrogenase, formyltetrahydrofolate synthetase, methenyltetrahydrofolate cyclodehydratase, methylenetetrahydrofolate dehydrogenase and methylenetetrahydrofolate reductase. The reactions in the carbonyl branch are catalyzed in order by the following enzymes or proteins: methyltetrahydrofolate: corrinoid protein methyltransferase (for example, AcsE), corrinoid iron-sulfur protein, nickel-protein assembly protein (for example, AcsF), ferredoxin, Ac—CoA synthase, carbon monoxide dehydrogenase and nickel-protein assembly protein (for example, CooC). Following the teachings and guidance provided herein for introducing a sufficient number of encoding nucleic acids to reduce, eliminate or substantially eliminate the generation of unwanted by-products, such as 3HB and other short-chain (e.g., C2-C4, C2-C6, or C2-C8) carboxylate by-products (such as, e.g., acetate), and enhanced or increased carbon flux through 3HB—CoA, and/or one or more acyl-CoA(s), and/or 3HBAld, and thereby increasing Ac—COA derived products, such as, e.g., 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, CPL, adipate, 6ACA, and/or HDO, those skilled in the art will understand that the same engineering design also can be performed with respect to introducing at least the nucleic acids encoding the Wood-Ljungdahl enzymes or proteins absent in the host organism. Therefore, introduction of one or more encoding nucleic acids into the microbial organisms of the invention such that the modified organism contains the complete Wood-Ljungdahl pathway will confer syngas utilization ability.

Additionally, the reductive (reverse) tricarboxylic acid cycle is and/or hydrogenase activities can also be used for the conversion of CO, CO₂ and/or H2 to Ac—COA and other products such as acetate. Organisms capable of fixing carbon via the reductive TCA pathway can utilize one or more of the following enzymes: ATP citrate-lyase, citrate lyase, aconitase, isocitrate dehydrogenase, alpha-ketoglutarate: ferredoxin oxidoreductase, succinyl-CoA synthetase, succinyl-CoA transferase, fumarate reductase, fumarase, malate dehydrogenase, NAD (P) H: ferredoxin oxidoreductase, carbon monoxide dehydrogenase, and hydrogenase. Specifically, the reducing equivalents extracted from CO and/or H2 by carbon monoxide dehydrogenase and hydrogenase are utilized to fix CO₂ via the reductive TCA cycle into Ac—CoA or acetate. Acetate can be converted to Ac—COA by enzymes such as Ac—CoA transferase, acetate kinase/phosphotransacetylase, and Ac—COA synthetase. Ac—COA can be converted to, for example, precursors of Ac—COA derived products, such as precursors of 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, CPL, adipate, 6ACA, HDO, or any other Ac—CoA derived product, glyceraldehyde-3-phosphate, phosphoenolpyruvate, and pyruvate, by pyruvate: ferredoxin oxidoreductase and the enzymes of gluconeogenesis. Following the teachings and guidance provided herein for introducing a sufficient number of encoding nucleic acids to generate, for example, a 1,3-BDO pathway, a (3R)-hydroxybutyl (3R)-hydroxybutyrate pathway, a MAA pathway, or any other biosynthesis pathway that utilizes Ac—CoA as a substrate in its pathway, those skilled in the art will understand that the same engineering design also can be performed with respect to introducing at least the nucleic acids encoding the reductive TCA pathway enzymes or proteins absent in the host organism. Therefore, introduction of one or more encoding nucleic acids into the microbial organisms of the invention such that the modified organism contains the complete reductive TCA pathway will confer syngas utilization ability.

Accordingly, given the teachings and guidance provided herein, those skilled in the art will understand that a non-naturally occurring microbial organism having decreased unwanted by-products, such as 3HB and/or other carboxylate by-products (e.g., acetate, acetoacetate, etc.), and enhanced or increased carbon flux through 3HB—CoA, and/or one or more acyl-CoA(s), and/or 3HBAld, can be produced that secretes the biosynthesized compounds of the invention when grown on a carbon source, such as a carbohydrate, syngas, CO and/or CO₂. Such compounds include, for example, an Ac—COA derived product, such as 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, CPL, adipate, 6ACA, and/or HDO, and any of the intermediate metabolites therefrom. All that is required is to engineer in one or more of the required enzyme or protein activities to achieve biosynthesis of the desired compound or intermediate including, for example, inclusion of some or all of the Ac—COA derived product biosynthetic pathways. Accordingly, the invention provides a non-naturally occurring microbial organism that produces and/or secretes, an Ac—COA derived product, such as 1,3-BDO, (R)-1,3-BDO, MAA, (3R)-hydroxybutyl (3R)-hydroxybutyrate, or any other Ac—COA derived product when grown on a carbohydrate or other carbon source and produces and/or secretes any of the intermediate metabolites in the Ac—COA derived product pathway when grown on a carbohydrate or other carbon source. The Ac—COA derived product producing microbial organisms of the invention can initiate synthesis from an intermediate.

In some embodiments, the non-naturally occurring microbial organisms of the invention are constructed using methods well known in the art as exemplified herein to exogenously express at least one nucleic acid encoding an enzyme or protein in sufficient amounts to eliminate, substantially eliminate, or reduce by-products, such as 3HB by-product and/or other carboxylate by-products as described herein, and/or to enhance or increase carbon flux through 3HB—CoA, and/or one or more other acyl-CoAs as described herein, and/or 3HBAld, and thereby increasing Ac—COA derived products, such as 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, CPL, adipate, 6ACA, and/or HDO. In other embodiments, the non-naturally occurring microbial organisms of the invention are constructed using methods well known in the art as exemplified herein to exogenously express at least one nucleic acid encoding an enzyme or protein of a 3HB recycling loop, and/or a short-chain carboxylate by-product recycling loop, and/or a 3HB to 3HBAld conversion step, in sufficient amounts to eliminate, substantially eliminate, or reduce by-products, such as 3HB by-product and/or one or more other carboxylate by-products, and/or to enhance or increase carbon flux through 3HB—CoA, and/or one or more other one or more acyl-CoAs, and/or 3HBAld, and thereby increasing Ac—COA derived products, such as 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, CPL, adipate, 6ACA, and/or HDO.

It is understood that the microbial organisms of the invention are cultured under conditions sufficient to produce Ac—COA derived products, such as 11,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, CPL, adipate, 6ACA, and/or HDO. Following the teachings and guidance provided herein, the non-naturally occurring microbial organisms of the invention can achieve biosynthesis of an Ac—COA derived product, such as 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, CPL, adipate, 6ACA, and/or HDO, or any other Ac—COA derived product, resulting in intracellular concentrations between about 0.1-200 mM or more. Generally, the intracellular concentration of the Ac—COA derived product, such as 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, CPL, adipate, 6ACA, and/or HDO, is between about 30-300 mM, particularly between about 50-200 mM and more particularly between about 70-150 mM, including about 70 mM, 80 mM, 90 mM, 100 mM, 110 mM, 120 mM, 130 mM, 140 mM, 150 mM or more. Intracellular concentrations between and above each of these exemplary ranges also can be achieved from the non-naturally occurring microbial organisms of the invention.

In some embodiments, culture conditions include anaerobic or substantially anaerobic growth or maintenance conditions. In other embodiments, the culture conditions include microaerobic or substantially microaerobic growth of maintenance conditions (see, e.g., Examples 1-2). Exemplary anaerobic conditions have been described previously and are well known in the art. Exemplary anaerobic conditions for fermentation processes are described herein and are described, for example, in U.S. publication 2009/0047719, filed Aug. 10, 2007. Any of these conditions can be employed with the non-naturally occurring microbial organisms as well as other anaerobic conditions well known in the art. Under such anaerobic, substantially anaerobic, microaerobic, or substantially microaerobic conditions, the products of Ac—CoA derived products, such as 11,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, CPL, adipate, 6ACA, and/or HDO, can synthesize the desired Ac—CoA derived product at intracellular concentrations of 5-10 mM or more as well as all other concentrations exemplified herein. It is understood that, even though the above description refers to intracellular concentrations, the Ac—COA derived product producing microbial organisms can produce the Ac—COA derived product, such as 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, CPL, adipate, 6ACA, and/or HDO, or any other Ac—COA derived product, intracellularly and/or secrete the product into the culture medium.

In addition to the culturing and fermentation conditions disclosed herein, growth condition for achieving biosynthesis of, for example, 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, CPL, adipate, 6ACA, and/or HDO, or any other Ac—COA derived product can include the addition of an osmoprotectant to the culturing conditions. In certain embodiments, the non-naturally occurring microbial organisms of the invention can be sustained, cultured or fermented as described herein in the presence of an osmoprotectant. Briefly, an osmoprotectant refers to a compound that acts as an osmolyte and helps a microbial organism as described herein survive osmotic stress. Osmoprotectants include, but are not limited to, betaines, amino acids, and the sugar trehalose. Non-limiting examples of such are glycine betaine, praline e betaine, dimethylthetin, dimethylslfonioproprionate, 3-dimethylsulfonio-2-methylproprionate, pipecolic acid, dimethylsulfonioacetate, choline, L-carnitine and ectoine. In one aspect, the osmoprotectant is glycine betaine. It is understood to one of ordinary skill in the art that the amount and type of osmoprotectant suitable for protecting a microbial organism described herein from osmotic stress will depend on the microbial organism used. The amount of osmoprotectant in the culturing conditions can be, for example, no more than about 0.1 mM, no more than about 0.5 mM, no more than about 1.0 mM, no more than about 1.5 mM, no more than about 2.0 mM, no more than about 2.5 mM, no more than about 3.0 mM, no more than about 5.0 mM, no more than about 7.0 mM, no more than about 10 mM, no more than about 50 mM, no more than about 100 mM or no more than about 500 mM.

The culture conditions can include, for example, liquid culture procedures as well as fermentation and other large scale culture procedures. As described herein, particularly useful yields of the biosynthetic products of the invention can be obtained under anaerobic or substantially anaerobic culture conditions, or under microaerobic or substantially microaerobic conditions.

As described herein, one exemplary growth condition for achieving biosynthesis of, for example, 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, CPL, adipate, 6ACA, and/or HDO, or any other Ac—COA derived product includes anaerobic culture or fermentation conditions. In certain embodiments, the non-naturally occurring microbial organisms of the invention can be sustained, cultured or fermented under anaerobic or substantially anaerobic conditions. Briefly, anaerobic conditions refers to an environment devoid of oxygen. Substantially anaerobic conditions include, for example, a culture, batch fermentation or continuous fermentation such that the dissolved oxygen concentration in the medium remains between 0 and 10% of saturation. Substantially anaerobic conditions also includes growing or resting cells in liquid medium or on solid agar inside a sealed chamber maintained with an atmosphere of less than 1% oxygen. The percent of oxygen can be maintained by, for example, sparging the culture with an N2/CO₂ mixture or other suitable non-oxygen gas or gases. For strains where growth is not observed anaerobically, microaerobic or substantially anaerobic conditions can be applied by perforating the septum with a small hole for limited aeration.

The culture conditions described herein can be scaled up and grown continuously for manufacturing of, for example, 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, CPL, adipate, 6ACA, and/or HDO, or any other Ac—COA derived product. Exemplary growth procedures include, for example, fed-batch fermentation and batch separation; fed-batch fermentation and continuous separation, or continuous fermentation and continuous separation. All of these processes are well known in the art. Fermentation procedures are particularly useful for the biosynthetic production of commercial quantities of, for example, 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, CPL, adipate, 6ACA, and/or HDO, or any other Ac—COA derived product. Generally, and as with non-continuous culture procedures, the continuous and/or near-continuous production of, for example, 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, CPL, adipate, 6ACA, and/or HDO, or any other Ac—CoA derived product will include culturing a non-naturally occurring 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, CPL, adipate, 6ACA, and/or HDO, or any other Ac—CoA derived product producing organism of the invention in sufficient nutrients and medium to sustain and/or nearly sustain growth in an exponential phase. Continuous culture under such conditions can be include, for example, growth for 1 day, 2, 3, 4, 5, 6 or 7 days or more. Additionally, continuous culture can include longer time periods of 1 week, 2, 3, 4 or 5 or more weeks and up to several months. Alternatively, organisms of the invention can be cultured for hours, if suitable for a particular application. It is to be understood that the continuous and/or near-continuous culture conditions also can include all time intervals in between these exemplary periods. It is further understood that the time of culturing the microbial organism of the invention is for a sufficient period of time to produce a sufficient amount of product for a desired purpose.

Fermentation procedures are well known in the art. Briefly, fermentation for the biosynthetic production of, for example, 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, CPL, adipate, 6ACA, and/or HDO, or any other Ac—COA derived product can be utilized in, for example, fed-batch fermentation and batch separation; fed-batch fermentation and continuous separation, or continuous fermentation and continuous separation. Examples of batch and continuous fermentation procedures are well known in the art.

In addition to the above fermentation procedures using the 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, CPL, adipate, 6ACA, and/or HDO, or any other Ac—CoA derived product producers of the invention for continuous production of substantial quantities of 11,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, CPL, adipate, 6ACA, and/or HDO, or any other Ac—COA derived product, the 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, CPL, adipate, 6ACA, and/or HDO, or any other Ac—CoA derived product producers also can be, for example, simultaneously subjected to chemical synthesis procedures to convert the product to other compounds or the product can be separated from the fermentation culture and sequentially subjected to chemical conversion to convert the product to other compounds, if desired.

To generate better producers, metabolic modeling can be utilized to optimize growth conditions. Modeling can also be used to design gene knockouts that additionally optimize utilization of the pathway (see, for example, U.S. patent publications US 2002/0012939, US 2003/0224363, US 2004/0029149, US 2004/0072723, US 2003/0059792, US 2002/0168654 and US 2004/0009466, and U.S. Pat. No. 7,127,379). Modeling analysis allows reliable predictions of the effects on cell growth of shifting the metabolism towards more efficient production of 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, CPL, adipate, 6ACA, and/or HDO, or any other Ac—COA derived product.

One computational method for identifying and designing metabolic alterations favoring biosynthesis of a desired product is the OptKnock computational framework (Burgard et al., Biotechnol. Bioeng. 84:647-657 (2003)). OptKnock is a metabolic modeling and simulation program that suggests gene deletion or disruption strategies that result in genetically stable microorganisms which overproduce the target product. Specifically, the framework examines the complete metabolic and/or biochemical network of a microorganism in order to suggest genetic manipulations that force the desired biochemical to become an obligatory by-product of cell growth. By coupling biochemical production with cell growth through strategically placed gene deletions or other functional gene disruption, the growth selection pressures imposed on the engineered strains after long periods of time in a bioreactor lead to improvements in performance as a result of the compulsory growth-coupled biochemical production. Lastly, when gene deletions are constructed there is a negligible possibility of the designed strains reverting to their wild-type states because the genes selected by OptKnock are to be completely removed from the genome. Therefore, this computational methodology can be used to either identify alternative pathways that lead to biosynthesis of a desired product or used in connection with the non-naturally occurring microbial organisms for further optimization of biosynthesis of a desired product.

Briefly, OptKnock is a term used herein to refer to a computational method and system for modeling cellular metabolism. The OptKnock program relates to a framework of models and methods that incorporate particular constraints into flux balance analysis (FBA) models. These constraints include, for example, qualitative kinetic information, qualitative regulatory information, and/or DNA microarray experimental data. OptKnock also computes solutions to various metabolic problems by, for example, tightening the flux boundaries derived through flux balance models and subsequently probing the performance limits of metabolic networks in the presence of gene additions or deletions. OptKnock computational framework allows the construction of model formulations that allow an effective query of the performance limits of metabolic networks and provides methods for solving the resulting mixed-integer linear programming problems. The metabolic modeling and simulation methods referred to herein as OptKnock are described in, for example, U.S. publication 2002/0168654, filed Jan. 10, 2002, in International Patent No. PCT/US02/00660, filed Jan. 10, 2002, and U.S. publication 2009/0047719, filed Aug. 10, 2007, which are incorporated by reference in their entirety.

Another computational method for identifying and designing metabolic alterations favoring biosynthetic production of a product is a metabolic modeling and simulation system termed SimPheny®. This computational method and system is described in, for example, U.S. publication 2003/0233218, filed Jun. 14, 2002, and in International Patent Application No. PCT/US03/18838, filed Jun. 13, 2003. SimPheny® is a computational system that can be used to produce a network model in silico and to simulate the flux of mass, energy or charge through the chemical reactions of a biological system to define a solution space that contains any and all possible functionalities of the chemical reactions in the system, thereby determining a range of allowed activities for the biological system. This approach is referred to as constraints-based modeling because the solution space is defined by constraints such as the known stoichiometry of the included reactions as well as reaction thermodynamic and capacity constraints associated with maximum fluxes through reactions. The space defined by these constraints can be interrogated to determine the phenotypic capabilities and behavior of the biological system or of its biochemical components.

These computational approaches are consistent with biological realities because biological systems are flexible and can reach the same result in many different ways. Biological systems are designed through evolutionary mechanisms that have been restricted by fundamental constraints that all living systems must face. Therefore, constraints-based modeling strategy embraces these general realities. Further, the ability to continuously impose further restrictions on a network model via the tightening of constraints results in a reduction in the size of the solution space, thereby enhancing the precision with which physiological performance or phenotype can be predicted.

Given the teachings and guidance provided herein, those skilled in the art will be able to apply various computational frameworks for metabolic modeling and simulation to design and implement biosynthesis of a desired compound in host microbial organisms. Such metabolic modeling and simulation methods include, for example, the computational systems exemplified above as SimPheny® and OptKnock. For illustration of the invention, some methods are described herein with reference to the OptKnock computation framework for modeling and simulation. Those skilled in the art will know how to apply the identification, design and implementation of the metabolic alterations using OptKnock to any of such other metabolic modeling and simulation computational frameworks and methods well known in the art.

The methods described above will provide one set of metabolic reactions to disrupt. Elimination of each reaction within the set or metabolic modification can result in a desired product as an obligatory product during the growth phase of the organism. Because the reactions are known, a solution to the bilevel OptKnock problem also will provide the associated gene or genes encoding one or more enzymes that catalyze each reaction within the set of reactions. Identification of a set of reactions and their corresponding genes encoding the enzymes participating in each reaction is generally an automated process, accomplished through correlation of the reactions with a reaction database having a relationship between enzymes and encoding genes.

Once identified, the set of reactions that are to be disrupted in order to achieve production of a desired product are implemented in the target cell or organism by functional disruption of at least one gene encoding each metabolic reaction within the set. One particularly useful means to achieve functional disruption of the reaction set is by deletion of each encoding gene. However, in some instances, it can be beneficial to disrupt the reaction by other genetic aberrations including, for example, mutation, deletion of regulatory regions such as promoters or cis binding sites for regulatory factors, or by truncation of the coding sequence at any of a number of locations. These latter aberrations, resulting in less than total deletion of the gene set can be useful, for example, when rapid assessments of the coupling of a product are desired or when genetic reversion is less likely to occur.

To identify additional productive solutions to the above described bilevel OptKnock problem which lead to further sets of reactions to disrupt or metabolic modifications that can result in the biosynthesis, including growth-coupled biosynthesis of a desired product, an optimization method, termed integer cuts, can be implemented. This method proceeds by iteratively solving the OptKnock problem exemplified above with the incorporation of an additional constraint referred to as an integer cut at each iteration. Integer cut constraints effectively prevent the solution procedure from choosing the exact same set of reactions identified in any previous iteration that obligatorily couples product biosynthesis to growth. For example, if a previously identified growth-coupled metabolic modification specifies reactions 1, 2, and 3 for disruption, then the following constraint prevents the same reactions from being simultaneously considered in subsequent solutions. The integer cut method is well known in the art and can be found described in, for example, Burgard et al., Biotechnol. Prog. 17:791-797 (2001). As with all methods described herein with reference to their use in combination with the OptKnock computational framework for metabolic modeling and simulation, the integer cut method of reducing redundancy in iterative computational analysis also can be applied with other computational frameworks well known in the art including, for example, SimPheny®.

The methods exemplified herein allow the construction of cells and organisms that biosynthetically produce a desired product, including the obligatory coupling of production of a target biochemical product to growth of the cell or organism engineered to harbor the identified genetic alterations. Therefore, the computational methods described herein allow the identification and implementation of metabolic modifications that are identified by an in silico method selected from OptKnock or SimPheny®. The set of metabolic modifications can include, for example, addition of one or more biosynthetic pathway enzymes and/or functional disruption of one or more metabolic reactions including, for example, disruption by gene deletion.

As discussed above, the OptKnock methodology was developed on the premise that mutant microbial networks can be evolved towards their computationally predicted maximum-growth phenotypes when subjected to long periods of growth selection. In other words, the approach leverages an organism's ability to self-optimize under selective pressures. The OptKnock framework allows for the exhaustive enumeration of gene deletion combinations that force a coupling between biochemical production and cell growth based on network stoichiometry. The identification of optimal gene/reaction knockouts requires the solution of a bilevel optimization problem that chooses the set of active reactions such that an optimal growth solution for the resulting network overproduces the biochemical of interest (Burgard et al., Biotechnol. Bioeng. 84:647-657 (2003)).

An in silico stoichiometric model of E. coli metabolism can be employed to identify essential genes for metabolic pathways as exemplified previously and described in, for example, U.S. patent publications US 2002/0012939, US 2003/0224363, US 2004/0029149, US 2004/0072723, US 2003/0059792, US 2002/0168654 and US 2004/0009466, and in U.S. Pat. No. 7,127,379. As disclosed herein, the OptKnock mathematical framework can be applied to pinpoint gene deletions leading to the growth-coupled production of a desired product. Further, the solution of the bilevel OptKnock problem provides only one set of deletions. To enumerate all meaningful solutions, that is, all sets of knockouts leading to growth-coupled production formation, an optimization technique, termed integer cuts, can be implemented. This entails iteratively solving the OptKnock problem with the incorporation of an additional constraint referred to as an integer cut at each iteration, as discussed above.

In some embodiments, microaerobic designs can be used based on the growth-coupled formation of the desired product. To examine this, production cones can be constructed for each strategy by first maximizing and, subsequently minimizing the product yields at different rates of biomass formation feasible in the network. If the rightmost boundary of all possible phenotypes of the mutant network is a single point, it implies that there is a unique optimum yield of the product at the maximum biomass formation rate possible in the network. In other cases, the rightmost boundary of the feasible phenotypes is a vertical line, indicating that at the point of maximum biomass the network can make any amount of the product in the calculated range, including the lowest amount at the bottommost point of the vertical line. Such designs are given a low priority.

Serial culture involves repetitive transfer of a small volume of grown culture to a much larger vessel containing fresh growth medium. When the cultured organisms have grown to saturation in the new vessel, the process is repeated. This method has been used to achieve the longest demonstrations of sustained culture in the literature (Lenski and Travisano, Proc. Natl. Acad. Sci. USA 91:6808-6814 (1994)) in experiments which clearly demonstrated consistent improvement in reproductive rate over a period of years. Typically, transfer of cultures is usually performed during exponential phase, so each day the transfer volume is precisely calculated to maintain exponential growth through the next 24 hour period. Manual serial dilution is inexpensive and easy to parallelize.

In continuous culture the growth of cells in a chemostat represents an extreme case of dilution in which a very high fraction of the cell population remains. As a culture grows and becomes saturated, a small proportion of the grown culture is replaced with fresh media, allowing the culture to continually grow at close to its maximum population size. Chemostats have been used to demonstrate short periods of rapid improvement in reproductive rate (Dykhuizen, Methods Enzymol. 613-631 (1993)). The potential usefulness of these devices was recognized, but traditional chemostats were unable to sustain long periods of selection for increased reproduction rate, due to the unintended selection of dilution-resistant (static) variants. These variants are able to resist dilution by adhering to the surface of the chemostat, and by doing so, outcompete less adherent individuals, including those that have higher reproductive rates, thus obviating the intended purpose of the device (Chao and Ramsdell, J. Gen. Microbiol 20:132-138 (1985)). One possible way to overcome this drawback is the implementation of a device with two growth chambers, which periodically undergo transient phases of sterilization, as described previously (Marliere and Mutzel, U.S. Pat. No. 6,686,194).

Evolugator™ is a continuous culture device developed by Evolugate, LLC (Gainesville, FL) and exhibits significant time and effort savings over traditional evolution techniques (de Crecy et al., Appl. Microbiol. Biotechnol. 77:489-496 (2007)). The cells are maintained in prolonged exponential growth by the serial passage of batch cultures into fresh medium before the stationary phase is attained. By automating optical density measurement and liquid handling, the Evolugator™ can perform serial transfer at high rates using large culture volumes, thus approaching the efficiency of a chemostat in evolution of cell fitness. For example, a mutant of Acinetobacter sp. ADP1 deficient in a component of the translation apparatus, and having severely hampered growth, was evolved in 200 generations to 80% of the wild-type growth rate. However, in contrast to the chemostat which maintains cells in a single vessel, the machine operates by moving from one “reactor” to the next in subdivided regions of a spool of tubing, thus eliminating any selection for wall-growth. The transfer volume is adjustable, and normally set to about 50%. A drawback to this device is that it is large and costly, thus running large numbers of evolutions in parallel is not practical. Furthermore, gas addition is not well regulated, and strict anaerobic conditions are not maintained with the current device configuration. Nevertheless, this is an alternative method to adaptively evolve a production strain.

As disclosed herein, a nucleic acid encoding a desired activity of an enzyme that eliminates, substantially eliminates, or reduces by-products and/or increases or enhances carbon flux through 3HB—CoA, and/or one or more acyl-CoA(S), and/or 3HBAld, and thereby increasing Ac—COA derived products, such as 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, CPL, adipate, 6ACA, and/or HDO, can be introduced into a host organism. In some cases, it can be desirable to modify an activity of an enzyme or protein that decreases unwanted by-products or protein to increase carbon flux through Ac—COA and thereby increasing Ac—COA derived products, such as 1,3-BDO, R-1,3 BDO, S-1,3 BDO, (3R)-hydroxybutyl (3R)-hydroxybutyrate, butyrate, butanol, propane, formate, isopropanol, MAA, MAA esters, HMD, CPL, adipate, 6ACA, and/or HDO. For example, known mutations that increase the activity of a protein or enzyme can be introduced into an encoding nucleic acid molecule. Additionally, optimization methods can be applied to increase the activity of an enzyme or protein and/or decrease an inhibitory activity, for example, decrease the activity of a negative regulator.

One such optimization method is directed evolution. Directed evolution is a powerful approach that involves the introduction of mutations targeted to a specific gene in order to improve and/or alter the properties of an enzyme. Improved and/or altered enzymes can be identified through the development and implementation of sensitive high-throughput screening assays that allow the automated screening of many enzyme variants (for example, >104). Iterative rounds of mutagenesis and screening typically are performed to afford an enzyme with optimized properties. Computational algorithms that can help to identify areas of the gene for mutagenesis also have been developed and can significantly reduce the number of enzyme variants that need to be generated and screened. Numerous directed evolution technologies have been developed (for reviews, see Hibbert et al., Biomol. Eng 22:11-19 (2005); Huisman and Lalonde, In Biocatalysis in the pharmaceutical and biotechnology industries pgs. 717-742 (2007), Patel (ed.), CRC Press; Otten and Quax. Biomol. Eng 22:1-9 (2005).; and Sen et al., Appl Biochem. Biotechnol 143:212-223 (2007)) to be effective at creating diverse variant libraries, and these methods have been successfully applied to the improvement of a wide range of properties across many enzyme classes. Enzyme characteristics that have been improved and/or altered by directed evolution technologies include, for example: selectivity/specificity, for conversion of non-natural substrates; temperature stability, for robust high temperature processing; pH stability, for bioprocessing under lower or higher pH conditions; substrate or product tolerance, so that high product titers can be achieved; binding (Kₘ), including broadening substrate binding to include non-natural substrates; inhibition (Kᵢ), to remove inhibition by products, substrates, or key intermediates; activity (kcat), to increases enzymatic reaction rates to achieve desired flux; expression levels, to increase protein yields and overall pathway flux; oxygen stability, for operation of air sensitive enzymes under aerobic conditions; and anaerobic activity, for operation of an aerobic enzyme in the absence of oxygen.

A number of exemplary methods have been developed for the mutagenesis and diversification of genes to target desired properties of specific enzymes. Such methods are well known to those skilled in the art. Any of these can be used to alter and/or optimize the activity of an enzyme or protein that eliminates, substantially eliminates or reduces unwanted by-products, such as 3HB by-product, and enhances or increases carbon flux through 3HB—CoA. Such methods include, but are not limited to EpPCR, which introduces random point mutations by reducing the fidelity of DNA polymerase in PCR reactions (Pritchard et al., J Theor. Biol. 234:497-509 (2005)); Error-prone Rolling Circle Amplification (epRCA), which is similar to epPCR except a whole circular plasmid is used as the template and random 6-mers with exonuclease resistant thiophosphate linkages on the last 2 nucleotides are used to amplify the plasmid followed by transformation into cells in which the plasmid is re-circularized at tandem repeats (Fujii et al., Nucleic Acids Res. 32: e145 (2004); and Fujii et al., Nat. Protoc. 1:2493-2497 (2006)); DNA or Family Shuffling, which typically involves digestion of two or more variant genes with nucleases such as Dnase I or EndoV to generate a pool of random fragments that are reassembled by cycles of annealing and extension in the presence of DNA polymerase to create a library of chimeric genes (Stemmer, Proc Natl Acad Sci USA 91:10747-10751 (1994); and Stemmer, Nature 370:389-391 (1994)); Staggered Extension (StEP), which entails template priming followed by repeated cycles of 2 step PCR with denaturation and very short duration of annealing/extension (as short as 5 sec) (Zhao et al., Nat. Biotechnol. 16:258-261 (1998)); Random Priming Recombination (RPR), in which random sequence primers are used to generate many short DNA fragments complementary to different segments of the template (Shao et al., Nucleic Acids Res 26:681-683 (1998)).

Additional methods include Heteroduplex Recombination, in which linearized plasmid DNA is used to form heteroduplexes that are repaired by mismatch repair (Volkov et al, Nucleic Acids Res. 27: e18 (1999); and Volkov et al., Methods Enzymol. 328:456-463 (2000)); Random Chimeragenesis on Transient Templates (RACHITT), which employs Dnase I fragmentation and size fractionation of single stranded DNA (ssDNA) (Coco et al., Nat. Biotechnol. 19:354-359 (2001)); Recombined Extension on Truncated templates (RETT), which entails template switching of unidirectionally growing strands from primers in the presence of unidirectional ssDNA fragments used as a pool of templates (Lee et al., J. Molec. Catalysis 26:119-129 (2003)); Degenerate Oligonucleotide Gene Shuffling (DOGS), in which degenerate primers are used to control recombination between molecules; (Bergquist and Gibbs, Methods Mol. Biol 352:191-204 (2007); Bergquist et al., Biomol. Eng 22:63-72 (2005); Gibbs et al., Gene 271:13-20 (2001)); Incremental Truncation for the Creation of Hybrid Enzymes (ITCHY), which creates a combinatorial library with 1 base pair deletions of a gene or gene fragment of interest (Ostermeier et al., Proc. Natl. Acad. Sci. USA 96:3562-3567 (1999); and Ostermeier et al., Nat. Biotechnol. 17:1205-1209 (1999)); Thio-Incremental Truncation for the Creation of Hybrid Enzymes (THIO-ITCHY), which is similar to ITCHY except that phosphothioate dNTPs are used to generate truncations (Lutz et al., Nucleic Acids Res 29: E16 (2001)); SCRATCHY, which combines two methods for recombining genes, ITCHY and DNA shuffling (Lutz et al., Proc. Natl. Acad. Sci. USA 98:11248-11253 (2001)); Random Drift Mutagenesis (RNDM), in which mutations made via epPCR are followed by screening/selection for those retaining usable activity (Bergquist et al., Biomol. Eng. 22:63-72 (2005)); Sequence Saturation Mutagenesis (SeSaM), a random mutagenesis method that generates a pool of random length fragments using random incorporation of a phosphothioate nucleotide and cleavage, which is used as a template to extend in the presence of “universal” bases such as inosine, and replication of an inosine-containing complement gives random base incorporation and, consequently, mutagenesis (Wong et al., Biotechnol. J. 3:74-82 (2008); Wong et al., Nucleic Acids Res. 32:e26 (2004); and Wong et al., Anal. Biochem. 341:187-189 (2005)); Synthetic Shuffling, which uses overlapping oligonucleotides designed to encode “all genetic diversity in targets” and allows a very high diversity for the shuffled progeny (Ness et al., Nat. Biotechnol. 20:1251-1255 (2002)); Nucleotide Exchange and Excision Technology NexT, which exploits a combination of dUTP incorporation followed by treatment with uracil DNA glycosylase and then piperidine to perform endpoint DNA fragmentation (Muller et al., Nucleic Acids Res. 33: e117 (2005)).

Further methods include Sequence Homology-Independent Protein Recombination (SHIPREC), in which a linker is used to facilitate fusion between two distantly related or unrelated genes, and a range of chimeras is generated between the two genes, resulting in libraries of single-crossover hybrids (Sieber et al., Nat. Biotechnol. 19:456-460 (2001)); Gene Site Saturation Mutagenesis™ (GSSM™), in which the starting materials include a supercoiled double stranded DNA (dsDNA) plasmid containing an insert and two primers which are degenerate at the desired site of mutations (Kretz et al., Methods Enzymol. 388:3-11 (2004)); Combinatorial Cassette Mutagenesis (CCM), which involves the use of short oligonucleotide cassettes to replace limited regions with a large number of possible amino acid sequence alterations (Reidhaar-Olson et al. Methods Enzymol. 208:564-586 (1991); and Reidhaar-Olson et al. Science 241:53-57 (1988)); Combinatorial Multiple Cassette Mutagenesis (CMCM), which is essentially similar to CCM and uses epPCR at high mutation rate to identify hot spots and hot regions and then extension by CMCM to cover a defined region of protein sequence space (Reetz et al., Angew. Chem. Int. Ed Engl. 40:3589-3591 (2001)); the Mutator Strains technique, in which conditional ts mutator plasmids, utilizing the mutD5 gene, which encodes a mutant subunit of DNA polymerase III, to allow increases of 20 to 4000-X in random and natural mutation frequency during selection and block accumulation of deleterious mutations when selection is not required (Selifonova et al., Appl. Environ. Microbiol. 67:3645-3649 (2001)); Low et al., J. Mol. Biol. 260:359-3680 (1996)).

Additional exemplary methods include Look-Through Mutagenesis (LTM), which is a multidimensional mutagenesis method that assesses and optimizes combinatorial mutations of selected amino acids (Rajpal et al., Proc. Natl. Acad. Sci. USA 102:8466-8471 (2005)); Gene Reassembly, which is a DNA shuffling method that can be applied to multiple genes at one time or to create a large library of chimeras (multiple mutations) of a single gene (Tunable GeneReassembly™ (TGR™) Technology supplied by Verenium Corporation), in Silico Protein Design Automation (PDA), which is an optimization algorithm that anchors the structurally defined protein backbone possessing a particular fold, and searches sequence space for amino acid substitutions that can stabilize the fold and overall protein energetics, and generally works most effectively on proteins with known three-dimensional structures (Hayes et al., Proc. Natl. Acad. Sci. USA 99:15926-15931 (2002)); and Iterative Saturation Mutagenesis (ISM), which involves using knowledge of structure/function to choose a likely site for enzyme improvement, performing saturation mutagenesis at chosen site using a mutagenesis method such as Stratagene QuikChange (Stratagene; San Diego CA), screening/selecting for desired properties, and, using improved clone(s), starting over at another site and continue repeating until a desired activity is achieved (Reetz et al., Nat. Protoc. 2:891-903 (2007); and Reetz et al., Angew. Chem. Int. Ed Engl. 45:7745-7751 (2006)). Any of the aforementioned methods for mutagenesis can be used alone or in any combination. Additionally, any one or combination of the directed evolution methods can be used in conjunction with adaptive evolution techniques, as described herein.

### EXAMPLES

### Example 1

### Expression of Acyl-CoA Synthetase (ACS) Results in Improved 1,3-Butanediol (1,3-BDO) Titer and Reduced Levels of 3-Hydroxybutyrate (3HB)

The following example demonstrates that bacterial strains expressing a 1,3-BDO pathway and an acyl-CoA synthetase (ACS; also referred to herein as a CoA ligase) (GenBank Accession No. WP_011046428.1; see, SEQ ID NO:1) exhibit increased production of 1,3-butanediol (1,3-BDO) and reduced production of 3-hydroxybutyrate (3HB). The E. coli control strain L20952, which does not express an ACS, and the E. coli strains L20929, L20928, and L20990, which express varying levels of ACS (see, TABLE 4), were cultured, and the production of 1,3-BDO and 3HB were measured.

TABLE 4
Strains Expressing Varying Levels of ACS (CoA ligase)

| Strain | Description |
|---|---|
| L20952 | Control strain without expression of ACS (CoA ligase) |
| L20929 | Weak overexpression of ACS (CoA ligase) (p115 promoter) |
| L20928 | Medium overexpression of ACS (CoA ligase) (p108 promoter) |
| L20990 | Strong overexpression of ACS (CoA ligase) (p100 promoter) |

E. coli strains L20952 (control), L20929, L20928, and L20990 (see, Tables 4 and 5) were cultured under microaerobic conditions. Briefly, twenty-milliliter bottle cultivations for metabolite production or bioconversion were performed in M9 minimal salts medium (6.78 g/L Na₂HPO₄, 3.0 g/L KH₂PO₄, 0.5 g/L NaCl, 1.0 g/L NH₄Cl, 1 mM MgSO₄, 0.1 mM CaCl₂)), supplemented with 10 mM NaHCO₃, 20 g/L D-glucose, 100 mM MOPS (3-(N-morpholino) propanesulfonic acid; to improve the buffering capacity), 10 μg/mL thiamine, and the appropriate antibiotics for plasmid maintenance.

Microaerobic conditions were used for all strains, and were established by initially flushing capped anaerobic bottles with nitrogen for 5 minutes, and then piercing the septum with a 23 gauge needle (Becton Dickinson) after inoculation. The needle was retained in the septum-during growth to allow a small amount of air to enter. All cultures were grown at 35° C. Fermentations were performed with 1 L initial culture volume in 2 L Biostat® B plus bioreactors (Sartorius Stedim Biotech) using modified M9 minimal medium (6.78 g/L Na₂HPO₄, 3.0 g/L KH₂PO₄, 0.5 g/L NaCl, 1.0 g/L NH₄Cl, 1 mM MgSO₄, 0.1 mM CaCl₂)), supplemented with 20 g/L D-glucose. The temperature was held at 35° C., and the pH was held at 6.95 using 2M NH₄OH (aerobic phase), or 2M Na₂CO₃ (microaerobic phase). The agitation rate was set to 700 RPM. Concentrated glucose was fed in to maintain the glucose concentration in the vessel at between 0.5 and 5.0 g/L.

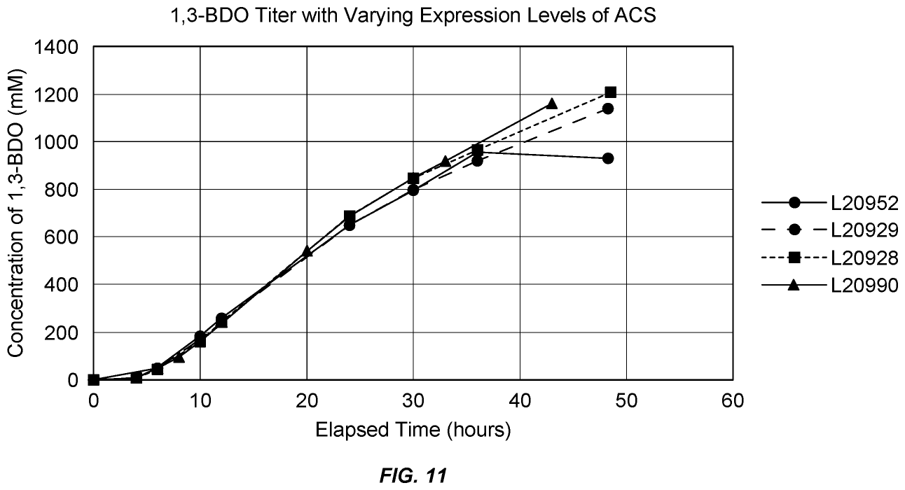

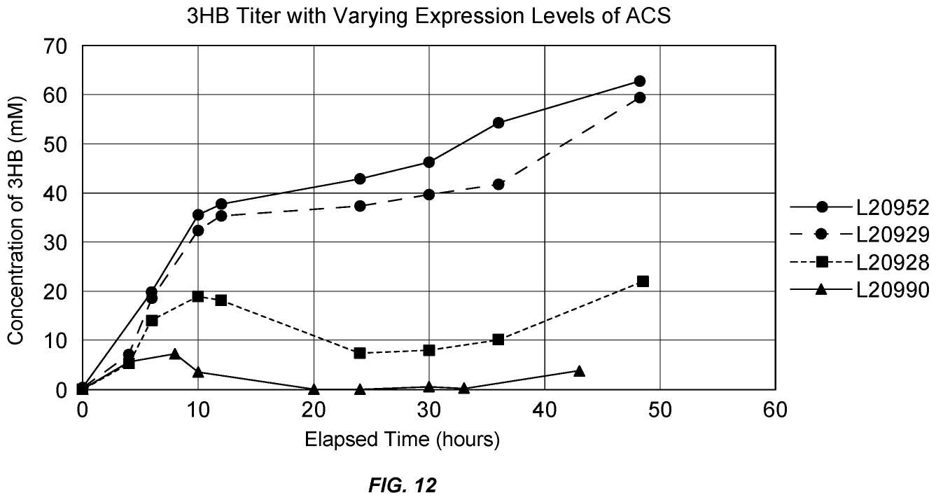

The strains were cultured for 50 hours, after which the titers of 1,3-BDO and 3HB were measured using HPLC. The results, which are summarized in TABLE 5 below, show that the strains expressing the ACS resulted in an increased 1,3-BDO titer and a reduced 3HB titer, compared to the control strain not expressing the ACS (L20952). The reduction of 3HB production and increase in 1,3-BDO production correlated with the expression levels of the ACS protein (see, also, FIGS. 11 and 12). For example, strain L20990, with a strong overexpression of ACS, resulted in the lowest 3HB titer, followed by strain L20928, with medium overexpression of ACS, and strain L20929, with weak overexpression of ACS, while control strain L20952, without expression of ACS, resulted in the highest 3HB titer. These results demonstrate that overexpression of ACS improves the titer of 1,3-butanediol (1,3-BDO) and results in the reduced production of 3-hydroxybutyrate (3HB) by-product.

Thus, the expression (or overexpression) of an ACS (e.g., an AMP-forming ACS), in a strain containing an acetyl-CoA derived pathway with a 3HB—CoA intermediate, such as a 1,3-BDO pathway as exemplified herein, results in a 3HB recycling loop that converts 3HB by-product to 3HB—CoA intermediate, thereby increasing the carbon flux through the 3HB—CoA intermediate, increasing the titer of the acetyl-CoA derived product (exemplified herein by 1,3-BDO), and reducing the titer of 3HB by-product.

TABLE 5
Titers of 1,3 BDO and 3HB Produced by Strains
Expressing Varying Amounts of ACS

| | | 1,3-BDO | 3HB |
|---|---|---|---|
| | | Titer | Titer |
| Strain | Description | (mM) | (mM) |
| L20952 | Control strain without ACS | 929 | 62.7 |
| L20929 | Weak overexpression of ACS (p115 | 1137 | 59.3 |
| | promoter) | | |
| L20928 | Medium overexpression of ACS (p108 | 1207 | 21.9 |
| | promoter) | | |
| L20990 | Strong overexpression of ACS (p100 | 1158 | 3.71 |
| | promoter) | | |

### Example 2

### Expression of a Carboxylic Acid Reductase (CAR) Results in Improved 1,3-Butanediol (1,3-BDO) Titer and Reduced Levels of 3-Hydroxybutyrate (3HB)

The following example demonstrates that bacterial strains expressing an enzyme with carboxylic acid reductase (CAR) activity exhibit increased production of 1,3-butanediol (1,3-BDO) and reduced production of 3-hydroxybutyrate (3HB). The E. coli control strain L29031, which does not express a CAR, and E. coli strain L29309 (see, TABLE 6), which overexpresses a variant (see, SEQ ID NO: 3) of the wild-type CAR from Mycobacterium avium (GenBank Accession No. WP_003872682.1; SEQ ID NO: 2), were cultured, and the production of 1,3-BDO and 3HB were measured. Both the control strain (L29031) and the strain overexpressing CAR (L29309) also expressed a 1,3-BDO product pathway, and expressed the ACS of SEQ ID NO: 1 (medium overexpression), as described in Example 1. Therefore, any differences in the 1,3-BDO and 3HB titers produced by the two strains is dependent on the absence or presence of CAR expression.

TABLE 6
Strain Descriptions

| Strain | Description |
|---|---|
| L29031 | Control strain without expression of CAR |
| L29309 | Overexpression of CAR |

The E. coli strains L29031 (control) and L29309 (see, TABLE 6) were cultured under microaerobic conditions. Briefly, twenty-milliliter bottle cultivations for metabolite production or bioconversion were performed in M9 minimal salts medium (6.78 g/L Na₂HPO₄, 3.0 g/L KH₂PO₄, 0.5 g/L NaCl, 1.0 g/L NH₄Cl, 1 mM MgSO₄, 0.1 mM CaCl₂)), supplemented with 10 mM NaHCO₃, 20 g/L D-glucose, 100 mM MOPS (3-(N-morpholino) propanesulfonic acid, to improve the buffering capacity), 10 μg/mL thiamine, and the appropriate antibiotics for plasmid maintenance.

Microaerobic conditions were used for all strains and were established by initially flushing capped anaerobic bottles with nitrogen for 5 minutes, and then piercing the septum with a 23 gauge needle (Becton Dickinson) after inoculation. The needle was retained in the septum during growth to allow a small amount of air to enter. All cultures were grown at 35° C. Fermentations were performed with a 1 L initial culture volume in 2 L Biostat® B plus bioreactors (Sartorius Stedim Biotech), using modified M9 minimal medium (6.78 g/L Na₂HPO₄, 3.0 g/L KH₂PO₄, 0.5 g/L NaCl, 1.0 g/L NH₄Cl, 1 mM MgSO₄, 0.1 mM CaCl₂)), supplemented with 20 g/L D-glucose. The temperature was held at 35° C., and the pH was held at 6.7 using 2M NH₄OH (aerobic phase), or 2M Na₂CO₃ (microaerobic phase). The agitation rate was set to 700 RPM. Concentrated glucose was fed in to maintain the glucose concentration in the vessel at between 0.5 and 5.0 g/L.

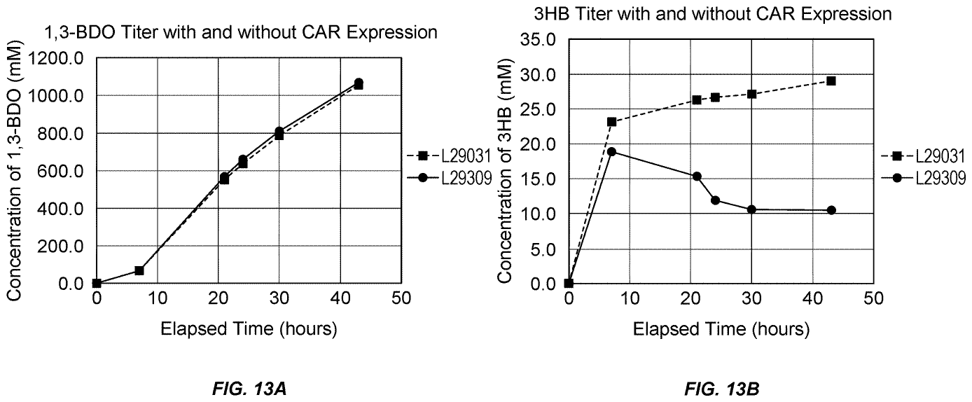

The strains were cultured for 43-44 hours, after which the titers of 1,3-BDO and 3HB were measured using HPLC. The results, which are summarized in TABLE 7 below and depicted in FIGS. 13A and 13B, show that the strain expressing the CAR (L29309) resulted in an increased 1,3-BDO titer and a reduced 3HB titer, compared to the control strain not expressing the CAR (L29031). These results demonstrate that overexpression of an enzyme with carboxylic acid reductase activity improves the titer of 1,3-butanediol (1,3-BDO) and results in the reduced production of 3-hydroxybutyrate (3HB).

Thus, the expression (or overexpression) of a CAR (or a variant or a homolog thereof with CAR activity), in a strain containing an acetyl-CoA derived product pathway having a 3HB-derived intermediate, such as 3HB—CoA and/or 3HBAld, exemplified herein by a 1,3-BDO pathway, results in the recycling or reintroduction of 3HB by-product back into the 1,3-BDO biosynthetic pathway, by converting the 3HB by-product to 3-hydroxybutyraldehyde (3HB—Ald) intermediate, thereby increasing the carbon flux through the 3HB—Ald intermediate, increasing the titer of 1,3-BDO product, and reducing the titer of 3HB byproduct.

These results, together with the results from Example 1, demonstrate that:
(a) the expression of ACS reduces the amount of 3HB by-product and increases the production of an acetyl-CoA derived product (e.g., 1,3-BDO) with a 3HB-derived intermediate (e.g., 3HB—CoA, and optionally, 3HBAld);
(b) the expression of CAR reduces the amount of 3HB by-product and increases the production of an acetyl-CoA derived product (e.g., 1,3-BDO) with a 3HB-derived intermediate (e.g., 3HB—CoA and/or a 3HBAld); and
(c) the expression of both ACS and CAR in a strain expressing an acetyl-CoA derived product (e.g., 1,3-BDO) pathway with 3HB-derived intermediates (e.g., 3HB—CoA and 3HBAld), results in a greater reduction in 3HB by-product and/or a greater increase in the production of an acetyl-CoA derived product, compared to a strain expressing only an ACS or only a CAR.

TABLE 7
Effects of CAR expression on the Titers of 1,3 BDO and 3HB

| | | 1,3-BDO | 3HB |
|---|---|---|---|
| | | Titer | Titer |
| Strain | Description | (mM) | (mM) |
| L29031 | Control strain without expression of CAR | 1055.4 | 29.0 |
| L29309 | Overexpression of CAR | 1066.8 | 10.5 |

### Example 3

### Carboxylic Acid Reductase (CAR) Catalyzes the Reduction of 3-Hydroxybutyrate (3HB) to 3-Hydroxybutyraldehyde (3HD-Ald)

This example demonstrates that an enzyme with carboxylic acid reductase (CAR) activity catalyzes the reduction of 3HB to 3HB—Ald, and is thus useful for increasing the production (e.g., titer, rate, and/or yield) of acetyl-CoA derived products, such as 1,3-BDO, having biosynthetic pathways that include 3HB—CoA and 3HB—Ald intermediates, and for decreasing the production of 3HB by-product.

In vitro CAR activity for the conversion of 3HB to 3HB—Ald was determined by direct measurement of the decrease in NADPH concentration, by measuring NADPH absorbance at 340 nm over time. The reactions were carried out in 96-well absorbance plates, and varying concentrations of 3HB substrate (see, TABLE 8 below), 0.5 mM of NADPH, 1 mM of ATP, 5 mM of MgCl₂, and 1 μM of CAR enzyme (see, SEQ ID NO: 3) were added. A negative control reaction was included, in which addition of 3HB substrate was omitted. CAR activity was measured as μM of NADPH oxidized per minute per μM of CAR enzyme.

The results, which are summarized in TABLE 8 below, show that CAR activity increases as the concentration of 3HB substrate increases. These results demonstrate that CAR has activity for the conversion of 3HB to 3HB—Ald, and that microbial strains expressing CAR and a biosynthetic product pathway that includes 3HB—CoA and/or 3HB—Ald intermediates result in the increased production (e.g., titer, rate, and/or yield) of the product (e.g., 1,3-BDO), and decreased production of 3HB by-product (see, Example 2).

TABLE 8
CAR Activity on 3HB Substrate

| 3HB Substrate | CAR Activity |
|---|---|
| Concentration (mM) | (μM NADPH oxidized/min/μM CAR) |
| 0.00 | 0.00 |
| 0.78125 | 0.47 |
| 1.5625 | 1.30 |
| 3.125 | 2.61 |
| 6.25 | 4.24 |
| 12.5 | 5.87 |
| 25 | 8.81 |
| 50 | 10.25 |

### Example 4

### Acyl-CoA Synthetase (ACS) Catalyzes the Activation of Short-Chain Carboxylic Acids to CoA Thioesters (Acyl-CoAs)

This example demonstrates that the acyl-CoA synthetase (ACS) described herein has activity on other substrates besides 3HB. In particular, it is shown herein that the ACS has activity on a broad range of short-chain carboxylic acid (or carboxylate) substrates, and is thus useful for reducing the production of short-chain carboxylate by-products and increasing the production of a wide range of products having biosynthetic pathways that include CoA thioesters of short-chain carboxylic acids (i.e., short-chain acyl-CoAs), including all acetyl-CoA derived products.

Purified acyl-CoA synthetase (ACS) (GenBank Accession No. WP_011046428.1; see, SEQ ID NO: 1) was assayed for activity on a range of substrates of various chain lengths. The substrates tested, which were conjugate bases (or carboxylates) of C2-C8 carboxylic acids, included acetate (C2), acetoacetate (C4), butyrate (C4), (R)- and(S)-3-hydroxybutryate ((R)-3-HB and(S)-3HB; C4), succinate (C4), hexanoate (C6), adipate (C6), 6-aminocaproic acid (6ACA; C6), and octanoate (C8).

An enzymatic coupled assay for detection of AMP, which is a co-product of the synthetase/ligase enzymatic reaction catalyzed by the ACS/CoA ligase, was used. The coupling enzymes used were adenylate kinase, pyruvate dehydrogenase, and lactate dehydrogenase, which catalyze reactions resulting in the oxidation of 2 NADH molecules per molecule of AMP produced by the ACS/CoA ligase enzymatic reaction. The coupling reagents for AMP detection were phospho (enol) pyruvate and NADH. The ACS/CoA ligase enzymatic reactions were followed by monitoring NADH oxidation over time, by measuring absorbance at 340 nm.

Reactions were set up in 96-well absorbance plates, in aqueous buffer at pH 7.5, and 5 mM of conjugate base substrate, 250 μM of Coenzyme A (CoA), 1 mM of ATP, 5 mM of MgCl₂, 1 mM of phospho (enol) pyruvate, 500 μM of NADH, and non-rate limiting concentrations of the three coupling enzymes (adenylate kinase, pyruvate dehydrogenase, and lactate dehydrogenase), were added for each reaction. The ACS enzyme was added at a concentration that was appropriate for measuring a linear change on absorbance over up to 20 minutes. For example, 100 nM of ACS was added for the reactions with butyrate or 3-HB as the substrates. Negative control reactions omitted the substrate (conjugate base), the CoA, or the ACS enzyme. No activity was observed for the negative control reactions.

The results are summarized in TABLE 9 below, which provides units of activity as μM of AMP formed per minute per μM of ACS. The results demonstrate that the ACS not only has activity on both the (R) and(S) forms of 3HB, but that it also exhibits activity on a broad range of other short-chain carboxylate substrates, including, e.g., acetate, acetoacetate, butyrate, succinate, hexanoate, and 6ACA. Thus, the ACS is also useful for reducing the amount of acetate by-product and increasing the production (e.g., in terms of titer, rate, and/or yield) of all acetyl-CoA derived products, by recycling acetate by-product and converting it to acetyl-CoA. In addition, the ACS is useful for increasing the production (e.g., titer, rate, and/or yield) of acetyl-CoA products having a biosynthetic pathway that includes one or more CoA thioesters of short-chain carboxylic acids (i.e., short-chain acyl-CoAs) as precursors and/or intermediates. Such CoA thioesters/acyl-CoAs include, for example, CoA thioesters/acyl-CoAs of C2-C8, particularly of C2-C4 or C2-C6, carboxylic acids/carboxylates, such as, for example, acetyl-CoA, acetoacetyl-CoA, butyryl-CoA, succinyl-CoA, hexanoyl-CoA, and 6-aminocaproyl-CoA.

Thus, the ACS, when expressed in strains expressing an acetyl-CoA derived product pathway comprising one or more short-chain acyl-CoA precursors and/or intermediates (which result in the generation of one or more short-chain carboxylate by-products), reduces the production of one or more short-chain carboxylate by-products (e.g., C2-C8, particularly, C2-C4 or C2-C6 by-products), and/or increases the carbon flux through one or more short-chain acyl-CoA precursors and/or intermediates, and/or increases the production (e.g., titer, yield, and/or productivity) of the acetyl-CoA derived product.

TABLE 9
Activity of ACS on Substrates of Various Chain Lengths

| Substrate | Activity (μM AMP/min/μM ACS) |
|---|---|
| Acetate | 38 |
| Acetoacetate | 34 |
| Butyrate | 70 |
| (S)-3HB | 222 |
| (R)-3HB | 322 |
| Succinate | 0.06 |
| Hexanoate | 31 |
| Adipate | No activity |
| 6ACA | 0.015 |
| Octanoate | No activity |

### Example 5

### Expression of Acyl-CoA Synthetase (ACS) Results in Reduced Levels of Acetate

As described in Example 4 and elsewhere herein, the ACS described herein has activity to convert (or recycle) acetate by-product back to acetyl-CoA, which can reduce, eliminate, or substantially eliminate the production of acetate by-product, and/or increase the carbon flux through acetyl-CoA, and/or increase the production of acetyl-CoA derived products. This example demonstrates that a strain expressing a 1,3-BDO pathway and an acyl-CoA synthetase (ACS; also referred to herein as a CoA ligase) (GenBank Accession No. WP_011046428.1; see, SEQ ID NO: 1) exhibits increased production of 1,3-butanediol (1,3-BDO) and reduced production of acetate by-product over time, compared to a control strain that does not express the ACS.

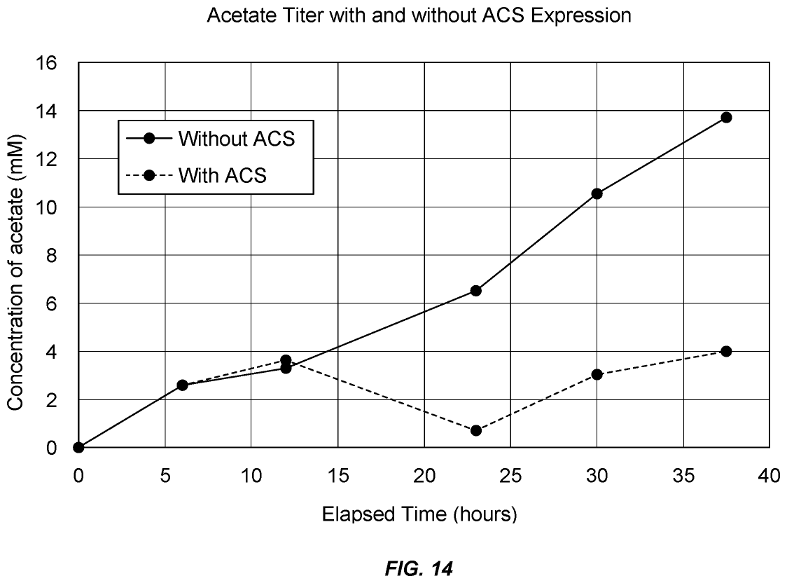

An E. coli control strain, which does not express ACS, and an E. coli strain which expresses ACS, were cultured as described in Example 1, for 37.5 hours, and the production of 1,3-BDO and acetate were measured by HPLC. The results, which are summarized in TABLE 10 below, show that expression of ACS results in an increased titer of 1,3-BDO product, and a reduced titer of acetate by-product, compared to the absence of ACS expression (see, also, FIG. 14).

Since acetyl-CoA is a precursor or intermediate for all acetyl-CoA derived products, expression of ACS, in a strain comprising an acetyl-CoA derived product pathway, can increase the titer, yield, and/or productivity of any acetyl-CoA derived product. For example, a strain expressing ACS and a 1,3-BDO pathway has increased production of 1,3-BDO, and decreased production of acetate and 3HB by-products.

TABLE 10
Titers of 1,3 BDO and Acetate Produced
with and without ACS Expression

| | | | Strain with ACS | |
|---|---|---|---|---|
| | Control Strain (without | | Expression | |
| Elapsed | ACS Expression) | | 1,3-BDO | |
| Time | 1,3-BDO | Acetate Titer | Titer | Acetate Titer |
| (Hours) | Titer (mM) | (mM) | (mM) | (mM) |
| 0 | 0.907 | 0 | 0.788 | 0 |
| 6 | 34.837 | 2.59 | 40.336 | 2.588 |
| 12 | 274.714 | 3.303 | 301.639 | 3.626 |
| 23 | 715.386 | 6.518 | 755.289 | 0.718 |
| 30 | 888.481 | 10.55 | 924.746 | 3.037 |
| 37.5 | 1050.502 | 13.712 | 1083.821 | 4.012 |

What is claimed is:

1. A non-naturally occurring microbial organism having eliminated, substantially eliminated, or decreased production of at least one carboxylate by-product, wherein:
a. the non-naturally occurring microbial organism comprises at least one carboxylate by-product recycling loop and at least one acetyl-CoA derived product pathway;
b. the at least one carboxylate by-product is generated from at least one acyl-CoA precursor or intermediate of the acetyl-CoA derived product pathway; and
c. the carboxylate by-product recycling loop converts the carboxylate by-product back to the acyl-CoA precursor or intermediate of the acetyl-CoA derived product pathway.

2. The non-naturally occurring microbial organism of claim 1, wherein:
a. the carboxylate by-product recycling loop comprises an acyl-CoA synthetase (ACS) or a homolog thereof, or comprises at least one exogenous nucleic acid encoding an acyl-CoA synthetase (ACS) or homolog thereof, wherein the ACS or homolog thereof has activity to convert the carboxylate by-product back to the acyl-CoA precursor or intermediate of the acetyl-CoA derived product pathway.

3. The non-naturally occurring microbial organism of claim 2, wherein the ACS or homolog thereof has activity corresponding to EC 6.2.1.-, EC 6.2.1.1, and/or EC 6.2.2.2.

4. The non-naturally occurring microbial organism of claim 2 or claim 3, wherein the ACS or homolog thereof is heterologous to the microbial organism and is expressed or overexpressed in the microbial organism, or wherein the ACS or homolog thereof is native to the microbial organism and is overexpressed in the microbial organism.

5. The non-naturally occurring microbial organism of any one of claims 2-4, wherein the ACS comprises the amino acid sequence set forth in SEQ ID NO: 1, or is a variant or homolog of an ACS comprising the amino acid sequence set forth in SEQ ID NO: 1, or is a variant or homolog comprising at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%, sequence identity to SEQ ID NO: 1.

6. The non-naturally occurring microbial organism of any one of claims 2-5, wherein the enzymatic reaction catalyzed by the ACS or the homolog thereof results in the formation of AMP.

7. The non-naturally occurring microbial organism of any one of claims 2-6, wherein the ACS or homolog thereof has activity to convert a short-chain carboxylate to an acyl-CoA precursor or intermediate of the acetyl-CoA derived product pathway.

8. The non-naturally occurring microbial organism of any one of claims 1-7, wherein the at least one carboxylate by-product is a short-chain carboxylate.

9. The non-naturally occurring microbial organism of claim 7 or claim 8, wherein the short-chain carboxylate is a C2-C4, a C2-C6, or a C2-C8 carboxylate.

10. The non-naturally occurring microbial organism of any one of claims 1-9, wherein the non-naturally occurring microbial organism has eliminated, substantially eliminated, or decreased production of at least one, at least, two, at least three, at least four, at least five, at least six, at least seven, at least eight, at least nine, or at least 10 carboxylate by-products.

11. The non-naturally occurring microbial organism of any one of claims 1-10, wherein the carboxylate by-product is one or more of acetate, acetoacetate, succinate, crotonate, butyrate, isobutyrate, 3-hydroxybutryate (3HB), (S)-3HB, (R)-3HB, malonate, and/or hexanoate.

12. The non-naturally occurring microbial organism of any one of claims 1-11, wherein the carboxylate by-product is 3-hydroxybutyrate (3HB) or (R)-3HB.

13. The non-naturally occurring microbial organism of any one of claims 1-11, wherein the carboxylate by-products are (i) 3HB or (R)-3HB; and (ii) acetate.

14. The non-naturally occurring microbial organism of any one of claims 1-13, wherein the non-naturally occurring microbial organism has increased or enhanced carbon flux through at least one acyl-CoA precursor or intermediate.

15. The non-naturally occurring microbial organism of any one of claims 1-14, wherein the acyl-CoA precursor or intermediate is one or more of acetyl-CoA, acetoacetyl-CoA, succinyl-CoA, crotonyl-CoA, butyryl-CoA, isobutyryl-CoA, 3HB—CoA, (S)-3HB—CoA, (R)-3HB—CoA, malonyl-CoA, and/or hexanoyl-CoA.

16. The non-naturally occurring microbial organism of claim 14, wherein the non-naturally occurring microbial organism has increased or enhanced carbon flux through (i) 3HB—CoA or (R)-3HB—CoA; or (ii) acetyl-CoA; or (iii) both (i) and (ii).

17. The non-naturally occurring microbial organism of any one of claims 1-16, wherein the at least one carboxylate by-product recycling loop is any one or more of a 3HB recycling loop, an (R)-3HB recycling loop, an (S)-3HB recycling loop, an acetate recycling loop, an acetoacetate recycling loop, a succinate recycling loop, a crotonate recycling loop, a butyrate recycling loop, an isobutyrate recycling loop, a malonate recycling loop, and/or a hexanoate recycling loop.

18. The non-naturally occurring microbial organism of any one of claims 1-17, wherein the non-naturally occurring microbial organism has increased production of, or produces an increased amount, titer, and/or yield of, at least one acetyl-CoA derived product.

19. The non-naturally occurring microbial organism of any one of claims 1-18, wherein the acetyl-CoA derived product is one or more of 1,3-butanediol (1,3-BDO), (3R)-hydroxybutyl (3R)-hydroxybutyrate, methacrylic acid (MAA), MAA ester, butyrate, butanol, propane, formate, hexamethylenediamine (HMD), caprolactam (CPL), adipate (adipic acid), 6-aminocaproic acid (6-ACA), and/or 1,6-hexanediol (HDO).

20. The non-naturally occurring microbial organism of any one of claims 1-19, further comprising a 3HB to 3-hydroxybutyraldehyde (3HBAld) conversion step, and/or an (R)-3HB to (R)-3HBAld conversion step.

21. The non-naturally occurring microbial organism of claim 20, wherein the 3HB to 3HBAld conversion step or the (R)-3HB to (R)-3HBAld conversion step comprises a carboxylic acid reductase (CAR), or a variant or a homolog thereof, or comprises at least one exogenous nucleic acid encoding a CAR or a variant or a homolog thereof.

22. The non-naturally occurring microbial organism of claim 21, wherein the CAR or variant or homolog thereof has activity characterized by EC 1.2.1.- and/or EC 1.2.1.30.

23. The non-naturally occurring microbial organism of claim 21 or claim 22, wherein the CAR or variant or homolog thereof has activity to convert 3HB to 3HBAld and/or has activity to convert (R)-3HB to (R)-3HBAld.

24. The non-naturally occurring microbial organism of any one of claims 21-23, wherein the CAR or variant or homolog thereof is heterologous to the microbial organism and is expressed or overexpressed in the microbial organism, or wherein the CAR or variant or homolog thereof is native to the microbial organism and is overexpressed in the microbial organism.

25. The non-naturally occurring microbial organism of any one of claims 21-24, wherein the CAR comprises the amino acid sequence set forth in SEQ ID NO: 2, or SEQ ID NO: 3, or is a variant or homolog thereof, or wherein the CAR is a variant or homolog comprising at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%, sequence identity to SEQ ID NO: 2 or SEQ ID NO: 3.

26. The non-naturally occurring microbial organism of any one of claims 20-25, wherein the 3HB to 3HBAld conversion step or the (R)-3HB to (R)-3HBAld conversion step reduces the amount, production, titer, yield, and/or productivity of 3HB or (R)-3HB by-product; and/or increases or enhances the carbon flux through 3HBAld or (R)-3HBAld, and/or increases the amount, production, titer, yield, and/or productivity of an acetyl-CoA derived product that is 1,3-BDO or (R)-1,3-BDO.

27. The non-naturally occurring microbial organism of any one of claims 1-26, wherein the non-naturally occurring microbial organism has eliminated, substantially eliminated, or decreased production of at least one carboxylate by-product, and/or has increased production, titer, yield and/or productivity of at least one acetyl-CoA derived product, and/or has increased or enhanced carbon flux through an acyl-CoA precursor or intermediate, compared to a microbial organism that lacks a carboxylate by-product recycling loop.

28. The non-naturally occurring microbial organism of any one of claims 2-26, wherein the non-naturally occurring microbial organism has eliminated, substantially eliminated, or decreased production of at least one carboxylate by-product, and/or has increased production, titer, yield and/or productivity of at least one acetyl-CoA derived product, and/or has increased or enhanced carbon flux through an acyl-CoA precursor or intermediate, compared to a microbial organism that does not express an ACS or a homolog or variant thereof and/or compared to a microbial organism with wild-type expression of an ACS or a homolog or a variant thereof.

29. The non-naturally occurring microbial organism of any one of claims 20-28, wherein the non-naturally occurring microbial organism has eliminated, substantially eliminated, or decreased production of 3HB and/or (R)-3HB, and/or has increased or enhanced carbon flux through 3HBAld and/or (R)-3HBAld, and/or has increased production, titer, yield and/or productivity of at least one acetyl-CoA derived product having a 3HB—CoA, (R)-3HB—CoA, 3HBAld, and/or (R)-3HBAld intermediate or precursor, compared to a microbial organism that does not comprise a 3HB to 3HBAld conversion step or a (R)-3HB to (R)-3HBAld conversion step, and/or compared to a microbial organism that does not express a CAR or a variant or homolog thereof, and/or compared to a microbial organism with wild-type expression of a CAR or a variant or a homolog thereof.

30. The non-naturally occurring microbial organism of any one of claims 27-29 wherein:
a. the non-naturally occurring microbial organism has eliminated, substantially eliminated, or decreased production of 3HB, (R)-3HB, and/or acetate by-products;
b. the non-naturally occurring microbial organism has increased or enhanced carbon flux through 3HB—CoA, (R)-3HB—CoA, acetyl-CoA, 3HBAld, and/or (R)-3HBAld; and
c. the non-naturally occurring microbial organism has increased production, titer, yield and/or productivity of 1,3-BDO and/or (R)-1,3-BDO.

31. The non-naturally occurring microbial organism of claim 1, wherein:
a. the at least one carboxylate by-product recycling loop is any one or more of a 3HB recycling loop, an (R)-3HB recycling loop, an (S)-3HB recycling loop, an acetate recycling loop, an acetoacetate recycling loop, a succinate recycling loop, a crotonate recycling loop, a butyrate recycling loop, an isobutyrate recycling loop, a malonate recycling loop, and/or a hexanoate recycling loop;
b. the at least one acetyl-CoA derived product pathway is one or more of a 1,3-butanediol (1,3-BDO), (R)-1,3-BDO, (3R)-hydroxybutyl (3R)-hydroxybutyrate, methacrylic acid (MAA), MAA ester, butyrate, butanol, propane, formate, hexamethylenediamine (HMD), caprolactam (CPL), adipate (adipic acid), 6-aminocaproic acid (6-ACA), and/or 1,6-hexanediol (HDO) product pathway;
c. the at least one carboxylate by-product is one or more of acetate, acetoacetate, succinate, crotonate, butyrate, isobutyrate, 3-hydroxybutryate (3HB), (S)-3HB, (R)-3HB, malonate, and/or hexanoate; and
d. the at least one acyl-CoA precursor or intermediate is one or more of acetyl-CoA, acetoacetyl-CoA, succinyl-CoA, crotonyl-CoA, butyryl-CoA, isobutyryl-CoA, (S)-3HB—CoA, (R)-3HB—CoA, malonyl-CoA, and/or hexanoyl-CoA.

32. The non-naturally occurring microbial organism of claim 31, comprising an (R)-3HB recycling loop and an (R)-1,3-BDO product pathway, wherein the microbial organism has eliminated, substantially eliminated, or decreased production of (R)-3HB by-product.

33. The non-naturally occurring microbial organism of claim 32, wherein the (R)-3HB recycling loop comprises an acyl-CoA synthetase (ACS) or a homolog thereof, or comprises at least one exogenous nucleic acid encoding an acyl-CoA synthetase (ACS) or homolog thereof, wherein
a. the ACS or homolog thereof comprises activity to convert (R)-3HB to (R)-3HB—CoA; and
b. the ACS or homolog thereof is heterologous to the non-naturally occurring microbial organism and is expressed or overexpressed, or is native to the non-naturally occurring microbial organism and is overexpressed.

34. The non-naturally occurring microbial organism of claim 33, wherein the microbial organism has enhanced or increased carbon flux through (R)-3HB—CoA, and/or has increased production, titer, yield, and/or productivity of (R)-1,3-BDO.

35. The non-naturally occurring microbial organism of claim 34, wherein the microbial organism has enhanced or increased carbon flux through (R)-3HB—CoA, and/or increased production, titer, yield, and/or productivity of (R)-1,3-BDO, compared to a microbial organism without an (R)-3HB recycling loop and/or compared to a microbial organism without expression of an ACS or homolog thereof, and/or compared to a microbial organism with wild-type expression of an ACS or homolog thereof.

36. The non-naturally occurring microbial organism of claim 35, wherein:
a. the microbial organism additionally comprises an acetate recycling loop, wherein the microbial organism has eliminated, substantially eliminated, or decreased production of acetate by-product and/or has increased or enhanced carbon flux through acetyl-CoA; and/or
b. the microbial organism additionally comprises an (R)-3HB to (R)-3HBAld conversion step, wherein the microbial organism optionally has eliminated, substantially eliminated, or decreased production of (R)-3HB by-product and/or increased or enhanced carbon flux through (R)-3HBAld.

37. The non-naturally occurring microbial organism of claim 28, wherein the (R)-3HB to (R)-3HBAld conversion step comprises a carboxylic acid reductase (CAR), or a variant or a homolog thereof, or comprises at least one exogenous nucleic acid encoding a CAR or a variant or a homolog thereof, and wherein the CAR or variant or homolog thereof is heterologous to the non-naturally occurring microbial organism and is expressed or overexpressed, or the CAR or variant or homolog thereof is native to the non-naturally occurring microbial organism and is overexpressed.

38. A non-naturally occurring microbial organism having eliminated, substantially eliminated, or decreased 3-hydroxybutyrate (3HB) by-product, wherein the non-naturally occurring microbial organism comprises (i) an acetyl Coenzyme A (acetyl-CoA) derived product pathway; (ii) an enhanced or increased carbon flux through 3-hydroxybutyryl Coenzyme-A (3HB—CoA); and (iii) a 3HB recycling loop.

39. The non-naturally occurring microbial organism of claim 38, wherein the 3HB recycling loop comprises at least one exogenous nucleic acid encoding an acyl-CoA synthetase (ACS) or homolog thereof, or comprises an acyl-CoA synthetase or homolog thereof.

40. The non-naturally occurring microbial organism of claim 39, wherein said ACS or homolog thereof is expressed in a sufficient amount to reduce production of 3HB.

41. The non-naturally occurring microbial organism of claim 39 or claim 40, wherein said ACS or homolog thereof is expressed in a sufficient amount to increase the yield of the acetyl-CoA derived product.

42. The non-naturally occurring microbial organism of any one of claims 39-41, wherein the at least one exogenous nucleic acid is a heterologous nucleic acid; or the ACS is heterologous to the non-naturally occurring microbial organism and is expressed or overexpressed; or the ACS is native to the non-naturally occurring microbial organism and is overexpressed.

43. The non-naturally occurring microbial organism of any one of claims 38 to 42, wherein the non-naturally occurring microbial organism has reduced concentration of intracellular 3HB.

44. The non-naturally occurring microbial organism of any one of claims 38 to 43, wherein the non-naturally occurring microbial organism has reduced concentration of intracellular 3HB compared to a microbial organism without a 3HB recycling loop.

45. The non-naturally occurring microbial organism of any one of claims 39 to 43, wherein the non-naturally occurring microbial organism has reduced concentration of intracellular 3HB compared to a microbial organism with wild-type expression of ACS.

46. The non-naturally occurring microbial organism of any one of claims 38 to 45, wherein the non-naturally occurring microbial organism has increased yield of the acetyl-CoA derived product.

47. The non-naturally occurring microbial organism of claim 46, wherein the non-naturally occurring microbial organism has increased yield of the acetyl-CoA derived product compared to a microbial organism without a 3HB recycling loop.

48. The non-naturally occurring microbial organism of any one of claims 39 to 47, wherein the non-naturally occurring microbial organism has increased yield of the acetyl-CoA derived product compared to a microbial organism with wild-type expression of ACS.

49. The non-naturally occurring microbial organism of any one of claims 38-48, further comprising one or more of an acetate recycling loop, an acetoacetate recycling loop, a succinate recycling loop, a crotonate recycling loop, a butyrate recycling loop, an isobutyrate recycling loop, a malonate recycling loop, and/or a hexanoate recycling loop.

50. The non-naturally occurring microbial organism or any one of claims 38-48, further comprising an acetate recycling loop, wherein:
a. the acetate recycling loop comprises at least one exogenous nucleic acid encoding an acyl-CoA synthetase (ACS) or homolog thereof, or comprises an acyl-CoA synthetase or homolog thereof;
b. the ACS or homolog thereof catalyzes the conversion of acetate by-product to acetyl-CoA;
c. the non-naturally occurring microbial organism has reduced concentration of intracellular acetate compared to a microbial organism without an acetate recycling loop;
d. the non-naturally occurring microbial organism has enhanced or increased carbon flux through acetyl-CoA; and/or
e. the non-naturally occurring microbial organism has increased production of an acetyl-CoA derived product.

51. The non-naturally occurring microbial organism of any one of claims 38-50, wherein the non-naturally occurring microbial organism further comprises a 3HB to 3HBAld conversion step or an R-3HB to R-3HAld conversion step.

52. The non-naturally occurring microbial organism of claim 51, wherein the 3HB to 3HBAld conversion step or the R-3HB to R-3HAld conversion step comprises an exogenous nucleic acid encoding a CAR or a variant or a homolog thereof, or comprises a CAR or a variant or a homolog thereof, and wherein the CAR or variant or homolog thereof is heterologous to the non-naturally occurring microbial organism and is expressed or overexpressed, or the CAR or variant or homolog thereof is native to the non-naturally occurring microbial organism and is overexpressed.

53. The non-naturally occurring microbial organism of claim 51 or claim 52, wherein the non-naturally occurring microbial organism produces reduced amounts of 3HB or R-3HB, and/or has increased or enhanced carbon flux through 3HBAld or R-3HBAld, and/or has increased production, titer, yield, and/or productivity of an acetyl-CoA derived product, compared to a microbial organism without a 3HB to 3HBAld or R-3HB to R-3HBAld conversion step.

54. The non-naturally occurring microbial organism of any one of claims 38 to 53 wherein the acetyl-CoA derived product is 1,3-BDO, optionally an R-1,3 BDO, (3R)-hydroxybutyl (3R)-hydroxybutyrate, butyrate, butanol, propane, formate, isopropanol, MAA, an MAA ester, HMD, CPL, adipate, 6ACA, or HDO.

55. The non-naturally occurring microbial organism of any one of claims 38 to 54, further comprising a 1,3-BDO, optionally an R-1,3 BDO, an (3R)-hydroxybutyl (3R)-hydroxybutyrate, a butyrate, a butanol, a propane, a formate, an isopropanol, an MAA, an MAA ester, an HMD, a CPL, an adipate, a 6ACA, or an HDO pathway.

56. The non-naturally occurring microbial organism of claim 55, wherein said microbial organism comprises a 1,3-BDO, optionally an R-1,3 BDO, pathway.

57. The non-naturally occurring microbial organism of claim 56, wherein said 1,3-BDO pathway comprises:
a. AcAc—COA reductase (a CoA-dependent, aldehyde forming oxidoreductase); 3-oxobutyraldehyde reductase (a ketone reducing/alcohol forming oxidoreductase); 3HB-Ald reductase (an aldehyde reducing/alcohol forming oxidoreductase), acetaldehyde-alcohol dehydrogenase, 1,3-BDO or 1,3-butylene glycol dehydrogenase, or ADH; AcAc—COA reductase (a CoA-dependent, alcohol forming oxidoreductase); 3-oxobutyraldehyde reductase (an aldehyde reducing/alcohol forming oxidoreductase); 4-hydroxy-2-butanone reductase (a ketone reducing/alcohol forming oxidoreductase); AcAc—COA reductase (ketone reducing/alcohol forming oxidoreductase); 3HB—CoA reductase (a CoA-dependent, aldehyde forming oxidoreductase), ALD, or acetaldehyde-alcohol dehydrogenase; 3HB—CoA reductase (an alcohol forming, CoA dependent oxidoreductase); 3HB—CoA transferase or a 3HB—CoA hydrolase; 3HB dehydrogenase (a ketone reducing, alcohol forming oxidoreductase); AcAc—COA transferase, AcAc—COA hydrolase, AcAc—COA synthetase; acetoacetate reductase (an aldehyde forming oxidoreductase); and a Ac—CoA thiolase;
b. AcAc—COA thiolase; HBD, optionally a R—HBD, or AcAc—COA reductase (ketone reducing/alcohol forming oxidoreductase); butyraldehyde dehydrogenase or acetaldehyde-alcohol dehydrogenase; acetaldehyde-alcohol dehydrogenase; PTB; and BUK;
c. AcAc—COA thiolase; HBD, optionally R—HBD; (R)-3HB—CoA reductase; (R)-3HB—Ald reductase; Ac—COA carboxylase; AcAc—COA synthase; HBD, optionally S—HBD; and 3HB—CoA epimerase;
d. 4HB—CoA dehydratase (a hydro-lyase); CRT (a hydro-lyase); 3HB—CoA reductase (a Co-A dependent, aldehyde forming oxidoreductase); 3HB—Ald reductase (an alcohol forming oxidoreductase); reductase (an alcohol forming oxidoreductase); and/or
e. a SucCoA transferase, SucCoA hydrolase, and/or a SucCoA synthetase (or SucCoA ligase); a SucCoA reductase (aldehyde forming); a 4HB dehydrogenase; a 4HB kinase; a phosphotrans-4-hydroxybutyrylase; a Succ reductase; a SucCoA reductase (alcohol forming); a 4HB—CoA transferase, or a 4HB—CoA synthetase, or a 4HB—CoA ligase; an AKG decarboxylase; a 4HB—CoA dehydratase; a CRT; a 3HB—CoA reductase (aldehyde forming); a 3-hydroxybutanal reductase; a 3HB—CoA reductase (alcohol forming); a 3HB—CoA hydrolase and/or a 3HB—CoA transferase; an alcohol forming reductase; a glutamate dehydrogenase and/or a glutamate transaminase; a glutamate decarboxylase; a 4-aminobutyrate dehydrogenase and/or a 4-aminobutyrate transaminase; and an AKGD.

58. The non-naturally occurring microbial organism of claim 55, wherein said microbial organism comprises an (3R)-hydroxybutyl (3R)-hydroxybutyrate pathway.

59. The non-naturally occurring microbial organism of claim 58, wherein said (3R)-hydroxybutyl (3R)-hydroxybutyrate pathway comprises:
a. AcAc—CoA thiolase; HBD, optionally R—HBD; (R)-3HB—CoA reductase; (R)-3HB—Ald reductase; Ac—CoA carboxylase; AcAc—COA synthase; HBD, optionally S—HBD; 3HB—CoA epimerase; and (3R)-hydroxybutyryl-CoA:(R)-1,3-butanediol alcohol transferase; and/or
b. AcAc—COA thiolase; HBD, optionally a R—HBD, or AcAc—CoA reductase (ketone reducing/alcohol forming oxidoreductase); butyraldehyde dehydrogenase or acetaldehyde-alcohol dehydrogenase; acetaldehyde-alcohol dehydrogenase; PTB; BUK; and (3R)-hydroxybutyryl-CoA:(R)-1,3-butanediol alcohol transferase.

60. The non-naturally occurring microbial organism of claim 55, wherein said microbial organism comprises a butyrate, butanol, propane, and/or formate pathway.

61. The non-naturally occurring microbial organism of claim 60, wherein said butyrate, butanol, propane, and/or formate pathway comprises: AcAc—COA thiolase; HBD, optionally, S—HBD; CRT; BCD; ALDH; AHR; PTB; BUK; and ADO.

62. The non-naturally occurring microbial organism of claim 55, wherein said microbial organism comprises an isopropanol pathway.

63. The non-naturally occurring microbial organism of claim 62, wherein said isopropanol pathway comprises: CRT; HBD; acetoacetyl-CoA synthetase, acetyl-CoA: acetoacetate-CoA transferase, and/or acetoacetyl-CoA hydrolase; acetoacetate decarboxylase; and acetone reductase.

64. The non-naturally occurring microbial organism of claim 55, wherein said microbial organism comprises an MAA pathway.

65. The non-naturally occurring microbial organism of claim 64, wherein said MAA pathway comprises:
a. AcAc—COA thiolase; AcAc—COA reductase (ketone reducing/alcohol forming oxidoreductase); 3HB—CoA mutase; 2HIB—CoA dehydratase; methacrylyl-CoA transferase, methacrylyl-CoA hydrolase and/or methacrylyl-CoA synthetase; 4HB—CoA dehydratase; vinylacetyl-CoA A-isomerase; and/CRT; and/or
b. AcAc—COA thiolase; AcAc—COA reductase (ketone reducing/alcohol forming oxidoreductase); methacrylyl-CoA transferase, methacrylyl-CoA hydrolase and/or methacrylyl-CoA synthetase; CRT; butyryl-CoA dehydrogenase; isobutyryl-CoA mutase; and isobutyryl-CoA dehydrogenase.

66. The non-naturally occurring microbial organism of claim 55, wherein said microbial organism comprises a MAA ester pathway.

67. The non-naturally occurring microbial organism of claim 66, wherein said MAA ester pathway comprises:
a. a methacrylyl-CoA transferase or a methacrylyl-CoA synthetase; and an alcohol transferase; and/or
b. or a methacrylate ester-forming enzyme.

68. A non-naturally occurring microbial organism having eliminated, substantially eliminated, or decreased 3-hydroxybutyrate (3HB) by-product, wherein the non-naturally occurring microbial organism comprises (i) at least one an acetyl Coenzyme A (acetyl-CoA) derived product pathway; (ii) an enhanced or increased carbon flux through 3-hydroxybutyraldehyde (3HBAld); and (iii) a 3HB to 3HBAld conversion step.

69. The non-naturally occurring microbial organism of claim 68, wherein the 3HB to 3HBAld conversion step comprises an exogenous nucleic acid encoding a CAR or a variant or a homolog thereof, or comprises a CAR or a variant or a homolog thereof.

70. The non-naturally occurring microbial organism of claim 69, wherein the CAR or variant or homolog thereof has activity characterized by EC 1.2.1.- and/or EC 1.2.1.30.

71. The non-naturally occurring microbial organism of claim 69 or claim 70, wherein the CAR or variant or homolog thereof has activity to convert 3HB to 3HBAld and/or has activity to convert (R)-3HB to (R)-3HBAld.

72. The non-naturally occurring microbial organism of any one of claims 69-71, wherein the CAR or variant or homolog thereof is heterologous to the microbial organism and is expressed or overexpressed in the microbial organism, or wherein the CAR or variant or homolog thereof is native to the microbial organism and is overexpressed in the microbial organism.

73. The non-naturally occurring microbial organism of any one of claims 69-72, wherein the CAR comprises the amino acid sequence set forth in SEQ ID NO: 2 or SEQ ID NO: 3, or is a variant or homolog thereof, or wherein the CAR is a variant or homolog comprising at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%, sequence identity to SEQ ID NO: 2 or SEQ ID NO: 3.

74. The non-naturally occurring microbial organism of any one of claims 68-73, wherein the 3HB to 3HBAld conversion step reduces the amount, production, titer, yield, and/or productivity of 3HB by-product; and/or increases or enhances the carbon flux through 3HBAld; and/or increases the amount, production, titer, yield, and/or productivity of an acetyl-CoA derived product.

75. The non-naturally occurring microbial organism of claim 74, wherein the acetyl-CoA derived product is 1,3-BDO.

76. The non-naturally occurring microbial organism of any one of claims 68-74, wherein the non-naturally occurring microbial organism has eliminated, substantially eliminated, or decreased production of 3HB by-product, and/or has increased production, titer, yield and/or productivity of at least one acetyl-CoA derived product, and/or has increased or enhanced carbon flux through 3HBAld, compared to a microbial organism that lacks a 3HB to 3HB Ald conversion step.

77. The non-naturally occurring microbial organism of any one of claims 69-74, wherein the non-naturally occurring microbial organism has eliminated, substantially eliminated, or decreased production of 3HB, and/or has increased production, titer, yield and/or productivity of at least one acetyl-CoA derived product, and/or has increased or enhanced carbon flux through 3HBAld, compared to a microbial organism that does not express a CAR or a homolog or variant thereof, and/or compared to a microbial organism with wild-type expression of a CAR or a homolog or a variant thereof.

78. The non-naturally occurring microbial organism of any one of claims 68-77, wherein the at least one acetyl-CoA derived product pathway comprises a 3HBAld intermediate or precursor.

79. The non-naturally occurring microbial organism of any one of claims 68-78 wherein the 3HB is R-3HB and the 3HBAld is R-3HBAld.

80. The non-naturally occurring microbial organism of any one of claims 68-77, wherein the at least one acetyl-CoA derived product pathway is one or more of a 1,3-BDO, an R-1,3 BDO, an (3R)-hydroxybutyl (3R)-hydroxybutyrate, a butyrate, a butanol, a propane, a formate, an isopropanol, an MAA, or an MAA ester product pathway.

81. The non-naturally occurring microbial organism of claim 80, wherein the acetyl-CoA derived product pathway is a 1,3-BDO, optionally an R-1,3-BDO, product pathway.

82. The non-naturally occurring microbial organism of claim 81, wherein the 1,3-BDO or R-1,3-BDO product pathway comprises:
a. AcAc—COA reductase (a CoA-dependent, aldehyde forming oxidoreductase); 3-oxobutyraldehyde reductase (a ketone reducing/alcohol forming oxidoreductase); 3HB-Ald reductase (an aldehyde reducing/alcohol forming oxidoreductase), acetaldehyde-alcohol dehydrogenase, 1,3-BDO or 1,3-butylene glycol dehydrogenase, or ADH; AcAc—COA reductase (a CoA-dependent, alcohol forming oxidoreductase); 3-oxobutyraldehyde reductase (an aldehyde reducing/alcohol forming oxidoreductase); 4-hydroxy-2-butanone reductase (a ketone reducing/alcohol forming oxidoreductase); AcAc—COA reductase (ketone reducing/alcohol forming oxidoreductase); 3HB—CoA reductase (a CoA-dependent, aldehyde forming oxidoreductase), ALD, or acetaldehyde-alcohol dehydrogenase; 3HB—CoA reductase (an alcohol forming, CoA dependent oxidoreductase); 3HB—CoA transferase or a 3HB—CoA hydrolase; 3HB dehydrogenase (a ketone reducing, alcohol forming oxidoreductase); AcAc—COA transferase, AcAc—COA hydrolase, AcAc—COA synthetase; acetoacetate reductase (an aldehyde forming oxidoreductase); and a Ac—COA thiolase;
b. AcAc—COA thiolase; HBD, optionally a R—HBD, or AcAc—COA reductase (ketone reducing/alcohol forming oxidoreductase); butyraldehyde dehydrogenase or acetaldehyde-alcohol dehydrogenase; acetaldehyde-alcohol dehydrogenase; PTB; and BUK;
c. AcAc—CoA thiolase; HBD, optionally R—HBD; (R)-3HB—CoA reductase; (R)-3HB—Ald reductase; Ac—COA carboxylase; AcAc—COA synthase; HBD, optionally S—HBD; and 3HB—CoA epimerase;
d. 4HB—CoA dehydratase (a hydro-lyase); CRT (a hydro-lyase); 3HB—CoA reductase (a Co-A dependent, aldehyde forming oxidoreductase); 3HB—Ald reductase (an alcohol forming oxidoreductase); and 3HB—CoA reductase (an alcohol 1 forming oxidoreductase); and/or
e. a SucCoA transferase, SucCoA hydrolase, and/or a SucCoA synthetase (or SucCoA ligase); a SucCoA reductase (aldehyde forming); a 4HB dehydrogenase; a 4HB kinase; a phosphotrans-4-hydroxybutyrylase; a Succ reductase; a SucCoA reductase (alcohol forming); a 4HB—CoA transferase, or a 4HB—CoA synthetase, or a 4HB—CoA ligase; an AKG decarboxylase; a 4HB—CoA dehydratase; a CRT; a 3HB—CoA reductase (aldehyde forming); a 3-hydroxybutanal reductase; a 3HB—CoA reductase (alcohol forming); a 3HB—CoA hydrolase and/or a 3HB—CoA transferase; an alcohol forming reductase; a glutamate dehydrogenase and/or a glutamate transaminase; a glutamate decarboxylase; a 4-aminobutyrate dehydrogenase and/or a 4-aminobutyrate transaminase; and an AKGD.

83. The non-naturally occurring microbial organism of any one of claims 1 to 82, wherein the non-naturally occurring microbial organism is a species of bacteria, yeast, or fungus.

84. The non-naturally occurring microbial organism of any one of claims 1 to 83, wherein said non-naturally occurring microbial organism is in a substantially anaerobic culture medium, or is in a microaerobic or substantially microaerobic culture medium.

85. A method for increasing the production of 1,3-BDO, the method comprising culturing the non-naturally occurring microbial organism of any one of claims 1-84, under conditions and for a sufficient period of time to increase the availability or production or titer or yield of 1,3-BDO.

86. The method of claim 85, wherein the 1,3-BDO is (R)-1,3-BDO.

87. A method for increasing the production of 3R-hydroxybutyric acid-3R-hydroxybutryrate, the method comprising culturing the non-naturally occurring microbial organism of any one of claims 1-84, under conditions and for a sufficient period of time to increase the availability or production or titer or yield of 3R-hydroxybutyric acid-3R-hydroxybutryrate.

88. A method for increasing the production of butyrate, butanol, propane, and/or formate, the method comprising culturing the non-naturally occurring microbial organism of any one of claims 1-84, under conditions and for a sufficient period of time to increase the availability of butyrate, butanol, propane, and/or formate.

89. A method for increasing the production of isopropanol, the method comprising culturing the non-naturally occurring microbial organism of any one of claims 1-84, under conditions and for a sufficient period of time to increase the availability or production or titer or yield of isopropanol.

90. A method for increasing the production of MAA, the method comprising culturing the non-naturally occurring microbial organism of any one of claims 1-84, under conditions and for a sufficient period of time to increase the availability or production or titer or yield of MAA.

91. A method for increasing the production of an MAA ester, the method comprising culturing the non-naturally occurring microbial organism of any one of claims 1-84, under conditions and for a sufficient period of time to increase the availability or production or titer or yield of an MAA ester.

92. A method for enhancing or increasing the carbon flux through 3HB—CoA in a non-naturally occurring microbial organism to increase the production, titer and/or yield of an acetyl-CoA derived product and to eliminate, substantially eliminate, or reduce the production of 3HB by-product, the method comprising culturing the non-naturally occurring microbial organism of any one of claims 1 to 84 under conditions and for a sufficient period of time to produce the acetyl-CoA derived product.

93. The method of claim 92, wherein the acetyl-CoA derived product is 1,3-BDO, R-1,3 BDO, S-1,3-BDO, (3R)-hydroxybutyl (3R)-hydroxybutyrate, butyrate, butanol, propane, formate, MAA, MAA ester, HMD, CPL, adipate, 6ACA, or HDO.

94. The method of claim 93, wherein the acetyl-CoA derived product comprises 1,3-BDO, optionally R-1,3-BDO.

95. The method of claim 93, wherein the acetyl-CoA derived product comprises 3R-hydroxybutyric acid-3R-hydroxybutryrate.

96. The method of claim 93, wherein the acetyl-CoA derived product comprises butyrate, butanol, propane, and/or formate.

97. The method of claim 93, wherein the acetyl-CoA derived product comprises isopropanol.

98. The method of claim 93, wherein the acetyl-CoA derived product comprises MAA.

99. The method of claim 93, wherein the acetyl-CoA derived product comprises an MAA ester.

100. The method of claim 93, wherein the acetyl-CoA derived product comprises HMD.

101. The method of claim 93, wherein the acetyl-CoA derived product comprises CPL.

102. The method of claim 93, wherein the acetyl-CoA derived product comprises adipate.

103. The method of claim 73, wherein the acetyl-CoA derived product comprises 6ACA.

104. The method of claim 93, wherein the acetyl-CoA derived product comprises HDO.

105. A method for increasing the production, titer, yield, and/or productivity of an acetyl-CoA derived product, the method comprising culturing the non-naturally occurring microbial organism of any one of claims 1-84.

106. A non-naturally occurring microbial organism, comprising:
a. an acetyl Coenzyme A (acetyl-CoA) derived product pathway;
b. a 3HB recycling loop; and
c. a 3HB to 3HBAld conversion step.

107. The non-naturally occurring microbial organism of claim 106, wherein:
a. the acetyl-CoA derived product pathway is one or more of a 1,3-BDO, an R-1,3 BDO, an S-1,3-BDO, a (3R)-hydroxybutyl (3R)-hydroxybutyrate, a butyrate, a butanol, a propane, a formate, an MAA, or an MAA ester product pathway;
b. the 3HB recycling loop comprises at least one exogenous nucleic acid encoding an acyl-CoA synthetase (ACS) or homolog thereof, or comprises an acyl-CoA synthetase or homolog thereof; and
c. the 3HB to 3HB Ald conversion step comprises a carboxylic acid reductase (CAR), or a variant or a homolog thereof, or comprises at least one exogenous nucleic acid encoding a CAR or a variant or a homolog thereof.

108. The non-naturally occurring microbial organism of claim 107, wherein:
a. the non-naturally occurring microbial organism has eliminated, substantially eliminated, or decreased 3HB by-product; and/or
b. the non-naturally occurring microbial organism has increased or enhanced carbon flux through 3HB—CoA and/or 3HBAld; and/or
c. the non-naturally occurring microbial organism has increased production, titer, yield, and/or productivity of an acetyl-CoA derived product,
compared to a microbial organism that does not have a 3HB recycling loop and/or a 3HB to 3HB Ald conversion step.

109. The non-naturally occurring microbial organism of any one of claims 106-108, further comprising an acetate recycling loop, wherein the acetate recycling loop comprises at least one exogenous nucleic acid encoding an acyl-CoA synthetase (ACS) or homolog thereof, or comprises an acyl-CoA synthetase or homolog thereof.

110. The non-naturally occurring microbial organism of claim 109, wherein the non-naturally occurring microbial organism further has eliminated, substantially eliminated, or decreased acetate by-product; and/or further has increased carbon flux through acetyl-CoA; and/or further has increased production, titer, yield, and/or productivity of an acetyl-CoA derived product, compared to microbial organism that does not have any one or more of: a 3HB recycling loop, a 3HB to 3HBAld conversion step, and/or an acetate recycling loop.

111. The non-naturally occurring microbial organism of any one of claims 106-110, wherein the acetyl-CoA derived product pathway is a 1,3-BDO product pathway, or an R-1,3-BDO product pathway.

112. The non-naturally occurring microbial organism of claim 111, wherein the 1,3-BDO or R-1,3-BDO product pathway comprises:
a. AcAc—COA reductase (a CoA-dependent, aldehyde forming oxidoreductase); 3-oxobutyraldehyde reductase (a ketone reducing/alcohol forming oxidoreductase); 3HB-Ald reductase (an aldehyde reducing/alcohol forming oxidoreductase), acetaldehyde-alcohol dehydrogenase, 1,3-BDO or 1,3-butylene glycol dehydrogenase, or ADH; AcAc—COA reductase (a CoA-dependent, alcohol forming oxidoreductase); 3-oxobutyraldehyde reductase (an aldehyde reducing/alcohol forming oxidoreductase); 4-hydroxy-2-butanone reductase (a ketone reducing/alcohol forming oxidoreductase); AcAc—COA reductase (ketone reducing/alcohol forming oxidoreductase); 3HB—CoA reductase (a CoA-dependent, aldehyde forming oxidoreductase), ALD, or acetaldehyde-alcohol dehydrogenase; 3HB—CoA reductase (an alcohol forming, CoA dependent oxidoreductase); 3HB—CoA transferase or a 3HB—CoA hydrolase; 3HB dehydrogenase (a ketone reducing, alcohol forming oxidoreductase); AcAc—COA transferase, AcAc—COA hydrolase, AcAc—COA synthetase; acetoacetate reductase (an aldehyde forming oxidoreductase); and a Ac—CoA thiolase;
b. AcAc—COA thiolase; HBD, optionally a R—HBD, or AcAc—COA reductase (ketone reducing/alcohol forming oxidoreductase); butyraldehyde dehydrogenase or acetaldehyde-alcohol dehydrogenase; acetaldehyde-alcohol dehydrogenase; PTB; and BUK;
c. AcAc—CoA thiolase; HBD, optionally R—HBD; (R)-3HB—CoA reductase; (R)-3HB—Ald reductase; Ac—COA carboxylase; AcAc—COA synthase; HBD, optionally S—HBD; and 3HB—CoA epimerase;
d. 4HB—CoA dehydratase (a hydro-lyase); CRT (a hydro-lyase); 3HB—CoA reductase (a Co-A dependent, aldehyde forming oxidoreductase); 3HB—Ald reductase (an alcohol forming oxidoreductase); reductase (an alcohol forming oxidoreductase); and/or
e. a SucCoA transferase, SucCoA hydrolase, and/or a SucCoA synthetase (or SucCoA ligase); a SucCoA reductase (aldehyde forming); a 4HB dehydrogenase; a 4HB kinase; a phosphotrans-4-hydroxybutyrylase; a Succ reductase; a SucCoA reductase (alcohol forming); a 4HB—CoA transferase, or a 4HB—CoA synthetase, or a 4HB—CoA ligase; an AKG decarboxylase; a 4HB—CoA dehydratase; a CRT; a 3HB—CoA reductase (aldehyde forming); a 3-hydroxybutanal reductase; a 3HB—CoA reductase (alcohol forming); a 3HB—CoA hydrolase or a 3HB—CoA transferase; an alcohol forming reductase; a glutamate dehydrogenase and/or a glutamate transaminase; a glutamate decarboxylase; a 4-aminobutyrate dehydrogenase and/or a 4-aminobutyrate transaminase; and an AKGD.

113. The non-naturally occurring microbial organism of claim 110, wherein the expression of the 3HB recycling loop, the 3HB to 3HBAld conversion step, and the acetate recycling loop is additive and/or synergistic and can result in the increased production, titer, yield, and/or productivity of an acetyl-CoA derived product compared to microbial organism that does not have any one or more of: a 3HB recycling loop, a 3HB to 3HBAld conversion step, and an acetate recycling loop.

114. The non-naturally occurring microbial organism of claim 113, wherein the expression of the 3HB recycling loop, the 3HB to 3HBAld conversion step, and the acetate recycling loop is additive and/or synergistic compared to microbial organism that does not have any two or more, or all of: a 3HB recycling loop, a 3HB to 3HBAld conversion step, and an acetate recycling loop.

115. The non-naturally occurring microbial organism of claim 36, wherein:
the 3HB recycling loop and the acetate recycling loop are additive and/or synergistic;
the 3HB recycling loop and the 3HB to 3HBAld conversion step are additive and/or synergistic; and/or
the 3HB recycling loop, the acetate recycling loop, and the 3HB to 3HBAld conversion step are additive and/or synergistic.

116. The non-naturally occurring microbial organism of any one of claims 1-19, wherein the non-naturally occurring microbial organism comprises more than one carboxylate by-product recycling loop, and the more than one carboxylate recycling loops are additive and/or synergistic, and result in a greater reduction of carboxylate by-products, and/or a greater increase in carbon flux through the acyl-CoA intermediate or precursor, and/or an increased production of an acetyl-CoA derived product, compared to a microbial organism comprising one carboxylate by-product recycling loop.

117. The non-naturally occurring microbial organism of claim 20, wherein the at least one carboxylate by-product recycling loop and the 3HB to 3HB Ald conversion step are additive and/or synergistic, and result in a greater reduction of carboxylate by-products, and/or a greater increase in carbon flux through one or more intermediates or precursors of the acetyl-CoA derived product pathway, and/or an increased production of an acetyl-CoA derived product, compared to a microbial organism comprising only the at least one carboxylate by-product recycling loop or only the 3HB to 3HBAld conversion step.

118. The non-naturally occurring microbial organism of claim 49, wherein (i) the 3HB recycling loop, and (ii) the one or more of an acetate recycling loop, an acetoacetate recycling loop, a succinate recycling loop, a crotonate recycling loop, a butyrate recycling loop, an isobutyrate recycling loop, a malonate recycling loop, and/or a hexanoate recycling loop are additive and/or synergistic, and result in a greater reduction of carboxylate by-products, and/or a greater increase in carbon flux through one or more intermediates or precursors of the acetyl-CoA derived product pathway, and/or an increased production of an acetyl-CoA derived product, compared to a microbial organism comprising only (i) or (ii).

119. The non-naturally occurring microbial organism of claim 51, wherein (i) the 3HB recycling loop; (ii) the one or more of an acetate recycling loop, an acetoacetate recycling loop, a succinate recycling loop, a crotonate recycling loop, a butyrate recycling loop, an isobutyrate recycling loop, a malonate recycling loop, and/or a hexanoate recycling loop; and/or (iii) the 3HB to 3HBAld conversion step, are additive and/or synergistic, and result in a greater reduction of carboxylate by-products, and/or a greater increase in carbon flux through one or more intermediates or precursors of the acetyl-CoA derived product pathway, and/or an increased production of an acetyl-CoA derived product, compared to a microbial organism comprising only (i), only (ii), only (iii), or only two of (i), (ii), and (iii).

120. The non-naturally occurring microbial organism of claim 106, wherein the 3HB recycling loop and the 3HB to 3HBAld conversion step are additive and/or synergistic, and result in a greater decrease in 3HB by-product, and/or a greater increase in carbon flux through 3HB—CoA and/or 3HBAld, and/or a greater increase in the production of an acetyl-CoA derived product, compared to a microbial organism that only comprises a 3HB recycling loop or that only comprises a 3HB to 3HBAld conversion step.


SEQUENCE LISTING (3 sequences)

SEQ ID NO: 1 - protein, 542 aa, Ruegeria pomeroyi
MTGSTIYDQHLDRTPANFTPLSPLSYIERTAAVYPDYPSVVYGDRRYTWAETYTRCRRLASALAGRGLGKGDTVSIIAANIPEMYEAHFGVPMAGAVLNAINTRLDAPIIAFILSHAEARVLIVDPEFSEVVRDALAQIDRPDLLVVDIEDPSFAGGAPVGTLSYDALLAEGDPDFDWSLPGDEWDAIALNYTSGTTGNPKGVVYHHRGAALNATSNILTWGMPQHAVYLWTLPMFHCNGWCFPWTMAANAGVSVCLRAVRDEPIYRAFREEKVTHFCGAPIVLNMLANAPDHMKDFDHQIKVMTAGAPPPAAVIEKMEAMGVDVTHVYGLTETYGPSVVCAWKEEWDGRPGAERAALKVRQGVRYVALSGLMVADPETLEPVPADGETMGEIFMQGNIVMKGYLKNPDATDRAFRGGWFASGDLGVMHPDGYIALKDRSKDIIISGGENISSVEVEDVLYKHPAVMEAAVVARPDEKWGETPCAFVELKPGQSVEAADLIAHCRANMAHFKAPKTVVFGELPKTSTGKIQKFLLRERARAL

SEQ ID NO: 2 - protein, 1173 aa, Mycobacterium avium
MSTATHDERLDRRVHELIATDPQFAAAQPDPAITAALEQPGLRLPQIIRTVLDGYADRPALGQRVVEFVTDAKTGRTSAQLLPRFETITYSEVAQRVSALGRALSDDAVHPGDRVCVLGFNSVDYATIDMALGAIGAVSVPLQTSAAISSLQPIVAETEPTLIASSVNQLSDAVQLITGAEQAPTRLVVFDYHPQVDDQREAVQDAAARLSSTGVAVQTLAELLERGKDLPAVAEPPADEDSLALLIYTSGSTGAPKGAMYPQSNVGKMWRRGSKNWFGESAASITLNFMPMSHVMGRSILYGTLGNGGTAYFAARSDLSTLLEDLELVRPTELNFVPRIWETLYGEFQRQVERRLSEAGDAGERRAVEAEVLAEQRQYLLGGRFTFAMTGSAPISPELRNWVESLLEMHLMDGYGSTEAGMVLFDGEIQRPPVIDYKLVDVPDLGYFSTDRPHPRGELLLRTENMFPGYYKRAETTAGVFDEDGYYRTGDVFAEIAPDRLVYVDRRNNVLKLAQGEFVTLAKLEAVFGNSPLIRQIYVYGNSAQPYLLAVVVPTEEALASGDPETLKPKIADSLQQVAKEAGLQSYEVPRDFIIETTPFSLENGLLTGIRKLAWPKLKQHYGERLEQMYADLAAGQANELAELRRNGAQAPVLQTVSRAAGAMLGSAASDLSPDAHFTDLGGDSLSALTFGNLLREIFDVDVPVGVIVSPANDLAAIASYIEAERQGSKRPTFASVHGRDATVVRAADLTLDKFLDAETLAAAPNLPKPATEVRTVLLTGATGFLGRYLALEWLERMDMVDGKVIALVRARSDEEARARLDKTFDSGDPKLLAHYQQLAADHLEVIAGDKGEANLGLGQDVWQRLADTVDVIVDPAALVNHVLPYSELFGPNALGTAELIRLALTSKQKPYTYVSTIGVGDQIEPGKFVENADIRQMSATRAINDSYANGYGNSKWAGEVLLREAHDLCGLPVAVFRCDMILADTTYAGQLNLPDMFTRLMLSLVATGIAPGSFYELDADGNRQRAHYDGLPVEFIAAAISTLGSQITDSDTGFQTYHVMNPYDDGVGLDEYVDWLVDAGYSIERIADYSEWLRRFETSLRALPDRQRQYSLLPLLHNYRTPEKPINGSIAPTDVFRAAVQEAKIGPDKDIPHVSPPVIVKYITDLQLLGLL

SEQ ID NO: 3 - protein, 1173 aa, synthetic construct
MSTATHDERLDRRVHELIATDPQFAAAQPDPAITAALEQPGLRLPQIIRTVLDGYADRPALGQRVVEFVTDAKTGRTSAQLLPRFETITYSEVAQRVSALGRALSDDAVHPGDRVCVLGFNSVDYATIDMALGAIGAVSVPLQTSAAISSLQPIVAETEPTLIASSVNQLSDAVQLITGAEQAPTRLVVFDYHPQVDDQREAVQDAAARLSSTGVAVQTLAELLERGKDLPAVAEPPADEDSLALLIYTSGSTGAPKGAMYPQSNVGKMWRRGSKNWFGESAASITLNFMPMSHVMGRSILYGTLGNGGTAYFAARSDLSTLLEDLELVRPTELNFVPRIWETLYGEFQRQVERRLSEAGDAGERRAVEAEVLAEQRQYLLGGRFTFAMTSSAPISPELRNWVESLLEMHLMDGYGSTEAGMVLFDGEIQRPPVIDYKLVDVPDLGYFSTDRPHPRGELLLRTENMFPGYYKRAETTAGVFDEDGYYRTGDVFAEIAPDRLVYVDRRNNVLKLAQGEFVTLAKLEAVFGNSPLIRQIYVYGNSAQPYLLAVVVPTEEALASGDPETLKPKIADSLQQVAKEAGLQSYEVPRDFIIETTPFSLENGLLTGIRKLAWPKLKQHYGERLEQMYADLAAGQANELAELRRNGAQAPVLQTVSRAAGAMLGSAASDLSPDAHFTDLGGDSLSALTFGNLLREIFDVDVPVGVIVSPANDLAAIASYIEAERQGSKRPTFASVHGRDATVVRAADLTLDKFLDAETLAAAPNLPKPATEVRTVLLTGATGFLGRYLALEWLERMDMVDGKVIALVRARSDEEARARLDKTFDSGDPKLLAHYQQLAADHLEVIAGDKGEANLGLGQDVWQRLADTVDVIVDPAALVNHVLPYSELFGPNALGTAELIRLALTSKQKPYTYVSTIGVGDQIEPGKFVENADIRQMSATRAINDSYANGYGNSKWAGEVLLREAHDLCGLPVAVFRCDMILADTTYAGQLNLPDMFTRLMLSLVATGIAPGSFYELDADGNRQRAHYDGLPVEFIAAAISTLGSQITDSDTGFQTYHVMNPYDDGVGLDEYVDWLVDAGYSIERIADYSEWLRRFETSLRALPDRQRQYSLLPLLHNYRTPEKPINGSIAPTDVFRAAVQEAKIGPDKDIPHVSPPVIVKYITDLQLLGLL